(19) United States
(12) Patent Application Publication
(10) Pub. No.: US 2026/0306460 A1
(43) Pub. Date: Oct. 8, 2026

(54) COMPOSITION FOR REMOVING AND INHIBITING SENESCENT CELLS, AND USES THEREOF

(71) Applicant: LIFECEUTIX CO., LTD., Gyeonggi-do (KR)

(72) Inventors: Tae Kook KIM, Gyeonggi-do (KR); Hyung Ju JANG, Gyeonggi-do (KR); Kang Sik CHOO, Gyeonggi-do (KR)

(21) Appl. No.: 19/479,164

(22) PCT Filed: May 7, 2024
(86) PCT No.: PCT/KR2024/006112

§ 371 Date: Oct. 27, 2025

(30) Foreign Application Priority Data
May 4, 2023 (KR) 10-2023-0058711

(51) Int. Cl. A61K 31/4196; A61K 31/145; A61K 31/197; A61K 31/28; A61K 31/353; A61K 31/365; A61K 31/404; A61K 31/4045; A61K 31/405; A61K 31/416; A61K 31/4174; A61K 31/4184; A61K 31/42; A61K 31/437; A61K 31/438; A61K 31/439; A61K 31/4406; A61K 31/4409; A61K 31/4418; A61K 31/4439; A61K 31/454; A61K 31/4545; A61K 31/472; A61K 31/4725; A61K 31/495; A61K 31/496; A61K 31/497; A61K 31/498; A61K 31/501; A61K 31/502; A61K 31/5025; A61K 31/506; A61K 31/519; A61K 31/52; A61K 31/55; A61K 31/5513; A61K 31/56; A61K 31/58; A61K 31/585; A61K 31/606; A61K 31/675; A61K 31/69; A61K 31/7048; A61K 31/7056; A61K 31/706; A61K 31/737; A61K 38/06; A61K 38/12; A61K 38/15; A61K 39/395; A61K 45/06; A61P 1/00; A61P 1/16; A61P 3/04; A61P 7/02; A61P 9/04; A61P 9/12; A61P 11/00; A61P 13/12; A61P 17/02; A61P 19/02; A61P 19/08; A61P 19/10; A61P 21/00; A61P 25/22; A61P 25/24; A61P 25/28; A61P 27/12; A61P 35/00

(52) U.S. Cl. CPC A61K 31/4196; A61K 31/145; A61K 31/197; A61K 31/28; A61K 31/353; A61K 31/365; A61K 31/404; A61K 31/4045; A61K 31/405; A61K 31/416; A61K 31/4174; A61K 31/4184; A61K 31/42; A61K 31/437; A61K 31/438; A61K 31/439; A61K 31/4406; A61K 31/4409; A61K 31/4418; A61K 31/4439; A61K 31/454; A61K 31/4545; A61K 31/472; A61K 31/4725; A61K 31/495; A61K 31/496; A61K 31/497; A61K 31/498; A61K 31/501; A61K 31/502; A61K 31/5025; A61K 31/506; A61K 31/519; A61K 31/52; A61K 31/55; A61K 31/5513; A61K 31/56; A61K 31/58; A61K 31/585; A61K 31/606; A61K 31/675; A61K 31/69; A61K 31/7048; A61K 31/7056; A61K 31/706; A61K 31/737; A61K 38/06; A61K 38/12; A61K 38/15; A61K 39/3955; A61K 45/06; A61P 1/00; A61P 1/16; A61P 3/04; A61P 7/02; A61P 9/04; A61P 9/12; A61P 11/00; A61P 13/12; A61P 17/02; A61P 19/02; A61P 19/08; A61P 19/10; A61P 21/00; A61P 25/22; A61P 25/24; A61P 25/28; A61P 27/12; A61P 35/00

(57) ABSTRACT

The present disclosure provides a composition for removing senescent cells that is useful in senescent cell removal. The composition of the present invention induces apoptosis of senescent cells, and thus can be effectively applied to the alleviation, treatment, or prevention of various aging-associated diseases and disorders caused by senescent cells. In addition, the composition of the present invention can be effectively applied as pharmaceuticals, cosmetics, health functional foods, animal products, and the like in relation to aging-associated diseases and disorders caused by senescent cells.

### TECHNICAL FIELD

This application is based on and claims priority from Korean Patent Application No. 10-2023-0058711, filed on May 4, 2023, with the Korean Intellectual Property Office, the disclosure of which is incorporated herein in its entirety by reference.

The present invention relates to a composition that has the effect of removing senescent cells. The composition of the present invention comprises as an active agent a compound having excellent ability of removing senescent cells or its pharmaceutically acceptable salt; secreted fluid from cells killed by treatment with such a compound or its pharmaceutically acceptable salt; or cells killed by treatment with such a compound or its pharmaceutically acceptable salt. The invention also relates to various uses, in particular medical uses of the compositions of the invention.

### BACKGROUND ART

Aging refers to the process of gradual physical and cognitive decline as one ages, leading to death after humans are born and grow for a certain period of time. The World Health Organization (WHO) assigned a disease code (MG2A) to old age itself in 2018, and several research and development and clinical trials are being conducted to treat (e.g., slow down) aging.

In particular, the target of aging is being narrowed from ‘individual’ to ‘cell’ through various research and development results. Accordingly, it was revealed that ‘senescent cells’ are the fundamental cause of various diseases and disorders related to aging. Senescent cells occur in the human body due to various factors such as aging, drug side effects, stress, obesity, diabetes, wounds, transplants, and infections. These senescent cells are removed by the body's immune cells. However, as immune cells do not function properly due to various reasons due to aging, or more fundamentally, immune cells also age together, they do not function properly and eventually senescent cells accumulate in the body.

Accumulated senescent cells secret a large number of senescence-promoting factors as well as senescence-associated pathogenic factors such as senescence-associated inflammation and senescence-associated fibrosis. The role of these senescence-associated secretory phenotypes (SASP) is known (i) to gradually spread senescent cells by changing surrounding normal cells into senescent cells, (ii) to change the structure and function in all organs and tissues, (iii) to cause various underlying pathological phenotypes such as chronic inflammation (immuno-aging) and fibro-aging due to aging, and (iv) accordingly, to ultimately cause various aging-related diseases and disorders throughout the body.

Accordingly, recently, just as cancer is treated by removing cancer cells, various research and development efforts are underway to treat aging-related diseases and disorders by removing senescent cells (Robbins P D et al., Annual Review of Pharmacological Toxicology 61, 779-803, 2021). For example, ABT-263/737 has been recently reported as an innovative anti-aging treatment, but many side effects such as toxicity exist. Therefore, it is important to develop new compounds and/or compositions that eliminate senescent cells through a mechanism different from ABT-263/737.

### DISCLOSURE

### Technical Problem

Therefore, the problem to be solved by the present invention is to provide compositions useful for removing senescent cells and uses of such compositions for removing senescent cells.

Another problem to be solved by the present invention is to provide compositions that can improve, treat or prevent various diseases and disorders related to senescent cells by removing senescent cells via comprising the active agent according to the present invention. That is, another problem to be solved by the present invention is to provide uses, preferably medical uses, of the composition according to the present disclosure for improving, treating or preventing diseases and disorders related to senescent cells.

### Technical Solution

In order to achieve the above object, one aspect of the present invention provides compositions for removing senescent cells, comprising as active agent (a) the compound of Table 1 or 2 below or its pharmaceutically acceptable salt; (b) secreted fluid from cells killed by treatment with such compound or pharmaceutically acceptable salt thereof; or (c) cells killed by treatment with such compound or pharmaceutically acceptable salt thereof. That is, one aspect of the present invention provides uses, preferably medical uses, for removing senescent cells by treatment with the compound of Table 1 or 2 below or its pharmaceutically acceptable salt; secreted fluid from cells killed by treatment with such compound or pharmaceutically acceptable salt thereof; or cells killed by treatment with such compound or pharmaceutically acceptable salt thereof.

In one embodiment of the present invention, the compounds in Table 1 or 2 below have the effect of increasing the secretion of HMGN1 (High Mobility Group Nucleosome Binding Domain 1) protein (HMG14) in senescent cells.

TABLE 1

| Compound Name | Structure, CAS Number, or |
|---|---|
| Compound No. | IUPAC name |
| TVB-2640 LX-305 | 1399177-37-7 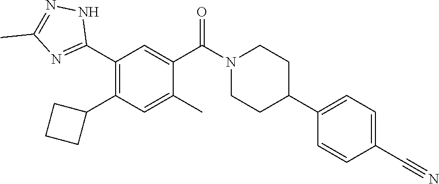 [SMILES: Cc1n[nH]c(-c2cc(C(=O)N3CCC(c4ccc(C#N)cc4)CC3)c(C)cc2C2CCC2)n1] |
| ML210 (DPI10) LX-401 | 1360705-96-9 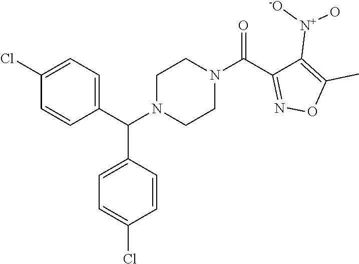 |
| JKE1674 LX-403 | 2421119-60-8 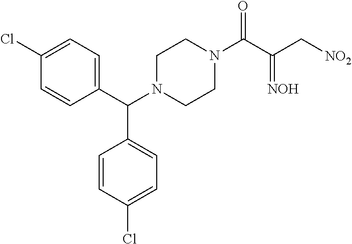 |
| RSL3 LX-405 | 1219810-16-8 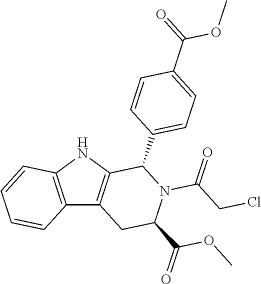 [SMILES: COC(=O)c1ccc([C@H]2c3[nH]c4ccccc4c3C[C@H](C(=O)OC)N2C(=O)CCl)cc1] |
| COTI-2 | 1039455-84-9 |
| LX-801 | |
| PC14586 LX-905 | 2649340-85-0 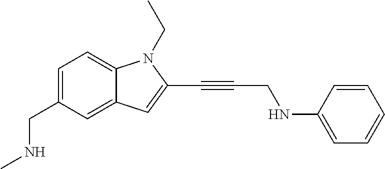 [SMILES: CCn1c(C#CCNc2ccccc2)cc2cc(CNC)ccc21] |
| Siramesine LX-1102 | 147817-50-3 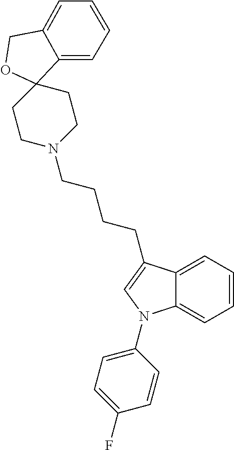 [SMILES: Fc1ccc(-n2cc(CCCCN3CCC4(CC3)OCc3ccccc34)c3ccccc32)cc1] |
| 5-Aminolevulinic | 106-60-5 |
| acid | |
| LX-1103 | |
| Sulfasalazine | 599-79-1 |
| LX-1302 | |
| Disulfiram | 97-77-8 |
| LX-1601 | |
| Leflunomide | 75706-12-6 |
| LX-1603 | |
| Ferroptocide | (2S,3aS,4S,5R,7R,8S,8aS,11R)- |
| LX-1801 | 7-(1,3-Dioxo-2-phenyl- |
| | 2,3,5,8-tetrahydro-1H- |
| | [1,2,4]triazolo[1,2-a]pyridazin- |
| | 6-yl)-2,3a-dihydroxy-4,8,11- |
| | trimethyl-3-oxooctahydro-1H- |
| | 4,8a-propanoazulen-5-yl 2- |
| | Chloroacetate |
| | 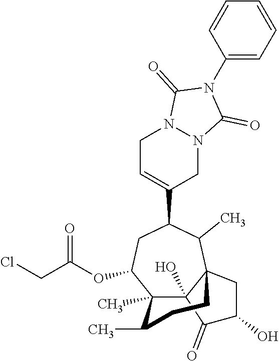 |
| Pralsetinib | 2097132-94-8 |
| LX-1902 | |
| Erdafitinib | 1346242-81-6 |
| LX-1903 | |
| Ursolic acid | 77-52-1 |
| LX-2001 | |
| Corosolic acid | 4547-24-4 |
| LX-2002 | |
| Oleanolic acid | 508-02-1 |
| LX-2003 | |
| Betulinic acid | 472-15-1 |
| LX-2005 | |
| Moronic acid | 6713-27-5 |
| LX-2007 | |
| Momordin | 96990-18-0 |
| Omaveloxolone | 1474034-05-3 |
| (RTA-408) | |
| LX-2101 | |
| Bardoxolone | 218600-53-4 |
| methyl (RTA- | |
| 402, CDDO-Me) | |
| LX-2102 | |
| TX63682 LX-2201 | 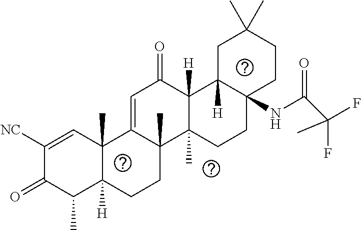 |
| TP-321 LX-2311 | 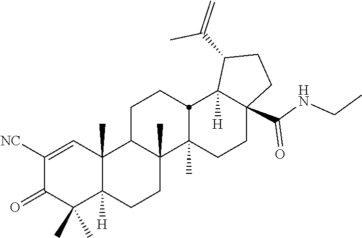 |
| 63101 (dh402, 402-02) LX-2301 | 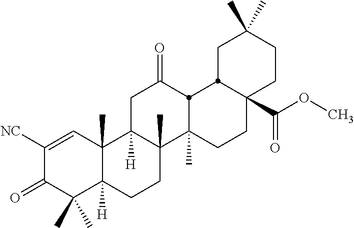 |
| 63102 (dh404, 404-02) LX-2302 | 1191265-33-4 Dihydro-CDDO-Trifluoroethyl Amide |
| | 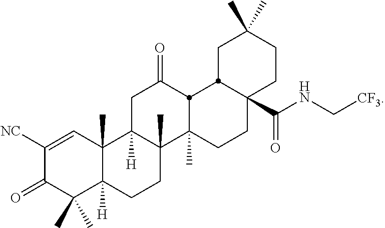 |
| 63167 (402-12) LX-2305 | 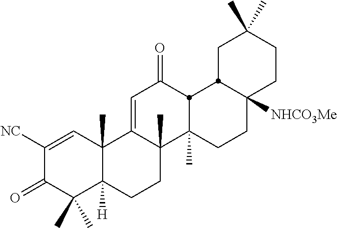 |
| 63175 (402-19) LX-2306 | 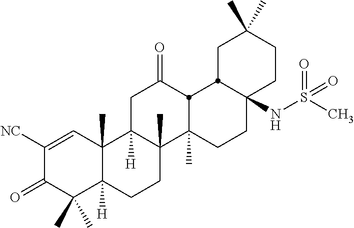 |
| 63194 (402-50) LX-2307 | 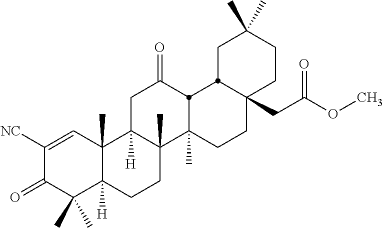 |
| 63311 LX-2308 | 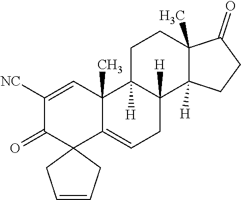 |
| 63165 (402-09) LX-2309 | 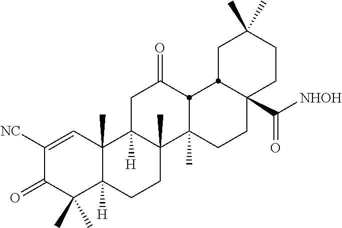 |
| 63166 (402-10) LX-2310 | 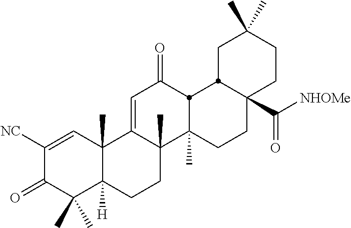 |
| TX63541 LX-2312 | 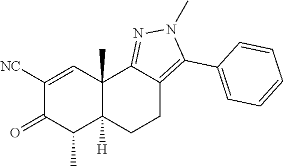 [SMILES: C[C@@H]1C(=O)C(C#N)=C[C@]2(C)c3nn(C)c(-c4ccccc4)c3CC[C@@H]12] |
| TX63724 LX-2315 | 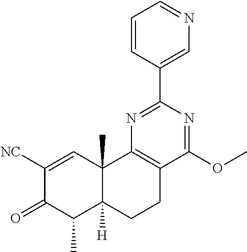 [SMILES: COc1nc(-c2cccnc2)nc2c1CC[C@H]1[C@H](C)C(=O)C(C#N)=C[C@]21C] |
| TX63435 LX-2316 | 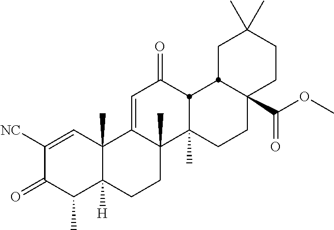 |
| TX63545 LX-2317 | 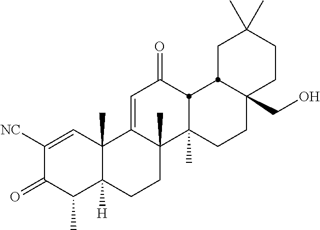 |
| TX63537 LX-2318 | 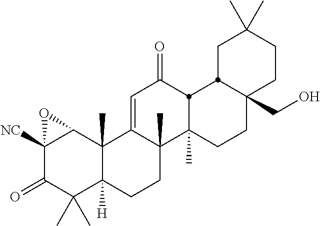 |
| TX63946 LX-2319 | 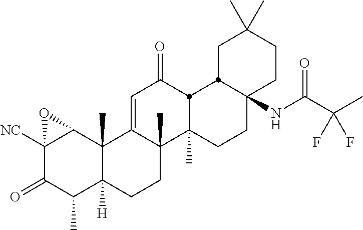 |
| TX63501 LX-2401 | 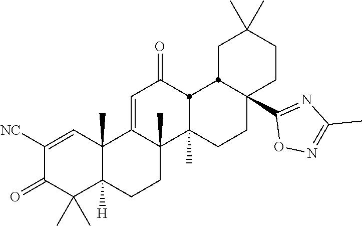 |
| TX63768 LX-2402 | 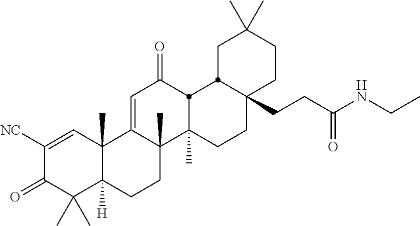 |
| TX63770 LX-2403 | 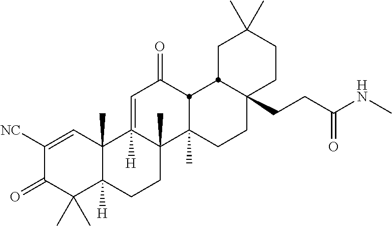 |
| TX63490 LX-2405 | 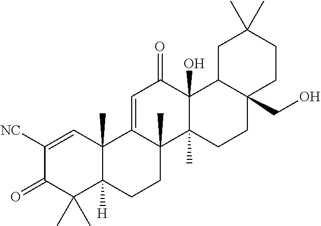 |
| TX63530 LX-2406 | 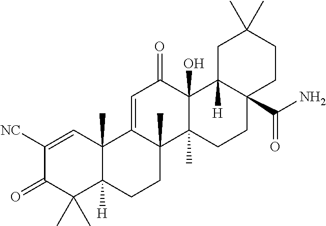 |
| LX-2407 | (5aS,6S,9aR)-1,6-dimethyl-7- |
| | oxo-9a-phenyl-3-(pyridin-3- |
| | yl)-4,5,5a,6,7,9a-hexahydro- |
| | 1H-benzo[g]indazole-8- |
| | carbonitrile |
| LX-2408 | (5aS,6S,9aR)-1,6-dimethyl-7- |
| | oxo-9a-phenyl-3-(pyrimidin-5- |
| | yl)-4,5,5a,6,7,9a-hexahydro-H- |
| | benzo[g]indazole-8- |
| | carbonitrile) |
| T12 LX-2409 | 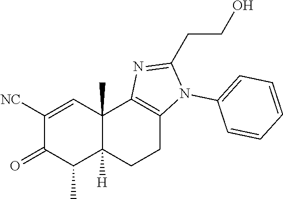 [SMILES: C[C@@H]1C(=O)C(C#N)=C[C@]2(C)c3nc(CCO)n(-c4ccccc4)c3CC[C@@H]12] |
| T48 LX-2501 | 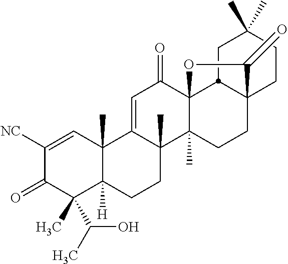 |
| T158 LX-2502 | 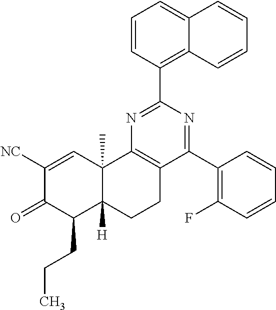 [SMILES: CCC[C@H]1C(=O)C(C#N)=C[C@@]2(C)c3nc(-c4cccc5ccccc45)nc(-c4ccccc4F)c3CC[C@H]12] |
| T159 LX-2503 | 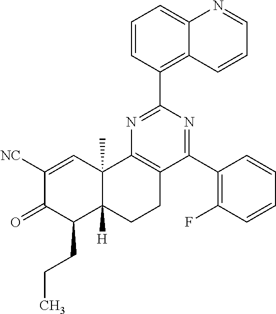 [SMILES: CCC[C@H]1C(=O)C(C#N)=C[C@@]2(C)c3nc(-c4cccc5ncccc45)nc(-c4ccccc4F)c3CC[C@H]12] |
| T45 LX-2505 | 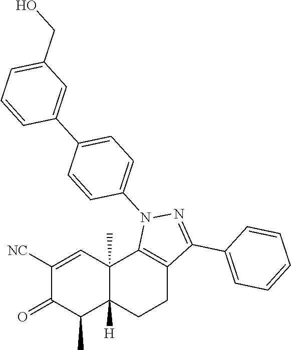 [SMILES: C[C@H]1C(=O)C(C#N)=C[C@@]2(C)c3c(c(-c4ccccc4)nn3-c3ccc(-c4cccc(CO)c4)cc3)CC[C@H]12] |
| T83 LX-2506 | 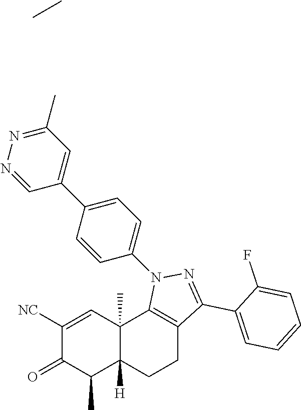 |
| T38 LX-2507 | 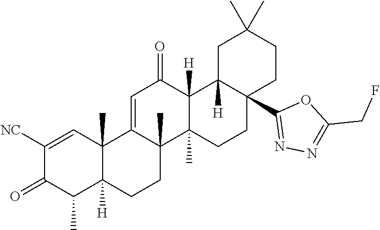 |
| T42 LX-2508 | 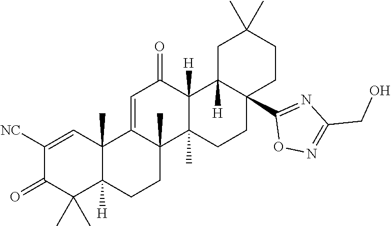 |
| T16 LX-2509 | 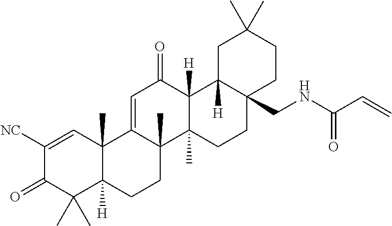 |
| T53 LX-2510 | 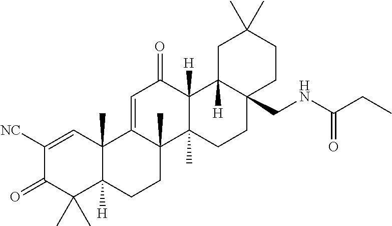 |
| T5 LX-2511 | 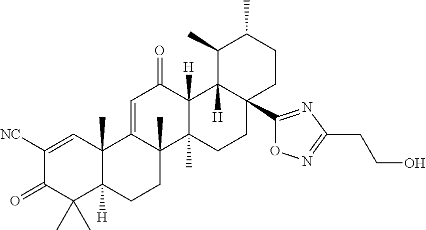 |
| T13 LX-2512 | 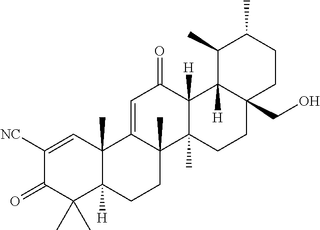 |
| Soloxolone methyl LX-2513 | 1032131-00-2 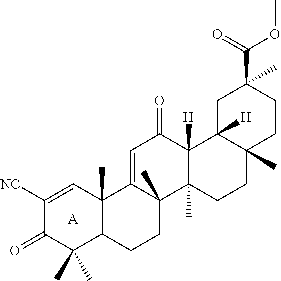 |
| Soloxolone tryptamide LX-2517 | 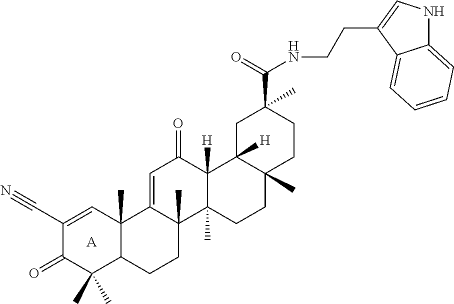 |
| ATN-224 | 649749-10-0 |
| LX-2601 | |
| Ezatiostat | 168682-53-9 |
| LX-2602 | |
| Polyunsaturated | e.g. Arachidonic acid (LX- |
| fatty acid | 2701) (506-32-1), |
| | Docosahexaenoic acid (LX- |
| | 2702) (6217-54-5), Linoleic |
| | acid (LX-2703) (60-33-3), |
| | Dihomogamma-linolenic acid |
| | (LX-2705) (1783-84-2), |
| | Punicic acid (LX-2707) (544- |
| | 72-9), Eicosapentaenoic acid |
| | (10417-94-4) |
| Methotrexate | 59-05-2 |
| LX-2901 | |
| Tocofersolan | 9002-96-4 |
| (TPGS) | |
| LX-3001 | |
| APATONE ® | 50-81-7 + 58-27-5 |
| LX-3002 | |
| Opaganib | 915385-81-8 |
| (ABC294640) | |
| LX-3106 | |
| Numidargistat | 2095732-06-0 |
| (INCB01158, | |
| CB-1158) | |
| LX-3107 | |
| IACS-010759 | 1570496-34-2 |
| LX-3200 | |
| Sirpiglenastat | 2079939-05-0 |
| (DRP-104) | |
| LX-3201 | |
| AZD3965 | 1448671-31-5 |
| LX-3203 | |
| Gilteritinib | 1254053-43-4 |
| (ASP2215) | |
| LX-3303 | |
| PD-1 inhibitor | |
| LX-3403 | |
| SIMR1281 (Compound 1t) LX-5201 | 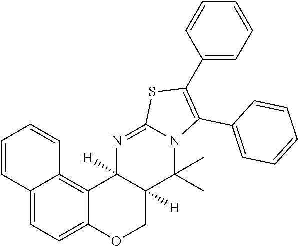 [SMILES: CC1(C)[C@H]2COc3ccc4ccccc4c3[C@H]2N=C2SC(c3ccccc3)=C(c3ccccc3)N21] |
| Motexafin | 246252-06-2 |
| Gadolinium | |
| LX-5211 | |
| Compound 31 LX-5328 | 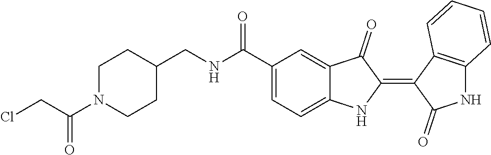 [SMILES: O=C1Nc2ccccc2/C1=C1/Nc2ccc(C(=O)NCC3CCN(C(=O)CCl)CC3)cc2C1=O] |
| | [symbol] |
| DA-5 LX-5341 | 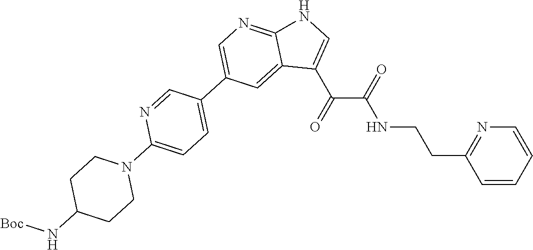 [SMILES: CC(C)(C)OC(=O)NC1CCN(c2ccc(-c3cnc4[nH]cc(C(=O)C(=O)NCCc5ccccn5)c4c3)cn2)CC1] |
| Tubastatin A | 1252003-15-8 |
| LX-5352 | |
| Compound 1a LX-5362 | 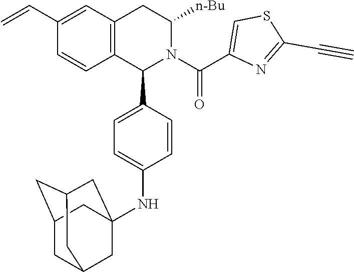 |
| Compound 24 LX-5401 | 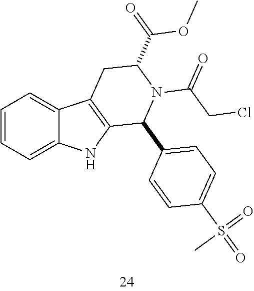 [SMILES: COC(=O)[C@H]1Cc2c([nH]c3ccccc23)[C@H](c2ccc(S(C)(=O)=O)cc2)N1C(=O)CCl] |
| | methyl (1S,3R)-2-(2- |
| | chloroacetyl)-1-(4- |
| | (methylsulfonyl)phenyl)- |
| | 2,3,4,9-tetrahydro-1H- |
| | pyrido[3,4-b]indole-3- |
| | carboxylate |
| Compound 28 LX-5411 | 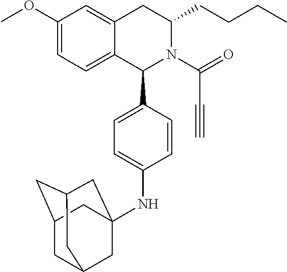 [SMILES: C#CC(=O)N1[C@@H](CCCC)Cc2cc(OC)ccc2[C@@H]1c1ccc(NC23CC4CC(CC(C4)C2)C3)cc1] |
| Vancomycin | 1404-90-6 |
| LX-5424 | |
| Lupeol | 545-47-1 |
| LX-5433 | |
| Lorlatinib | 1454846-35-5 |
| LX-5449 | |
| Example 4 LX-5701 | 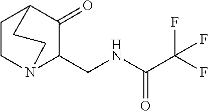 [SMILES: O=C1C2CCN(CC2)C1CNC(=O)C(F)(F)F] |
| Example 1 LX-5702 | 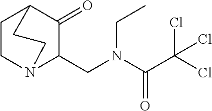 [SMILES: CCN(CC1C(=O)C2CCN1CC2)C(=O)C(Cl)(Cl)Cl] |
| Example 21 LX-5706 | 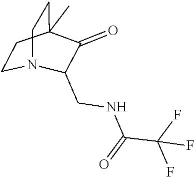 [SMILES: CC12CCN(CC1)C(CNC(=O)C(F)(F)F)C2=O] |
| Example 22 LX-5707 | 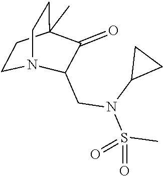 [SMILES: CC12CCN(CC1)C(CN(C1CC1)S(C)(=O)=O)C2=O] |
| Example 15 LX-5711 | 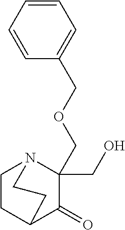 [SMILES: O=C1C2CCN(CC2)C1(CO)COCc1ccccc1] |
| Example 3 LX-5721 | 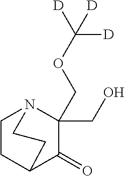 [SMILES: [2H]C([2H])([2H])OCC1(CO)C(=O)C2CCN1CC2] |
| Example 19 LX-5722 | 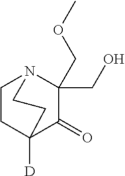 |
| Example 17 | (1R,2R,4S,6R)-2- |
| LX-5731 | (hydroxymethyl)-2- |
| | (methoxymethyl)-6- |
| | methylquinuclidin-3-one |
| Compound 1A LX-5904 | 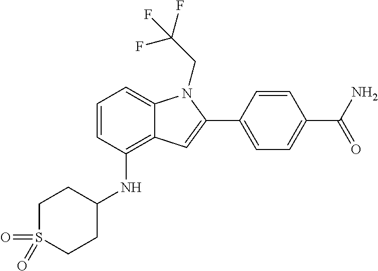 [SMILES: NC(=O)c1ccc(-c2cc3c(NC4CCS(=O)(=O)CC4)cccc3n2CC(F)(F)F)cc1] |
| Compound 54 LX-5906 | 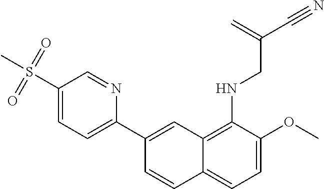 [SMILES: C=C(C#N)CNc1c(OC)ccc2ccc(-c3ccc(S(C)(=O)=O)cn3)cc12] |
| Compound 684 LX-5907 | 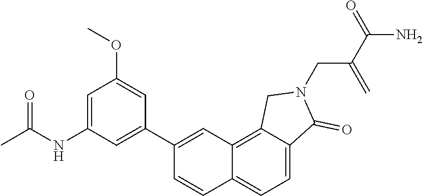 [SMILES: C=C(CN1Cc2c(ccc3ccc(-c4cc(NC(C)=O)cc(OC)c4)cc23)C1=O)C(N)=O] |
| Pevonedistat | 905579-51-3 |
| LX-6202 | |
| Alisertib | 1028486-01-2 |
| LX-6203 | |
| Barasertib | 722543-31-9 |
| (AZD1152) | |
| LX-6205 | |
| Volasertib | 755038-65-4 |
| LX-6207 | |
| Plitidepsin | 137219-37-5 |
| LX-6212 | |
| Troriluzole | 1926203-09-9 |
| LX-6216 | |
| Nanvuranlat | 1037592-40-7 |
| (JPH203) | |
| LX-6233 | |
| Elesclomol (STA- | 488832-69-5 |
| 4783) | |
| LX-6501 | |
| AL102 (BMS- | 1584647-27-7 |
| 986115) | |
| LX-6514 | |
| SBP-101 | 748119-79-1 |
| (Ivospemin) | |
| LX-6516 | |
| Thioguanine | 154-42-7 |
| LX-6530 | |
| Ribavirin | 36791-04-5 |
| LX-6537 | |
| Andrographolide | 5508-58-7 |
| LX-6800 | |
| Paeoniflorin | 23180-57-6 |
| LX-6804 | |
| Roxadustat | 808118-40-3 |
| LX-6806 | |
| Dexmedetomidine | 113775-47-6 |
| LX-6807 | |
| Chidamide | 1616493-44-7 |
| (Tucidinostat) | |
| LX-6809 | |
| Pirtobrutinib | 2101700-15-4 |
| (LOXO-305) | |
| LX-6981 | |
| Batiraxcept | 2268717-61-7 |
| (AVB-S6-500) | |
| LX-6982 | |
| Selpercatinib | 2152628-33-4 |
| (LOXO-292) | |
| LX-6988 | |
| Sulforaphane | 4478-93-7 |
| LX-7103 | |
| Sulforadex (SFX-01) LX-7104 | 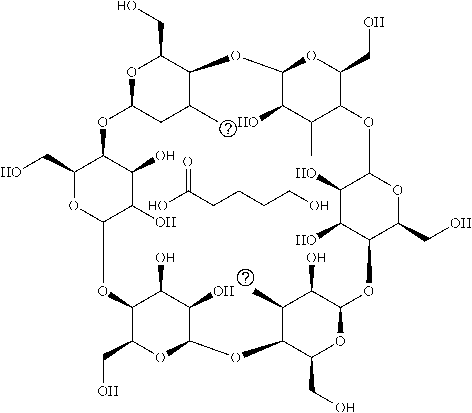 |
| 10-Nitrooleic acid | 875685-46-4 |
| (CXA10) | |
| LX-7111 | |
| Tideglusib | 865854-05-3 |
| LX-7121 | |
| Terameprocol | 24150-24-1 |
| LX-7122 | |
| Enzastaurin | 170364-57-5 |
| LX-7135 | |
| ONC201 (TIC10, | 1616632-77-9 |
| Dordaviprone) | |
| LX-7261 | |
| ONC206 | 1638178-87-6 |
| LX-7262 | |
| ONC212 | 1807861-48-8 |
| LX-7263 | |
| 4′-thio-5-aza-2′- deoxycytidine LX-7288 | 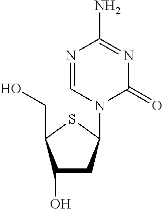 [SMILES: Nc1ncn([C@H]2C[C@@H](O)[C@@H](CO)S2)c(=O)n1] |
| Lixumistat | 1422365-93-2 |
| (IM156) | (free base) |
| LX-7403 | 1422365-94-3 |
| | (acetate) |
| | 1422365-52-3 |
| | (hydrochloride) |
| Pralatrexate | 146464-95-1 |
| LX-7405 | |
| Belzutifan | 1672668-24-4 |
| LX-7451 | |
| Telcyta (TLK-286, | 158382-37-7 |
| Canfosfamide) | |
| LX-7463 | |
| Farletuzumab | 896723-44-7 |
| LX-7621 | |
| Mirvetuximab | 1453084-36-0 |
| LX-7622 | |
| PD-L1 inhibitor | |
| LX-8401 | |
| CTLA-4 | |
| inhibitor | |
| LX-8402 | |
| AG-270 | 2201056-66-6 |
| LX-8721 | |
| Vorasidenib | 1644545-52-7 |
| (AG-881) | |
| LX-8726 | |
| Indoximod (1- | 110117-83-4 |
| Methyl-D- | |
| tryptophan) | |
| LX-8801 | |
| Epacadostat | 1204669-58-8 |
| (INCB024360) | |
| LX-8804 | |
| Linrodostat (BMS- | 1923833-60-6 |
| 986205, ONO- | |
| 7701) LX-8805 | |
| Padnarsertib | 1643913-93-2 |
| (KPT-9274) | |
| LX-8812 | |
| Alendronate | 66376-36-1 |
| LX-9105 | (acid) |
| | 129318-43-0 |
| | (sodium) |
| | 121268-17-5 |
| | (sodium salt) |
| Metronidazole | 443-48-1 |
| (Metrogel) | |
| LX-9017 | |
| Trimethoprim | 738-70-5 |
| LX-9114 | |
| 5-ASA (5- | 89-57-6 |
| aminosalicylic | |
| acid, | |
| Mesalamine) | |
| LX-9118 | |
| Pregabalin | 148553-50-8 |
| LX-9131 | |
| Eritoran | 185954-98-7 |
| tetrasodium | |
| LX-9136 | |
| Resatorvid (TAK- | 243984-11-4 |
| 242; CLI-095) | |
| LX-9137 | |
| Repaglinide | 135062-02-1 |
| LX-9302 | |
| Tacrolimus | 104987-11-3 |
| LX-9314 | |
| Ezetimibe | 163222-33-1 |
| LX-9321 | |
| glyburide | 10238-21-8 |
| (Glibenclamide) | |
| LX-9335 | |
| Thioctic Acid (α- | 1077-28-7 |
| Lipoic Acid) | |
| LX-9336 | |
| Ruboxistaurin | 169939-94-0 |
| LX-9339 | |
| Icomidocholic | 246529-22-6 |
| acid (Aramchol) | |
| LX-9343 | |
| Arhalofenate | 24136-23-0 |
| (MBX-102) | |
| LX-9409 | |
| PBT434 | 1232840-87-7 |
| LX-9421 | (free base) |
| | 2387898-69-1 |
| | (mesylate) |
| Pioglitazone | 111025-46-8 |
| LX-9422 | |
| Rosiglitazone | 122320-73-4 |
| LX-9423 | |
| N-methyl | 872-50-4 |
| pyrrolidine | |
| LX-9425 | |
| MK-0616 LX-9561 | 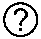 |
| Alirocumab | 1245916-14-6 |
| (Praluent) | |
| LX-9562 | |
| Evolocumab | 1256937-27-5 |
| (Repatha) | |
| LX-9563 | |
| Ranolazine | 95635-55-5 |
| LX-9565 | |
| Dazdotuftide | 2522933-44-2 |
| (TRS01) | |
| LX-9601 | |
| Bempedoic acid | 738606-46-7 |
| (ETC-1002) | |
| LX-9602 | |
| Treprostinil | 81846-19-7 |
| LX-9713 | |
| Saroglitazar | 1639792-20-3 |
| Magnesium | |
| LX-9717 | |
| Mycophenolic | 24280-93-1 |
| acid | |
| LX-9725 | |
| Obeticholic Acid | 459789-99-2 |
| LX-9732 | |
| Setanaxib | 1218942-37-0 |
| LX-9744 | |

[symbol]

indicates data missing or illegible when filed

TABLE 2
TVB-3664 (2097262-58-1) having a similar structure to TVB-2640 in Table 1, and compounds included
on pages 458 to 521 of WO2012122391A1
DPI2/3/4/6/8/9/15/13/19/17/18/12 described in WO2014011973A2, JKE1708, JKE1716 (2421118-05-8),
Isopropyl-ML210, and GPX4-IN-3 (26a) (2761004-85-5) described in FIG. 5 and FIG. 3 of the paper
(10.1038/s41589-020-0501-5), and LOC1886/880/577/957/4873/5893/1250, QW-
003/010/011/017/020/023/029/034/050 compounds described on pages 54-57 of WO2021183908A1,
which have a similar structure to ML210 (DPI10), ML162 (DPI7), JKE1674 (intracellular conversion
compound of ML210), RSL3, LOC880, or QW-017 in Table 1

| The compounds on the right described in WO2014011973A2 | 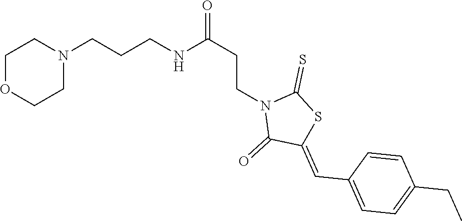 [SMILES: CCc1ccc(/C=C2\SC(=S)N(CCC(=O)NCCCN3CCOCC3)C2=O)cc1] |
|---|---|
| | DPI2 |
| | 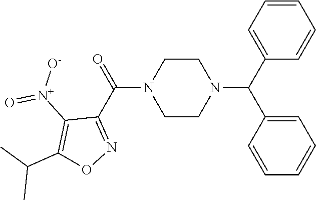 |
| | DPI10 |
| | 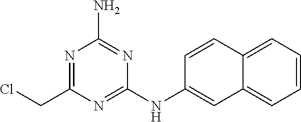 |
| | DPI17 |
| | 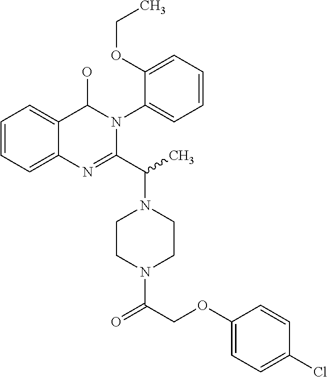 |
| | Erastin |
| | 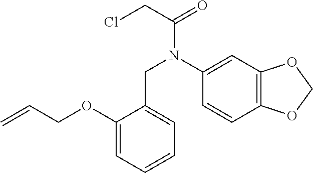 |
| | DPI13 |
| | 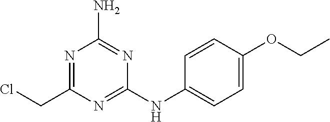 |
| | DPI18 |
| | 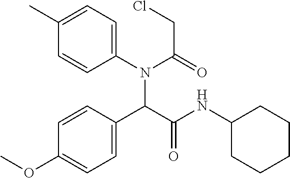 |
| | DPI8 |
| | 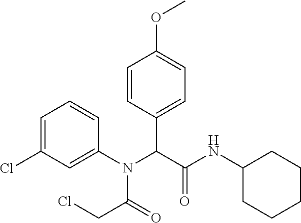 |
| | DPI9 |
| | 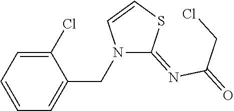 |
| | DPI12 |
| | 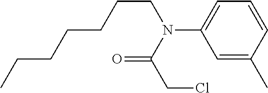 |
| | DPI19 |
| | 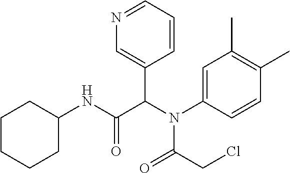 |
| | DPI6 |
| | 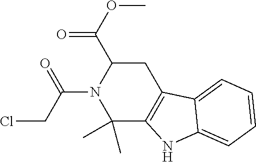 |
| | DPI15 |
| | 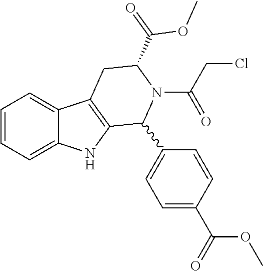 |
| | RSL3 |
| | 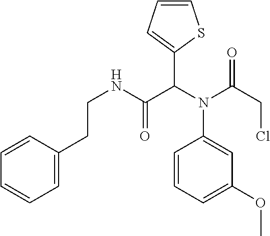 |
| | DPI3 |
| | 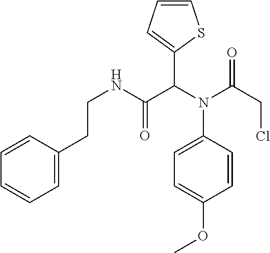 |
| | DPI4 |
| | 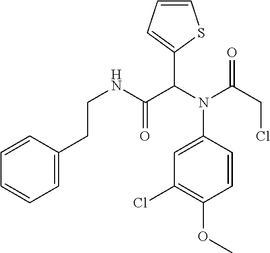 |
| | DPI7 |
| | 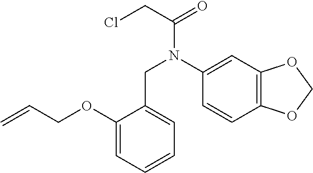 |
| | DPI13 |
| | 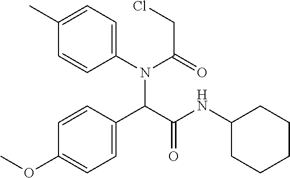 |
| | DPI8 |
| | 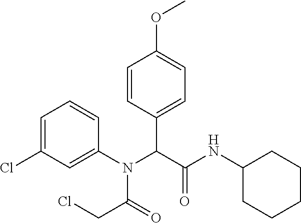 |
| | DPI9 |
| The compounds on the right described in the paper (10.1038/s41589-020- 0501-5, FIG. 5) | 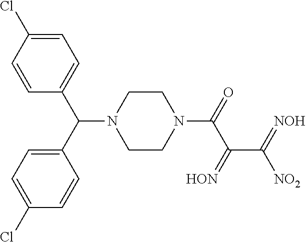 |
| | JKE-1708 |
| | (55) |

| The compounds on the | | | Molecu- | |
|---|---|---|---|---|
| right described in | | | lar | MST |
| WO2021183908A1 | Name | Structure | Weight | K_{d} |
| | QW-003 | 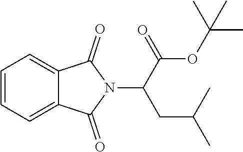 [SMILES: CC(C)CC(C(=O)OC(C)(C)C)N1C(=O)c2ccccc2C1=O] | 317.39 | Not de- ter- mined |
| | QW-010 | 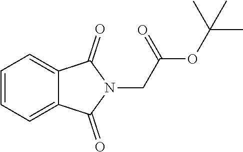 [SMILES: CC(C)(C)OC(=O)CN1C(=O)c2ccccc2C1=O] | 261.28 | 2.9 μM |
| | QW-011 | 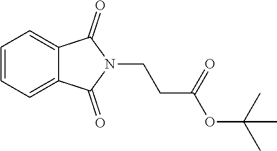 [SMILES: CC(C)(C)OC(=O)CCN1C(=O)c2ccccc2C1=O] | 275.30 | 926 nM |
| | QW-017 | 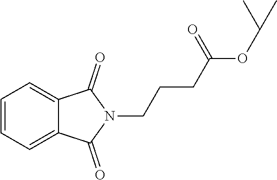 [SMILES: CC(C)OC(=O)CCCN1C(=O)c2ccccc2C1=O] | 289.33 | 399 nM |
| | QW-020 | 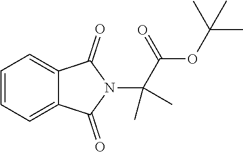 [SMILES: CC(C)(C)OC(=O)C(C)(C)N1C(=O)c2ccccc2C1=O] | 289.33 | 2.14 μM |
| | QW-023 | 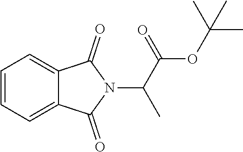 [SMILES: CC(C(=O)OC(C)(C)C)N1C(=O)c2ccccc2C1=O] | 275.30 | 1.78 μM |
| | QW-029 | 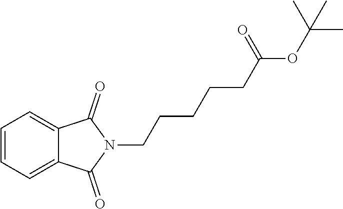 [SMILES: CC(C)(C)OC(=O)CCCCCN1C(=O)c2ccccc2C1=O] | 319.39 | 2.1 μM |
| | QW-034 | 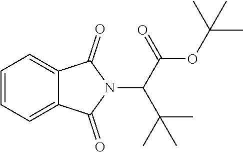 [SMILES: CC(C)(C)OC(=O)C(N1C(=O)c2ccccc2C1=O)C(C)(C)C] | 317.39 | 979 nM |
| | QW-050 | 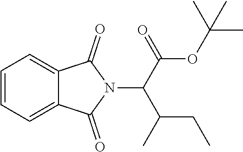 [SMILES: CCC(C)C(C(=O)OC(C)(C)C)N1C(=O)c2ccccc2C1=O] | 317.39 | 2.43 μM |

COTI-5, COTI-217, COTI-219, and COTI-220, which have a similar structure to COTI-2, COTI-NMe2 of
Table 1, described in WO2010006438A1 or Triapine, di-2-pyridylketone 4-cyclohexyl-4-methyl-3-
thiosemicarbazone (DpC), COTI-NH2, COTI-NMe2, COTI-NMeCy compounds described in the paper
(10.3390/cancers14184455)
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97/98/
99/100/101/102/103/104/105/106/107/108/109/110/111/112/113/114/115/116/117/118/119/120/121/122/
123/124/125/126/127/128/129/130/131/132/133/134/135/136/137/138/139/140/141/142/143/144/145/146/
147/148/149/150/151/152/153/154/155/156/157/158/159/160/161/162/163/164/165/166/167/168/169/
170/171/172/173/174/175/176/177/178/179/180/181/182/183/184/185/186/187/188/189/190/191/192/
193/194/195/196/197/198/199/200/201/202/203/204/205/206/207/208/209/210/211/212/213/214/215/216/
217/218/219/220/221/222/223/224/225/226/227/228/299/230/231/232/233/234/235/236/237/238/239/
240/241/242/243/244/245/246/247/248/249/250/251/252/253/254/255/256/257/258/259/260/261/262/
263/264/265/266/267/268/269/270/271/272/273/274/275/276/277/278/279/280/281/282/283/284/285/286/
287/288/289/290/291/292/293/294/295/296/297/298/299/300/301/302/303/304/305/306/307/308/309/
310/311/312/313/314/315/316/317/318/319/320/321/322/323/324/325/326/327/328/329/330/331/332/
333/334/335/336/337/338/339/340/341/342/343/344/345/346/347/348/349/350/351/352/353/354/355/356/
357/358/359/360/361/362/363/364/365/366/367/368/369/370/371/372/373/374/375/376/377/378/379/
380/381/382/383/384/385/386/387/388/389/390/391/392/393/394/395/396/397/398/399/400/401/402/
403/404/405/406/407/408/409/410/411/412/413/414/415/416/417/418/419/420/421/422/423/424/425/426/
427/428/429/430/431/432/433/434/435/436/437/438/439/440/441/442/443/444/445/446/447/448/449/
450/451/452/453/454/455/456/457/458/459/460/461/462/463/464/465/466/467/468/469/470/471/472/
473/474/475/476/477/478/479/480/481/482/483/484/485/486/487/488/489/490/491/492/493/494/495/496/
497/498/499/500/501/502/503/504/505/506/507/508/509/510/511/512/513/514/515/516/517/518/519/
520/521/522/523/524/525/526/527/528/529/530/531/532/533/534/535/536/537/538/539/540/541/542/
543/544/545/546/547/548/549/550/551/552/553/554/555/556/557/558/559/560/561/562/563/564/565/566/
567/568/569/570/571/572/573/574/575/576/577/578/579/580/581/582/583/584/585/586/587/588/589/
590/591/592/593/594/595/596/597/598/599/600/601/602/603/604/605/606/607/608/609/610/611/612/
613/614/615/616/617/618/619/620/621/622/623/624/625/626/627/628/629/630/631/632/633/634/635/636/
637/638/639/640/641/642/643/644/645/646/647/648/649/650/651/652/653/654/655/656/657/658/659/
660/661/662/663/664/665/666/667/668/669/670/671/672/673/674/675/676/677/678/679/680/681/682/
683/684/685/686/687/688/689/690/691/692/693/694/695/696/697/698/699/700/701/702/703/704/705/706/
707/708/709/710/711/712/713/714/715/716/717/718/719/720/721/722/723/724/725/726/727/728/729/
730/731/732/733/734/735/736/737/738/739/740/741/742/743/744/745/746/747/748/749/750/751/752/
753/754/755/756/757/758/759/760/761/762/763/764/765/766/767/768/769/770/771/772/773/774/775/776/
777/778/779/780/781/782/783/784/785/786/787/788/789/790/791/792/793/794/795/796/797/798/799/
800/801/802/803/804/805/806/807/808/809/810/811/812/813/814/815/816/817/818/819/820/821/822/
823/824/825/826/827/828/829/830/831/832/833/834/835/836/837/838/839/840/841/842/843/844/845/846/
847/848/849/850/851/852/853/854/855/856/857/858/859/860/861/862/863/864/865/866/867/868/869/
870/871/872/873/874/875/876/877/878/879/880/881/882/883/884/885/886/887/888/889/890/891/892/
893/894/895/896/897/898/899/900/901/902/903/904/905/906/907/908/909/910/911/912/913/914/915/916/
917/918/919/920/921/922/923/924/925/926/927/928/929/930/931/932/933/934/935/936/937/938/939/
940/941/942/943/944/945/946/947/948/949/950/951/952/953/954/955/956/957/958/959/960/961/962/
963/964/965/966/967/968/969/970/971/972/973/974/975/976/977/978/979/980/981/982/983/984/985/986/
987/988/989/990/991/992/993/994/995/996/997/998/999/1000/1001/1002/1003/1004/1005/1006/1007/
1008/1009/1010/1011/1012/1013/1014/1015/1016/1017/1018/1019/1020/1021/1022/1023/1024/1025/
1026/1027/1028/1029/1030/1031/1032/1033/1034/1035/1036/1037/1038/1039/1040/1041/1042/1043/
1044/1045/1046/1047/1048/1049/1050/1051/1052/1053/1054/1055/1056/1057/1058/1059/1060/1061/
1062/1063/1064/1065/1066/1067/1068/1069/1070/1071/1072/1073/1074/1075/1076/1077/1078/1079/1080/
1081/1082/1083/1084/1085/1086/1087/1088/1089/1090/1091/1092/1093/1094/1095/1096/1097/1098/
1099/1100/1101/1102/1103/1104/1105/1106/1107/1108/1109/1110/1111/1112/1113/1114/1115/1116/1117/
1118/1119/1120/1121/1122/1123/1124/1125/1126/1127/1128/1129/1130/1131/1132/1133/1134/1135/1136/
1137/1138/1139/1140/1141/1142/1143/1144/1145/1146/1147/1148/1149/1150/1151/1152/1153/1154/
1155/1156/1157/1158/1159/1160/1161/1162/1163/1164/1165/1166/1167/1168/1169/1170/1171/1172/1173/
1174/1175/1176/1177/1178/1179/1180/1181/1182/1183/1184/1185/1186/1187/1188/1189/1190/1191/1192/
1193/1194/1195/1196/1197/1198/1199/1200/1201/1202/1203/1204/1205/1206/1207/1208/1209/1210/
1211/1212/1213/1214/1215/1216/1217/1218/1219/1220/1221/1222/1223/1224/1225/1226/1227/1228/
1229/1230/1231/1232/1233/1234/1235/1236/1237/1238/1239/1240/1241/1242/1243/1244/1245/1246/1247/
1248/1249/1250/1251/1252/1253/1254/1255/1256/1257/1258/1259/1260/1261/1262/1263/1264/1265/
1266/1267/1268/1269/1270/1271/1272/1273/1274/1275/1276/1277/1278/1279/1280/1281/1282/1283/
1284/1285/1286/1287/1288/1289/1290/1291/1292/1293/1294/1295/1296/1297/1298/1299/1300/1301/
1302/1303/1304/1305/1306/1307/1308/1309/1310/1311/1312/1313/1314/1315/1316/1317/1318/1319/
1320/1321/1322/1323/1324/1325/1326/1327/1328/1329/1330/1331/1332/1333/1334/1335/1336/1337/1338/
1339/1340/1341/1342/1343/1344/1345/1346/1347/1348/1349/1350/1351/1352/1353/1354/1355/1356/
1357/1358/1359/1360/1361/1362/1363/1364/1365/1366/1367/1368/1369/1370/1371/1372/1373/1374/
1375/1376/1377/1378/1379/1380/1381/1382/1383/1384/1385/1386/1387/1388/1389/1390/1391/1392/
1393/1394/1395/1396/1397/1398/1399/1400/1401/1402/1403/1404/1405/1406/1407/1408/1409/1410/
1411/1412/1413/1414/1415/1416/1417/1418/1419/1420/1421/1422/1423/1424/1425/1426/1427/1428/1429/
1430/1431/1432/1433/1434/1435/1436/1437/1438/1439/1440/1441/1442/1443/1444/1445/1446/1447/
1448/1449/1450/1451/1452/1453/1454/1455/1456/1457/1458/1459/1460/1461/1462/1463/1464/1465/
1466/1467/1468/1469/1470/1471/1472/1473/1474/1475/1476/1477/1478/1479/1480/1481/1482/1483/
1484/1485/1486/1487/1488/1489/1490/1491/1492/1493/1494/1495/1496/1497/1498/1499/1500/1501/
1502/1503/1504/1505/1506/1507/1508/1509/1510/1511/1512/1513/1514/1515/1516/1517/1518/1519/1520/
1521/1522/1523/1524/1525/1526/1527/1528/1529/1530/1531/1532/1533/1534/1535/1536/1537/1538/
1539/1540/1541/1542/1543/1544/1545/1546/1547/1548/1549/1550/1551/1552/1553/1554/1555/1556/
1557/1558/1559/1560/1561/1562/1563/1564/1565/1566/1567/1568/1569/1570/1571/1572/1573/1574/
1575/1576/1577/1578/1579/1580/1581/1582/1583/1584/1585/1586/1587/1588/1589/1590/1591/1592/
1593/1594/1595/1596/1597/1598/1599/1600/1601/1602/1603/1604/1605/1606/1607/1608/1609/1610/1611/
1612/1613/1614/1615/1616/1617/1618/1619/1620/1621/1622/1623/1624/1625/1626/1627/1628/1629/
1630/1631/1632/1633/1634/1635/1636/1637/1638/1639/1640/1641/1642/1643/1644/1645/1646/1647/
1648/1649/1650/1651/1652/1653/1654/1655/1656/1657/1658/1659/1660/1661/1662/1663/1664/1665/
1666/1667/1668/1669/1670/1671/1672/1673/1674/1675/1676/1677/1678/1679/1680/1681/1682/1683/
1684/1685/1686/1687/1688/1689/1690/1691/1692/1693/1694/1695/1696/1697/1698/1699/1700/1701/1702/
1703/1704/1705/1706/1707/1708/1709/1710/1711/1712/1713/1714/1715/1716/1717/1718/1719/1720/
1721/1722/1723/1724/1725/1726/1727/1728/1729/1730/1731/1732/1733/1734/1735/1736/1737/1738/
1739/1740/1741/1742/1743/1744/1745/1746/1747/1748/1749/1750/1751/1752/1753/1754/1755/1756/
1757/1758/1759/1760/1761/1762/1763/1764/1765/1766/1767/1768/1769/1770/1771/1772/1773/1774/
1775/1776/1777/1778/1779/1780/1781/1782/1783/1784/1785/1786/1787/1788/1789/1790/1791/1792/1793/
1794/1795/1796/1797/1798/1799/1800/1801/1802/1803/1804/1805/1806/1807/1808/1809/1810/1811/
1812/1813/1814/1815/1816/1817/1818/1819/1820/1821/1822/1823/1824/1825/1826/1827/1828/1829/
1830/1831/1832/1833/1834/1835/1836/1837/1838/1839/1840/1841/1842/1843/1844/1845/1846/1847/
1848/1849/1850/1851/1852/1853/1854/1855/1856/1857/1858/1859/1860/1861/1862/1863/1864/1865/
1866/1867/1868/1869/1870/1871/1872/1873/1874/1875/1876/1877/1878/1879/1880/1881/1882/1883/1884/
1885/1886/1887/1888/1889/1890/1891/1892/1893/1894/1895/1896/1897/1898/1899/1900/1901/1902/
1903/1904/1905/1906/1907/1908/1909/1910/1911/1912/1913/1914/1915/1916/1917/1918/1919/1920/
1921/1922/1923/1924/1925/1926/1927/1928/1929/1930/1931/1932/1933/1934/1935/1936/1937/1938/
1939/1940/1941/1942/1943/1944/1945/1946/1947/1948/1949/1950/1951/1952/1953/1954/1955/1956/
1957/1958/1959/1960/1961/1962/1963/1964/1965/1966/1967/1968/1969/1970/1971/1972/1973/1974/1975/
1976/1977/1978/1979/1980/1981/1982/1983/1984/1985/1986/1987/1988/1989/1990/1991/1992/1993/
1994/1995/1996/1997/1998/1999/2000/2001/2002/2003/2004/2005/2006/2007/2008/2009/2010/2011/
2012/2013/2014/2015/2016/2017/2018/2019/2020/2021/2022/2023/2024/2025/2026/2027/2028/2029/
2030/2031/2032/2033/2034/2035/2036/2037/2038/2039/2040/2041/2042/2043/2044/2045/2046/2047/
2048/2049/2050/2051/2052/2053/2054/2055/2056/2057/2058/2059/2060/2061/2062/2063/2064/2065/2066/
2067/2068/2069/2070/2071/2072/2073/2074/2075/2076/2077/2078/2079/2080/2081/2082/2083/2084/
2085/2086/2087/2088/2089/2090/2091/2092/2093/2094/2095/2096/2097/2098/2099/2100/2101/2102/
2103/2104/2105/2106/2107/2108/2109/2110/2111/2112/2113/2114/2115/2116/2117/2118/2119/2120/2121/
2122/2123/2124/2125/2126/2127/2128/2129/2130/2131/2132/2133/2134/2135/2136/2137/2138/2139/
2140/2141/2142/2143/2144/2145/2146/2147/2148/2149/2150/2151/2152/2153/2154/2155/2156/2157/
2158/2159/2160/2161/2162/2163/2164/2165/2166/2167/2168/2169/2170/2171/2172/2173/2174/2175/
2176/2177/2178/2179/2180/2181/2182/2183/2184/2185/2186/2187/2188/2189/2190/2191/2192/2193/
2194/2195/2196/2197/2198/2199/2200/2201/2202/2203/2204/2205/2206/2207/2208/2209/2210/2211/2212/
2213/2214/2215/2216/2217/2218/2219/2220/2221/2222/2223/2224/2225/2226/2227/2228/2229/2230/
2231/2232/2233/2234/2235/2236/2237/2238/2239/2240/2241/2242/2243/2244/2245/2246/2247/2248/
2249/2250/2251/2252/2253/2254/2255/2256/2257/2258/2259/2260/2261/2262/2263/2264/2265/2266/
2267/2268/2269/2270/2271/2272/2273/2274/2275/2276/2277/2278/2279/2280/2281/2282/2283/2284/
2285/2286/2287/2288/2289/2290/2291/2292/2293/2294/2295/2296/2297/2298/2299/2300/2301/2302/2303/
2304/2305/2306/2307/2308/2309/2310/2311/2312/2313/2314/2315/2316/2317/2318/2319/2320/2321/
2322/2323/2324/2325/2326/2327/2328/2329/2330/2331/2332/2333/2334/2335/2336/2337/2338/2339/
2340/2341/2342/2343/2344/2345/2346/2347/2348/2349/2350/2351/2352/2353/2354/2355/2356/2357/
2358/2359/2360/2361/2362/2363/2364/2365/2366/2367/2368/2369/2370/2371/2372/2373/2374/2375/
2376/2377/2378/2379/2380/2381/2382/2383/2384/2385/2386/2387/2388/2389/2390/2391/2392/2393/2394/
2395/2396/2397/2398/2399/2400/2401/2402/2403/2404/2405/2406/2407/2408/2409/2410/2411/2412/
2413/2414/2415/2416/2417/2418/2419/2420/2421/2422/2423/2424/2425/2426/2427/2428/2429/2430/
2431/2432/2433/2434/2435/2436/2437/2438/2439/2440/2441/2442/2443/2444/2445/2446/2447/2448/
2449/2450/2451/2452/2453/2454/2455/2456/2457/2458/2459/2460/2461/2462/2463/2464/2465/2466/
2467/2468/2469/2470/2471/2472/2473/2474/2475/2476/2477/2478/2479/2480/2481/2482/2483/2484/2485/
2486/2487/2488/2489/2490/2491/2492/2493/2494/2495/2496/2497/2498/2499/2500/2501/2502/2503/
2504/2505/2506/2507/2508/2509/2510/2511/2512/2513/2514/2515/2516/2517/2518/2519/2520/2521/
2522/2523/2524/2525 compounds having a similar structure to PC14586 (Compound 2) in Table 1, as
described in WO2021262483A1.
1-(4-fluorophenyl)-3-[4-[4-(4-fluorophenyl)-1 piperidinyl]-1-butyl]-1Hindole, 11a (code no.Lu29-253), 1-(4-
fluorophenyl)-3-[4-[3-(4-fluorophenyl)-8-azabicyclo[3.2.1]oct-2en-8-yl]-1-butyl]-1H-indole, 15a
compounds having a similar structure to Siramesine in Table 1, described in the paper
(10.1021/jm00011a019, abstract)
Methylaminolevulinate (33320-16-0), Hexylaminolevulinate (140898-97-1), Benzylaminolevulinate
compounds having a similar structure to 5-Aminolevulinic acid in Table 1
Compound P1/P2/P3/P4/P5/P6/P7/P8/P9/P10/P11/P12/P13/P14/P15/P16/P17/P18/P32/P33/P34
compounds having a similar structure to Ferroptocide in Table 1, as described in Claim 14 or pages 48
to 86 of WO2020210158A1
Compound100/101/102/103/104/105/106/107/108/109/110/111/112/113/114/115/116/117/118/119/120/121/
122/123/124/125/126/127/128/129/130/131/132/133/134/135/136/137/138/139/140/141/142/143/144/
145/146/147/148/149/150/151 having a similar structure to Pralsetinib in Table 1, as described in pages
21 to 28 of WO2017079140A1
Compound100/101/102/103/104/105/106/107/108/109/110/111/112/113/114/115/116/117/118/119/120/121/
122/123/124/125/126/127/128/129/130/131/132/133/134/135/136/137/138/139/140/141/142/143/144/
145/146/147/148/149/150/151/152/153/154/155/156/157/158/159/160/161/162/163/164/165/166/167/
168/169/170/171/172/173/174/175/176/177/178/179/180/181/182/183/184/185/186/187/188/189/190/191/
192/193/194/195/196/197/198/199/200/201/202/203/204/205/206/207/208/209/210/211/212/213/214/
215/216/217/218/219/220/221/222/223/224/225/226/227/228/229 as described in pages 30 to 50 of
WO2018017983A1
Compound100/101/102/103/104/105/106/107/108/109/110/111/112/113/114/115/116/117/118/119/120/121/
122/123/124/125/126/127/128/129 as described in pages 29 to 34 of WO2018022761A1
Compound100/101/102/103/104/105/106/107/108/109/110/111/112/113/114/115/116/117/118/119/120/121/
122/123/124/125/126/127/128/129/130/131 as described in pages 24 to 28 of WO2017161269A1
Compound1/2/3/4/5/6/7/8/9/10/11/12/13/14/15/16/17/18/19/20/21/22/23/24/25/26/27/28/29/30/31/32/33/
34/35 as described in pages 13 to 19 of WO2016127074A1
Compounds having a structure similar to Erdafitinib in Table 1, described on pages 181 to 409 of
WO2011135376A1; compounds described in Table 1 on pages 287 to 324 of WO2013061080A1;
compounds described in Table A1 on pages 234 to 253 of WO2013061081A1; compounds described in
Table A1 on pages 195 to 205 of WO2013061074A1; compounds described in Table A1 on pages 190
to 201 of WO2013061077A1; compounds 1 to 76 on pages 176 to 189 of WO2014174307A1;
compounds described on pages 108 to 150 of WO2013179034A1; compounds described on pages 116
to 129 of WO2013179033A1; Compounds 1/2/3/4/5/6/7/8/9 described on pages 54 to 68 of
WO2015144803A1; and compounds described on pages 130 to 170 or Table A1 of WO2012073017A1
Ursolic acid, Corosolic acid, Oleanolic acid, Betulinic acid, Moronic acid, Momordin, TX63682, which
have a similar structure to Omaveloxolone (RTA-408) and Bardoxolone methyl (RTA-402, CDDO-Me) in
Table 1,
CDDO/CDDO-Im/CDDO-MA/CDDO-TFEA/CDDO-EA compounds described in WO2008136838A1,
Compound63101/63102/63250/63197/63195/63196/63252/63205/63206/63207/63219/63229/63230/63227/
63219/63223/63237/63268/63274/63289/63295/63296/63308/63323/63325/63326 described in
WO2009129545A1,
Compound63167/63175/63169/63198/63245/63215/63193/63243/63247/63192/63187/63170/63189/63183/
63246/63182/63244/63171/63179/63176/63172/63173/63180/63181/63249/63185/63148/63178/
63188/63168/63184/63129/63174/63208/63254/63222/63236/63238/63265/63321/63322/63327/63328
described in WO2009129546A1,
Compound63194/63199/63202/63204/63213/63214/63214
salt/63218/63220/63221/63224/63225/63226/63228/63231/63232/63233/63225/63239/63253/63255/63266/
63269/63273/63275/63276/63282/63283/63284/63285/63286/63287/63288/63294/63297/63298/
63310/63303/63332/63333/63334/63335/63336/63337 described in WO2009129548A1,
63311/C0009/C0007-5/C0010/C0008/C00142/H0001/R00141/R00142/R00142-
1/D0018/D0017/D0016/D0014/D0015/63290/63291/63304/63313/63317/63318/63312/63314/63329
compounds described in WO2009146218A2,
Compound63165/63166/63161/63162/63164/63129/63163/63177/63190/63200/63251/
63301/63302/63307/63309/63316/63320 described in WO2009146216A2,
TP-321/TP-292/TP-342/TP-343/TP-344/TP-345/TP-346 described in WO2010053817A1,
TX63541/TX63724/TX63341/TX63342/TX63363/TX63364/TX63432/TX63436/TX63444/TX63445/TX63450/
TX63451/TX63454/TX63455/TX63456/TX63462/TX63463/TX63464/TX63465/TX63466/TX63467/
TX63468/TX63485/TX63486/TX63487/TX63491/TX63503/TX63504/TX63505/TX63506/TX63507/TX63508/
TX63509/TX63512/TX63513/TX63514/TX63515/TX63519/TX63524/TX63528/TX63529/TX63531/
TX63533/TX63534/TX63540/TX63542/TX63544/TX63547/TX63550/TX63551/TX63552/TX63553/TX63554/
TX63559/TX63561/TX63567/TX63568/TX63569/TX63570/TX63578/TX63579/TX63582/TX63583/
TX63584/TX63586/TX63587/TX63588/TX63589/TX63590/TX63591/TX63594/TX63595/TX63598/TX63599/
TX63600/TX63601/TX63603/TX63604/TX63605/TX63606/TX63607/TX63611/TX63612/TX63613/
TX63615/TX63617/TX63619/TX63625/TX63626/TX63627/TX63628/TX63629/TX63630/TX63631/TX63636/
TX63637/TX63641/TX63642/TX63646/TX63649/TX63650/TX63651/TX63655/TX63656/TX63659/
TX63663/TX63664/TX63665/TX63666/TX63667/TX63676/TX63690/TX63691/TX63692/TX63714/TX63718/
TX63719/TX63720/TX63721/TX63722/TX63723/TX63725/TX63727/TX63728/TX63729/TX63730/3773/
TX63731/TX63734/TX63735/TX63736/TX63748/TX63757/TX63758/TX63760/TX63761/TX63769/TX6
TX63774/TX63776/TX63791/TX63792/TX63793/TX63794/TX63803/TX63804/TX63827/TX63829/
TX63834/TX63836/TX63848/TX63849/TX63850/TX63851/TX63852/TX63853/TX63857/TX63871/TX63872/
TX63874/TX63900/TX63905 compounds described in WO2012083306A2,
TX63435/TX63545/TX63435/TX63448/TX63520/TX63521/TX63522/TX63523/TX63545/TX63546/TX63555/
TX63556/TX63557/TX63558/TX63597/TX63614/TX63616/TX63618/TX63620/TX63621/TX63622/
TX63680/TX63681/TX63693/TX63716/TX63717/TX63749/TX63778/TX63779/TX63784/TX63785/TX63786/
TX63787/TX63788/TX63789/TX63790/TX63795/TX63797/TX63798/TX63799/TX63800/TX63807/
TX63811/TX63812/TX63814/TX63815/TX63816/TX63817/TX63818/TX63819/TX63820/TX63821/TX63822/
TX63823/TX63824/TX63825/TX63826/TX63830/TX63831/TX63832/TX63833/TX63839/TX63840/
TX63841/TX63842/TX63843/TX63858/TX63859/TX63860/TX63862/TX63863/TX63864/TX63865/TX63866/
TX63867/TX63869/TX63870/TX63875/TX63876/TX63877/TX63878/TX63880/TX63881/TX63882/
TX63886/TX63887/TX63888/TX63889/TX63890/TX63891/TX63892/TX63893/TX63901/TX63904/TX63907/
TX63908/TX63909/TX63910/TX63911/TX63914/TX63915/TX63916/TX63918/TX63919/TX63920/
TX63923/TX63925/TX63928/TX63929/TX63936/TX63982/TX63984 compounds described in
WO2012125488A1,
TX63537/TX63946/TX63098/TX63100/TX63158/TX63160/TX63210/TX63211/TX63212/TX63241/TX63315/
TX63392/TX63538/TX63539/TX63647/TX63654/TX63813/TX63924/TX63947/TX63960/TX63962/
TX63966/TX63967/TX64343 compounds described in WO2013188818A1,
TX63501/TX63384/TX63475/TX63476/TX63477/TX63478/TX63479/TX63593 compounds described in
WO2014040056A1,
TX63768/TX63770/TX63403/TX63608/TX63609/TX63610/TX63742/TX63743/TX63744/TX63762/TX63763/
TX63764/TX63868/TX63926/TX63927/TX63930/TX63949/TX63950/TX63981/TX63983/TX64042/
TX64043/TX64044/TX64045/TX64046/TX64047/TX64048/TX64049/TX64052/TX64053/TX64054/TX64055/
TX64056/TX64057/TX64058/TX64059/TX64060/TX64064/TX64065/TX64066/TX64067/TX64068/
TX64074/TX64124/TX64135 compounds described in WO2014040060A1,
TX63490/TX63530/TX63421/TX63469/TX63489/TX63498/TX63499/TX63536/TX63481/TX63484/TX63496
compounds described in WO2014040073A1,
Example26/179/1/2/3/4/5/6/7/8/9/10/11/12/13/14/15/16/17/18/19/20/21/22/23/24/25/27/28/29/30/31/32/
33/34/35/36/37/38/39/40/41/42/43/44/45/46/47/48/49/50/51/52/53/54/55/56/57/58/59/60/61/62/63/64/65/
66/67/68/69/70/71/72/73/74/75/76/77/78/79/80/81/82/83/84/85/86/87/88/89/90/91/92/93/94/95/96/97/98/
99/100/101/102/103/104/105/106/107/108/109/110/111/112/113/114/115/116/117/118/119/120/121/122/
123/124/125/126/127/128/129/130/131/132/133/134/135/136/137/138/139/140/141/142/143/144/145/
146/147/148/149/150/151/152/153/154/155/156/157/158/159/160/161/162/163/164/165/166/167/168/169/
170/171/172/173/174/175/176/177/178 compounds described in WO2015112792A1,
Compound
T12/T1/T2/T3/T4/T5/T6/T7/T8/T9/T10/T11/T13/T14/T15/T16/T17/T18/T19/T20/T21/T22/T23/T24/T25/T26/
T27/T28 described in WO2016130927A1,
Compound
T48/T1/T2/T3/T4/T5/T6/T7/T8/T9/T10/T11/T12/T13/T14/T15/T16/T17/T18/T19/T20/T21/T22/T23/T24/T25/
T26/T27/T28/T29/T30/T31/T32/T33/T34/T35/T36/T37/T38/T39/T40/T41/T42/T43/T44/T45/T46/T47/
T49/T50/T51 described in WO2017053868A1,
Compound
T158/T159/T1/T2/T3/T4/T5/T6/T7/T8/T9/T10/T11/T12/T13/T14/T15/T16/T17/T18/T19/T20/T21/T22/T23/
T24/T25/T26/T27/T28/T29/T30/T31/T32/T33/T34/T35/T36/T37/T38/T39/T40/T41/T42/T43/T44/T45/T46/
T47/T48/T49/T50/T51/T52/T53/T54/T55/T56/T57/T58/T59/T60/T61/T62/T63/T64/T65/T66/T67/T68/
T69/T70/T71/T72/T73/T74/T75/T76/T77/T78/T79/T80/T81/T82/T83/T84/T85/T86/T87/T88/T89/T90/T91/
T92/T93/T94/T95/T96/T97/T98/T99/T100/T101/T102/T103/T104/T105/T106/T107/T108/T109/T110/T111/
T112/T13/T114/T115/T116/T117/T118/T119/T120/T121/T122/T123/T124/T125/T126/T127/T128/T129/
T130/T131/T132/T133/T134/T135/T136/T137/T138/T139/T140/T141/T142/T143/T144/T145/T146/T147/
T148/T149/T150/T151/T152/T153/T154/T155/T156/T157/T160/T161/T162/T163/T164/T165/T166/
T167/T168/T169/T170/T171/T172/T173/T174/T175/T176/T177/T178/T179/T180/T181/T182/T183/T184/
T185/T186/T187 described in WO2018111315A1,
Compound
T45/T83T1/T2/T3/T4/T5/T6/T7/T8/T9/T10/T11/T12/T13/T14/T15/T16/T17/T18/T19/T20/T21/T22/T23/T24/
T25/T26/T27/T28/T29/T30/T31/T32/T33/T34/T35/T36/T37/T38/T39/T40/T41/T42/T43/T44/T46/T47/
T48/T49/T50/T51/T52/T53/T54/T55/T56/T57/T58/T59/T60/T61/T62/T63/T64/T65/T66/T67/T68/T69/T70/
T71/T72/T73/T74/T75/T76/T77/T78/T79/T80/T81/T82/T84/T85/T86/T87/T88/T89/T90/T91/T92/T93/T94/
T95/T96/T97/T98/T99/T100/T101/T102/T103/T104/T105/T106/T107/T108/T109/T110/T111/T112/T13/
T114/T115/T116/T117/T118/T119/T120/T121/T122/T123/T124/T125/T126/T127/T128/T129/T130/T131/
T132/T133/T134/T135/T136/T137/T138/T139/T140/T141/T142/T143/T144/T145/T146/T147/T148/T149/
T150/T151/T152/T153/T154/T155/T156/T157/T158/T159/T160/T161/T162/T163/T164/T165/T166/T167/
T168/T169/T170/T171/T172/T173/T174/T175/T176/T177/T178/T179/T180/T181/T182/T183/T184/
T185/T186 described in WO2019241796A1,
Compound
T38/T42/T1/T2/T3/T4/T5/T6/T7/T8/T9/T10/T11/T12/T13/T14/T15/T16/T17/T18/T19/T20/T21/T22/T23/T24/
T25/T26/T27/T28/T29/T30/T31/T32/T33/T34/T35/T36/T37/T39/T40/T41/T43/T44/T45/T46
described in WO2021016191A1,
Compound
T16/T53/T3/T4/T5/T6/T7/T8/T9/T10/T11/T12/T13/T14/T15/T17/T18/T19/T20/T21/T22/T23/T24/T25/T26/
T27/T28/T29/T30/T31/T32/T33/T34/T35/T36/T37/T38/T39/T40/T41/T42/T43/T44/T45/T46/T47/T48/T49/
T50/T51/T52/T54/T55/T56/T57/T58/T59/T60/T61/T62/T63/T64/T65/T66/T67/T68/P3 described in
WO2021127480A1,
Compound
T5/T13/T1/T2/T3/T4/T6/T7/T8/T9/T10/T11/T12/T14/T15/T16/T17/T18/T19/T20/T21/T22/T23/T24/T25/T26/
T27/T28/T29/T30/T31/T32/T33/T34/T35/T36/T37/T38/T39/T40/T41/T42/T43/T44/T45/T46/T47/T48/
T49/T50/T51/T52/T53/T54/T55/T56/T57/T58/T59/T60/T61/T62/T63/T64/T65/T66/T67/T68/T69/T70/T71/
T72/T73/T74/T75/T76/T77/T78/T79/T80/T81/T82/T83/T84/T85/T86/T87/T88/T89/T90/T91/T92/T93/T94/
T95/T96/T97/T98/T99/T100/T101/T102/T103/T104/T105/T106/T107 described in WO2022155614A1, and
Soloxolone methyl, Soloxolone tryptamide (Compound 12) described in the paper
(10.3390/ph15050603) and Compounds 1/2/3/4/5/6/7/8/9/10/11/12 described in Scheme 1
Compound63250/63197/63195/63196/63252/63205/63206/63207/63219/63229/63230/63227/63219/6
3223/63237/63268/63274/63289/63295/63296/63308/63323/63325/63326 having a similar structure to
63101 (dh402, 402-02) and 63102 (dh404, 404-02) of Table 1, as described in WO2009129545A1.
Compound63169/63198/63245/63215/63193/63243/63247/63192/63187/63170/63189/63183/63246/6
3182/63244/63171/63179/63176/63172/63173/63180/63181/63249/63185/63148/63178/63188/63168/
63184/63129/63174/63208/63254/63222/63236/63238/63265/63321/63322/63327/63328 having a
similar structure to 63167 (402-12) and 63175 (402-19) of Table 1, as described in WO2009129546A1.
Compound63199/63202/63204/63213/63214/63214
salt/63218/63220/63221/63224/63225/63226/63228/63231/63232/63233/63225/63239/63253/63255/63266/
63269/63273/63275/63276/63282/63283/63284/63285/63286/63287/63288/63294/63297/63298/
63310/63303/63332/63333/63334/63335/63336/63337 having a similar structure to 63194 (402-50) of
Table 1, as described in WO2009129548A1.
C0009/C0007-5/C0010/C0008/C00142/H0001/R00141/R00142/R00142-
1/D0018/D0017/D0016/D0014/D0015/63290/63291/63304/63313/63317/63318/63312/63314/63329
having a similar structure to 63311 of Table 1, as described in WO2009146218A2.
Compound63161/63162/63164/63129/63163/63177/63190/63200/63251/
63301/63302/63307/63309/63316/63320 having a similar structure to 63165 (402-09) and 63166 (402-
10) of Table 1, as described in WO2009146216A2.
TP-292/TP-342/TP-343/TP-344/TP-345/TP-346 compounds having a similar structure to TP-321 of
Table 1, as described in WO2010053817A1.
TX63341/TX63342/TX63363/TX63364/TX63432/TX63436/TX63444/TX63445/TX63450/TX63451/TX63454/
TX63455/TX63456/TX63462/TX63463/TX63464/TX63465/TX63466/TX63467/TX63468/TX63485/
TX63486/TX63487/TX63491/TX63503/TX63504/TX63505/TX63506/TX63507/TX63508/TX63509/TX63512/
TX63513/TX63514/TX63515/TX63519/TX63524/TX63528/TX63529/TX63531/TX63533/TX63534/
TX63540/TX63542/TX63544/TX63547/TX63550/TX63551/TX63552/TX63553/TX63554/TX63559/TX63561/
TX63567/TX63568/TX63569/TX63570/TX63578/TX63579/TX63582/TX63583/TX63584/TX63586/
TX63587/TX63588/TX63589/TX63590/TX63591/TX63594/TX63595/TX63598/TX63599/TX63600/TX63601/
TX63603/TX63604/TX63605/TX63606/TX63607/TX63611/TX63612/TX63613/TX63615/TX63617/
TX63619/TX63625/TX63626/TX63627/TX63628/TX63629/TX63630/TX63631/TX63636/TX63637/TX63641/
TX63642/TX63646/TX63649/TX63650/TX63651/TX63655/TX63656/TX63659/TX63663/TX63664/
TX63665/TX63666/TX63667/TX63676/TX63690/TX63691/TX63692/TX63714/TX63718/TX63719/TX63720/
TX63721/TX63722/TX63723/TX63725/TX63727/TX63728/TX63729/TX63730/TX63731/TX63734/
TX63735/TX63736/TX63748/TX63757/TX63758/TX63760/TX63761/TX63769/TX63773/TX63774/TX63776/
TX63791/TX63792/TX63793/TX63794/TX63803/TX63804/TX63827/TX63829/TX63834/TX63836/
TX63848/TX63849/TX63850/TX63851/TX63852/TX63853/TX63857/TX63871/TX63872/TX63874/TX63900/
TX63905 compounds having a similar structure to TX63541 and TX63724 of Table 1, as described
in WO2012083306A2.
TX63435/TX63448/TX63520/TX63521/TX63522/TX63523/TX63545/TX63546/TX63555/TX63556/TX63557/
TX63558/TX63597/TX63614/TX63616/TX63618/TX63620/TX63621/TX63622/TX63680/TX63681/
TX63693/TX63716/TX63717/TX63749/TX63778/TX63779/TX63784/TX63785/TX63786/TX63787/TX63788/
TX63789/TX63790/TX63795/TX63797/TX63798/TX63799/TX63800/TX63807/TX63811/TX63812/
TX63814/TX63815/TX63816/TX63817/TX63818/TX63819/TX63820/TX63821/TX63822/TX63823/TX63824/
TX63825/TX63826/TX63830/TX63831/TX63832/TX63833/TX63839/TX63840/TX63841/TX63842/
TX63843/TX63858/TX63859/TX63860/TX63862/TX63863/TX63864/TX63865/TX63866/TX63867/TX63869/
TX63870/TX63875/TX63876/TX63877/TX63878/TX63880/TX63881/TX63882/TX63886/TX63887/
TX63888/TX63889/TX63890/TX63891/TX63892/TX63893/TX63901/TX63904/TX63907/TX63908/TX63909/
TX63910/TX63911/TX63914/TX63915/TX63916/TX63918/TX63919/TX63920/TX63923/TX63925/
TX63928/TX63929/TX63936/TX63982/TX63984 compounds having a similar structure to TX63435 and
TX63545 of Table 1, as described in WO2012125488A1.
TX63098/TX63100/TX63158/TX63160/TX63210/TX63211/TX63212/TX63241/TX63315/TX63392/TX63538/
TX63539/TX63647/TX63654/TX63813/TX63924/TX63947/TX63960/TX63962/TX63966/TX63967/
TX64343 compounds having a similar structure to TX63537 and TX63946 of Table 1, as described in
WO2013188818A1.
TX63384/TX63475/TX63476/TX63477/TX63478/TX63479/TX63593 compounds having a similar
structure to TX63501 of Table 1, as described in WO2014040056A1.
TX63403/TX63608/TX63609/TX63610/TX63742/TX63743/TX63744/TX63762/TX63763/TX63764/TX63868/
TX63926/TX63927/TX63930/TX63949/TX63950/TX63981/TX63983/TX64042/TX64043/TX64044/
TX64045/TX64046/TX64047/TX64048/TX64049/TX64052/TX64053/TX64054/TX64055/TX64056/TX64057/
TX64058/TX64059/TX64060/TX64064/TX64065/TX64066/TX64067/TX64068/TX64074/TX64124/
TX64135 compounds having a similar structure to TX63768 and TX63770of Table 1, as described in
WO2014040060A1.
TX63421/TX63469/TX63489/TX63498/TX63499/TX63536/TX63481/TX63484/TX63496
compounds
having a similar structure to TX63490 and TX63530 of Table 1, as described in WO2014040073A1.
Example 1/2/3/4/5/6/7/8/9/10/11/12/13/14/15/16/17/18/19/20/21/22/23/24/25/27/28/29/30/31/32/33/34/35/
36/37/38/39/40/41/42/43/44/45/46/47/48/49/50/51/52/53/54/55/56/57/58/59/60/61/62/63/64/65/66/67/
68/69/70/71/72/73/74/75/76/77/78/79/80/81/82/83/84/85/86/87/88/89/90/91/92/93/94/95/96/97/98/99/100/
101/102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/
148/149/150/151/152/153/154/155/156/157/158/159/160/161/162/163/164/165/166/167/168/169/170/
171/172/173/174/175/176/177/178 compounds having a similar structure to Example 26 and
Example179 of Table 1, as described in WO2015112792A1.
Compound
T1/T2/T3/T4/T5/T6/T7/T8/T9/T10/T11/T13/T14/T15/T16/T17/T18/T19/T20/T21/T22/T23/T24/T25/T26/T27/
T28 compounds having a similar structure to T12 of Table 1, as described in WO2016130927A1.
Compound
T1/T2/T3/T4/T5/T6/T7/T8/T9/T10/T11/T12/T13/T14/T15/T16/T17/T18/T19/T20/T21/T22/T23/T24/T25/T26/
T27/T28/T29/T30/T31/T32/T33/T34/T35/T36/T37/T38/T39/T40/T41/T42/T43/T44/T45/T46/T47/T49/
T50/T51 compounds having a similar structure to T48 of Table 1, as described in WO2017053868A1.
Compound
T1/T2/T3/T4/T5/T6/T7/T8/T9/T10/T11/T12/T13/T14/T15/T16/T17/T18/T19/T20/T21/T22/T23/T24/T25/T26/
T27/T28/T29/T30/T31/T32/T33/T34/T35/T36/T37/T38/T39/T40/T41/T42/T43/T44/T45/T46/T47/T48/
T49/T50/T51/T52/T53/T54/T55/T56/T57/T58/T59/T60/T61/T62/T63/T64/T65/T66/T67/T68/T69/T70/T71/
T72/T73/T74/T75/T76/T77/T78/T79/T80/T81/T82/T83/T84/T85/T86/T87/T88/T89/T90/T91/T92/T93/T94/
T95/T96/T97/T98/T99/T100/T101/T102/T103/T104/T105/T106/T107/T108/T109/T110/T111/T112/T13/
T114/T115/T116/T117/T118/T119/T120/T121/T122/T123/T124/T125/T126/T127/T128/T129/T130/T131/
T132/T133/T134/T135/T136/T137/T138/T139/T140/T141/T142/T143/T144/T145/T146/T147/T148/T149/
T150/T151/T152/T153/T154/T155/T156/T157/T160/T161/T162/T163/T164/T165/T166/T167/T168/T169/
T170/T171/T172/T173/T174/T175/T176/T177/T178/T179/T180/T181/T182/T183/T184/T185/T186/
T187 compounds having a similar structure to T158 and T159 of Table 1, as described in
WO2018111315A1.
Compound
T1/T2/T3/T4/T5/T6/T7/T8/T9/T10/T11/T12/T13/T14/T15/T16/T17/T18/T19/T20/T21/T22/T23/T24/T25/T26/
T27/T28/T29/T30/T31/T32/T33/T34/T35/T36/T37/T38/T39/T40/T41/T42/T43/T44/T46/T47/T48/T49/
T50/T51/T52/T53/T54/T55/T56/T57/T58/T59/T60/T61/T62/T63/T64/T65/T66/T67/T68/T69/T70/T71/T72/
T73/T74/T75/T76/T77/T78/T79/T80/T81/T82/T84/T85/T86/T87/T88/T89/T90/T91/T92/T93/T94/T95/T96/
T97/T98/T99/T100/T101/T102/T103/T104/T105/T106/T107/T108/T109/T110/T111/T112/T13/T114/T115/
T116/T117/T118/T119/T120/T121/T122/T123/T124/T125/T126/T127/T128/T129/T130/T131/T132/T133/
T134/T135/T136/T137/T138/T139/T140/T141/T142/T143/T144/T145/T146/T147/T148/T149/T150/
T151/T152/T153/T154/T155/T156/T157/T158/T159/T160/T161/T162/T163/T164/T165/T166/T167/T168/
T169/T170/T171/T172/T173/T174/T175/T176/T177/T178/T179/T180/T181/T182/T183/T184/T185/T186
compounds having a similar structure to T45 and T83 of Table 1, as described in WO2019241796A1.
Compound
T1/T2/T3/T4/T5/T6/T7/T8/T9/T10/T11/T12/T13/T14/T15/T16/T17/T18/T19/T20/T21/T22/T23/T24/T25/T26/
T27/T28/T29/T30/T31/T32/T33/T34/T35/T36/T37/T39/T40/T41/T43/T44/T45/T46 compounds having
a similar structure to T38 and T42 of Table 1, as described in WO2021016191A1.
Compound
T3/T4/T5/T6/T7/T8/T9/T10/T11/T12/T13/T14/T15/T17/T18/T19/T20/T21/T22/T23/T24/T25/T26/T27/T28/
T29/T30/T31/T32/T33/T34/T35/T36/T37/T38/T39/T40/T41/T42/T43/T44/T45/T46/T47/T48/T49/T50/T51/
T52/T54/T55/T56/T57/T58/T59/T60/T61/T62/T63/T64/T65/T66/T67/T68/P3 compounds having a
similar structure to T16 and T53 of Table 1, as described in WO2021127480A1.
Compound
T1/T2/T3/T4/T6/T7/T8/T9/T10/T11/T12/T14/T15/T16/T17/T18/T19/T20/T21/T22/T23/T24/T25/T26/T27/
T28/T29/T30/T31/T32/T33/T34/T35/T36/T37/T38/T39/T40/T41/T42/T43/T44/T45/T46/T47/T48/T49/T50/
T51/T52/T53/T54/T55/T56/T57/T58/T59/T60/T61/T62/T63/T64/T65/T66/T67/T68/T69/T70/T71/T72/T73/
T74/T75/T76/T77/T78/T79/T80/T81/T82/T83/T84/T85/T86/T87/T88/T89/T90/T91/T92/T93/T94/T95/
T96/T97/T98/T99/T100/T101/T102/T103/T104/T105/T106/T107 compounds having a similar structure
to T5 and T13 of Table 1, as described in WO2022155614A1.
Compound1/2/3/4/5/6/7/8/9/10/11/12 compounds having a similar structure to Soloxolone methyl and
Soloxolone tryptamide (Compound 12) of Table 1, as described in the paper (10.3390/ph15050603,
Scheme 1).
α-Tocopherol(α-TOH), β-Tocopherol(β-TOH), γ-Tocopherol(γ-TOH), δ-Tocopherol(δ-TOH), α-Tocopheryl
acetate(α-TOA, TA), α-Tocopheryl succinate(α-TOS, VES), α-Tocopheryl maleate(α-TOM), α-Tocopheryl
2-methylsuccinate(α-TO2MS), α-Tocopheryl glutarate(α-TOG), α-Tocopheryl 3-methylglutarate(α-
TO3MG), α-Tocopheryl 3,3 dimethylglutarate(α-TO33DMG), α-Tocopheryl 2,2-dimethylglutarate(α-
TO22DMG), α-Trolox, α-Trolox succinate(α-TroS), β-Tocopheryl succinate(β-ToS), γ-Tocopheryl
succinate(γ-ToS), δ-Tocopheryl succinate(α-ToS), α-Tocotrienol(α-T3H), γ-Tocotrienol(γ-T3H), δ-
Tocotrienol(δ-T3H), γ-Tocotrienyl succinate(γ-T3S), α-2-Geranylchromanol(α-T2H), α-Tocopheryl
succinyl methyl ester(α-TOSM), δ-Tocopheryl succinyl methyl ester(δ-TOSM) having a similar structure
to Tocofersolan (TPGS) of Table 1, as described in the paper (10.1038/sj.bjc.6600981); α-Tocopheryl
Oxalate(TO), α-Tocopheryl Malonate(TM) , α-Tocopheryl pimelate(TP), α-Tocopheryl nicotinate(TN), α-
Tocopheryl succinate ethylester(TSE), as described in the paper (10.1016/j.bbagen.2004.03.001); α-
Tocopheryl adipate(Tadi), α-Tocopheryl butyrate(Tbut) as described in the paper
(10.1016/j.bbrep.2022.101329); δ-Tocopheryl maleate(δ-TOM), α-Tocopheryl amide succinic acid(α-
TAS), δ-Tocopheryl amide succinic acid(δ-TAS), α-Tocopheryl maleamide(α-TAM), δ-Tocopheryl
maleamide(δ-TAM), methyl ester of tocopheryl amide succinic acid(α-TASM) as described in the paper
(10.1002/ijc.21171); Vitamin E analog3/4/5/6/7/8/9 as described in the paper (10.1016/s0960-
894x(98)00438-7); α-Tocopheryl metyl eter(Me-α-TOH) as described in the paper (10.1096/fj.00-
0251com); 6-O-carboxypropyl-α-tocotrienol(T3E), 6-O-carboxypropyl-α-tocopherol(TE) as described in
the paper (10.1002/ijc.20809); α-Tocopheryloxybutyrate(α-TOB) as described in the paper
(pubmed.ncbi.nlm.nih.gov/15736414/); 2,5,7,8-tetramethyl-2R-(4R,8R, 12- trimethyltridecyl)chroman-6-
yloxyacetic acid](α-TEA) as described in the paper (10.1207/s15327914nc4801_13); TS-1/2/3/4/5 as
described in the paper (10.1074/jbc.M511015200); α-Tocopheryl-2-phenylselenyl succinate
(Compound 7a, 7b), γ-Tocopheryl-2-phenylselenyl succinate (Compound 8a, 8b), γ-Tocotrienyl 2-
phenylselenyl succinate (Compound 9a, 9b) as described in the paper (10.1016/j.bmc.2005.11.045);
Treinoin tocoferil, 9-cis retinoic acid a tocopherol ester as described in the paper
(10.1182/blood.V87.8.3384.bloodjournal8783384); α-Tocopheryl phosphate ester (TP), di-α-Tocopheryl
phosphate (T2P) as described in the paper (10.1196/annals. 1331.058); α-Carboxyethyl
hydroxychroman(α-CEHC), γ-Carboxyethyl hydroxychroman(γ-CEHC) as described in the paper
(10.1016/j.abb.2003.11.014); and Desmethyl tocotrienol, Didesmethyl tocotrienol, Marine-derived
tocopherol(MDT), α-Tokomonoenol, α-Tokodienol, δ-Tokomonoenol, Sargochromanol A,
Plastochromanol-8 as described in the paper (10.1007/978-3-030-61879-7_6).
ABC747080 and Compound I/II/III/IV/V having a similar structure to Opaganib (ABC294640) of Table 1,
as described in FIG. 1 of U.S. Pat. No. 8,324,237B2,
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97/98/
99/100/101/102/103/104/105/106/107/108/109/110/111/112/113/114/115/116/117/118/119/120/121/122/
123/124/125/126/127/128/129/130/131/132/133/134/135/136/137 described in Tables 1 and 2 on pages
44 to 53 of WO2006138660A2,
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97
described in Tables 1 to 3 on pages 44 to 52 of WO2007019251A2
Compounds described in claim 36 of WO2017075363A1, compounds described in claims 32, 33, 34, 35,
36, 37 of WO2018119440A1, and compounds described in claims 53, 54, 73, 84 of WO2016210106A1,
having a structure similar to Numidargistat (INCB01158, CB-1158) of Table 1
The compound described in Example80 on pages 63 to 73 of WO2015130790A2 or the compounds
described in claims 12 and 13 of WO2015130790A2, or
Example 1/2/3/4/5/6/7/8/9/10/11/12/13/14/15/16/17/18/19/20/21/22/23/24/25/26/27/28/29/30/31/32/33/34/
35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97/98/99/
100/101/102/103/104/105/106/107/108/109/110/111/112/113/114/115/116/117/118/119/120/121/122/123/
124/125/126/127/128/129/130/131/132/133/134/135/136/137/138/139/140/141/142/143/144/145/146/
147/148/149/150/151/152/153/154/155/156/157/158/159/160/161/162/163 described on pages 74 to
187 of WO2014031936A2, which have a structure similar to IACS-010759 in Table 1
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/A/B/C/D/E/F/G
and LTP073/JAM0351/JAM0359 compounds described in Table 1 on pages 10 to 20 of
WO2017023774A1, Compound1/2/3/4/5/6/7/8/9/10/11/12/13/14/15/16/17/18/19/20/21 described in
Table 1 on pages 9 to 13 of WO201907111OA1,
Compound1/2/3/4/5/6/7/8/9/10/11/12/13/14/15/16/17/18/19/20/21/22/23/24/25/26/27/28/29/5a/5b/5c/5d/
5e/5f/5g/5h/5i/5j/5k/51/5m/8a/8b/8c/8d/8e/8f/8g/8h/8i/8j/8k/81/8m/8n/80/8p/11a/11b/11c/11d/12a/12b/12c/
12d/12e/12f/12g/12h/12i/12j/12k/13a/14a/14b/14c/14d/15a/15b/15c/16a/17a/17b/18a/18b/19a/20a/21a/
22a/23a/24a/25a/26a/28a/28b/28c/30a/30b/31a/31b/31c/31d/31e/31f described in Table1~2 on pages
5 to 28 of WO2022232565A1, which have a structure similar to Sirpiglenastat (DRP-104) in Table 1
Rex1/2/3/4/5/6/7/8/9/10/11/12/13/14/15/16/17/18/19/20/21/22/23/24/25/26/27/28/29/30/31/32/33/34/35/
36/37/38/39/40/41/42/43/44/45/46/47/48/49/50/51/52/53/54/55/56/57/58/59/60/61/62/63/64/65/66/67/68/
69/70/71/72/73/74/75/76/77/78/79/80/81/82/83/84/85/86/87/88/89/90/91/92/93/94/95/96/97/98/99/100/
101/102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/148/
149/150/151/152/153/154/155/156/157/158/159/160/161/162/163/164/165/166/167/168/169/170/171/
172/173/174/175/176/177/178/179/180/181/182/183/184/185/186/187/188/189/190/191/192/193/194/
195/196/197/198/199/200/201/202/203/204/205/206/207/208/209/210/211/212/213/214/215/216/217/218/
219/220/221/222/223/224/225/226/227/228/229/230/231/232/233/234/235/236/237/238/239/240/241/
242/243/244/245/246/247/248/249/250/251/252/253/254/255/256/257/258/259/260/261/262/263/264/
265/266/267/268/269/270/271/272/273/274/275/276/277/278/279/280/281/282/283/284/285/286/287/288/
289/290/291/292/293/294/295/296/297/298/299/300/301/302/303/304/305/306/307/308/309/310/311/
312/313/314/315/316/317/318/319/320/321/322/323/324/325/326/327/328/329/330/331/332/333/334/
335/336/337/338/339/340/341/342/343/344/345/346/347/348/349/350/351/352/353/354/355/356/357/358/
359/360/361/362/363/364/365/366/367/368/369/370/371/372/373/374/375/376/377/378/379/380/381/
382/383/384/385/386/387/388/389/390/391/392/393/394/395/396/397/398/399/400/401/402/403/404/
405/406/407/408/409/410/411/412/413/414/415/416/417/418/419/420/421/422/423/424/425/426/427/428/
429/430/431/432/433/434/435/436/437/438/439/440/441/442/443/444/445/446/447/448/449/450/451/
452/453/454/455/456/457/458/459/460/461/462/463/464/465/466/467/468/469/470/471/472/473/474/
475/476/477/478/479/480/481/482/483/484/485/486/487/488/489/490/491/492/493/494/495/496/497/498/
499/500/501/502/503/504/505/506/507/508/509/510/511/512/513/514/515/516/517/518/519/520/521/
522/523/524/525/526/527/528/529/530/531/532/533/534/535/536/537/538/539/540/541/542/543/544/
545/546/547/548/549/550/551/552/553/554/555/556/557/558/559/560/561/562 compounds described in
Claims 7, 11, and 12 or on pages 85 to 125 of WO201012859A1, or
Ex1/2/3/4/5/6/7/8/9/10/11/12/13/14/15/16/17/18/19/20/21/22/23/24/25/26/27/28/29/30/31/32/33/34/35/36/
37/38/39/40/41/42/43/44/45/46/47/48/49/50/51/52/53/54/55/56/57/58/59/60/61/62/63/64/65/66/67/68/
69/70/71/72/73/74/75/76/77/78/79/80/81/82/83/84/85/86/87/88/89/90/91/92/93/94/95/96/97/98/99/100/101/
102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/148/
149/150/151/152/153/154/155/156/157/158/159/160/161/162/163/164/165/166/167/168/169/170/171/
172/173/174/175/176/177/178/179/180/181/182/183/184/185/186/187/188/189/190/191/192/193/194/
195/196/197/198/199/200/201/202/203/204/205/206/207/208/209/210/211/212/213/214/215/216/217/218/
219/220/221/222/223/224/225/226/227/228/229/230/231/232/233/234/235/236/237/238/239/240/241/
242/243/244/245/246/247/248/249/250/251/252/253/254/255/256/257/258/259/260/261/262/263/264/
265/266/267/268/269/270/271/272/273/274/275/276/277/278/279/280/281/282/283/284/285/286/287/288/
289/290/291/292/293/294/295/296/297/298/299/300/301/302/303/304/305/306/307/308/309/310/311/
312/313/314/315/316/317/318/319/320/321/322/323/324/325/326/327/328/329/330/331/332/333/334/
335/336/337/338/339/340/341/342/343/344/345/346/347/348/349/350/351/352/353/354/355/356/357/358/
359/360/361/362/363/364/365/366/367/368/369/370/371/372/373/374/375/376/377/378/379/380/381/
382/383/384/385/386/387/388/389/390/391/392/393/394/395/396/397/398/399/400/401/402/403/404/
405/406/407/408/409/410/411/412/413/414/415/416/417/418/419/420/421/422/423/424/425/426/427/428/
429/430/431/432/433/434/435/436/437/438/439/440/441/442/443/444/445/446/447/448/449/450/451/
452/453/454/455/456/457/458/459/460/461/462/463/464/465/466/467/468/469/470/471/472/473/474/
475/476/477/478/479/480/481/482/483/484/485/486/487/488/489/490/491/492/493/494/495/496/497/498/
499/500/501/502/503/504/505/506/507/508/509/510/511/512/513/514/515/516/517/518/519/520/521/
522/523/524/525/526/527/528/529/530/531/532/533/534/535/536/537/538/539/540/541/542/543/544/
545/546/547/548/549/550/551/552/553/554/555/556/557/558/559/560/561/562/563/564/565/566/567/568/
569/570/571/572/573/574/575/576/577/578/579/580/581/582 compounds described on pages 163 to
242 of WO2010128659A1, or compounds described on pages 262 to 279 of WO2010128659A1,
Rex1/2/3/4/5/6/7/8/9/10/11/12/13/14/15/16/17/18/19/20/21/22/23/24/25/26/27/28/29/30/31/32/33/34/35/
36/37/38/39/40/41/42/43/44/45/46/47/48/49/50/51/52/53/54/55/56/57/58/59/60/61/62/63/64/65/66/67/68/
69/70/71/72/73/74/75/76/77/78/79/80/81/82/83/84/85/86/87/88/89/90/91/92/93/94/95/96/97/98/99/100/
101/102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/148/
149/150/151/152/153/154/155/156/157/158/159/160/161/162/163/164/165/166/167/168/169/170/171/
172/173/174/175/176/177/178/179/180/181/182/183/184/185/186/187/188/189/190/191/192/193/194/
195/196/197/198/199/200/201/202/203/204/205/206/207/208/209/210/211/212/213/214/215/216/217/218/
219/220/221/222/223/224/225/226/227/228/229/230/231/232/233/234/235/236/237/238/239/240/241/
242/243/244/245/246/247/248/249/250/251/252/253/254/255/256/257/258/259/260/261/262/263/264/
265/266/267/268/269/270/271/272/273/274/275/276/277/278/279/280/281/282/283/284/285/286/287/288/
289/290/291/292/293/294/295/296/297/298/299/300/301/302/303/304/305/306/307/308/309/310/311/
312/313/314/315/316/317/318/319/320/321/322/323/324/325/326/327/328/329/330/331/332/333/334/
335/336/337/338/339/340/341/342/343/344/345/346/347/348/349/350/351/352/353/354/355/356/357/358/
359/360/361/362/363/364/365/366/367/368/369/370/371/372/373/374/375/376/377/378/379/380/381/
382/383/384/385/386/387/388/389/390/391/392/393/394/395/396/397/398/399/400/401/402/403/404/
405/406/407/408/409/410/411/412/413/414/415/416/417/418/419/420/421/422/423/424/425/426/427/428/
429/430/431/432/433/434/435/436/437/438/439/440/441/442/443/444/445/446/447/448/449/450/451/
452/453/454/455/456/457/458/459/460/461/462/463/464/465/466/467/468/469/470/471/472/473/474/
475/476/477/478/479/480/481/482/483/484/485/486/487/488/489/490/491/492/493/494/495/496/497/498/
499/500/501/502/503/504/505/506/507/508/509/510/511/512/513/514/515/516/517/518/519/520/521/
522/523 compounds in Claims 2, 11, 12, 13, 14, 15, 16, 17, 18, 19, or on pages 91-156 of
WO2012053606A1, or
Ex1/2/3/4/5/6/7/8/9/10/11/12/13/14/15/16/17/18/19/20/21/22/23/24/25/26/27/28/29/30/31/32/33/34/35/36/
37/38/39/40/41/42/43/44/45/46/47/48/49/50/51/52/53/54/55/56/57/58/59/60/61/62/63/64/65/66/67/68/
69/70/71/72/73/74/75/76/77/78/79/80/81/82/83/84/85/86/87/88/89/90/91/92/93/94/95/96/97/98/99/100/101/
102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/148/
149/150/151/152/153/154/155/156/157/158/159/160/161/162/163/164/165/166/167/168/169/170/171/
172/173/174/175/176/177/178/179/180/181/182/183/184/185/186/187/188/189/190/191/192/193/194/
195/196/197/198/199/200/201/202/203/204/205/206/207/208/209/210/211/212/213/214/215/216/217/218/
219/220/221/222/223/224/225/226/227/228/229/230/231/232/233/234/235/236/237/238/239/240/241/
242/243/244/245/246/247/248/249/250/251/252/253/254/255/256/257/258/259/260/261/262/263/264/
265/266/267/268/269/270/271/272/273/274/275/276/277/278/279/280/281/282/283/284/285/286/287/288/
289/290/291/292/293/294/295/296/297/298/299/300/301/302/303/304/305/306/307/308/309/310/311/
312/313/314/315/316/317/318/319/320/321/322/323/324/325/326/327/328/329/330/331/332/333/334/
335/336/337/338/339/340/341/342/343/344/345/346/347/348/349/350/351/352/353/354/355/356/357/358/
359/360/361/362/363/364/365/366/367/368/369/370/371/372/373/374/375/376/377/378/379/380/381/
382/383/384/385/386/387/388/389/390/391/392/393/394/395/396/397/398/399/400/401/402/403/404/
405/406/407/408/409/410/411/412/413/414/415/416/417/418/419/420/421/422/423/424/425/426/427/428/
429/430/431/432/433/434/435/436/437/438/439/440/441/442/443/444/445/446/447/448/449/450/451/
452/453/454/455/456/457/458/459/460/461/462/463/464/465/466/467/468/469/470/471/472/473/474/
475/476/477/478/479/480/481/482/483/484/485/486/487/488/489/490/491/492/493/494/495/496/497/498/
499/500/501/502/503/504/505/506/507/508/509/510/511/512/513/514/515/516/517/518/519/520/521/
522/523/524/525/526/527/528/529/530/531/532/533/534/535/536/537/538/539/540/541/542/543/544/
545/546/547/548/549/550/551/552/553/554/555/556/557/558/559/560/561/562/563/564/565/566/567/568/
569/570/571/572/573/574/575/576/577/578/579/580/581/582/583/584/585/586/587/588/589/590/591/
592/593/594/595/596/597/598/599/600/601/602/603/604/605/606/607/608/609/610/611/612/613/614/
615/616/617/618/619/620/621/622/623/624/625/626/627/628/629/630/631/632/633/634/635/636/637/638/
639/640/641/642/643/644/645/646/647/648/649/650/651/652/653/654/655/656/657/658/659/660/661/
662/663/664/665/666/667/668/669/670/671/672/673/674/675/676/677/678/679/680/681/682/683/684/
685/686/687/688/689/690/691/692/693/694/695/696/697/698/699/700/701/702/703/704/705/706/707/708/
709/710/711/712/713/714/715/716/717/718/719/720/721/722/723/724/725/726/727/728/729/730/731/
732/733/734/735/736/737/738/739/740/741/742/743/744/745/746/747/748/749/750/751/752/753/754/
755/756/757/758/759/760/761/762/763/764/765/766/767/768/769/770/771/772/773/774/775/776/777/778/
779/780/781/782/783/784/785/786/787/788/789/790/791/792/793/794/795/796/797/798/799/800/801/
802/803/804/805/806/807/808/809/810/811/812/813/814/815/816/817/818/819/820/821/822/823/824/
825/826/827/828/829/830/831/832/833/834/835/836/837/838/839/840/841/842/843/844/845/846/847/848/
849/850/851/852/853/854/855/856/857/858/859/860/861/862/863/864/865/866/867/868/869/870/871/
872/873/874/875/876/877/878/879/880/881/882/883/884/885/886/887/888/889/890/891/892/893/894/
895 compounds on pages 184 to 309 of WO2012053606A1, or compounds on pages 326 to 349 of
WO2012053606A1,
PEx1/2/3/4/5/6/7/8/9/10/11/12/13/14/15/16/17/18/19/20/21/22/23/24/25/26/27/28/29/30/31/32/33/34/35/
36/37/38/39/40/41/42/43/44/45/46/47/48/49/50/51/52/53/54/55/56/57/58/59/60/61/62/63/64/65/66/67/68/
69/70/71/72/73/74/75/76/77/78/79/80/81/82/83/84/85/86/87/88/89/90/91/92/93/94/95/96/97/98/99/100/
101/102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/148/
149/150/151/152/153/154/155/156/157/158/159/160/161/162/163/164/165/166/167/168/169/170/171/
172/173/174/175/176/177/178/179/180/181/182/183/184/185/186/187/188/189/190/191/192/193/194/
195/196/197/198/199/200/201/202/203/204/205/206/207/208/209/210/211/212/213/214/215/216/217/218/
219/220/221/222/223/224/225/226/227/228/229/230/231/232/233/234/235/236/237/238/239/240/241/
242/243/244/245/246/247/248/249/250/251/252/253/254/255/256/257/258/259/260/261/262/263/264/
265/266/267/268/269/270/271/272/273/274/275/276/277/278/279/280/281/282/283/284/285/286/287/288/
289/290/291/292/293/294/295/296/297/298/299/300/301/302/303/304/305/306/307/308/309/310/311/
312/313/314/315/316/317/318/319/320/321/322/323/324/325/326/327/328/329/330/331/332/333/334/
335/336/337/338/339/340/341/342/343/344/345/346/347/348/349/350/351/352/353/354/355/356/357/358/
359/360/361/362/363/364/365/366/367/368/369/370/371/372/373/374/375/376/377/378/379/380/381/
382/383/384/385/386/387/388/389/390/391/392/393/394/395/396/397/398/399/400/401/402/403/404/
405/406/407/408/409/410/411/412/413/414/415/416/417/418/419/420/421/422/423/424/425/426/427/428/
429/430/431/432/433/434/435/436/437/438/439/440/441/442/443/444/445/446/447/448/449/450/451/
452/453/454/455/456/457/458/459/460/461/462/463/464/465/466/467/468/469/470/471 compounds
in Claim 12 or on pages 101 to 192 of WO2013108754A1, or
Ex1/2/3/4/5/6/7/8/9/10/11/12/13/14/15/16/17/18/19/20/21/22/23/24/25/26/27/28/29/30/31/32/33/34/35/36/
37/38/39/40/41/42/43/44/45/46/47/48/49/50/51/52/53/54/55/56/57/58/59/60/61/62/63/64/65/66/67/68/
69/70/71/72/73/74/75/76/77/78/79/80/81/82/83/84/85/86/87/88/89/90/91/92/93/94/95/96/97/98/99/100/101/
102/103/104/105/106/107/108/109/110/111/112/113/114/115/116/117/118/119/120/121/122/123/124/
125/126/127/128/129/130/131/132/133/134/135/136/137/138/139/140/141/142/143/144/145/146/147/148/
149/150/151/152/153/154/155/156/157/158/159/160/161/162/163/164/165/166/167/168/169/170/171/
172/173/174/175/176/177/178/179/180/181/182/183/184/185/186/187/188/189/190/191/192/193/194/
195/196/197/198/199/200/201/202/203/204/205/206/207/208/209/210/211/212/213/214/215/216/217/218/
219/220/221/222/223/224/225/226/227/228/229/230/231/232/233/234/235/236/237/238/239/240/241/
242/243/244/245/246/247/248/249/250/251/252/253/254/255/256/257/258/259/260/261/262/263/264/
265/266 compounds described on pages 193 to 247 of WO2013108754A1 or No A1/A2/A3 compounds
described on page 257 of WO2013108754A1,
which have a similar structure to Gilteritinib (ASP2215) of Table 1.
CTLA4 inhibitor (e.g., KN046, AK104, BMS-986288, MK-1308, ADG116, AGEN1181, ALPN-202,
MEDI5752, MGD019, ONC-392, Alpha-D-Mannose, Fucose, Tremelimumab, Concatameric CTLA4lg,
CTLA-4-XTEN, AGEN1884, Ipilimumab, BCD-217), PD-1 inhibitor (e.g., Pembrolizumab, Cemiplimab,
Nivolumab, AMP-224, CBT-501, Dostarlimab, Sintilimab, Tislelizumab, Sasanlimab, INCSHR1210,
Retifanlimab, Balstilimab, AK104, JTX-4014, Vopratelimab, RO7121661, 609A, ADPT01, AMG 404, APL-
501, BI 754091, CDX-527, GSK2661380, IBI318, MEDI5752, MGD019, RG6139, 1,2,4-oxadiazole
derivative 1, 1,2,4-oxadiazole derivative 2, 1,3,4-oxadiazole derivative 1, 1,3,4-oxadiazole derivative 2,
which are described in the paper (10.1080/13543776.2018.1512706), Cyclic compound 1, Cyclic
compound 2, Cyclic compound 3, PMID30107136-Compound-Example15/16, AUNP-12, PDR001, CC-
90006, PMID30247903-Compound-General structure 13/14/15/16/17/20/21/22/23/24/25, which is
desribed in the paper (10.1021/acs.jmedchem.8b00990), BCD-217, BCD-100, PD-1 CAR-T cells, which
is described in Table 1 of the paper (10.3389/fimmu.2021.788211)), PD-L1 inhibitor (e.g., Bavencio,
KN046, CX-072, INCB86550, M7824, GS-4224, ALPN-202, BMS-986189, CA-170, FAZ053, IBI318,
INBRX-105, KD033, LY3415244, CA-327, PMID30107136-Compound-Example1/2, which is described
in the paper (10.1080/13543776.2018.1512706), PMID30247903-Comound-General
structure5/6/7/8/9/10/12, which is described in the paper (10.1021/acs.jmedchem.8b00990),
Durvalumab, MEDI4736, MPDL-3280A, NM21-1480, PD-L1 CAR T cells, which is described in the paper
(10.1186/s40364-020-00198-0)), which are immune checkpoint inhibitors, that is, CTLA4 inhibitor, PD-1
inhibitor, PD-L1/2 inhibitor that binds to PD-1, having similar efficacy with IFNgamma (Interferon gamma)
and PD-1 inhibitor of Table 1.
Compound
1a/1b/1c/1d/1e/1f/1g/1h/1i/1j/1k/11/1m/1n/10/1p/1q/1r/1s/1t/1u/1v/1w/1x/1y/1z/2a/2b/2c/2d/2e/2f/2g/2h/
2i/2j/2k/21/2m/2n having a similar structure to SIMR1281 of Table 1, as described in Claim 29 of
WO2019186384A2
Compound1/2/3/4/5/6/7/8/9/10/11/12/13/14/15/16/17/18/19/20/21/22/23/24/25/26/27/28/29/30/31/32
having a similar structure to Compound 31 of Table 1, as described in the paper
(10.1016/j.ejmech.2023.115829)
DA-6 compound having a similar structure to DA-5 of Table 1, as described in Claim 1 of CN115057856A
Compound
1/1a/2/2a/3/3a/4/4a/5/5a/6/6a/7/7a/8/8a/9/9a/10/10a/11/11a/12/12a/13/13a/14/14a/15/15a/16/16a/17/17a/
18/18a/19/19a/20/20a/21/21a/22/22a/23/23a/24/24a/25/25a/26/26a/27/27a/28/28a/29/29a/30/30a
having a similar structure to Compound1a of Table 1, as described in Table1 of WO2024015637A1
Compound 8/9/10/11/12/13/14/15/16/17/18/19/20/21/22/23/24/25/26/27/28/29/30 having a similar
structure to Compound24 of Table 1, as described in Supporting Information of the paper
(10.1021/acs.jmedchem.2c01415)
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97/98/
99/100/101/102 having a similar structure to Compound 28 of Table 1, as described on pages 40-64 of
U. S. PAT. No. 11,040,964B2
Example 1/2/3/4/5/6/7/8/9/10/11/12/13/14/15/16/17/18/19/20/21/23/24/25/26/27/28/29/30/31/32/33/34
compounds having a similar structure to Example 4, 1, 21, 22 of Table 1, as described on pages 32 to 57,
in Table 1, or Claim 11 of WO2022175402A1, and
Example1/2/3/4/5/6/7/8/9/10/11/12/13/14/15/16/17/18/19/20/21/22/23/24/25/26/27/28/29/30/31/32
compounds described on pages 19 to 22 of US20220071970A1
Example 1/2/3/4/5/6/7/8/9/10/11/12/13/14/15/16/17/18/19 compounds having a similar structure to
Example15 of Table 1, as described in Claims 5 and 6 of WO2019015689A1
Example 1/2/3/4/5/6/7/8/9/10/11/12/13/14/15/16/17/18/19/20/21/22/23/24 compounds having a similar
structure to Example 3 and 19 of Table 1, as described in Claims 7 and 8 of WO2019015690A1
Example 1/2/3/4/5/6/7/8/9/10/11/12/13/14/15/16/17/18/19/20/21/22/23/24/25/26/27/28/29/30/31/32
compounds having a similar structure to Example17 of Table 1, as described on pages 8 to 40 of
WO2022204109A1
Compound1A/2A/3A/4A/5A/6A/7A/8A/9A/10A/11A/12A/13A/14A/15A/16A/17A/18A/19A/20A/21A/22A/23A/
24A/25A/26A/27A/28A/29A/30A/31A/32A/33A/34A/35A/36A/37A/38A/39A/40A/41A/42A/43A/44A/45A/
46A/47A/48A/49A/50A/51A/1B/2B/3B/4B/5B/6B/7B/8B/9B/10B/11B/12B/13B/14B/15B/16B/17B/18B/
19B/20B/21B/22B/23B/24B/25B/26B/27B/28B/29B/30B/31B/32B/33B/34B/35B/36B/37B/38B/39B/40B/
41B/42B/43B/44B/45B/46B/47B/48B/49B/50B/51B/52B/53B/54B/55B/56B/57B/58B/59B/60B/61B/62B/
63B/64B/65B/66B/67B/68B/69B/70B/71B/72B/73B/74B/75B/76B/77B/78B/79B/80B/81B/82B/83B/84B/
85B/86B/87B/88B/89B/90B/91B/92B/93B/94B/95B/96B/97B/98B/99B/100B/101B/102B/103B/104B/105B/
106B/107B/108B/109B/110B/111B/112B/113B/114B/115B/116B/117B/118B/119B/120B/121B/122B/123B/
124B/125B/126B/127B/128B/129B/130B/131B/132B/133B/134B/135B/136B/137B/138B/139B/140B/
141B/142B/143B/144B/145B/146B/147B/148B/149B/150B/151B/152B/153B/154B/155B/156B/157B/158B/
159B/160B/161B/162B/163B/164B/165B/166B/167B/168B/169B/170B/171B/172B/173B/174B/175B/
176B/177B/178B/179B/180B/181B/182B/183B/184B/185B/186B/187B/188B/189B/190B/191B/192B/
193B/194B/195B/196B/197B/198B/199B/200B/201B/202B/203B/204B/205B/206B/207B/208B/209B/210B/
211B/212B/213B/214B/215B/216B/217B/218B/219B/220B/221B/222B/223B/224B/225B/226B/227B/
228B/229B/230B/231B/232B/233B/234B/235B/236B/237B/238B/239B/240B/241B/242B/243B/244B/245B/
246B/247B/248B/249B/250B/251B/252B/253B/254B/255B/256B/257B/258B/259B/260B/261B/262B/
263B/264B/265B/266B/267B/268B/269B/270B/271B/272B/273B/274B/275B/276B/277B/278B/279B/
280B/281B/282B/283B/284B/285B/286B/287B/288B/289B/290B/291B/292B/293B/294B/295B/296B/297B/
298B/299B/300B/301B/302B/303B/304B/305B/306B/307B/308B/309B/310B/311B/312B/313B/314B/
315B/316B/317B/318B/319B/320B/321B/322B/323B/324B/325B/326B/327B/328B/329B/330B/331B/332B/
333B/334B/335B/336B/337B/338B/339B/340B/341B/342B/343B/344B/345B/346B/347B/348B/349B/
350B/351B/352B/353B/354B/355B/356B/357B/358B/359B/360B/361B/362B/363B/364B/365B/366B/
367B/368B/369B/370B/371B/372B/373B/374B/375B/376B/377B/378B/379B/380B/381B/382B/383B/38
4B/385B/386B/387B/388B/389B/390B/391B/392B/393B/394B/395B/396B/397B/398B/399B/400B/401B/
402B/403B/404B/405B/406B/407B/408B/409B/410B/411B/412B/413B/414B/415B/416B/417B/418B/419B/
420B/421B/422B/423B/424B/425B/426B/427B/428B/429B/430B/431B/432B/433B/434B/435B/436B/
437B/438B/439B/440B/441B/442B/443B/444B/445B/446B/447B/448B/449B/450B/451B/452B/453B/
454B/455B/456B/457B/458B/459B/460B/461B/462B/463B/464B/465B/466B/467B/468B/469B/470B/471B/
472B/473B/474B/475B/476B/477B/478B/479B/480B/481B/482B/483B/484B/485B/486B/487B/488B/
489B/490B/491B/492B/493B/494B/495B/496B/497B/498B/499B/500B/501B/502B/503B/504B/505B/506B/
507B/508B/509B/510B/511B/512B/513B/514B/515B/516B/517B/518B/519B/520B/521B/522B/523B/
524B/525B/526B/527B/528B/529B/530B/531B/532B/533B/534B/535B/536B/537B/538B/539B/540B/
541B/542B/543B/544B/545B/546B/547B/548B/549B/550B/551B/552B/553B/554B/555B/556B/557B/558B
having a similar structure to Compound1A of Table 1, as described in Table1~9 on pages 73 to 486
of WO2021231474A1
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97/98/
99/100/101/102/103/104/105/106/107/108/109/110/111/112/113/114/115/116/117/118/119/120/121/122/
123/124/125/126/127/128/129/130/131/132/133/134/135/136/137/138/139/140/141/142/143/144/145/146/
147/148/149/150/151/152/153/154/155/156/157/158/159/160/161/162/163/164/165/166/167/168/169/
170/171/172/173/174/175/176/177/178/179/180/181/182/183/184/185/186/187/188/189/190/191/192/
193/194/195/196/197/198/199/200/201/202/203/204/205/206/207/208/209/210/211/212/213/214/215/216/
217/218/219/220/221/222/223/224/225/226/227/228/229/230/231/232/233/234/235/236/237/238/239/
240/241/242/243/244/245/246/247/248/249/250/251/252/253/254/255/256/257/258/259/260/261/262/
263/264/265/266/267/268/269/270/271/272/273/274/275/276/277/278/279/280/281/282/283/284/285/286/
287/288/289/290/291/292/293/294/295/296/297/298/299/300/301/302/303/304/305/306/307/308/309/
310/311/312/313/314/315/316/317/318/319/320/321/322/323/324/325/326/327/328/329/330/331/332/
333/334/335/336/337/338/339/340/341/342/343/344/345/346/347/348/349/350/351/352/353/354/355/356/
357/358/359/360/361/362/363/364/365/366/367/368/369/370/371/372/373/374/375/376/377/378/379/
380/381/382/383/384/385/386/387/388/389/390/391/392/393/394/395/396/397/398/399/400/401/402/
403/404/405/406/407/408/409/410/411/412/413/414/415/416/417/418/419/420/421/422/423/424/425/426/
427/428/429/430/431/432/433/434/435/436/437/438/439/440/441/442/443/444/445/446/447/448/449/
450/451/452/453/454/455/456/457/458/459/460/461/462/463/464/465/466/467/468/469/470/471/472/
473/474/475/476/477/478/479/480/481/482/483/484/485/486/487/488/489/490/491/492/493/494/495/496/
497/498/499/500/501/502/503/504/505/506/507/508/509/510/511/512/513/514/515/516/517/518/519/
520/521/522/523/524/525/526/527/528/529/530/531/532/533/534/535/536/537/538/539/540/541/542/
543/544/545/546/547/548/549/550/551/552/553/554/555/556/557/558/559/560/561/562/563/564/565/566/
567/568/569/570/571/572/573/574/575/576/577/578/579/580/581/582/583/584/585/586/587/588/589/
590/591/592/593/594/595/596/597/598/599/600/601/602/603/604/605/606/607/608/609/610/611/612/
613/614/615/616/617/618/619/620/621/622/623/624/625/626/627/628/629/630/631/632/633/634/635/636/
637/638/639/640/641/642/643/644/645/646/647/648/649/650/651/652/653/654/655/656/657/658/659/
660/661/662/663/664/665/666/667/668/669/670/671/672/673/674/675/676/677/678/679/680/681/682/
683/684/685/686/687/688/689/690/691/692/693/694/695/696/697/698/699/700/701/702/703/704/705/706/
707/708/709/710/711/712/713/714/715/716/717/718/719/720/721/722/723/724/725/726/727/728/729/
730/731/732/733/734/735/736/737/738/739/740/741/742/743/744/745/746/747/748/749/750/751/752/
753/754/755/756/757/758/759/760/761/762/763/764/765/766/767/768/769/770/771/772/773/774/775/776/
777/778/779/780/781/782/783/784/785/786/787/788/789/790/791/792/793/794/795/796/797/798/799/
800/801/802/803/804/805/806/807/808/809/810/811/812/813/814/815/816/817/818/819/820/821/822/
823/824/825/826/827/828/829/830/831/832/833/834/835/836/837/838/839/840/841/842/843/844/845/846/
847/848/849/850/851/852/853/854/855/856/857/858/859/860/861/862/863/864/865/866/867/868/869/
870/871/872/873/874/875/876/877/878/879/880/881/882/883/884/885/886/887/888/889/890/891/892/
893/894/895/896/897/898/899/900/901/902/903/904/905/906/907/908/909/910/911/912/913/914/915/916/
917/918/919/920/921/922/923/924/925/926/927/928/929/930/931/932/933/934/935/936/937/938/939/
940/941/942/943/944/945/946/947/948/949/950/951/952/953/954/955/956/957/958/959/960/961/962/
963/964/965/966/967/968/969/970/971/972/973/974/975/976/977/978/979/980/981/982/983/984/985/986/
987/988/989/990/991/992/993/994/995/996/997/998/999/1000/1001/1002/1003/1004/1005/1006/1007/
1008/1009/1010/1011/1012/1013/1014/1015/1016/1017/1018/1019/1020/1021/1022/1023/1024/1025/
1026/1027/1028/1029/1030/1031/1032/1033/1034/1035/1036/1037/1038/1039/1040/1041/1042/1043/
1044/1045/1046/1047/1048/1049/1050/1051/1052/1053/1054/1055/1056/1057/1058/1059/1060/1061/
1062/1063/1064/1065/1066/1067/1068/1069/1070/1071/1072/1073/1074/1075/1076/1077/1078/1079/1080/
1081/1082/1083/1084/1085/1086/1087/1088/1089/1090/1091/1092/1093/1094/1095/1096/1097/1098/
1099/1100/1101/1102/1103/1104/1105/1106/1107/1108/1109/1110/1111/1112/1113/1114/1115/1116/1117/
1118/1119/1120/1121/1122/1123/1124/1125/1126/1127/1128/1129/1130/1131/1132/1133/1134/1135/1136/
1137/1138/1139/1140/1141/1142/1143/1144/1145/1146/1147/1148/1149/1150/1151/1152/1153/1154/
1155/1156/1157/1158/1159/1160/1161/1162/1163/1164/1165/1166/1167/1168/1169/1170/1171/1172/1173/
1174/1175/1176/1177/1178/1179/1180/1181/1182/1183/1184/1185/1186/1187/1188/1189/1190/1191/1192/
1193/1194/1195/1196/1197/1198/1199/1200/1201/1202/1203/1204/1205/1206/1207/1208/1209/1210/
1211/1212/1213/1214/1215/1216/1217/1218/1219/1220/1221/1222/1223/1224/1225/1226/1227/1228/
1229/1230/1231/1232/1233/1234/1235/1236/1237/1238/1239/1240/1241/1242/1243/1244/1245/1246/1247/
1248/1249/1250/1251/1252/1253/1254/1255/1256/1257/1258/1259/1260/1261/1262/1263/1264/1265/
1266/1267/1268/1269/1270/1271/1272/1273/1274/1275/1276/1277/1278/1279/1280/1281/1282/1283/
1284/1285/1286/1287/1288/1289/1290/1291/1292/1293/1294/1295/1296/1297/1298/1299/1300/1301/
1302/1303/1304/1305/1306/1307/1308/1309/1310/1311/1312/1313/1314/1315/1316/1317/1318/1319/
1320/1321/1322/1323/1324/1325/1326/1327/1328/1329/1330/1331/1332/1333/1334/1335/1336/1337/1338/
1339/1340/1341/1342/1343/1344/1345/1346/1347/1348/1349/1350/1351/1352/1353/1354/1355/1356/
1357/1358/1359/1360/1361/1362/1363/1364/1365/1366/1367/1368/1369/1370/1371/1372 having a
similar structure to Compound 54 of Table 1, as described in Table1~16 on pages 50 to 540 of
WO2021262596A1
Compound1/2/3/4/5/6/7/8/9/10/11/12/13/14/15/16/17/18/19/20/21/22/23/24/25/26/27/28/29/30/31/32/33/
34/35/36/37/38/39/40/41/42/43/44/45/46/47/48/49/50/51/52/53/54/55/56/57/58/59/60/61/62/63/64/65/66/
67/68/69/70/71/72/73/74/75/76/77/78/79/80/81/82/83/84/85/86/87/88/89/90/91/92/93/94/95/96/97/98/
99/100/101/102/103/104/105/106/107/108/109/110/111/112/113/114/115/116/117/118/119/120/121/122/
123/124/125/126/127/128/129/130/131/132/133/134/135/136/137/138/139/140/141/142/143/144/145/146/
147/148/149/150/151/152/153/154/155/156/157/158/159/160/161/162/163/164/165/166/167/168/169/
170/171/172/173/174/175/176/177/178/179/180/181/182/183/184/185/186/187/188/189/190/191/192/
193/194/195/196/197/198/199/200/201/202/203/204/205/206/207/208/209/210/211/212/213/214/215/216/
217/218/219/220/221/222/223/224/225/226/227/228/229/230/231/232/233/234/235/236/237/238/239/
240/241/242/243/244/245/246/247/248/249/250/251/252/253/254/255/256/257/258/259/260/261/262/
263/264/265/266/267/268/269/270/271/272/273/274/275/276/277/278/279/280/281/282/283/284/285/286/
287/288/289/290/291/292/293/294/295/296/297/298/299/300/301/302/303/304/305/306/307/308/309/
310/311/312/313/314/315/316/317/318/319/320/321/322/323/324/325/326/327/328/329/330/331/332/
333/334/335/336/337/338/339/340/341/342/343/344/345/346/347/348/349/350/351/352/353/354/355/356/
357/358/359/360/361/362/363/364/365/366/367/368/369/370/371/372/373/374/375/376/377/378/379/
380/381/382/383/384/385/386/387/388/389/390/391/392/393/394/395/396/397/398/399/400/401/402/
403/404/405/406/407/408/409/410/411/412/413/414/415/416/417/418/419/420/421/422/423/424/425/426/
427/428/429/430/431/432/433/434/435/436/437/438/439/440/441/442/443/444/445/446/447/448/449/
450/451/452/453/454/455/456/457/458/459/460/461/462/463/464/465/466/467/468/469/470/471/472/
473/474/475/476/477/478/479/480/481/482/483/484/485/486/487/488/489/490/491/492/493/494/495/496/
497/498/499/500/501/502/503/504/505/506/507/508/509/510/511/512/513/514/515/516/517/518/519/
520/521/522/523/524/525/526/527/528/529/530/531/532/533/534/535/536/537/538/539/540/541/542/
543/544/545/546/547/548/549/550/551/552/553/554/555/556/557/558/559/560/561/562/563/564/565/566/
567/568/569/570/571/572/573/574/575/576/577/578/579/580/581/582/583/584/585/586/587/588/589/
590/591/592/593/594/595/596/597/598/599/600/601/602/603/604/605/606/607/608/609/610/611/612/
613/614/615/616/617/618/619/620/621/622/623/624/625/626/627/628/629/630/631/632/633/634/635/636/
637/638/639/640/641/642/643/644/645/646/647/648/649/650/651/652/653/654/655/656/657/658/659/
660/661/662/663/664/665/666/667/668/669/670/671/672/673/674/675/676/677/678/679/680/681/682/
683/684/685/686/687/688/689/690/691/692/693/694/695/696/697/698/699/700/701/702/703/704/705/706/
707/708/709/710/711/712/713/714/715/716/717/718/719/720/721/722/723/724/725/726/727/728/729/
730/731/732/733/734/735/736/737/738/739/740/741/742/743/744/745/746/747/748/749/750/751/752/
753/754/755/756/757/758/759/760/761/762/763/764/765/766/767/768/769/770/771/772/773/774/775/776/
777/778/779/780/781/782/783/784/785/786/787/788/789/790/791/792/793/794/795/796/797/798/799/
800/801/802/803/804/805/806/807/808/809/810/811/812/813/814/815/816/817/818/819/820/821/822/
823/824/825/826/827/828/829/830/831/832/833/834/835/836/837/838/839/840/841/842/843/844/845/846/
847/848/849/850/851/852/853/854/855/856/857/858/859/860/861/862/863/864/865/866/867/868/869/
870/871/872/873/874/875/876/877/878/879/880/881/882/883/884/885/886/887/888/889/890/891/892/
893/894/895/896/897/898/899/900/901/902/903/904/905/906/907/908/909/910/911/912/913/914/915/916/
917/918/919/920/921/922/923/924/925/926/927/928/929/930/931/932/933/934/935/936/937/938/939/
940/941/942/943/944/945/946/947/948/949/950/951/952/953/954/955/956/957/958/959/960/961/962/
963/964/965/966/967/968/969/970/971/972/973/974/975/976/977/978/979/980/981/982/983/984/985/986/
987/988/989/990/991/992/993/994/995/996/997/998/999/1000/1001/1002/1003/1004/1005/1006/1007/
1008/1009/1010/1011/1012/1013/1014/1015/1016/1017/1018/1019/1020/1021/1022/1023/1024/1025/
1026/1027/1028/1029/1030/1031/1032/1033/1034/1035/1036/1037/1038/1039/1040/1041/1042/1043/
1044/1045/1046/1047/1048/1049/1050/1051/1052/1053/1054/1055/1056/1057/1058/1059/1060/1061/
1062/1063/1064/1065/1066/1067/1068/1069/1070/1071/1072/1073/1074/1075/1076/1077/1078/1079/1080/
1081/1082/1083/1084/1085/1086/1087/1088/1089/1090/1091/1092/1093/1094/1095/1096/1097/1098/
1099/1100/1101/1102/1103/1104/1105/1106/1107/1108/1109/1110/1111/1112/1113/1114/1115/1116/1117/
1118/1119/1120/1121/1122/1123/1124/1125/1126/1127/1128/1129/1130/1131/1132/1133/1134/1135/1136/
1137/1138/1139/1140/1141/1142/1143/1144/1145/1146/1147/1148/1149/1150/1151/1152/1153/1154/
1155/1156/1157/1158/1159/1160/1161/1162/1163/1164/1165/1166/1167/1168/1169/1170/1171/1172/1173/
1174/1175/1176/1177/1178/1179/1180/1181/1182/1183/1184/1185/1186/1187/1188/1189/1190/1191/1192/
1193/1194/1195/1196/1197/1198/1199/1200/1201/1202/1203/1204/1205/1206/1207/1208/1209/1210/
1211/1212/1213/1214/1215/1216/1217/1218/1219/1220/1221/1222/1223/1224/1225/1226/1227/1228/
1229/1230/1231/1232/1233/1234/1235/1236/1237/1238/1239/1240/1241/1242/1243/1244/1245/1246/1247/
1248/1249/1250/1251/1252/1253/1254/1255/1256/1257/1258/1259/1260/1261/1262/1263/1264/1265/
1266/1267/1268/1269/1270/1271/1272/1273/1274/1275/1276/1277/1278/1279/1280/1281/1282/1283/
1284/1285/1286/1287/1288/1289/1290/1291/1292/1293/1294/1295/1296/1297/1298/1299/1300/1301/
1302/1303/1304/1305/1306/1307/1308/1309/1310/1311/1312/1313/1314/1315/1316/1317/1318/1319/
1320/1321/1322/1323/1324/1325/1326/1327/1328/1329/1330/1331/1332/1333/1334/1335/1336/1337/1338/
1339/1340/1341/1342/1343/1344/1345/1346/1347/1348/1349/1350/1351/1352/1353/1354/1355/1356/
1357/1358/1359/1360/1361/1362/1363/1364/1365/1366/1367/1368/1369/1370/1371/1372/1373/1374/
1375 having a similar structure to Compound 684 of Table 1, as described in Table1~14 on pages 98
to 527 of WO2021262684A1
Thio-FAC compound having a similar structure to 4'-thio-5-aza-2'-deoxycytidine of Table 1, as described
in Claim 7 of WO2022031150A1

A preferred embodiment of the present invention provides a composition for removing senescent cells, comprising as an active agent (a) the compound of Table 1 or its pharmaceutically acceptable salt; (b) secreted fluid from cells killed by treatment with the compound of Table 1 or its pharmaceutically acceptable salt; or (c) cells killed by treatment with the compound of Table 1 or its pharmaceutically acceptable salt.

In a preferred embodiment of the present invention, the compound having the effect of removing senescent cells is at least one selected from the group consisting of the compounds specified in Table 1 above. For many purposes of the present invention, the compounds disclosed in Table 1 were more preferred.

In one embodiment of the invention, the CTLA4 inhibitor includes, for example, KN046, AK104, BMS-986288, MK-1308, ADG116, AGEN1181, ALPN-202, MEDI5752, MGD019, ONC-392, Alpha-D-Mannose, Fucose, Tremelimumab, Concatameric CTLA4Ig, CTLA-4-XTEN, AGEN1884, Ipilimumab, BCD-217, etc.

In one embodiment of the present invention, the PD-1 inhibitor includes, for example, Pembrolizumab, Cemiplimab, Nivolumab, AMP-224, CBT-501, Dostarlimab, Sintilimab, Tislelizumab, Sasanlimab, INCSHR1210, Retifanlimab, Balstilimab, AK104, JTX-4014, Vopratelimab, RO7121661, 609A, ADPT01, AMG 404, APL-501, BI 754091, CDX-527, GSK2661380, IBI318, MEDI5752, MGD019, RG6139, 1,2,4-oxadiazole derivative 1, 1,2,4-oxadiazole derivative 2, 1,3,4-oxadiazole derivative 1, 1,3,4-oxadiazole derivative 2, Cyclic compound 1, Cyclic compound 2, Cyclic compound 3, PMID30107136-Compound-Example 15/16, AUNP-12, PDR001, CC-90006, PMID30247903-Compound-General structure 13/14/15/16/17/20/21/22/23/24/25, BCD-217, BCD-100, PD-1 CAR T cells, etc. The structures of some of these compounds are as follows:

The paper (10.1080/13543776.2018.1512706) = PMID30107136

| | |
|---|---|
| 1,2,4-oxadiazole derivative 1 = PMID30107136- Compound- Example 39 | 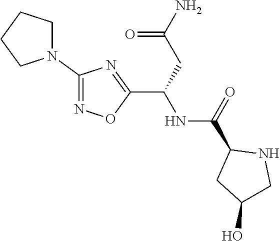 |
| | Example 39 |
| 1,2,4-oxadiazole derivative 2 = PMID30107136- Compound- Example 40 | 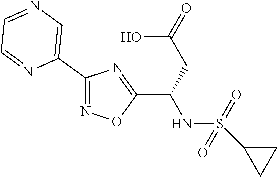 |
| | Example 40 |
| 1,3,4-oxadiazole derivative 1 = PMID30107136- Compound- Example 37 | 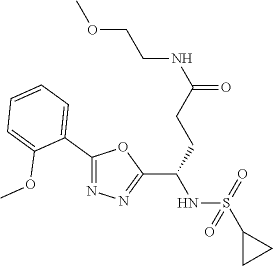 |
| | Example 37 |
| 1,3,4-oxadiazole derivative 2 = PMID30107136- Compound- Example 38 | 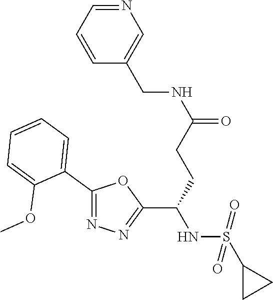 |
| | Example 38 |
| | 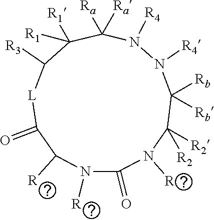 |
| | General structure 39 |
| Cyclic compound 1 = PMID30107136- Compound- Example 80 | 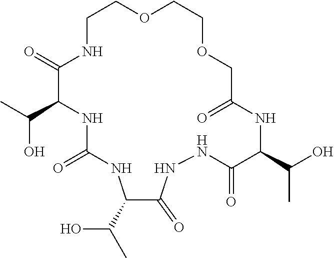 |
| | Example 80 |
| | Splnocyte proliferation recovery 91% |
| Cyclic compound 2 = PMID30107136- Compound- Example 81 | 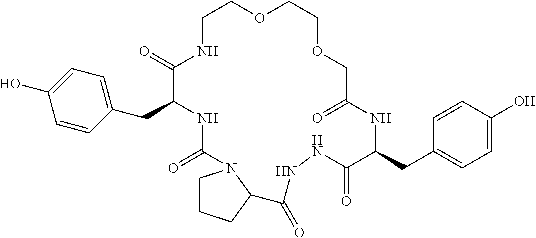 |
| | Example 81 |
| Cyclic compound 3 = PMID30107136- Compound- Example 82 | 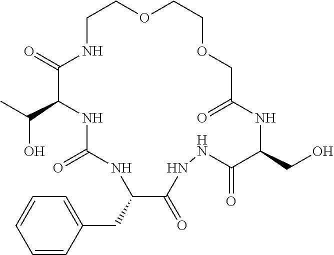 |
| | Example 82 |
| PMID30107136- Compound- Example15 | 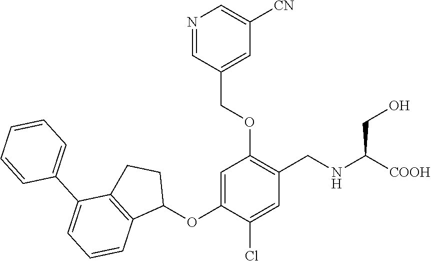 |
| | Example 15 |
| PMID30107136- Compound- Example 16 | 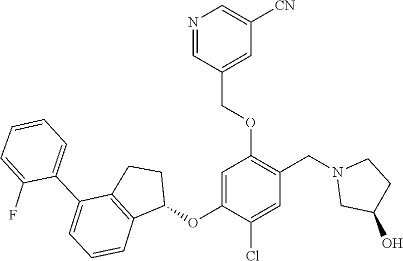 |
| | Example 16 |

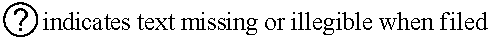

The paper (10.1021/acs.jmedchem.8b00990) = PMID30247903

| PMID30247903- Compound-General structure13 | 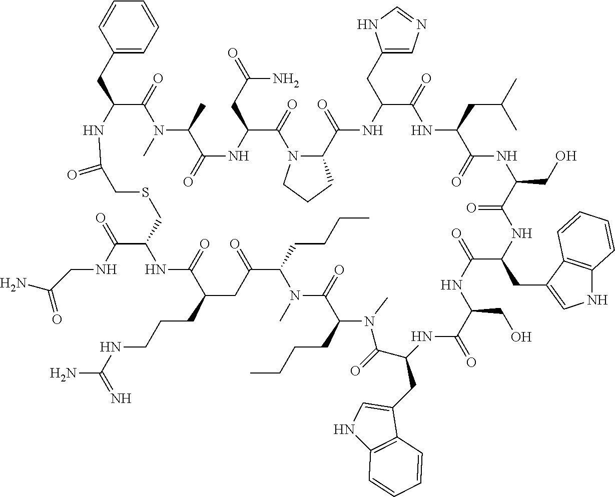 |
|---|---|
| | 13 |
| PMID30247903- Compound-General structure 14 | 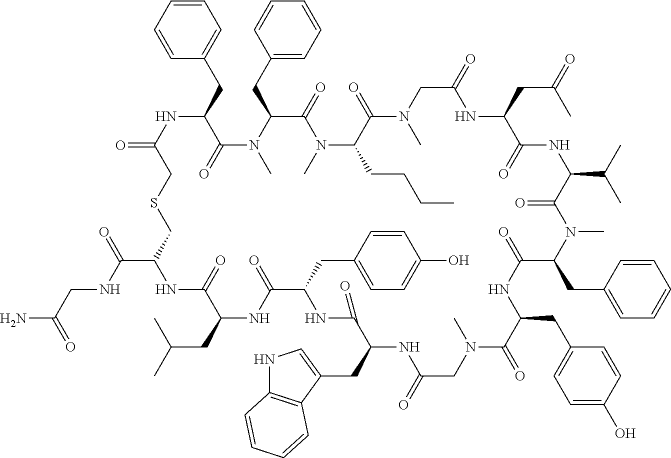 |
| | 14 |
| PMID30247903- Compound-General structure 15 | 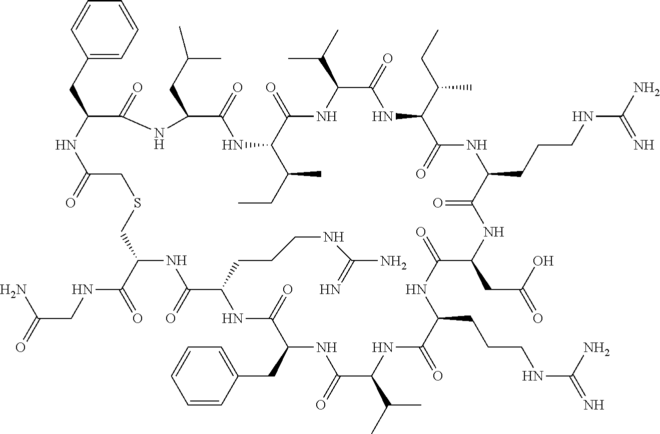 [SMILES: CC[C@H](C)[C@@H]1NC(=O)[C@H](CC(C)C)NC(=O)[C@H](Cc2ccccc2)NC(=O)CSC[C@@H](C(=O)NCC(N)=O)NC(=O)[C@H](CCCNC(=N)N)NC(=O)[C@H](Cc2ccccc2)NC(=O)[C@H](C(C)C)NC(=O)[C@H](CCCNC(=N)N)NC(=O)[C@H](CC(=O)O)NC(=O)[C@H](CCCNC(=N)N)NC(=O)[C@H]([C@@H](C)CC)NC(=O)[C@H](C(C)C)NC1=O] |
| | 15 |
| PMID30247903- Compound-General structure 16 | 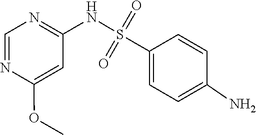 [SMILES: COc1cc(NS(=O)(=O)c2ccc(N)cc2)ncn1] |
| | 16 |
| PMID30247903- Compound-General structure 17 | 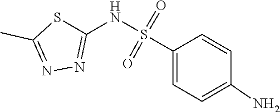 [SMILES: Cc1nnc(NS(=O)(=O)c2ccc(N)cc2)s1] |
| | 17 |
| PMID30247903- Compound-General structure20 | 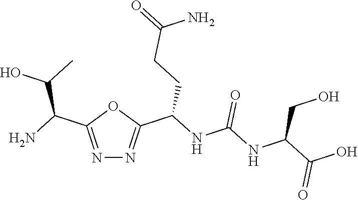 [SMILES: CC(O)[C@H](N)c1nnc([C@H](CCC(N)=O)NC(=O)N[C@@H](CO)C(=O)O)o1] |
| | 20 |
| PMID30247903- Compound-General structure 21 | 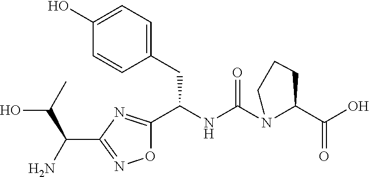 [SMILES: CC(O)[C@H](N)c1noc([C@H](Cc2ccc(O)cc2)NC(=O)N2CCC[C@H]2C(=O)O)n1] |
| | 21 |
| PMID30247903- Compound-General structure 22 | 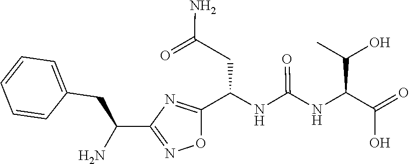 [SMILES: CC(O)[C@H](NC(=O)N[C@@H](CCC(N)=O)c1nc([C@@H](N)Cc2ccccc2)no1)C(=O)O] |
| | 22 |
| PMID30247903- Compound-General structure 23 | 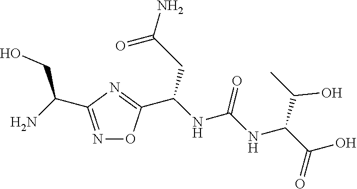 [SMILES: CC(O)[C@@H](NC(=O)N[C@@H](CCC(N)=O)c1nc([C@@H](N)CO)no1)C(=O)O] |
| | 23 |
| PMID30247903- Compound-General structure 24 | 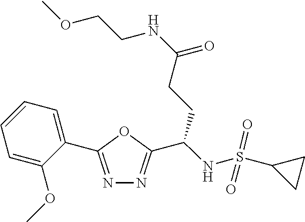 [SMILES: COCCNC(=O)CC[C@H](NS(=O)(=O)C1CC1)c1nnc(-c2ccccc2OC)o1] |
| | 24 |
| PMID30247903- Compound-General structure 25 | 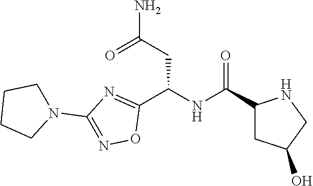 |
| | 25 |

In one embodiment of the present invention, the PD-L1 inhibitor includes, for example, Bavencio, KN046, CX-072, INCB86550, M7824, GS-4224, ALPN-202, BMS-986189, CA-170, FAZ053, IBI318, INBRX-105, KD033, LY3415244, CA-327, PMID30107136-Compound-Example 1/2, PMID30247903-Compound-General structure5/6/7/8/9/10/12, Durvalumab, MEDI4736, MPDL-3280A, NM21-1480, PD-L1 CAR T cells, etc. The structures of some of these compounds are as follows:

The paper (10.1080/13543776.2018.1512706) = PMID30107136

| | |
|---|---|
| PMID30107136- Compound-Example 1 | 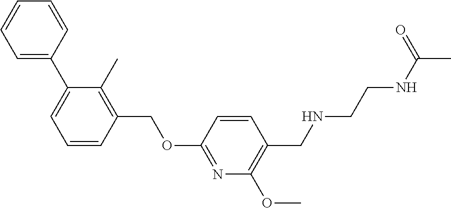 [SMILES: COc1nc(OCc2cccc(-c3ccccc3)c2C)ccc1CNCCNC(C)=O] |
| | Example 1 |
| | IC [symbol] 18 nm |
| PMID30107136- Compound-Example 2 | 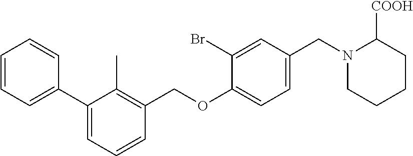 [SMILES: Cc1c(COc2ccc(CN3CCCCC3C(=O)O)cc2Br)cccc1-c1ccccc1] |
| | Example 2 |
| | IC [symbol] 146 nM |

The paper (10.1021/acs.jmedchem.8b00990) = PMID30247903

| | |
|---|---|
| PMID30247903- Compound-General structure 5 | 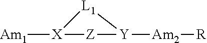 |
| | 5 |
| PMID30247903- Compound-General structure 6 | 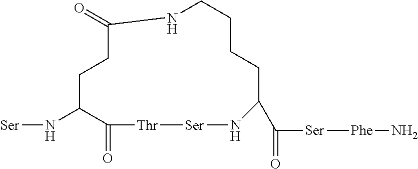 |
| | 6 |
| PMID30247903- Compound-General structure 7 | 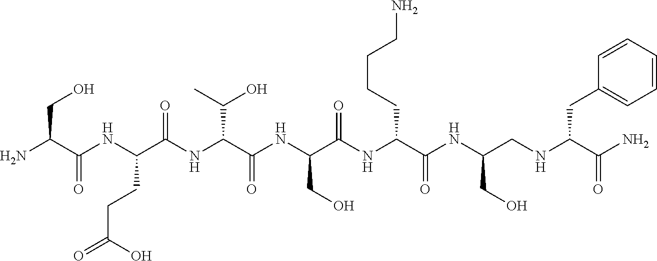 [SMILES: CC(O)[C@@H](NC(=O)[C@H](CCC(=O)O)NC(=O)[C@@H](N)CO)C(=O)N[C@H](CO)C(=O)N[C@H](CCCCN)C(=O)N[C@H](CO)CN[C@H](Cc1ccccc1)C(N)=O] |
| | 7 |
| PMID30247903- Compound-General structure 8 | 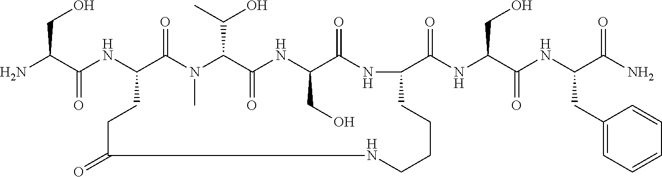 [SMILES: CC(O)[C@@H]1C(=O)N[C@H](CO)C(=O)N[C@H](C(=O)N[C@@H](CO)C(=O)N[C@@H](Cc2ccccc2)C(N)=O)CCCCNC(=O)CC[C@H](NC(=O)[C@@H](N)CO)C(=O)N1C] |
| | 8 |
| PMID30247903- Compound-General structure 9 | 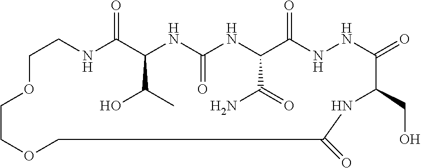 [SMILES: CC(O)[C@@H]1NC(=O)N[C@@H](C(N)=O)C(=O)NNC(=O)[C@@H](CO)NC(=O)COCCOCCNC1=O] |
| | 9 |
| PMID30247903- Compound-General structure 10 | 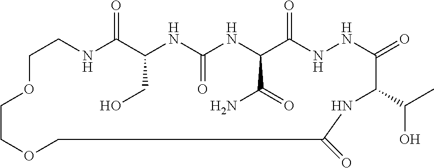 [SMILES: CC(O)[C@@H]1NC(=O)COCCOCCNC(=O)[C@@H](CO)NC(=O)N[C@H](C(N)=O)C(=O)NNC1=O] |
| | 10 |
| PMID30247903- Compound-General structure 12 | 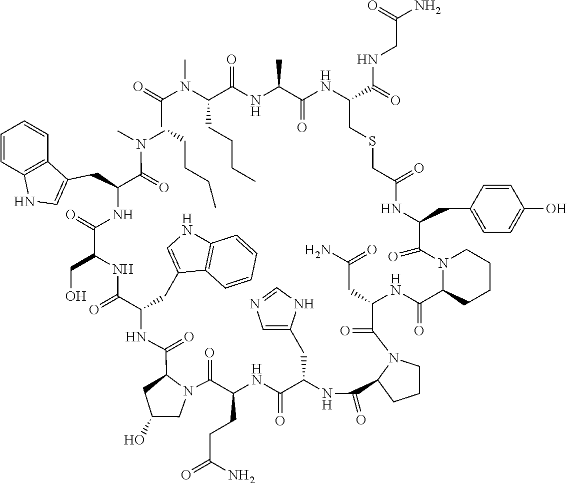 |
| | 12 |

[symbol]

indicates data missing or illegible when filed

In one embodiment of the present invention, anti-PD-L2 antibodies, etc. may be used as the PD-L2 inhibitor.

The composition for removing senescent cells according to the present invention, that is, the composition comprising as an active agent (a) a compound of Table 1 or Table 2 (preferably, Table 1) or its pharmaceutically acceptable salt, (b) secreted fluid of cells killed by treatment with a compound of Table 1 or Table 2 (preferably, Table 1) or its pharmaceutically acceptable salt, or (c) cells killed by treatment with a compound of Table 1 or Table 2 (preferably, Table 1) or its pharmaceutically acceptable salt is useful for improving, treating or preventing diseases and disorders related to senescent cells by removing senescent cells.

That is, one embodiment of the present invention provides the use of the composition according to the present invention as a medicine, health functional food, cosmetics or animal product for removing senescent cells. Another embodiment of the present invention provides the use of the composition according to the present invention to improve, treat or prevent diseases and disorders related to senescent cells through removal of senescent cells.

Accordingly, another embodiment of the present invention provides a method of removing senescent cells, comprising contacting with senescent cells or administering or applying to a subject in need of treatment, improvement, or cosmetic effect for diseases and disorders related to senescent cells therapeutically, health-improvingly, or cosmetically effective amount of (a) a compound of Table 1 or Table 2 (preferably, Table 1) or its pharmaceutically acceptable salt, (b) secreted fluid of cells killed by treatment with a compound of Table 1 or Table 2 (preferably, Table 1) or its pharmaceutically acceptable salt, or (c) cells killed by treatment with a compound of Table 1 or Table 2 (preferably, Table 1) or its pharmaceutically acceptable salt.

Accordingly, another embodiment of the present invention provides a method of improving, treating or preventing diseases and/or disorders related to senescent cells, comprising administering or applying to a subject in need of treatment, improvement, or cosmetic effect for diseases and disorders related to senescent cells therapeutically, health-improvingly, or cosmetically effective amount of (a) a compound of Table 1 or Table 2 (preferably, Table 1) or its pharmaceutically acceptable salt, (b) secreted fluid of cells killed by treatment with a compound of Table 1 or Table 2 (preferably, Table 1) or its pharmaceutically acceptable salt, or (c) cells killed by treatment with a compound of Table 1 or Table 2 (preferably, Table 1) or its pharmaceutically acceptable salt.

In one embodiment of the present invention, the contact with senescent cells may be performed in vitro or ex vivo.

In one embodiment of the present invention, the composition according to the present invention is applied in vitro or ex vivo to a tissue, organ or cells before their transplantation or administration in the case of transplantation of tissue, organ or (stem) cells and administration of related therapeutic agents. The composition of the present invention is used to remove senescent cells from tissues, organs or cells that are transplanted or administered through such treatment.

As mentioned above, the composition of the present invention may be a pharmaceutical composition, health functional food composition, cosmetic composition, or animal product composition depending on its purpose. Therefore, another embodiment of the present invention provides a use of the active agent (a), (b), and/or (c) for the manufacture of medicine, health functional food, cosmetic or animal product which is useful for improving, treating or preventing diseases and disorders related to senescent cells.

In the present invention, aging refers to, but is not limited to, natural aging due to passage of time; aging caused by stress from chemotherapy, radiation therapy, etc.; aging due to diets high in fat, high in sugar, or high in salt, or due to nutritional deficiencies; aging due to infections such as viruses and bacteria; or aging caused by physiological disturbances caused by diseases such as obesity, diabetes, cancer, fibrosis, etc. In the present invention, aging refers to a phenomenon in the body that promotes the production and accumulation of senescent cells or activates the function of senescent cells.

The senescent cells can be derived from various cells, such as somatic cells, stem cells, and cells that have changed abnormally due to disease. The senescent cells may also be cells of organ tissue derived from liver, kidney, spleen, brain, lung, muscle, skin, fat, etc. The disease may be selected from the group consisting of inflammation, fibrosis, cancer, etc.

The senescent cells may exhibit any one or more of the following characteristics: (i) Cell division and growth are almost permanently halted. (ii) Compared to non-aging normal cells, the overall size of the cells is increased. In particular, the contents of lysosomes are increased and abnormal mitochondria are increased. (iii) Nuclear structural proteins such as Lamin B1 and chromatin-binding proteins such as HMGB1 are reduced. (iv) β-galactosidase (SA-β-gal) is expressed due to aging. (v) Lipofuscin is produced. (vi) CDK (cyclin-dependent kinase) inhibitory factors such as p16 and p21 are activated and expressed in large quantities. (vii) As a continuous stress response, signal transduction systems such as p53 are activated according to DDR (DNA damage response), and the expression of related target genes is regulated accordingly. In this way, it can be said that cell division and growth stop as a common feature of senescent cells, and that senescence-associated markers such as SA-β-gal, lipofuscin, p16, p21, and p53 are expressed. (See doi.org/10.1016/j.tcb.2018.02.001).

The compound according to the present invention or its pharmaceutically acceptable salt promotes the death of senescent cells or inhibits the expression and secretion of aging-related markers, aging-promoting factors, or senescence-associated secretory factors. In one embodiment of the present invention, since these factors are expressed and secreted in senescent cells, the expression and secretion of these factors can be fundamentally blocked as the senescent cells are removed. Additionally, by removing senescent cells, the subject maintains resilience and resistance to various stresses.

The senescence-associated secretory factor (SASP) includes various aging-related pathological factors, and may be, for example, IL1α, IL1β, IL6, IL10, MCP1, PAI1 VCAM1, MMP1, MMP2, MMP3, MMP12, MMP13, TGFB, ACTA2, COL1A1, COL1A2, FN1, CXCL1, CXCL10, GM-CSF, GROa, GROB, GROy, IGFBP7, IL7, IL8, MIP1a, ENA78, GCP2, GITR, HGF, ICAM1, IGFBP2, IGFBP4, IGFBP5, IGFBP6, IL13, MCP4, MIF, MIP3a, MMP14, NAP2, PIGF, RANTES, sgp130, TIMP2, TRAILR3, Acrp30, Axl, bFGF, BLC, BTC, CTACK, EGFR, Fas, FGF7, GCSF, GDNF, HCC4, 1309, IFNγ, IGFBP1, IGFBP3, IL11, IL15, IL2Ra, IL6R, ITAC, Leptin, LIF, MSPa, PAI2, PDGF-BB, SCF, SDF1, STNF-RI, STNF-RII, TIMP-1, tPA, uPA, uPAR, VEGF, MCP3, IGF1, TGFB3, MIP1, IL4, FGF7, PDGF-BB, IL16, BMP4, MDC, MCP4, IL10, TIMP1, ICAM1, Axl, CNTF, INFγ, EGF, BMP6, etc. (See www.saspatlas.com).

One embodiment of the present invention is also based on the discovery that secreted fluid or substance from cells killed by treatment with a compound of Table 1 or Table 2 according to the present disclosure, preferably a compound of Table 1 or its pharmaceutically acceptable salt is useful in improving, treating or preventing various aging-related diseases by directly or indirectly inducing and/or promoting death of senescent cells, or directly or indirectly blocking and/or inhibiting the expression and secretion of aging-related markers, aging-promoting factors, or senescence-associated secretory factors (SASP).

In the present invention, “secreted fluid” includes components secreted and released from cells killed by treatment with the compound according to the present disclosure or its pharmaceutically acceptable salt. In one embodiment of the present invention, the “secreted fluid” may be a culture medium (e.g., medium) in which cells are cultured. In another preferred embodiment of the present invention, the secreted fluid of the present invention may be a culture filtrate obtained by removing the compound of the present disclosure from a culture medium (for example, medium) in which cells were cultured, or a purified separation liquid, a concentrate, or a dried product thereof. In the present invention, “culture filtrate” may refer to a culture solution in which cells are inoculated into a medium, treated with a compound of the present disclosure, cultured, and then the cells and compounds are removed. In one embodiment of the present invention, the culture filtrate can be obtained by (ultra) centrifuging the culture solution from which cells, compounds, etc. have been removed, and separating and purifying the obtained supernatant with (ultra) filtration, chromatography, etc. When administered into the body, such as the human body, dying cells producing culture filtrate or secreted fluid can be administered. In one embodiment of the present invention, the secreted fluid is contained in the culture filtrate or is produced and secreted from dying cells.

That is, one embodiment of the present invention is based on the discovery that (a) a compound that has the effect of killing or suppressing senescent cells or its salt, (b) secreted fluid from dying senescent cells, and/or (c) the dying senescent cells themselves can be used for suppressing or removing (other) senescent cells. The secreted fluid (substance) and dying senescent cells in the body may induce the death of senescent cells through activation of an immune response that activates and/or recruits immune cells, but the present invention is not limited to this theoretical mechanism.

In the present invention, “pharmaceutically acceptable salt” may be an acid addition salt formed with a pharmaceutically acceptable free acid. The free acid may be an inorganic acid or an organic acid. In this case, the inorganic acid may be hydrochloric acid, sulfuric acid, nitric acid, phosphoric acid, perchloric acid, hydrobromic acid, etc., and the organic acid may be acetic acid, methanesulfonic acid, ethanesulfonic acid, p-toluenesulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embonic acid, aspartic acid, glutamic acid, etc. The acid addition salt may be prepared by a conventional method, for example, by dissolving the compound of the present disclosure in an excessive amount of aqueous acid and precipitating the salt using a water-miscible organic solvent, such as methanol, ethanol, acetone, or acetonitrile. Additionally, when the compound of the present disclosure is acidic, the pharmaceutically acceptable salt can form base addition salts with various cations. Examples of such bases may be alkali metal or alkaline earth metal salts, for example calcium, magnesium, sodium, lithium, zinc, potassium and iron salts, among others. The alkali metal salt or alkaline earth metal salt can be obtained by dissolving the compound of the present disclosure in an excessive amount of alkali metal hydroxide or alkaline earth metal hydroxide solution, filtering the undissolved compound salt, and then evaporating and drying the filtrate. In addition, base addition salts include trimethylamine, triethylamine, dicyclohexylamine, ethanolamine, diethanolamine, triethanolamine, procaine, meglumine, ethylenediamine, choline, N, N-dibenzylethylenediamine, benzathine, benetamine, pyridine, picoline, quinoline, isoquinoline, tetramethylammonium, tetraethylammonium, benzyltrimethylammonium, benzyltriethylammonium, benzyltributylammonium, methyltrioctylammonium, tetrabutylammonium, and an organic salt such as, arginine, lysine, or histidine.

As used herein, the phrase “compound(s) of this/the present invention” includes any compound(s) of Table 1 or Table 2, as well as isotopic variants, clathrates, hydrates, solvates, or polymorphs thereof. And, even if the term “compound(s) of the invention” does not mention its pharmaceutically acceptable sat, the term includes salts thereof. In one embodiment, the compounds of this disclosure include stereo-chemically pure compounds, e.g., those substantially free (e.g., greater than 85% ee, greater than 90% ee, greater than 95% ee, greater than 97% ee, or greater than 99% ee) of other stereoisomers. That is, if the compounds of Chemical Formula 1 according to the present disclosure or salts thereof are tautomeric isomers and/or stereoisomers (e.g., geometrical isomers and conformational isomers), such isolated isomers and their mixtures also are included in the scope of this disclosure. If the compounds of the present disclosure or salts thereof have an asymmetric carbon in their structures, their active optical isomers and their racemic mixtures also are included in the scope of this disclosure.

As used herein, the term “isotopic variant” refers to a compound that contains unusual ratios of isotopes at one or more atoms that make up the compound. For example, isotopic variants of a compound may be radiolabeled. For example, the hydrogen atom may be selected from hydrogen, deuterium and tritium, and isotopic variants may contain carbon-13 (¹³C), nitrogen-15 (¹⁵N), etc.

If a compound (prodrug) is isolated in the body to produce a compound of the present disclosure or its salt, such compound is also included within the scope of the present invention. As used herein and unless otherwise indicated, the term “prodrug” refers to a compound that can be hydrolyzed, oxidized and subjected to other reactions under biological conditions (in vitro or in vivo) to provide an active compound, particularly a compound of the invention. Examples of prodrugs include compounds that contain biohydrolyzable moieties such as biohydrolyzable amides, biohydrolyzable esters, biohydrolyzable carbamates, biohydrolyzable carbonates, biohydrolyzable ureides, and biohydrolyzable phosphate analogs, and that biohydrolyze to produce the compounds of the invention, but are not limited to this specific examples. Preferably, the prodrug of the compound having a carboxyl group functional group is a lower alkyl ester of a carboxylic acid. Carboxylic esters are usually formed by esterifying a portion of the carboxylic acid present in the molecule. Prodrugs can be easily manufactured using methods well known in the field to which the present invention pertains.

As used herein, the term “polymorph” refers to solid crystalline forms of a compound of this disclosure or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound or its salt according to this disclosure that further includes a stoichiometric or non-stoichiometric amount of a solvent bound by non-covalent intermolecular forces. Preferred solvents are volatile, non-toxic, and acceptable for administration to humans in trace amounts.

As used herein, the term “hydrate” means a compound or its salt according to this disclosure that further includes a stoichiometric or non-stoichiometric amount of water bound by non-covalent intermolecular forces.

As used herein, the term “clathrate” means a compound or its salt in the form of a crystal lattice that contains spaces (e.g., channels) that have a guest molecule (e.g., a solvent or water) trapped within.
(a) A compound of Table 1 or Table 2, preferably a compound of Table 1, or its pharmaceutically acceptable salt, (b) secreted fluid from cells killed by treatment with a compound of Table 1 or Table 2, preferably a compound of Table 1, its pharmaceutically acceptable salt; or (c) cells killed by treatment with a compound of Table 1 or Table 2, preferably a compound of Table 1, or its pharmaceutically acceptable salt according to the present invention has (have) the effect of killing senescent cells or blocking the action of senescent cells, thereby being useful for improving, treating or preventing various diseases and disorders related to senescent cells.

In one embodiment of the present invention, the diseases or disorders that can be improved, treated, or prevented by removing senescent cells or inhibiting the action of senescent cells include, but are not limited to,
proliferative diseases and disorders such as cancer, pre-cancerous/pre-malignant condition, resistance, malignancy, relapse, and metastasis;
inflammatory or autoimmune diseases and disorders such as arthritis, osteoarthritis, rheumatoid arthritis, juvenile idiopathic arthritis, degenerative joint disorder, degenerative articular cartilage, inflammatory bowel disease, ulcerative colitis, Crohn's disease, stomatitis, mucositis, oral mucositis, pancreatitis, tendinopathy, tendinosis, tendonitis or tendinitis, synovitis, tenosynovitis, inflamm-aging, amplified hyperinflammation, cytokine release/storm syndrome, COVID19 infection, COVID19 complication, femoroacetabular impingement syndrome, tunnel syndrome, meniscus tear, meniscus derangement, meniscus lesion, meniscus disorder, sepsis, HIV infection, AIDS, rhinovirus infection, inflammatory response syndrome after surgery, spondylarthrites, autoimmune encephalomyelitis, and common variable immunodeficiency;
neurological diseases and disorders such as neurodegenerative disease, dementia, alzheimer's disease, memory disorder, cognitive decline dysfunction, parkinson's disease, huntington's disease, amyotrophic lateral sclerosis, motor neuron disease, primary lateral sclerosis, progressive muscular atrophy, lower motor neuron disease, spinal muscular atrophy, progressive bulbar paralysis, pseudobulbar palsy, post-polio syndrome, spastic paraplegia, progressive supranuclear palsy, spinal cord injury, chronic pain, allodynia, anxiety, depression, schizophrenia, neuropsychiatric dysfunction, post-traumatic stress disorder, gulf war illness, and sleep disorder;
metabolic diseases and disorders such as obesity, diabetes, diabetic ulcer, osteoporosis, intestinal bowel disease, intestinal bowel microbiome disorder, lipodystrophy, adipose atrophy, hypogonadism, and metabolic syndrome;
cardiovascular diseases and disorders such as atherosclerosis, heart failure, congestive heart failure, cardiomyopathy, heart functional disorder, cardiac dysfunction, angina pectoris, endocarditis, mitral valve prolapse, myocardial infarction, cardiomyocyte hypertrophy, loss of cardiac stress tolerance, idiopathic dilated cardiomyopathy, aneurysm, aortic aneurysm, coronary artery aneurysm, brain aneurysm, vascular hyporeactivity/calcification, aging-related vascular dysfunction, arteriovenous fistula, stenosis, renal artery stenosis, carotid artery stenosis, neointimal hyperplasia, hypertension, stroke, thrombosis, coagulation disorder, spontaneous cerebral microhemorrhages, dyslipidemia, atrial fibrillation, arrhythmia, and diabetic cardiomyopathy;
lung diseases and disorders such as chronic obstructive pulmonary disease, emphysema, chronic pneumonia, bronchiectasis, aging-related pulmonary dysfunction, respiratory failure, function pulmonary insufficient, hyperoxic lung injury, bronchopulmonary dysplasia, respiratory distress syndrome, pulmonary hypertension, asthma, pneumonia, viral pneumonia, interstitial pneumonia, cryptogenic organizing pneumonia, idiopathic pleuroparenchymal fibroelastosis, hypersensitivity pneumonitis, granulomatous-lymphocytic interstitial lung disease, and chronic bronchitis;
kidney diseases and disorders such as chronic kidney disease, nephropathy, renal failure/dysfunction, glomerulosclerosis, diabetic nephropathy, diabetic chronic kidney disease, polycystic kidney disease, immunoglobulin A nephropathy, kidney stone disease, nephrolithiasis, nephronophthisis, lupus nephritis, tubulointerstitial disease, and renal tubulointerstitial injury;
liver diseases and disorders such as cirrhosis, liver fibrosis, hepatitis, hepatopathy, fatty liver, nonalcoholic fatty liver disease, hepatic steatosis, nonalcoholic steatohepatitis, primary biliary cirrhosis, cholangitis, primary sclerosing cholangitis, biliary atresia, hepatic encephalopathy, and biliary injury;
eye diseases and disorders such as cataract, glaucoma, aging-related macular degeneration, macular degeneration, macular edema, retinal degenerative disease, aging-related vision loss, retinopathy, diabetic retinopathy, diabetic macular edema, dry eye disease, hyperemia, retinal microaneurysm, retinal microangiopathy, fuchs endothelial dystrophy, corneal dystrophies, and birdshot uveitis;
skin diseases and disorders such as skin aging, epidermal atrophy, loss of elasticity, wrinkle, skin photoaging, hair loss (alopecia), skin naevi, skin spot, pigmentation, hyperpigmentation, melasma, blemish, keloid, damage/wound healing disorder, keloid, psoriasis, lupus, systemic lupus erythematosus, dermatopolymyositis, immunobullous disease, pemphigus, bullous pemphigoid, blister, wart, human papilloma virus infection, dermatitis, eosinophilic dermatoses, atopic dermatitis, skin lymphoma, erythroderma, lichen planus, lichenoid dermatosis, febrile neutrophilic dermatosis, rosacea, dermatofibroma, keratosis pilaris, herpes, pruritus, sensory defect, eczema, eruption, urticaria, vitiligo, senescence in gingival tissues, dyskeratosis congenita, ichthyosis vulgaris, actinic keratosis, photosensitivity, and photodermatoses;
regenerative maintenance-related diseases and disorders such as various stem cell/bone marrow regeneration maintenance diseases and disorders including bone marrow stem cell functional disorder, various organs/tissues regeneration maintenance diseases and disorders including liver regeneration, kidney organ regeneration, heart organ regeneration, neuroregeneration, etc., immune regulation diseases and disorders including myelodysplastic syndromes, myelopathy, inflammation, etc., and damage and decline in function of various organs/tissues including sarcopenia, etc.;
transplantation-related diseases and disorders such as transplantation diseases and disorders of various stem cells/bone marrow/organs/tissues/cells including graft versus host disease, stem cell transplantation complication, bone marrow transplantation complication, cardiac organ transplantation complication, renal organ transplantation complication, corneal allograft rejection, chronic allograft nephropathy, etc. and transplantation of various foreign substances/materials diseases and disorders including foreign body response;
fibrosis diseases and disorders such as idiopathic pulmonary fibrosis, pulmonary fibrosis, cystic fibrosis, renal fibrosis, tubulointerstitial fibrosis, liver fibrosis, cardiac fibrosis, pancreatic fibrosis, retroperitoneal fibrosis, mediastinal fibrosis, uterine fibrosis, biliary fibrosis, oral submucous fibrosis, muscle fibrosis, neurofibroma, myelofibrosis, and fibrodysplasia ossificans progressiva;
sclerosis diseases and disorders such as systemic sclerosis, scleroderma, and multiple sclerosis;
β-galactosidase-related diseases and disorders such as progerias, hutchinson-gilford progeria syndrome, werner syndrome, bloom syndrome, marfan syndrome, wiedemann-rautenstrauch syndrome, rothmund-thomson syndrome, cockayne syndrome, down-syndrome, and dyskeratosis congenita;
geriatric diseases and disorders such as reduced lifespan/healthspan, frailty, inflammaging, chronic inflammation, late-life depression, sarcopenia, muscular dystrophy, muscle fibrosis, myotonic dystrophy type 1, amyotonia congenita, fibromyalgia, rotator cuff tear, osteoporosis, skeletal disorder, ataxia, wound healing disorder, delayed fracture healing, lipodystrophy, adipose atrophy, intervertebral disc degeneration, lumbar herniated intervertebral disc, kyphosis, low back pain, fibrodysplasia ossificans progressiva, menopause, endometriosis, polycystic ovary syndrome, tremor, urinary incontinence, urination disorder, prostatic hypertrophy, splenomeglay, hearing loss, olfactory dysfunction, ovarian aging, and periodontal disease;
various aging-related diseases and disorders include damages, side effects and sequelae, fatigue, weight loss, physical inactivity, ataxia, cachexia, boredom, depression, anxiety, neuropsychiatric dysfunction, lethargy, neurosis, anorexia nervosa, syncope, panic disorder, chronic inflammation, stomatitis, mucositis, oral mucositis, thrombocytopenia, radiation ulcers, bone marrow disorder, anemia, immune disorder, hormone disorder, menopause, nausea, vomiting, diarrhea, hepato/nephrocomplication, hepato-/nephro-/cardio-/gastrointestinal/ovarian/vascular/skin toxicity, heart functional disorder, cardiomyopathy, congestive heart failure, peripheral neuropathy, hair loss, allopecia, dermatitis, eruption, pain, fluid retention, urticaria, hyperpigmentation, lipodystrophy, adipose atrophy, bone-loss, osteoporosis, infertility, oligospermia, abnormal embryo implantation and placentation, reduced reproductive outcome from in vitro fertilization, preeclampsia, pregnancy loss, miscarriage, cognitive decline dysfunction, cancer relapse, metastasis, vision loss, hearing loss, ovarian injury, hyposalivation, pulmonary fibrosis, blue light toxicity, etc., wherein the various aging-related diseases and disorders are caused by any one of anticancer drugs such as radiation/chemotherapy/targeted/immunotherapy, drugs/substances with strong toxicity and side effects, physiological disturbances such as obesity/diabetes, diets such as high fat/high sugar/high salt, hemorrhagic shock, myocardial infarction, hypoxia, ischemia and reperfusion damage, nutritional deficiencies, and various acute and chronic stressors such as war, accidents, smoking, viral/bacterial infections such as HIV, surgery, foreign transplants, inflammation, tissue wounds, hyperthermia, etc.; and
rare aging-related diseases and disorders found in small numbers of the population such as acquired lipodystrophy, acromegaly, acute respiratory distress syndrome, alexander disease, alpha-1 antitrypsin deficiency, amyloidosis, autoimmune hemolytic anemia, aplastic anemia, anencephaly, antiphospholipid syndrome, takayasu arteritis, infectious arthritis, ataxia with vitamin E deficiency, behcet's syndrome, benign paroxysmal positional vertigo, beta thalassemia, biliary atresia, bloom syndrome, CDKL5 deficiency disorder, chronic granulomatous disease, common variable immune deficiency, corneal dystrophies, costello syndrome, creatine transporter deficiency, cystic fibrosis, cytochrome C oxidase deficiency, cytomegalovirus infection, danon disease, dengue fever, duchenne muscular dystrophy, dyskeratosis congenita, herpe simplex encephalitis, erdheim chester disease, fanconi anemia, fibrodysplasia ossificans progressiva, focal segmental glomerulosclerosis, friedreich's ataxia, frontotemporal degeneration, glucose-6-phosphate dehydrogenase deficiency, graft versus host disease, hepatic encephalopathy, hermansky pudlak syndrome, hodgkin's disease, huntington's disease, hutchinson-gilford progeria syndrome, progeria, hypoplastic left heart syndrome, idiopathic pulmonary fibrosis, immunoglobulin A nephropathy, immune thrombocytopenia, kawasaki disease, langerhans cell histiocytosis, leprosy, li-fraumeni syndrome, limb-girdle muscular dystrophies, lymphangioleiomyomatosis, malaria, marfan syndrome, measles, multiple myeloma, multiple sclerosis, becker muscular dystrophy, myelodysplastic syndromes, myhre syndrome, myotonic dystrophy, nontuberculous mycobacterial lung disease, pneumocystis pneumonia, primary biliary cholangitis, primary sclerosing cholangitis, pulmonary arterial hypertension, rett syndrome, rothmund-thomson syndrome, scleroderma, sialadenitis, ulcerative colitis, werner syndrome, wiedemann rautenstrauch syndrome, abdominal aortic aneurysm, ankylosing spondylitis, Ankyrin-B syndrome, anterior uveitis, PASLI disease, down syndrome, coronary artery aneurysm, dilated cardiomyopathy, doxorubicin induced cardiomyopathy, eclampsia, high molecular weight kininogen deficiency, interstitial cystitis, interstitial lung disease, kindler syndrome, lewy body dementia, lupus, lupus nephritis, neural tube defects, neuroferritinopathy, nijmegen breakage syndrome, nonalcoholic steatohepatitis, oral leukoplakia, oral submucous fibrosis, paraplegia, parkinson's disease, peripartum cardiomyopathy, psoriasis, silicosis, sjogren syndrome, tuberous sclerosis complex, xeroderma pigmentosum, sickle cell disease, acute myeloid leukemia, craniopharyngioma, glioblastoma, glioma, hepatocellular carcinoma, malignant melanoma, small cell lung cancer, thyroid cancer, acute monoblastic leukemia, diffuse large B-cell lymphoma, and oral squamous cell carcinoma.

In a preferred embodiment of the present invention, the proliferative disease or disorder is cancer. In a preferred embodiment of the present invention, the inflammatory or autoimmune disease or disorder is arthritis, osteoarthritis, rheumatoid arthritis, juvenile idiopathic arthritis, spondyloarthritis, degenerative cartilage disease, or kyphosis. In a preferred embodiment of the present invention, the neurological disease or disorder is a degenerative brain disease (dementia, Alzheimer's disease, memory loss, (mild) cognitive impairment, etc.). In a preferred embodiment of the present invention, the metabolic disease and disorder is obesity, diabetes and diabetic complications (such as diabetic ulcer, diabetic retinopathy, diabetic cardiomyopathy, and diabetic nephropathy), or age-related bone disease (such as osteoporosis, fracture healing disorder, alveolar bone loss, and periodontal disease). In a preferred embodiment of the present invention, the cardiovascular disease and disorder is atherosclerosis, aortic aneurysm, coronary artery aneurysm, cerebral aneurysm, age-related vascular dysfunction, decreased cardiac load resistance, cardiac hypertrophy, myocardial infarction, heart failure, or hypertension. In a preferred embodiment of the present invention, the pulmonary disease and disorder is idiopathic pulmonary fibrosis, chronic pulmonary inflammation, pulmonary dysfunction, pulmonary fibrosis, chronic obstructive pulmonary disease, or emphysema. In a preferred embodiment of the present invention, the renal disease and disorder is nephropathy, renal failure, glomerulosclerosis, or chronic kidney disease. In a preferred embodiment of the present invention, the liver disease and disorder is fatty liver (alcoholic/non-alcoholic fatty liver disease, hepatic steatosis, etc.), hepatopathy, liver fibrosis, cirrhosis, hepatitis, primary biliary cirrhosis, primary sclerosing cholangitis, biliary atresia, or bile duct damage. In a preferred embodiment of the present invention, the ocular disease and disorder is cataract, or retinal degenerative disease (age-related macular degeneration, macular edema, retinopathy, etc.). In a preferred embodiment of the present invention, the skin disease or disorder is skin aging, epidermal atrophy, loss of elasticity, wrinkles, moles, spots, freckles, pigmentation, blemishes, freckles, hair loss, or wound healing disorder. In a preferred embodiment of the present invention, the fibrosing disease or disorder is retroperitoneal fibrosis, idiopathic pulmonary fibrosis, pulmonary fibrosis, or hepatic fibrosis. In a preferred embodiment of the present invention, the sclerosing disease or disorder is systemic sclerosis (idiopathic pulmonary fibrosis, scleroderma). In a preferred embodiment of the present invention, the other geriatric disease or disorder is senility, sarcopenia, muscular dystrophy, lipoatrophy, age-related inflammation, or geriatric depression. In a preferred embodiment of the present invention, the side effects and sequelae due to stress and the age-related diseases and disorders resulting therefrom are fatigue and weakness, lethargy, weight loss, hepatotoxicity, nephrotoxicity, thrombosis, hair loss, or bone marrow dysfunction (myelodysplastic syndrome, myelopathy, bone marrow transplant failure, anemia, etc.). In a preferred embodiment of the present invention, the rare disease is lipodystrophy, idiopathic pulmonary fibrosis, Lewy body dementia, or non-alcoholic steatohepatitis.

In another preferred embodiment of the present invention, diseases and disorders that can be improved, treated or prevented by removing senescent cells or inhibiting the action of senescent cells include cancer (it is suppressed and treated by removing aged cancer cells through prosenescence anti-cancer therapy, which includes not only cancer but also early stage cancer, recurrence, metastasis, resistance, malignancy, or aftereffects), inflammation (it is inflamm-aging, immuno-senescence, or senescence-associated inflammation caused by senescent cells, and due to this, the progressive chronic and degenerative inflammation observed in the elderly, or it includes hyperamplified inflammation accompanied by cytokine storm observed in the elderly due to infections such as viruses), fibrosis and cirrhosis (including fibrosis and cirrhosis of the liver, lungs or kidneys, etc.), geriatric diseases (e.g., frailty (including decline in activity, endurance, muscle strength, and/or grip strength), sarcopenia, osteoporosis or lipoatrophy), kidney damage, liver damage, skin aging-related diseases and disorders (e.g., wrinkles, loss of elasticity, hair loss, deterioration of hair skin, freckles, blemishes, skin rash, skin spots, skin discoloration, pigmentation, warts, urticaria, worsening pores, wound healing disorder, etc.), and side effects and aftereffects (including fatigue, weakness, weight loss, anxiety, depression, lethargy, fatty liver, etc.) due to stress caused by anticancer drugs or obesity.

In relation to the above frailty, frailty is, for example, a specific disease to which the R54 code was recently assigned, which shows the same symptoms as aging-related fatigue, weakness, asthenia, inactivity, muscle weakness, and gait disorder that occur with or without cognitive and memory impairment due to aging. Frailty can be analyzed by symptom indicators (https://en.wikipedia.org/wiki/Frailty_syndrome).

As used herein, the term “treatment” refers to any action (alleviate, reverse or treat) in which the symptoms of aging-related diseases and disorders are improved, ameliorated, or beneficially changed by administration of the active agent of the present invention, and the term “prevention” refers to any action (prevent, inhibit or delay) that blocks, inhibits, or delays the progression of aging-related diseases and disorders by administering a composition according to the present invention. The term “removal” of senescent cells refers to any action that inhibits the function of senescent cells, such as their death.

In an embodiment of the present invention, (a) a compound of Table 1 or Table 2 (preferably, Table 1) or its pharmaceutically acceptable salt, (b) secreted fluid from cells killed by treatment with a compound of Table 1 or Table 2 (preferably, Table 1) or its pharmaceutically acceptable salt, or (c) cells killed by treatment with a compound of Table 1 or Table 2 (preferably, Table 1) or its pharmaceutically acceptable salt can be administered or applied to a patient in a therapeutically, cosmetically, or pharmaceutically effective amount.

One embodiment of the present invention provides a method of improving, treating or preventing diseases and/or disorders related to senescent cells, comprising contacting with senescent cells or administering or applying to a subject in need of treatment, improvement, or prevention for diseases and disorders related to senescent cells therapeutically, cosmetically or pharmaceutically effective amount of a substance that increases the secretion of HMGN1 (High Mobility Group Nucleosome Binding Domain 1) protein (HMG14) in senescent cells. In this method, the ‘substance that increases secretion of HMG14’ and ‘disease and disorders related to senescent cells’ are the same as previously described.

Here, “therapeutically effective amount” or “pharmaceutically effective amount” refers to the amount of a compound or composition effective in preventing or treating the target disease, which is sufficient to treat the disease with a reasonable benefit/risk ratio applicable to medical treatment and does not cause side effects. Additionally, the “effective amount” refers to an amount sufficient to inhibit or reduce the activity of senescent cells in aging-related diseases, either in vitro or in vivo. The level of the effective amount can be determined by factors including the patient's health status, type and severity of the disease, activity of the drug, sensitivity to the drug, administration method, administration time, administration route and excretion rate, treatment period, combination or concurrent use of drugs, and other factors well known in the medical field.

One embodiment of the present invention also provides a method of removing senescent cells by contacting them in vivo or ex vivo with (a) a compound of Table 1 or Table 2 (preferably, Table 1) or its pharmaceutically acceptable salt, (b) secreted fluid from cells killed by treatment with a compound of Table 1 or Table 2 (preferably, Table 1) or its pharmaceutically acceptable salt, or (c) cells killed by treatment with a compound of Table 1 or Table 2 (preferably, Table 1) or its salt.

In one embodiment of the present invention, the composition of the present invention can be used for use as described above (i.e., for removing senescent cells and/or improving, treating or preventing diseases and disorders related to senescent cells) by combining at least one active agent (different from the first active agent) selected from the group consisting of Roxadustat, Dexmedetomidine, 5-aminosalicylic acid, Pioglitazone, Rosiglitazone, Setanaxib, and an immune checkpoint inhibitor as a second active ingredient. That is, the composition of the present invention is more preferable for the purposes of the present invention when combined with at least one second active agent (different from the first active ingredient) selected from the group consisting of Roxadustat, Dexmedetomidine, 5-aminosalicylic acid, Pioglitazone, Rosiglitazone, Setanaxib, and an immune checkpoint inhibitor.

In one embodiment of the present invention, the second active agent may be comprised in the composition of the present invention, or may be administered simultaneously or sequentially as a separate composition (formulation). Accordingly, one embodiment of the present invention provides a kit or preparation for removing senescent cells, containing the composition (the first active agent) according to the present invention and the second active agent.

Accordingly, another embodiment of the present invention also provides a method for removing senescent cells, comprising contacting senescent cells with or administering or applying to a subject in need of improvement, treatment, or prevention diseases and disorders related to senescent cells the composition according to the present invention simultaneously or sequentially with one or more additional second agent (selected from the group consisting of Roxadustat, Dexmedetomidine, 5-aminosalicylic acid, Pioglitazone, Rosiglitazone, Setanaxib, and an immune checkpoint inhibitor). Other embodiment of the present invention also provides a method for improving, treating or preventing diseases and disorders related to senescent cells, comprising contacting senescent cells with or administering or applying to a subject in need of improvement, treatment, or prevention diseases and disorders related to senescent cells the composition according to the present invention simultaneously or sequentially with one or more additional second agent.

In this combination or combination method, the second active agent may be any one or more selected from the group consisting of Roxadustat, Dexmedetomidine, 5-aminosalicylic acid, Pioglitazone, Rosiglitazone, Setanaxib, and an immune checkpoint inhibitor. The immune checkpoint inhibitor may be any one or more of the aforementioned CTLA4 inhibitor, PD-1 inhibitor, PD-L1 inhibitor, and PD-L2 inhibitor. In this case, the compound comprised in the composition according to the present invention, which are used together or combined with the second active agent, and the compound used for treating cells to obtain the secreted fluid of cells or dying cells according to the present invention are a compound other than the second active agent.

In this combination or combination method, the composition of the present invention and the second active agent are administered in any order or simultaneously. If simultaneous, the compositions of the invention and the second active agent may optionally be provided in a single, unified form, or in multiple forms (e.g., as one pill or two separate pills).

Additionally, in this combination or combination method, the combination methods, compositions and preparations are not limited to the use of only two agents, and multiple therapeutic combinations are also contemplated. Dosage regimens for treating, preventing, or ameliorating pathological condition(s) may vary depending on various factors. These factors include the individual's age, weight, sex, diet, and medical condition, as well as any disorders the individual suffers from.

The pharmaceutical preparations that make up the combination treatment disclosed herein are optionally in combined formulations or in separate formulations primarily for simultaneous administration. The pharmaceutical preparations that make up the combination treatment can also be administered sequentially as either of the agents administered by a regimen requiring two-step administration. A two-step dosing regimen may require sequential administration of the active agents or spaced administration of separate active agents. The time period between multiple administration steps can range from several minutes to several hours depending on the properties of each pharmaceutical preparation such as potency, solubility, bioavailability, plasma half-life and kinetic profile of the pharmaceutical agent. It can last up to several hours. Circadian variation in target molecule concentration is used to determine the optimal dosing interval.

For the treatment of the diseases or conditions referred to above, the active agents and/or compositions described herein can be administered as follows:

### Oral Administration

The compounds of the present invention may be administered orally, including by swallowing, so that the compound enters the gastrointestinal tract, or absorbed into the blood stream directly from the mouth (e.g., buccal or sublingual administration).

Suitable compositions for oral administration include solid, liquid, gel or powder formulations, and have a dosage form such as tablet, lozenge, capsule, granule or powder.

Liquid formulations can include solutions, syrups and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

In a tablet dosage form the amount of drug, active agent, present may be from about 0.05% to about 95% by weight, more typically from about 2% to about 50% by weight of the dosage form. In addition, tablets may contain a disintegrant, comprising from about 0.5% to about 35% by weight, more typically from about 2% to about 25% of the dosage form. Examples of disintegrants include, but are not limited to, lactose, starch, sodium starch glycolate, crospovidone, croscarmellose sodium, maltodextrin, or mixtures thereof.

Suitable lubricants, for use in a tablet, may be present in amounts from about 0.1% to about 5% by weight, and include, but are not limited to, talc, silicon dioxide, stearic acid, calcium, zinc or magnesium stearate, sodium stearyl fumarate and the like.

Suitable binders, for use in a tablet, include, but are not limited to, gelatin, polyethylene glycol, sugars, gums, starch, polyvinyl pyrrolidone, hydroxypropyl cellulose, hydroxypropylmethyl cellulose and the like. Suitable diluents, for use in a tablet, include, but are not limited to, mannitol, xylitol, lactose, dextrose, sucrose, sorbitol, microcrystalline cellulose and starch.

Suitable solubilizers, for use in a tablet, may be present in amounts from about 0.1% to about 3% by weight, and include, but are not limited to, polysorbates, sodium lauryl sulfate, sodium dodecyl sulfate, propylene carbonate, diethyleneglycol monoethyl ether, dimethyl isosorbide, polyethylene glycol (natural or hydrogenated) castor oil, HCOR™ (Nikkol), oleyl ester, Gelucire™, caprylic/caprylic acid mono/diglyceride, sorbitan fatty acid esters, and Solutol HS™

### Parenteral Administration

The active agent of the present disclosure may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Most parenteral formulations are liquid compositions, and the liquid composition is an aqueous solution containing the active ingredient according to the present invention, a salt, a buffering agent, an isotonic agent, and the like.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

### Topical Administration

The active agent of the present invention may be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like.

When the composition of the present invention is a cosmetic composition, it may be in the liquid formulation mentioned above.

### Various Other Administration Methods

In addition to the above methods, the present invention includes various administration methods, and further, the composition of the present invention can be used to remove senescent cells in vitro, such as in the case of foreign transplantation of tissues/organs or (stem) cells and administration of therapeutic agents.

### Advantageous Effects

The active agent and composition according to the present invention can fundamentally block and suppress the expression and secretion of various aging-related pathological factors secreted from senescent cells by removing senescent cells. In addition, the secreted fluid from cells whose death was induced by treatment with the composition for removing senescent cells according to the present invention significantly reduces the survival rate of senescent cells, and thereby can fundamentally block and suppress the expression and secretion of various aging-related pathological factors secreted from these senescent cells. Therefore, the composition for removing senescent cells according to the present invention can be usefully used to improve, treat, or prevent aging-related diseases and disorders. Furthermore, the composition for removing senescent cells according to the present invention can be usefully used in cosmetics, health functional foods, animal products, etc. in relation to aging-related diseases and disorders related to senescent cells.

### MODE FOR INVENTION

Hereinafter, to aid understanding of the present invention, it will be described in detail through examples. However, the examples according to the present invention may be modified into various other forms, and the scope of the present invention should not be construed as being limited to the following examples. Examples of the present invention are provided to more completely explain the present invention to those with average knowledge in the field to which the present invention pertains.

The present inventors performed several evaluations using LX-112 (ethaselen, 1,2-[bis(1,2-benzisoselenazolone-3 (2H)-ketone)]ethane) as an exemplary compound to evaluate the effect of senescent cell removal on the improvement, treatment, or prevention of senescent cell-related diseases and disorders. The results of these experiments are disclosed in WO2023-085787 A1, the entire contents of which are hereby incorporated by reference herein.

### Example 11. Screening of Various Compounds that Remove Senescent Human Cells Through a Different Mechanism from Existing Senescent Cell Removers

Based on the results of Example 10 of WO2023-085787 A1, compounds that induce the death of senescent cells through a mechanism similar to LX-112, unlike existing senescent cell removers such as ABT-263, were screened based on HMGN1. In the same manner as in Example 1 of WO2023-085787 A1, cells were treated with doxorubicin or control (DMSO) and cultured with 30 μM test compound (except that LX-305, LX-1302, LX-1603, LX-2005, LX-2702, LX-2901, and LX-3106 were 90 UM, and LX-1103 and LX-2007 were 270 UM). The following test compounds were synthesized by and purchased from Medchemexpress, Selleckchem, Cayman, Medkoo, Sigma, Molport, Enzo, TCI, Santa Cruz, REONSIMI, Abcam, Alfa Aesar, APExBIO, BOCSCI, Merck, Thermo Fisher Scientific, R&D Systems, ChemFaces, Aobious, etc. In the same manner as Example 10 of WO2023-085787 A1, the amount of HMGN1 secreted in the cell culture was measured using an enzyme-linked immunosorbent assay (ELISA Kit, AssayGenie). In cells in which senescence was not induced by treatment with the control group (DMSO), the amount of HMGN1 above the effective value (sensitivity: 35 pg/ml), analysis limit, was not detected in both untreated and treated conditions with the test compound. On the other hand, in cells where senescence was induced by treatment with doxorubicin, the amount of HMGN1 was measured at 80 to 90 pg/ml. This amount was calculated as 100%, and the amount of HMGN1 was measured under conditions in which aged cells were treated with the test compound, and the results are shown in Table below.

TABLE 3

| | HMGN1 | | HMGN1 | | HMGN1 | | HMGN1 | | HMGN1 |
|---|---|---|---|---|---|---|---|---|---|
| DMSO | 100 | LX-305 | 199 | LX-401 | 263 | LX-403 | 275 | LX-405 | 285 |
| LX-801 | 265 | LX-905 | 318 | LX-1102 | 271 | LX-1103 | 172 | LX-1302 | 171 |
| LX-1601 | 154 | LX-1603 | 168 | LX-1801 | 291 | LX-1902 | 303 | LX-1903 | 281 |
| LX-2001 | 328 | LX-2002 | 305 | LX-2003 | 298 | LX-2005 | 212 | LX-2007 | 255 |
| LX-2101 | 274 | LX-2102 | 288 | LX-2601 | 263 | LX-2602 | 208 | LX-2702 | 206 |
| LX-2707 | 235 | LX-2901 | 167 | LX-3001 | 301 | LX-3002 | 335 | LX-3106 | 254 |
| LX-3107 | 196 | LX-3200 | 234 | LX-3201 | 286 | LX-3203 | 315 | LX-3303 | 275 |

As a result, the amount of HMGN1 secreted from senescent cells that die when treated with the test compound increased like LX-112. In addition, as shown in the results obtained by treating with LX-112 in Example 10 of WO2023-085787 A1, caspase inhibitors did not effectively inhibit the death of senescent cells by the test compound. Despite the presence of caspase inhibitors, the test compound still could eliminate senescent cells by inducing their death. From this, it was confirmed that the test compounds induce the death of senescent cells through a different mechanism from ABT-263.

Furthermore, the degree of death of senescent cells were analyzed based on the increase in the amount of HMGN1 in the same manner as in Example 1 of WO2023-085787 A1. Cells in which senescence was induced by treatment with doxorubicin were treated with the test compound at designated concentrations and measured using the CellTiter-Glo Luminescent Cell Viability Assay (Promega). The cell viability results are shown in Table below.

TABLE 4

| Survival (%) | 0 μM | | 1.1 μM | | 3.3 μM | | 10 μM | | 30 μM | | 90 μM | | 270 μM | |
|---|---|---|---|---|---|---|---|---|---|---|---|---|---|---|
| LX-305 | 100 | (±5.7) | 101.1 | (±5.4) | 97.2 | (±2.1) | 91 | (±4) | 78 | (±5.1) | 23.2 | (±3.5) | | |
| LX-401 | 100 | (±1) | 20.4 | (±18.4) | 12.4 | (±7.3) | 10.6 | (±5.7) | 7.8 | (±3.4) | | | | |
| LX-403 | 100 | (±4.8) | 36.9 | (±4.2) | 15.5 | (±3.2) | 6.5 | (±1.8) | | | | | | |
| LX-405 | 100 | (±3.3) | 4.9 | (±0.6) | | | | | | | | | | |
| LX-801 | 100 | (±4.4) | 50.5 | (±2.7) | 48.9 | (±5.1) | 28.2 | (±5.4) | | | | | | |
| LX-905 | 100 | (±7.3) | 97.6 | (±5.2) | 91.6 | (±9.7) | 42.5 | (±2.8) | 6.9 | (±0.1) | | | | |
| LX-1102 | 100 | (±1.5) | 94 | (±4.4) | 13.9 | (±4.4) | 3.1 | (±1.2) | | | | | | |
| LX-1103 | 100 | (±4.1) | 103.6 | (±4.2) | 98.9 | (±5.1) | 99.6 | (±7) | 95.2 | (±10.4) | 88 | (±10.8) | 61.3 | (±9) |
| LX-1302 | 100 | (±3.7) | 102 | (±2) | 105.2 | (±7.7) | 92.2 | (±2.4) | 79.6 | (±4.3) | 52.4 | (±4.6) | | |
| LX-1601 | 100 | (±6.4) | 93.7 | (±2.9) | 90 | (±3.5) | 95.6 | (±7.9) | 61.4 | (±0.8) | | | | |
| LX-1603 | 100 | (±8) | 100.8 | (±4.8) | 95.6 | (±7.3) | 91.1 | (±10) | 84.3 | (±8.5) | 48.3 | (±9.6) | | |
| LX-1801 | 100 | (±2) | 8.7 | (±9.4) | | | | | | | | | | |
| LX-1902 | 100 | (±1.1) | 59 | (±4.2) | 39.6 | (±4.1) | 11.9 | (±4.7) | 3.9 | (±1.2) | | | | |
| LX-1903 | 100 | (±0.8) | 73.4 | (±2.2) | 50.3 | (±1.2) | 22.4 | (±5.1) | 11.8 | (±1.7) | | | | |
| LX-2001 | 100 | (±7.5) | 75.2 | (±8.3) | 0.8 | (±0.0) | | | | | | | | |
| LX-2002 | 100 | (±10.7) | 100.9 | (±8.4) | 12.4 | (±1.7) | | | | | | | | |
| LX-2003 | 100 | (±9.1) | 89.6 | (±10.3) | 65.8 | (±6.7) | 34.6 | (±6.5) | 14 | (±2.6) | | | | |
| LX-2005 | 100 | (±10.2) | 97.5 | (±6.2) | 93.3 | (±4.2) | 87.2 | (±4.7) | 73.8 | (±3.2) | 31.5 | (±1.1) | | |
| LX-2007 | 100 | (±9.6) | 105.4 | (±8) | 98.9 | (±7.1) | 91.2 | (±6.1) | 83.9 | (±7.3) | 47.2 | (±1.4) | 1.3 | (±0.0) |
| LX-2101 | 100 | (±6.3) | 1.8 | (±0.0) | | | | | | | | | | |
| LX-2102 | 100 | (±8.4) | 9.4 | (±1.4) | | | | | | | | | | |
| LX-2601 | 100 | (±6.4) | 95.2 | (±7.4) | 81.8 | (±7.9) | 58.6 | (±2.3) | 21.8 | (±1.7) | | | | |
| LX-2602 | 100 | (±5) | 97.3 | (±2.3) | 92.5 | (±1.8) | 76.9 | (±1.1) | 47.6 | (±3.3) | | | | |
| LX-2702 | 100 | (±8.1) | 103.9 | (±7.3) | 99.1 | (±8.6) | 87.4 | (±4.4) | 49.6 | (±0.4) | 22.9 | (±9.1) | | |
| LX-2707 | 100 | (±8.2) | 95.1 | (±6.3) | 90.4 | (±6.8) | 64.8 | (±4.5) | 29 | (±4.8) | | | | |
| LX-2901 | 100 | (±9.3) | 103.3 | (±8.7) | 97.8 | (±5.4) | 104.8 | (±6.6) | 93.6 | (±8.7) | 49.9 | (±8.2) | | |
| LX-3001 | 100 | (±7.2) | 74.8 | (±6.7) | 1.1 | (±0.1) | | | | | | | | |
| LX-3002 | 100 | (±6.8) | 87.5 | (±5.7) | 1.2 | (±0.1) | | | | | | | | |
| LX-3106 | 100 | (±3.9) | 97.7 | (±3.8) | 87.4 | (±6.2) | 82.5 | (±4.8) | 59.7 | (±4.2) | 9.8 | (±0.5) | | |
| LX-3107 | 100 | (±5) | 87.7 | (±5.8) | 83.4 | (±4.8) | 74.2 | (±3.3) | 33.4 | (±1.7) | | | | |
| LX-3200 | 100 | (±4.5) | 92.2 | (±6.5) | 78.6 | (±3.9) | 14.3 | (±3.1) | | | | | | |
| LX-3201 | 100 | (±7.5) | 99.3 | (±6.1) | 91.9 | (±2.1) | 20.1 | (±2.1) | | | | | | |
| LX-3203 | 100 | (±10) | 106.9 | (±3) | 15.4 | (±0.7) | | | | | | | | |
| LX-3303 | 100 | (±8.8) | 83.9 | (±1) | 69.4 | (±7.4) | 13.9 | (±0.6) | | | | | | |

As a result, the higher the concentration of the test compound in senescence-induced cells, the faster the cell survival rate decreased. From this, it was confirmed again that the test compounds of the present invention can effectively eliminate senescent cells by inducing their death.

In this way, the cell survival rate decreases in senescent cells and the total number of cells decreases sharply. However, when cells in which senescence was not induced by treatment with the control group (DMSO) were treated with the test compound, the total number of cells continued to increase with growth. From this, it was reconfirmed that the test compounds can selectively remove senescent cells, as shown in the results of LX-112 in Example 1-2 of WO2023-085787 A1. The property of killing and removing senescent cells is not simply related to the generation of reactive oxygen species (ROS) within cells. This is because even though H₂O₂ (Deoksan Science), which has a strong oxidizing effect that increases oxygen radicals within cells, or NAC (N-acetyl cysteine, Sigma), which has a strong antioxidant effect that reduces reactive oxygen species, is used at excessive concentrations (1 or 20 mM), senescent cells were hardly removed even after treatment. Ultimately, it was confirmed that the test compounds of the present invention eliminate senescent cells through a unique target and a different death mechanism from apoptosis.

### Example 12. Removal of Senescent Cancer Cells by the Compound of the Present Invention

A549, a human-derived lung adenocarcinoma cancer cell line (ATCC), was cultured in the designated medium (RPMI-1640, GenDEPOT; 10% FBS, GenDEPOT; 100 U/ml Penicillin & Streptomycin, Invitrogen). After culturing, aging was induced in the same manner as Example 1 of WO2023-085787 A1. Cells were treated with doxorubicin or control (DMSO) and cultured. Then, cells were treated with the test compound at 30 μM (except that LX-305, LX-905, LX-1302, LX-1601, LX-1603, LX-2003, LX-2702, LX-2901, and LX-3106 were 90 UM, LX-1103, LX-2005, and LX-2007 were 270 UM). In the same manner as Example 10 of WO2023-085787 A1, the amount of HMGN1 secreted in the cell culture was measured using an enzyme-linked immunosorbent assay (ELISA Kit, AssayGenie). In cancer cells in which senescence was not induced by treatment with the control group (DMSO), the amount of HMGN1 above the detection limit could not be measured in both untreated and treated conditions with the test compound. On the other hand, the amount of HMGN1 measured in cancer cells in which senescence was induced by treatment with doxorubicin was not treated with the test compound, and the amount was calculated as 100%, and the amount of HMGN1 was measured in conditions in which the test compound were treated in senescent cancer cells. The results are shown in Table below.

TABLE 5

| | HMGN1 | | HMGN1 | | HMGN1 | | HMGN1 | | HMGN1 |
|---|---|---|---|---|---|---|---|---|---|
| DMSO | 100 | LX-305 | 227 | LX-401 | 277 | LX-403 | 320 | LX-405 | 310 |
| LX-801 | 329 | LX-905 | 217 | LX-1102 | 324 | LX-1103 | 158 | LX-1302 | 147 |
| LX-1601 | 257 | LX-1603 | 163 | LX-1801 | 295 | LX-1902 | 271 | LX-1903 | 267 |
| LX-2001 | 328 | LX-2002 | 314 | LX-2003 | 218 | LX-2005 | 252 | LX-2007 | 192 |
| LX-2101 | 297 | LX-2102 | 233 | LX-2601 | 193 | LX-2602 | 283 | LX-2702 | 237 |
| LX-2707 | 288 | LX-2901 | 189 | LX-3001 | 261 | LX-3002 | 314 | LX-3106 | 236 |
| LX-3107 | 267 | LX-3200 | 305 | LX-3201 | 309 | LX-3203 | 293 | LX-3303 | 239 |

As a result, the amount of HMGN1 secreted into aged cancer cells increased when cells died with treatment with the test compound. In addition, as shown in the results obtained by being treated with LX-112 in Example 10 of WO2023-085787 A1, caspase inhibitors did not effectively inhibit the death of senescent cells by the test compound. Despite the presence of caspase inhibitors, the test compound still could remove senescent cells by inducing cell death. From this, it was confirmed that the test compounds induce the death of aged cancer cells through a different mechanism from ABT-263.

Furthermore, the degree of death of aged cancer cells was analyzed based on the weight of HMGN1 in the same manner as in Example 1 of WO2023-085787 A1. Cancer cells in which aging was induced by treatment with doxorubicin were treated with the test compound at designated concentrations and measured using the CellTiter-Glo Luminescent Cell Viability Assay (Promega). The cell viability results are shown in Table below.

TABLE 6

| Survival (%) | 0 μM | | 1.1 μM | | 3.3 μM | | 10 μM | | 30 μM | | 90 μM | | 270 μM | |
|---|---|---|---|---|---|---|---|---|---|---|---|---|---|---|
| LX-305 | 100 | (±11.1) | 99.2 | (±6.7) | 93.8 | (±8.4) | 63.1 | (±1.8) | 41 | (±5.7) | 13.3 | (±1.1) | | |
| LX-401 | 100 | (±6.4) | 89.7 | (±8.1) | 68.6 | (±7.8) | 42.1 | (±4.7) | 16.8 | (±1.1) | | | | |
| LX-403 | 100 | (±8.8) | 40.2 | (±2.8) | 33.7 | (±2.1) | 3.5 | (±0.8) | | | | | | |
| LX-405 | 100 | (±8.1) | 23.8 | (±1.7) | | | | | | | | | | |
| LX-801 | 100 | (±5.9) | 78.1 | (±7.3) | 58.5 | (±2.1) | 12.7 | (±1.5) | | | | | | |
| LX-905 | 100 | (±9.3) | 100.8 | (±2.3) | 97.5 | (±3) | 83.1 | (±1.5) | 79.5 | (±3.3) | 22.7 | (±1.1) | | |
| LX-1102 | 100 | (±4.5) | 99.5 | (±2.9) | 87.7 | (±2.6) | 17.7 | (±2.4) | | | | | | |
| LX-1103 | 100 | (±3.2) | 96.6 | (±2.4) | 96.4 | (±1.7) | 95.1 | (±8.2) | 91 | (±2.6) | 91.1 | (±4.4) | 63.7 | (±6.8) |
| LX-1302 | 100 | (±4.2) | 95.5 | (±4.6) | 94.8 | (±4.7) | 102.2 | (±6.2) | 101.7 | (±3.2) | 69.6 | (±3.3) | 19.8 | (±1.4) |
| LX-1601 | 100 | (±3.9) | 114 | (±4.7) | 115 | (±8.3) | 117.5 | (±6.2) | 74.2 | (±4.2) | 3.1 | (±0.1) | | |
| LX-1603 | 100 | (±5.7) | 99.6 | (±2.8) | 94.3 | (±4.1) | 95.7 | (±5.5) | 96.5 | (±4.2) | 49.8 | (±2.7) | | |
| LX-1801 | 100 | (±3.3) | 60.6 | (±3.2) | 43.9 | (±3.4) | 25.1 | (±1.7) | | | | | | |
| LX-1902 | 100 | (±11.4) | 66.2 | (±6.6) | 40.1 | (±2.7) | 4.2 | (±0.2) | | | | | | |
| LX-1903 | 100 | (±9.8) | 93.1 | (±6.1) | 56.2 | (±4.8) | 12.3 | (±1) | | | | | | |
| LX-2001 | 100 | (±3.7) | 98.3 | (±3.2) | 77.7 | (±4.6) | 5.1 | (±0.0) | | | | | | |
| LX-2002 | 100 | (±8.7) | 99.6 | (±6.4) | 104 | (±5.6) | 10.4 | (±0.8) | | | | | | |
| LX-2003 | 100 | (±6.7) | 97.5 | (±5.2) | 94.8 | (±4.8) | 90.6 | (±3.2) | 52.7 | (±4.2) | 18.3 | (±1.1) | | |
| LX-2005 | 100 | (±7.9) | 101.5 | (±4.1) | 92.6 | (±3.7) | 95.7 | (±2.9) | 82.9 | (±6.6) | 57.2 | (±2.3) | 5.3 | (±0.2) |
| LX-2007 | 100 | (±2.8) | 111.3 | (±2.9) | 103.9 | (±2.4) | 96.3 | (±7.5) | 87.9 | (±2.9) | 62.8 | (±2.4) | 32.4 | (±2.2) |
| LX-2101 | 100 | (±4.1) | 71.6 | (±3.8) | 18.7 | (±1.2) | | | | | | | | |
| LX-2102 | 100 | (±6.1) | 88.4 | (±4.4) | 15.8 | (±0.8) | | | | | | | | |
| LX-2601 | 100 | (±3.3) | 98.6 | (±2.8) | 86 | (±3.9) | 59.7 | (±3.4) | 50.7 | (±2.8) | | | | |
| LX-2602 | 100 | (±4.1) | 102.7 | (±4.9) | 107.4 | (±3.7) | 96 | (±3.9) | 32.5 | (±1.1) | | | | |
| LX-2702 | 100 | (±4.5) | 96.7 | (±3.5) | 100.2 | (±7.6) | 94.4 | (±8.2) | 55.1 | (±5.1) | 9.5 | (±1.2) | | |
| LX-2707 | 100 | (±8.2) | 95.1 | (±6.3) | 90.4 | (±6.8) | 64.8 | (±4.5) | 29 | (±4.8) | | | | |
| LX-2901 | 100 | (±4.2) | 89 | (±3.7) | 87.8 | (±2.1) | 90.4 | (±2.8) | 92.3 | (±4.9) | 37.2 | (±1.4) | | |
| LX-3001 | 100 | (±4.8) | 101.6 | (±3.3) | 95.6 | (±3.8) | 22.6 | (±1.1) | | | | | | |
| LX-3002 | 100 | (±3.3) | 90.2 | (±3.3) | 9.9 | (±0.7) | | | | | | | | |
| LX-3106 | 100 | (±7.2) | 108.5 | (±8) | 98.1 | (±5.2) | 91.7 | (±8.8) | 76.8 | (±5.9) | 4.7 | (±0.0) | | |
| LX-3107 | 100 | (±2.3) | 96.5 | (±1.7) | 88.7 | (±4.8) | 77.8 | (±2.5) | 4.4 | (±0.2) | | | | |
| LX-3200 | 100 | (±3.5) | 94.9 | (±4.2) | 91.7 | (±7.1) | 61.6 | (±2.7) | 1.6 | (±0.0) | | | | |
| LX-3201 | 100 | (±0.4) | 96.5 | (±3.8) | 82.7 | (±4.4) | 68.1 | (±6.1) | 12.8 | (±0.5) | | | | |
| LX-3203 | 100 | (±0.9) | 105.4 | (±4.7) | 72.9 | (±1.4) | 21.6 | (±0.6) | | | | | | |
| LX-3303 | 100 | (±3.2) | 100.6 | (±3.6) | 93 | (±1.7) | 53.8 | (±5.9) | 11.7 | (±0.5) | | | | |

As a result, the higher the concentration of the test compound in senescence-induced cancer cells, the faster the cell survival rate decreased. From this, it was reconfirmed that the test compounds of the present invention can effectively eliminate aged cancer cells by inducing the death of aged cancer cells.

In this way, the cell survival rate decreases in aged cancer cells and the total number of cells decreases sharply. However, when cancer cells in which senescence was not induced by treatment with the control group (DMSO) were treated with the test compound, the total number of cells continued to increase with growth. From this, it was confirmed again that the test compounds can selectively remove aged cancer cells by inducing the death of aged cancer cells only.

Therefore, the compounds of the present invention can be usefully used in prosenescence anti-cancer therapy to senescent cancer cells and subsequently remove the senescent cancer cells. That is, the compounds of the present invention can be used for the treatment or prevention of proliferative diseases such as cancer.

### Example 13. Removal of Senescent Human Cells by Secreted Fluid from Cells Treated with the Compound of the Present Invention

In the same manner as in Example 1 of WO2023-085787 A1, senescence-induced foreskin cells treated with doxorubicin or control (DMSO) were treated with LX-401 or LX-405 at a concentration of 30 UM, respectively. After 3 days, cell viability was measured using CellTiter-Glo Luminescent Cell Viability Assay (Promega) or light microscopy. As a result of the measurement, it was confirmed that when the test compound was treated at high concentrations, they induced the death of most cells in both normally proliferating and senescent cells.

After treating cells with the test compound at a high concentration that induces the death of most cells in both non-senescence-induced and senescence-induced cells, the cells were washed three times with PBS to remove the test compound, and replaced with fresh medium and cultured for an additional 3 hours at the time when the compound has entered into the cell but has not yet induced cell death (approximately 1 to 3 hours after compound treatment). During this additional culture, by checking with an optical microscope, etc., the medium supernatants containing secreted fluid from normally proliferating and senescent cells in which cell death was induced were obtained. The obtained medium supernatant was replaced with the medium of normally proliferating and senescent cells, respectively, and cell survival rate was measured after 3 days. The results are shown in Table below.

TABLE 7

| | Supernatant from | Supernatant- | |
|---|---|---|---|
| | Death-Induced Cells | Treated Cells | Survival (%) |
| LX-401 | Proliferating Cells | Proliferating Cells | 88.1 (±4.2) |
| | | Senescent Cells | 48.2 (±4.2) |
| | Senescent Cells | Proliferating Cells | 95.1 (±5.7) |
| | | Senescent Cells | 42.2 (±4) |
| | No Cells | Proliferating Cells | 97.9 (±7.2) |
| | | Senescent Cells | 101.1 (±5.5) |
| LX-405 | Proliferating Cells | Proliferating Cells | 104 (±3) |
| | | Senescent Cells | 41.3 (±3.6) |
| | Senescent Cells | Proliferating Cells | 100.2 (±5.6) |
| | | Senescent Cells | 52.7 (±0.1) |
| | No Cells | Proliferating Cells | 99.4 (±2.4) |
| | | Senescent Cells | 102.8 (±4.6) |

As a result, both medium supernatants obtained from normally proliferating and senescent cells whose cell death was induced by the test compound selectively induced the death of senescent cells. However, both media failed to induce death of normally proliferating cells. In this way, both medium supernatants obtained from normally proliferating and senescent cells whose cell death was induced by LX-112 selectively induced the death of senescent cells.

In addition, the medium supernatant obtained through the same process as above from the medium treated with the test compound without culturing cells (No Cells) did not induce the death of both normally proliferating and senescent cells. From this, it was confirmed that the medium supernatant comprising substances secreted from cells whose death was induced by treatment with the test compound can induce the death of senescent cells and effectively remove senescent cells.

Furthermore, other test compounds were used in the same manner in Example 11 at high concentrations of 30 μM, 90 μM, and 270 μM, respectively, and the culture medium supernatant comprising secreted fluid from dying senescent cells were obtained to measure the degree of death of senescent cells. The cell viability results are shown in Table below.

TABLE 8

| | Survival | | Survival | | Survival | | Survival | | Survival |
|---|---|---|---|---|---|---|---|---|---|
| LX-305 | 89.5 | LX-401 | 42.2 | LX-403 | 45.1 | LX-405 | 52.7 | LX-801 | 52.7 |
| | (±5.2) | | (±4) | | (±1.5) | | (±0.1) | | (±3.7) |
| LX-905 | 46.5 | LX-1102 | 52.9 | LX-1103 | 101.8 | LX-1302 | 98.5 | LX-1601 | 81.4 |
| | (±1) | | (±3.2) | | (±4.9) | | (±3.5) | | (±3.4) |
| LX-1603 | 97.5 | LX-1801 | 41.6 | LX-1902 | 53.4 | LX-1903 | 62.8 | LX-2001 | 57.8 |
| | (±6.1) | | (±2.7) | | (±4.4) | | (±6.1) | | (±3.1) |
| LX-2002 | 58.3 | LX-2003 | 64.2 | LX-2005 | 91.6 | LX-2007 | 95.7 | LX-2101 | 39.7 |
| | (±2.5) | | (±3.9) | | (±2.5) | | (±3.7) | | (±2.5) |
| LX-2102 | 46.3 | LX-2601 | 67.3 | LX-2602 | 69.5 | LX-2702 | 68.6 | LX-2707 | 68.6 |
| | (±3.4) | | (±4.2) | | (±6.2) | | (±3.1) | | (±3.2) |
| LX-2901 | 97.5 | LX-3001 | 58.7 | LX-3002 | 46.8 | LX-3106 | 72.6 | LX-3107 | 62.1 |
| | (±5.6) | | (±3.6) | | (±4.1) | | (±1.3) | | (±5.3) |
| LX-3200 | 54.9 | LX-3201 | 55.2 | LX-3203 | 57.1 | LX-3303 | 53.5 | | |
| | (±0.7) | | (±1.7) | | (±4.1) | | (±5.6) | | |

As a result, it was confirmed again that the medium supernatant comprising secreted fluid from cells whose death was induced by treatment with the test compound can effectively remove senescent cells by inducing their death. The same results were confirmed in LX-112.

### Example 14. Removal of Senescent Cancer Cells by Secreted Fluid from Cells Treated with the Compound of the Present Invention

In the same way as Examples 12 and 13, senescence of cancer (A549) cells was induced by treatment with doxorubicin or the control group (DMSO), and the cells were cultured. The cells were treated with LX-405 at a concentration of 30 μM. 3 days later, cell viability was measured using CellTiter-Glo Luminescent Cell Viability Assay (Promega) or an optical microscope. In the same manner as in Example 13, the medium supernatants comprising secreted fluid from normally proliferating and senescent cancer cells was obtained. Normally proliferating and senescent-induced cancer cells were treated with the medium supernatants. Afterwards, the survival rate of cells was measured and the results are shown in Table below.

TABLE 9

| | | Supernatant from | Supernatant- | |
|---|---|---|---|---|
| | Cells | Death-Induced Cells | Treated Cells | Survival (%) |
| LX-405 | A549 | Proliferating Cells | Proliferating Cells | 98.6 (±4.8) |
| | | | Senescent Cells | 78.8 (±7.7) |
| | | Senescent Cells | Proliferating Cells | 105.2 (±4.9) |
| | | | Senescent Cells | 72.5 (±5.7) |
| | | No Cells | Proliferating Cells | 101.4 (±1.3) |
| | | | Senescent Cells | 98.2 (±2.8) |

As a result, it was confirmed that the medium supernatant comprising secreted fluid from cancer cells whose death was induced by treatment with the test compound could more effectively remove aged cancer cells compared to normally proliferating cancer cells.

Furthermore, other test compounds were used in the same manner in Example 12 above at high concentrations of 30 μM, 90 μM, and 270 μM, respectively, and the cultured medium supernatant comprising secreted fluid from aged cancer cells that were killed were obtained. As a result of measuring the degree of death of aged cancer cells, it was confirmed that aged cancer cells can be effectively removed, as shown in Example 12.

### Example 15. Removal of Senescent Cells and Related Anti-Aging Activity by the Compound of the Present Invention in Aging Mice

In the same manner as in Example 7-1 of WO2023-085787 A1, naturally aged mice were administered LX-405 (5% DMSO in PBS solution, 10 mg/kg, intraperitoneal administration), LX-801 (PBS, 100 mg/kg, intraperitoneal administration), LX-1102 (0.5% CMC-Na solution, 100 mg/kg, oral administration), LX-1801 (PBS, 50 mg/kg, intraperitoneal administration), LX-2101 (0.5% CMC-Na solution, 10 mg/kg, intraperitoneal administration), and LX-3403 (mouse anti-PD-1 antibody (BioXcell), 100 μg, intraperitoneal administration) for 5 days under the following conditions. The control group was administered only the solution in the same volume. After drug administration was completed, the method described in each Example below was performed. The mouse was sacrificed in the same manner as Example 7-2 and 7-6 of WO2023-085787 A1. Total RNA was extracted from liver tissue, and qRT-PCR was performed and analyzed using synthesized cDNA. The table below shows the mRNA amounts of p16, a aging-related marker, and MMP3 and IL6, which are aging-related secretory factors (SASP) in mice in which natural aging was induced and without or with the test compound treatment. The mRNA amounts of p16, MMP3, and IL6 after natural aging were induced were calculated as 100% and compared them with the mRNA amounts of p16, MMP3, and IL6 after treatment with the test compound. Furthermore, an open field test was performed to analyze aging-related frailty in mice using the same method as Example 7-7 of WO2023-085787 A1. The table below shows the quantitative results of activity evaluation, which is a criterion for determining frailty, under conditions in which natural aging-induced mice were not treated with and treated with the test compound. The amount of activity evaluated after inducing aging was calculated as 100% and compared it with the amount of activity evaluated after treatment with the test compound.

TABLE 10

| | p16 | MMP3 | IL6 | Open Field Test |
|---|---|---|---|---|
| | mRNA (%) | mRNA (%) | mRNA (%) | Time in Center (%) |
| LX-405 | 76.9 (±6.8) | 64.7 (±11.5) | 73.6 (±7.4) | 201.5 (±18.3) |
| LX-801 | 69.4 (±6.9) | 76.5 (±4.2) | 71.1 (±1.8) | 241.8 (±17.9) |
| LX-1102 | 75.2 (±5.5) | 89.7 (±12.5) | 84.5 (±8.9) | 163 (±7.9) |
| LX-1801 | 77.4 (±5.2) | 47.1 (±15.6) | 56.8 (±19.3) | 252.9 (±28) |
| LX-2101 | 62.5 (±13.1) | 53.6 (±14.3) | 64.1 (±8.8) | 215.3 (±30.8) |
| LX-3403 | 78.1 (±5.1) | 70.4 (±3.7) | 77.7 (±5.9) | 178.2 (±7.3) |

As a result, as shown in Example 7-2, Example 7-6, and Example 7-5 of WO2023-085787 A1, when mice naturally aged, the amounts of p16, MMP3, and IL6 increased in liver tissue. On the other side, when treated with the test compound, the mRNA amounts of p16, MMP3, and IL6 decreased. From this, it was confirmed that the test compounds effectively reduce the amount of p16, an aging marker, and MMP3 and IL6, aging-related secretory factors (SASP), by eliminating senescent cells that increase in mice due to natural aging.

Furthermore, as shown in Example 7-7 of WO2023-085787 A1, when mice naturally aged, activity decreased, but when treated with the test compound, activity increased. From this, it was confirmed that the test compounds improved frailty of mice by removing senescent cells that increase as mice age.

### Example 16. Removal of Senescent Cells and Related Anti-Aging Activity by Senescent Cells Killed by the Compound of the Present Invention in Aging Mice

To obtain preadipocytes, groin fat tissue was collected from an 8-week-old female C57BL6J mouse (KRIBB Laboratory Animal Resource Center). The fat tissue was cut into small pieces and treated with collagenase to decompose the fat tissue, then centrifuged to collect the stromal vascular fraction (SVF) containing preadipocytes. It was washed with a washing medium (HBSS, GenDEPOT; 3.5% Albumin Fraction V, GenDEPOT; 100 U/ml Penicillin & Streptomycin, Invitrogen), mixed again, and extracted by filtering through a 100 μm nylon mesh. The isolated preadipocytes were cultured in the designated medium (DMEM, GenDEPOT; 20% FBS, GenDEPOT; 100 U/ml Penicillin & Streptomycin, Invitrogen). In the same manner as Example 1 of WO2023-085787 A1, cells were treated with doxorubicin at a concentration of 250 nM for 24 hours and cultured for an additional 21 days to induce cell senescence. Cells were treated with doxorubicin at a concentration of 250 nM for 24 hours and cultured for an additional 21 days to induce cell senescence. By treating the test compound in the same manner as in Example 13, it was confirmed using an optical microscope that the death of most senescent cells was induced. The killed preadipocytes were washed using PBS (Welgene) and then mixed with PBS at a concentration of 5×10⁵ cells/200 μl. It was administered subcutaneously to 8-week-old female C57BL6J mice (KRIBB Laboratory Animal Resource Center). At 7 and 10 days after administration of the killed senescent cells, 2 million senescent preadipocytes were mixed in 200 μl of PBS and administered into the abdominal cavity of mice. After administration of killed senescent cells, obesity was induced in mice with a high-fat diet (DIO) for 8 weeks in the same manner as in Example 9-1 of WO2023-085787 A1. Afterwards, the mouse was sacrificed in the same manner as in Example 7-2 of WO2023-085787 A1, total RNA was extracted from the groin fat tissue, and qRT-PCR was performed and analyzed using the synthesized cDNA. The table below shows the mRNA amounts of p16, an aging marker, and MMP3 and IL6, which are aging-related secretory factors (SASP), under conditions of not administering and administering cells whose death was induced by the test compound. The mRNA amount of p16, MMP3, and IL6 without administration of senescent cells whose death was induced by the test compound were calculated as 100%, and the amount of p16, MMP3, and IL6 after administration of the senescent cells whose death was induced by the test compound were compared with them. Furthermore, an open field test was performed to analyze aging-related frailty in mice using the same method as Example 7-7 of WO2023-085787 A1. The table below shows the results quantitatively showing the activity, which is a criterion for determining frailty, under the conditions of not administering and administering senescent cells whose death was induced by the test compound. The amount of activity evaluation without administration of senescent cells whose death was induced by the test compound was calculated as 100% and compared it with the amount of activity evaluation after administration of senescent cells whose death was induced by the test compound.

TABLE 11

| | p16 | MMP3 | IL6 | Open Field Test |
|---|---|---|---|---|
| | mRNA (%) | mRNA (%) | mRNA (%) | Time in Center (%) |
| LX-405 | 72.4 (±3.9) | 80.5 (±2.8) | 68.7 (±9.1) | 174.2 (±12.5) |
| LX-801 | 79.8 (±3.4) | 81.7 (±2.9) | 79.4 (±4.6) | 216.8 (±12.8) |
| LX-1102 | 82.9 (±4.8) | 95.4 (±9.6) | 76.2 (±7.1) | 146.2 (±17.1) |
| LX-1801 | 62.5 (±9.4) | 67.8 (±13.1) | 56.4 (±12.4) | 173.4 (±18.6) |
| LX-2101 | 73.8 (±9.7) | 68.3 (±16.4) | 75.9 (±6.7) | 144.9 (±26.6) |

As a result, as shown in Example 9-2 and Example 9-3 of WO2023-085787 A1, when mice aged due to obesity, the mRNA amounts of p16, MMP3, and IL6 increased in adipose tissue, etc. However, the mRNA amounts of p16, MMP3, and IL6 decreased when senescent cells whose death was induced by the test compound were administered to mice and senescence was induced by obesity. From this, it was confirmed that when senescent cells induced to die by the test compound are administered to mice, they can effectively reduce the amount of p16, an aging marker, and MMP3 and IL6, aging-related secretory factors (SASP) by removing senescent cells that increase in mice due to aging such as obesity.

Furthermore, as shown in Example 9-4 of WO2023-085787 A1, activity decreased when mice aged due to obesity. However, when senescent cells induced to die by the test compound were administered to mice and aging was induced, activity increased in comparison. From this, it was confirmed that administering senescent cells whose death was induced by the test compound to mice improved frailty in mice by eliminating senescent cells that increase in mice due to aging such as obesity.

In this way, the effect of administration of senescent cells induced to die by the test compound, which improve frailty by removing senescent cells and reducing p16 and aging-related secretory factor (SASP), was not observed in the Rag-2 gene knockout C57BL6J mice (KRIBB Laboratory Animal Resource Center). Therefore, it was confirmed that the immune system in the body is involved in the effect of removing and reducing senescent cells using senescent cells whose death was induced by the test compound. Ultimately, combined administration of immune checkpoint inhibitors, cytokines (interferons, interleukins, etc.), and polysaccharide-K, which are known to contribute to this immune response process in the body, can further increase the effect of eliminating senescent cells. In this way, the combined administration of immune checkpoint inhibitors, cytokines, etc. along with HMGN1, which binds to the immune response receptor (Toll-like receptor), can synergistically activate the immune response. In this regard, in the above experiment in Example 16, it further reduced the amount of p16 mRNA by about 27% to intraperitoneally administering mouse anti-PD-1 (BioXcell), LX-3403, at a concentration of 100 μg each time twice a week for 8 weeks after 7 days of administration of senescent cells whose death was induced by the test compounds such as LX-405. From this, it was confirmed that senescent cells whose death was induced by the test compound could effectively remove increased senescent cells in mice with induced aging either alone or in combination with an immune response activator. Through this in vivo verification, the removal of senescent cells can be induced by contacting dying senescent cells with immune cells (dendritic cells, lymphocytes, macrophages, monocytes, neutrophils, epithelial cells, innate lymphoid cells, mast cells, eosinophils, basophils, natural killer cells, etc.) or immune tissues ex vivo.

### Example 17. Screening of Various Compounds that Remove Senescent Cells Through a Mechanism Different from that of Existing Senescent Cell Removal Agents

Using the same method as Example 11, compounds that induce senescent cell death through a mechanism similar to that of LX-112 (ethaselen (1,2-[bis(1,2-benzisoselenazolone-3 (2H)-ketone)]ethane)) were screened based on HMGN1 and others. The cells were treated with doxorubicin in the same manner as Example 1 of WO2023-085787 A1, and the test compound was treated at a concentration of 30 μM (except for LX-5211, LX-5433, LX-6530, LX-9105, LX-9118, LX-9321, and LX-9602 at 90 μM, and LX-9336 at 2.4 mM). The test compounds below were synthesized and purchased from Medchemexpress, Selleckchem, Cayman, Medkoo, TCI, Probechem, REONSIMI, etc. The amount of HMGN1 secreted into the cell culture medium was measured using an enzyme-linked immunosorbent assay (ELISA Kit, AssayGenie) in the same manner as Example 11. In non-senescent cells treated with the control group (DMSO), no amount of HMGN1 exceeding the sensitivity (35 μg/ml) for analysis was detected under both untreated and treated conditions with the test compound. On the other hand, in cells senescent with doxorubicin, an amount of HMGN1 of 80-90 μg/ml was measured. This amount was calculated as 100%, and the amount of HMGN1 was measured under conditions in which senescent cells were treated with the test compound, and the results are shown in the table below.

TABLE 12

| | HMGN1 |
|---|---|
| DMSO | 100 |
| LX-5201 | 294 |
| LX-5211 | 253 |
| LX-5328 | 242 |
| LX-5341 | 238 |
| LX-5352 | 194 |
| LX-5362 | 211 |
| LX-5401 | 214 |
| LX-5411 | 231 |
| LX-5424 | 327 |
| LX-5433 | 260 |
| LX-5449 | 309 |
| LX-5701 | 286 |
| LX-5702 | 279 |
| LX-5706 | 269 |
| LX-5707 | 258 |
| LX-5711 | 252 |
| LX-5721 | 255 |
| LX-5722 | 242 |
| LX-5731 | 235 |
| LX-5904 | 245 |
| LX-5906 | 277 |
| LX-5907 | 258 |
| LX-6202 | 241 |
| LX-6203 | 220 |
| LX-6205 | 293 |
| LX-6207 | 248 |
| LX-6212 | 306 |
| LX-6216 | 221 |
| LX-6233 | 241 |
| LX-6501 | 311 |
| LX-6514 | 281 |
| LX-6516 | 270 |
| LX-6530 | 225 |
| LX-6537 | 328 |
| LX-6800 | 239 |
| LX-6804 | 201 |
| LX-6806 | 311 |
| LX-6807 | 279 |
| LX-6809 | 325 |
| LX-6981 | 237 |
| LX-6982 | 234 |
| LX-6988 | 282 |
| LX-7103 | 318 |
| LX-7104 | 287 |
| LX-7111 | 271 |
| LX-7121 | 278 |
| LX-7122 | 278 |
| LX-7135 | 208 |
| LX-7261 | 251 |
| LX-7262 | 232 |
| LX-7263 | 267 |
| LX-7288 | 275 |
| LX-7403 | 225 |
| LX-7405 | 261 |
| LX-7451 | 219 |
| LX-7463 | 222 |
| LX-7621 | 304 |
| LX-7622 | 293 |
| LX-8721 | 208 |
| LX-8726 | 235 |
| LX-8801 | 274 |
| LX-8804 | 256 |
| LX-8805 | 259 |
| LX-8812 | 281 |
| LX-9105 | 281 |
| LX-9107 | 252 |
| LX-9114 | 218 |
| LX-9118 | 295 |
| LX-9131 | 330 |
| LX-9136 | 263 |
| LX-9137 | 226 |
| LX-9302 | 323 |
| LX-9314 | 320 |
| LX-9321 | 256 |
| LX-9335 | 239 |
| LX-9336 | 203 |
| LX-9339 | 222 |
| LX-9343 | 305 |
| LX-9409 | 221 |
| LX-9421 | 201 |
| LX-9422 | 244 |
| LX-9423 | 227 |
| LX-9425 | 218 |
| LX-9561 | 205 |
| LX-9562 | 267 |
| LX-9603 | 281 |
| LX-9565 | 204 |
| LX-9601 | 288 |
| LX-9602 | 287 |
| LX-9713 | 263 |
| LX-9717 | 226 |
| LX-9725 | 283 |
| LX-9732 | 224 |
| LX-9744 | 214 |

As a result, the amount of HMGN1 secreted from senescent cells that died when treated with the test compound increased, similar to LX-112. Furthermore, as with the results obtained by treating cells with LX-112 in Example 10 of WO2023-085787 A1, the caspase inhibitor did not effectively inhibit the test compound-induced senescent cell death. Even in the presence of the caspase inhibitor, the test compound was still able to induce and eliminate senescent cells. This confirmed that the test compound induces senescent cell death through a different mechanism than ABT-263.

Furthermore, the degree of senescent cell death, as analyzed based on the increased amount of HMGN1, was evaluated using the same method as Example 1 of WO2023-085787 A1. Cells induced to undergo senescence by doxorubicin treatment were treated with test compound at designated concentrations and measured using the CellTiter-Glo Luminescent Cell Viability Assay (Promega). The cell viability results are shown in the table below.

TABLE 13

| Survival (%) | 0 μM | | 1.1 μM | | 3.3 μM | | 10 μM | | 30 μM | | 90 μM | | 270 μM |
|---|---|---|---|---|---|---|---|---|---|---|---|---|---|
| LX-5201 | 100 | (±3.5) | 102.5 | (±9.9) | 98.5 | (±2.7) | 88.3 | (±8.9) | 37.7 | (±2.7) | | | |
| LX-5211 | 100 | (±9.6) | 97.6 | (±4.3) | 95.2 | (±7.5) | 92.7 | (±8.5) | 87.1 | (±4.5) | 22.7 | (±2.0) | |
| LX-5328 | 100 | (±4.3) | 54.3 | (±1.6) | 21.8 | (±0.7) | 2.0 | (±0.1) | | | | | |
| LX-5341 | 100 | (±3.9) | 99.1 | (±3.5) | 91.5 | (±5.7) | 48.5 | (±4.9) | 3.5 | (±0.2) | | | |
| LX-5352 | 100 | (±4.9) | 87.9 | (±6.3) | 61.6 | (±1.9) | 32.4 | (±2.4) | 1.9 | (±0.1) | | | |
| LX-5362 | 100 | (±5.7) | 42.5 | (±1.7) | 11.6 | (±0.2) | | | | | | | |
| LX-5401 | 100 | (±6.8) | 99.5 | (±9.1) | 96.3 | (±8.6) | 66.0 | (±6.4) | 24.6 | (±2.0) | | | |
| LX-5411 | 100 | (±4.3) | 75.4 | (±3.5) | 43.8 | (±1.7) | 2.8 | (±0.1) | | | | | |
| LX-5424 | 100 | (±4.7) | 105.8 | (±4.4) | 91.3 | (±9.7) | 62.5 | (±3.3) | 10.5 | (±1.4) | | | |
| LX-5433 | 100 | (±9.5) | 108.3 | (±4.6) | 99.4 | (±11.7) | 71.1 | (±5.3) | 51.8 | (±3.9) | 2.4 | (±0.1) | |
| LX-5449 | 100 | (±6.0) | 89.1 | (±4.5) | 75.8 | (±6.7) | 44.1 | (±4.6) | 8.7 | (±0.6) | | | |
| LX-5701 | 100 | (±8.0) | 91.4 | (±5.4) | 74.6 | (±6.9) | 19.3 | (±0.7) | | | | | |
| LX-5702 | 100 | (±4.7) | 97.5 | (±6.8) | 62.8 | (±6.1) | 5.2 | (±1.0) | | | | | |
| LX-5706 | 100 | (±3.8) | 96.5 | (±5.1) | 78.1 | (±3.3) | 12.7 | (±2.5) | | | | | |
| LX-5707 | 100 | (±7.3) | 102.8 | (±3.2) | 81.4 | (±5.1) | 27.3 | (±1.2) | | | | | |
| LX-5711 | 100 | (±4.1) | 95.1 | (±1.3) | 84.2 | (±8.4) | 63.7 | (±2.3) | 20.6 | (±2.3) | | | |
| LX-5721 | 100 | (±7.2) | 98.5 | (±3.6) | 77.3 | (±7.6) | 45.5 | (±3.4) | 13.4 | (±2.9) | | | |
| LX-5722 | 100 | (±11.8) | 91.5 | (±9.1) | 76.2 | (±6.0) | 34.1 | (±1.9) | 1.8 | (±0.1) | | | |
| LX-5731 | 100 | (±7.7) | 98.8 | (±6.5) | 89.8 | (±3.9) | 48.2 | (±7.3) | 11.7 | (±0.7) | | | |
| LX-5904 | 100 | (±5.2) | 103.1 | (±4.7) | 83.5 | (±5.6) | 25.9 | (±2.1) | 2.2 | (±0.2) | | | |
| LX-5906 | 100 | (±6.1) | 97.2 | (±4.1) | 78.4 | (±4.2) | 30.7 | (±3.0) | 0.4 | (±0.0) | | | |
| LX-5907 | 100 | (±6.7) | 95.5 | (±7.3) | 84.0 | (±7.5) | 19.5 | (±1.8) | 1.7 | (±0.1) | | | |
| LX-6202 | 100 | (±9.4) | 1.5 | (±0.1) | | | | | | | | | |
| LX-6203 | 100 | (±4.3) | 75.1 | (±8.4) | 53.7 | (±6.5) | 13.1 | (±0.7) | | | | | |
| LX-6205 | 100 | (±6.2) | 3.8 | (±0.2) | | | | | | | | | |
| LX-6207 | 100 | (±3.1) | 68.7 | (±8.5) | 56.1 | (±6.0) | 8.6 | (±1.7) | | | | | |
| LX-6212 | 100 | (±11.5) | 52.3 | (±7.0) | 5.5 | (±0.3) | | | | | | | |
| LX-6216 | 100 | (±4.8) | 94.5 | (±7.1) | 87.5 | (±2.4) | 68.8 | (±8.9) | 24.3 | (±2.1) | | | |
| LX-6233 | 100 | (±4.9) | 83.7 | (±4.4) | 61.2 | (±2.1) | 32.7 | (±2.1) | 11.3 | (±1.3) | | | |
| LX-6501 | 100 | (±4.4) | 94.5 | (±8.5) | 97.7 | (±5.0) | 71.5 | (±1.4) | 7.8 | (±1.1) | | | |
| LX-6514 | 100 | (±4.7) | 97.2 | (±5.1) | 96.2 | (±7.4) | 63.3 | (±6.1) | 2.7 | (±0.5) | | | |
| LX-6516 | 100 | (±7.2) | 93.3 | (±6.4) | 84.4 | (±7.0) | 71.1 | (±2.3) | 31.8 | (±2.8) | | | |
| LX-6530 | 100 | (±6.7) | 105.8 | (±8.1) | 106.3 | (±2.0) | 112.5 | (±7.9) | 46.1 | (±8.4) | | | |
| LX-6537 | 100 | (±5.7) | 90.9 | (±5.3) | 87.1 | (±8.5) | 67.3 | (±5.5) | 12.2 | (±1.5) | | | |
| LX-6800 | 100 | (±9.1) | 81.6 | (±2.3) | 73.4 | (±3.0) | 56.5 | (±5.9) | 2.2 | (±0.1) | | | |
| LX-6804 | 100 | (±8.5) | 87.5 | (±2.6 | 74.2 | (±2.7) | 28.5 | (±3.9) | | | | | |
| LX-6806 | 100 | (±6.1) | 95.4 | (±4.1) | 79.0 | (±3.8) | 61.2 | (±2.8) | 32.8 | (±3.8) | | | |
| LX-6807 | 100 | (±3.4) | 95.1 | (±8.9) | 93.3 | (±5.9) | 42.5 | (±1.2) | 6.9 | (±0.3) | | | |
| LX-6809 | 100 | (±7.9) | 87.5 | (±6.3) | 77.8 | (±4.4) | 53.3 | (±7.1) | 17.8 | (±2.8) | | | |
| LX-6981 | 100 | (±5.2) | 0.2 | (±0.0) | | | | | | | | | |
| LX-6982 | 100 | (±8.6) | 95.1 | (±5.0) | 86.8 | (±5.3) | 74.6 | (±3.9) | 10.5 | (±1.4) | | | |
| LX-6988 | 100 | (±3.5) | 93.5 | (±5.1) | 86.5 | (±7.2) | 53.7 | (±6.1) | 3.7 | (±0.6) | | | |
| LX-7103 | 100 | (±3.8) | 84.3 | (±3.2) | 60.0 | (±3.8) | 7.9 | (±0.3) | | | | | |
| LX-7104 | 100 | (±8.5) | 88.5 | (±6.2) | 75.7 | (±6.1) | 45.7 | (±6.2) | 2.8 | (±0.4) | | | |
| LX-7111 | 100 | (±2.4) | 78.1 | (±8.4) | 72.3 | (±8.8) | 22.0 | (±0.8) | | | | | |
| LX-7121 | 100 | (±8.8) | 94.7 | (±5.8) | 73.7 | (±2.2) | 57.9 | (±2.5) | 3.9 | (±0.8) | | | |
| LX-7122 | 100 | (±5.7) | 109.3 | (±10.6) | 111.1 | (±8.8) | 94.4 | (±4.6) | 39.3 | (±3.6) | | | |
| LX-7135 | 100 | (±7.4) | 75.7 | (±7.2) | 17.7 | (±1.6) | | | | | | | |
| LX-7261 | 100 | (±5.1) | 63.7 | (±5.4) | 12.2 | (±1.1) | | | | | | | |
| LX-7262 | 100 | (±6.4) | 52.8 | (±6.2) | 5.4 | (±0.3) | | | | | | | |
| LX-7263 | 100 | (±4.7) | 49.2 | (±3.6) | 0.9 | (±0.0) | | | | | | | |
| LX-7288 | 100 | (±5.5) | 41.8 | (±2.9) | 1.7 | (±0.1) | | | | | | | |
| LX-7403 | 100 | (±3.4) | 94.5 | (±5.7) | 90.7 | (±4.8) | 72.3 | (±7.6) | 38.4 | (±2.4) | | | |
| LX-7405 | 100 | (±3.0) | 100.4 | (±2.4) | 100.0 | (±3.2) | 79.0 | (±4.7) | 40.6 | (±4.8) | | | |
| LX-7451 | 100 | (±2.4) | 105.4 | (±13.1) | 96.4 | (±1.4) | 39.2 | (±5.4) | | | | | |
| LX-7463 | 100 | (±8.0) | 88.8 | (±2.5) | 78.3 | (±5.6) | 32.1 | (±4.5) | | | | | |
| LX-7621 | 100 | (±4.8) | 86.3 | (±7.4) | 82.1 | (±1.5) | 78.2 | (±5.9) | 50.2 | (±6.8) | | | |
| LX-7622 | 100 | (±2.6) | 99.1 | (±4.7) | 94.1 | (±9.2) | 75.7 | (±4.4) | 17.7 | (±2.5) | | | |
| LX-8721 | 100 | (±2.7) | 43.1 | (±5.3) | 13.5 | (±2.3) | | | | | | | |
| LX-8726 | 100 | (±6.5) | 52.3 | (±1.9) | 11.8 | (±0.9) | | | | | | | |
| LX-8801 | 100 | (±4.5) | 69.1 | (±3.6) | 32.5 | (±1.2) | 1.2 | (±0.2) | | | | | |
| LX-8804 | 100 | (±3.0) | 54.5 | (±1.8) | 22.7 | (±0.4) | | | | | | | |
| LX-8805 | 100 | (±5.9) | 99.1 | (±2.7) | 71.8 | (±3.2) | 42.1 | (±3.6) | 21.8 | (±1.5) | | | |
| LX-8812 | 100 | (±6.9) | 94.6 | (±4.3) | 89.5 | (±3.3) | 49.3 | (±2.8) | 29.2 | (±2.5) | | | |
| LX-9105 | 100 | (±6.4) | 102.3 | (±5.1) | 101.8 | (±6.5) | 96.0 | (±8.1) | 89.9 | (±8.6) | 4.2 | (±0.1) | |
| LX-9107 | 100 | (±8.7) | 105.8 | (±9.6) | 108.1 | (±6.9) | 87.3 | (±9.1) | 26.2 | (±1.2) | | | |
| LX-9114 | 100 | (±6.6) | 101.8 | (±6.3) | 104.2 | (±6.7) | 88.6 | (±6.9) | 16.1 | (±2.0) | | | |
| LX-9118 | 100 | (±4.4) | 102.1 | (±7.6) | 104.4 | (±4.4) | 94.1 | (±7.9) | 62.8 | (±3.6) | 4.9 | (±0.2) | |
| LX-9131 | 100 | (±4.7) | 81.9 | (±7.6) | 61.8 | (±5.5) | 47.7 | (±3.5) | 13.2 | (±1.6) | | | |
| LX-9136 | 100 | (±5.7) | 84.6 | (±1.8) | 76.9 | (±5.1) | 71.1 | (±2.5) | 23.1 | (±1.7) | | | |
| LX-9137 | 100 | (±3.9) | 90.5 | (±6.7) | 85.7 | (±9.3) | 43.8 | (±5.4) | 14.6 | (±2.5) | | | |
| LX-9302 | 100 | (±7.6) | 99.7 | (±2.5) | 96.0 | (±8.6) | 73.5 | (±5.4) | 34.3 | (±2.8) | | | |
| LX-9314 | 100 | (±9.6) | 84.7 | (±7.1) | 73.1 | (±9.4) | 69.2 | (±3.6) | 35.3 | (±2.5) | | | |
| LX-9321 | 100 | (±5.6) | 98.3 | (±6.5) | 95.3 | (±6.2) | 86.4 | (±4.8) | 79.6 | (±4.6) | 19.4 | (±2.4) | |
| LX-9335 | 100 | (±3.2) | 85.2 | (±8.0) | 73.8 | (±4.6) | 56.3 | (±5.9) | 12.2 | (±2.1) | | | |
| LX-9336 | 100 | (±8.0) | 96.2 | (±3.2) | 100.3 | (±7.3) | 103.7 | (±8.2) | 94.3 | (±8.8) | 90.5 | (±8.5) | 91.2 (±5.9) |
| LX-9339 | 100 | (±7.1) | 55.6 | (±2.6) | 17.5 | (±4.1) | | | | | | | |
| LX-9343 | 100 | (±3.5) | 101.2 | (±3.8) | 84.2 | (±4.7) | 74.2 | (±2.2) | 30.5 | (±1.4) | | | |
| LX-9409 | 100 | (±5.8) | 80.5 | (±4.7) | 75.6 | (±6.7) | 65.7 | (±7.4) | 15.3 | (±1.0) | | | |
| LX-9421 | 100 | (±4.2) | 92.1 | (±4.1) | 89.3 | (±2.2) | 67.9 | (±6.7) | 25.9 | (±2.7) | | | |
| LX-9422 | 100 | (±4.8) | 94.7 | (±3.0) | 74.9 | (±6.0) | 56.2 | (±3.4) | 6.7 | (±0.8) | | | |
| LX-9423 | 100 | (±9.2) | 79.5 | (±8.5) | 71.8 | (±9.6) | 25.4 | (±5.0) | 8.7 | (±1.5) | | | |
| LX-9425 | 100 | (±6.1) | 101.8 | (±6.0) | 98.6 | (±3.6) | 97.5 | (±6.7) | 66.8 | (±5.4) | | | |
| LX-9561 | 100 | (±6.7) | 100.3 | (±8.5) | 76.9 | (±7.6) | 63.8 | (±4.5) | 12.3 | (±1.0) | | | |
| LX-9562 | 100 | (±7.8) | 97.0 | (±2.9) | 97.3 | (±2.3) | 45.4 | (±1.9) | 14.0 | (±1.2) | | | |
| LX-9563 | 100 | (±6.9) | 93.9 | (±3.8) | 87.6 | (±5.7) | 58.2 | (±2.4) | 18.5 | (±1.0) | | | |
| LX-9565 | 100 | (±8.2) | 78.9 | (±5.7) | 70.2 | (±8.0) | 52.3 | (±5.1) | 6.1 | (±0.5) | | | |
| LX-9601 | 100 | (±7.4) | 96.9 | (±6.3) | 89.8 | (±13.2) | 78.8 | (±5.2) | 30.2 | (±2.5) | | | |
| LX-9602 | 100 | (±5.2) | 112.8 | (±5.9) | 102.7 | (±6.6) | 101.9 | (±7.1) | 92.2 | (±8.3) | 11.1 | (±1.6) | |
| LX-9713 | 100 | (±4.3) | 1.8 | (±0.1) | | | | | | | | | |
| LX-9717 | 100 | (±5.7) | 101.2 | (±3.8) | 92.7 | (±6.7) | 73.5 | (±3.6) | 21.5 | (±1.9) | | | |
| LX-9725 | 100 | (±3.9) | 49.3 | (±4.1) | 0.9 | (±0.0) | | | | | | | |
| LX-9732 | 100 | (±4.2) | 95.7 | (±4.9) | 83.5 | (±9.4) | 46.4 | (±3.6) | 3.8 | (±0.8) | | | |
| LX-9744 | 100 | (±6.6) | 100.8 | (±3.7) | 91.7 | (±3.1) | 69.8 | (±1.7) | 22.3 | (±2.2) | | | |

As a result, cell viability decreased rapidly as the concentration of the test compound increased in senescent cells. This reaffirmed that the test compound of the present invention can effectively remove senescent cells by inducing death of the senescent cells.

As such, while cell viability decreased in senescent cells, resulting in a sharp decrease in the total cell number, the total cell number continuously increased with growth when cells of the control group (DMSO) that were not senescent-induced were treated with the test compound. This reaffirmed that the test compound can selectively remove senescent cells, similar to the results of LX-112 in Example 1-2 of WO2023-085787 A1.

### Example 18. Removal of Senescent Cancer Cells by the Compound of the Present Invention

A549, a human lung adenocarcinoma cell line (ATCC), were cultured in a designated medium (RPMI-1640, GenDEPOT; 10% FBS, GenDEPOT; 100 U/ml Penicillin & Streptomycin, Invitrogen), and senescence was induced using the same method as in Example 1 of WO2023-085787 A1. The test compound was treated at a concentration of 30 μM (except for LX-5211, LX-5433, LX-6530, LX-6800, LX-9105, LX-9118, LX-9131, LX-9136, LX-9302, LX-9321, Lx-9565, and LX-9602 at 90 μM, and LX-9336 at 2.4 mM) in cells treated with doxorubicin. The amount of HMGN1 secreted into the cell culture medium was measured using an enzyme-linked immunosorbent assay (ELISA Kit, AssayGenie) in the same manner as in Example 10 of WO2023-085787 A1. In non-senescent cancer cells treated with the control group (DMSO) as in Example 11, no amount of HMGN1 exceeding the analyzable value was measured under both untreated and treated conditions with the test compound. On the other hand, in cancer cells treated with doxorubicin, where senescence was induced, the amount of HMGN1 measured under conditions where cells were not treated with the test compound was calculated as 100%, and the amount of HMGN1 was measured under conditions where the senescent cancer cells were treated with the test compound. The results are shown in the table below.

TABLE 14

| | HMGN1 |
|---|---|
| DMSO | 100 |
| LX-5201 | 269 |
| LX-5211 | 262 |
| LX-5328 | 261 |
| LX-5341 | 232 |
| LX-5352 | 210 |
| LX-5362 | 242 |
| LX-5401 | 300 |
| LX-5411 | 240 |
| LX-5424 | 280 |
| LX-5433 | 299 |
| LX-5449 | 305 |
| LX-5701 | 246 |
| LX-5702 | 263 |
| LX-5706 | 243 |
| LX-5707 | 237 |
| LX-5711 | 204 |
| LX-5721 | 268 |
| LX-5722 | 202 |
| LX-5731 | 307 |
| LX-5904 | 241 |
| LX-5906 | 256 |
| LX-5907 | 233 |
| LX-6202 | 311 |
| LX-6203 | 318 |
| LX-6205 | 213 |
| LX-6207 | 256 |
| LX-6212 | 327 |
| LX-6216 | 237 |
| LX-6233 | 228 |
| LX-6501 | 239 |
| LX-6514 | 326 |
| LX-6516 | 297 |
| LX-6530 | 270 |
| LX-6537 | 289 |
| LX-6800 | 302 |
| LX-6804 | 326 |
| LX-6806 | 223 |
| LX-6807 | 292 |
| LX-6809 | 275 |
| LX-6981 | 255 |
| LX-6982 | 306 |
| LX-6988 | 225 |
| LX-7103 | 241 |
| LX-7104 | 246 |
| LX-7111 | 204 |
| LX-7121 | 317 |
| LX-7122 | 259 |
| LX-7135 | 248 |
| LX-7261 | 266 |
| LX-7262 | 249 |
| LX-7263 | 283 |
| LX-7288 | 281 |
| LX-7403 | 234 |
| LX-7405 | 205 |
| LX-7451 | 232 |
| LX-7463 | 248 |
| LX-7621 | 237 |
| LX-7622 | 281 |
| LX-8721 | 289 |
| LX-8726 | 257 |
| LX-8801 | 281 |
| LX-8804 | 248 |
| LX-8805 | 226 |
| LX-8812 | 275 |
| LX-9105 | 250 |
| LX-9107 | 275 |
| LX-9114 | 250 |
| LX-9118 | 203 |
| LX-9131 | 276 |
| LX-9136 | 296 |
| LX-9137 | 264 |
| LX-9302 | 247 |
| LX-9314 | 291 |
| LX-9321 | 317 |
| LX-9335 | 246 |
| LX-9336 | 202 |
| LX-9339 | 263 |
| LX-9343 | 274 |
| LX-9409 | 218 |
| LX-9421 | 284 |
| LX-9422 | 310 |
| LX-9423 | 239 |
| LX-9425 | 235 |
| LX-9561 | 222 |
| LX-9562 | 269 |
| LX-9563 | 291 |
| LX-9565 | 329 |
| LX-9601 | 209 |
| LX-9602 | 303 |
| LX-9713 | 258 |
| LX-9717 | 231 |
| LX-9725 | 293 |
| LX-9732 | 241 |
| LX-9744 | 229 |

As a result, the amount of HMGN1 secreted from the mortal senescent cancer cells increased when treated with the test compound. Furthermore, as with the results obtained by treating LX-112 in Example 10 of WO2023-085787 A1, the caspase inhibitor did not effectively inhibit the death of senescent cells induced by the test compound. In contrast, even in the presence of the caspase inhibitor, the test compound was still able to induce the death of senescent cells and eliminate them. This confirmed that the test compound induces death of senescent cancer cells through a different mechanism than ABT-263.

Furthermore, the degree of death of senescent cancer cells, as analyzed based on the increase in HMGN1, was evaluated using the same method as in Example 1 of WO2023-085787 A1. Senescence-induced cancer cells treated with doxorubicin were treated with the test compound at designated concentrations, and cell viability was measured using the CellTiter-Glo Luminescent Cell Viability Assay (Promega). The results are shown in the table below.

TABLE 15

| Survival (%) | 0 μM | | 1.1 μM | | 3.3 μM | | 10 μM | | 30 μM | | 90 μM | | 270 μM |
|---|---|---|---|---|---|---|---|---|---|---|---|---|---|
| LX-5201 | 100 | (±7.0) | 102.4 | (±3.4) | 95.5 | (±3.2) | 61.5 | (±5.6) | 3.8 | (±0.1) | | | |
| LX-5211 | 100 | (±6.5) | 102.8 | (±5.5) | 98.8 | (±6.1) | 88.1 | (±4.5) | 49.2 | (±2.5) | 16.6 | (±1.1) | |
| LX-5328 | 100 | (±2.1) | 62.7 | (±4.3) | 33.3 | (±3.4) | 0.4 | (±0.0) | | | | | |
| LX-5341 | 100 | (±3.1) | 94.6 | (±6.9) | 95.1 | (±6.1) | 55.4 | (±3.2) | 13.7 | (±1.1) | | | |
| LX-5352 | 100 | (±4.3) | 97.6 | (±8.4) | 87.5 | (±4.4) | 51.8 | (±1.2) | 22.2 | (±2.9) | | | |
| LX-5362 | 100 | (±4.8) | 61.8 | (±2.5) | 33.4 | (±3.8) | | | | | | | |
| LX-5401 | 100 | (±12.9) | 92.7 | (±9.6) | 75.8 | (±6.4) | 43.8 | (±2.0) | 1.9 | (±0.1) | | | |
| LX-5411 | 100 | (±3.9) | 75.4 | (±2.8) | 40.1 | (±4.1) | 5.3 | (±0.8) | | | | | |
| LX-5424 | 100 | (±8.6) | 98.9 | (±2.0) | 91.1 | (±1.7) | 74.3 | (±3.0) | 21.8 | (±0.2) | | | |
| LX-5433 | 100 | (±7.7) | 97.8 | (±7.9) | 88.2 | (±9.3) | 76.5 | (±5.5) | 64.7 | (±3.3) | 19.3 | (±0.1) | |
| LX-5449 | 101.0 | (±7.4) | 91.3 | (±5.0) | 77.2 | (±7.1) | 44.3 | (±1.5) | 2.4 | (±0.1) | | | |
| LX-5701 | 100 | (±7.0) | 82.3 | (±9.5) | 72.4 | (±7.1) | 43.8 | (±1.1) | 16.2 | (±1.3) | | | |
| LX-5702 | 100 | (±5.1) | 91.8 | (±4.9) | 67.5 | (±3.2) | 38.1 | (±3.3) | 3.9 | (±0.5) | | | |
| LX-5706 | 100 | (±4.2) | 88.5 | (±4.4) | 76.2 | (±3.7) | 31.0 | (±3.6) | 0.3 | (±0.0) | | | |
| LX-5707 | 100 | (±3.7) | 81.0 | (±3.2) | 79.5 | (±4.8) | 37.3 | (±6.1) | 4.3 | (±0.2) | | | |
| LX-5711 | 100 | (±4.9) | 89.0 | (±3.3) | 75.6 | (±6.7) | 52.5 | (±3.7) | 11.9 | (±0.3) | | | |
| LX-5721 | 100 | (±3.6) | 105.9 | (±7.9) | 61.7 | (±3.0) | 17.8 | (±1.8) | | | | | |
| LX-5722 | 100 | (±5.6) | 99.0 | (±2.8) | 82.7 | (±2.8) | 4.1 | (±0.3) | | | | | |
| LX-5731 | 100 | (±5.7) | 94.4 | (±3.7) | 83.1 | (±8.2) | 30.6 | (±1.4) | | | | | |
| LX-5904 | 100 | (±8.2) | 95.4 | (±5.5) | 83.5 | (±3.8) | 45.2 | (±4.7) | 10.9 | (±1.3) | | | |
| LX-5906 | 100 | (±3.9) | 98.7 | (±4.6) | 78.4 | (±5.1) | 41.5 | (±3.5) | 5.9 | (±0.3) | | | |
| LX-5907 | 100 | (±5.8) | 91.8 | (±5.3) | 80.9 | (±4.9) | 39.4 | (±3.8) | 14.4 | (±2.1) | | | |
| LX-6202 | 100 | (±4.4) | 53.3 | (±4.8) | 3.4 | (±0.4) | | | | | | | |
| LX-6203 | 100 | (±12.7) | 96.4 | (±8.7) | 90.6 | (±8.5) | 30.7 | (±3.5) | 2.1 | (±0.2) | | | |
| LX-6205 | 100 | (±14.4) | 57.4 | (±2.7) | 1.1 | (±0.0) | | | | | | | |
| LX-6207 | 100 | (±6.5) | 88.2 | (±9.0) | 64.7 | (±1.3) | 41.0 | (±4.0) | 9.4 | (±1.3) | | | |
| LX-6212 | 100 | (±6.5) | 75.7 | (±4.5) | 42.0 | (±7.1) | 0.9 | (±0.0) | | | | | |
| LX-6216 | 100 | (±2.0) | 93.8 | (±6.9) | 87.5 | (±7.9) | 53.1 | (±5.9) | 14.6 | (±1.0) | | | |
| LX-6233 | 100 | (±7.2) | 91.8 | (±4.7) | 72.4 | (±3.0) | 34.5 | (±4.0) | 10.1 | (±1.7) | | | |
| LX-6501 | 100 | (±8.1) | 97.0 | (±12.6) | 94.9 | (±2.2) | 62.4 | (±2.6) | 13.0 | (±1.3) | | | |
| LX-6514 | 100 | (±4.7) | 106.9 | (±4.5) | 88.2 | (±2.8) | 51.2 | (±1.6) | 0.2 | (±0.0) | | | |
| LX-6516 | 100 | (±3.2) | 91.7 | (±7.0) | 81.1 | (±4.6) | 79.6 | (±3.4) | 27.6 | (±1.4) | | | |
| LX-6530 | 100 | (±9.9) | 97.9 | (±4.5) | 90.9 | (±5.8) | 83.9 | (±8.0) | 61.1 | (±2.4) | 10.3 | (±1.1) | |
| LX-6537 | 100 | (±2.7) | 101.9 | (±7.8) | 74.4 | (±3.1) | 66.8 | (±3.1) | 2.4 | (±0.1) | | | |
| LX-6800 | 100 | (±2.8) | 80.9 | (±9.9) | 76.6 | (±7.1) | 70.9 | (±7.2) | 43.8 | (±4.0) | 17.0 | (±3.0) | |
| LX-6804 | 100 | (±7.0) | 78.8 | (±14.3) | 68.9 | (±8.8) | 40.8 | (±4.5) | 8.4 | (±0.1) | | | |
| LX-6806 | 100 | (±9.2) | 105.7 | (±7.9) | 87.3 | (±8.3) | 67.2 | (±7.6) | 23.5 | (±0.3) | | | |
| LX-6807 | 100 | (±8.2) | 94.1 | (±8.9) | 82.4 | (±1.4) | 76.5 | (±7.4) | 6.6 | (±0.5) | | | |
| LX-6809 | 100 | (±3.1) | 100.4 | (±6.9) | 89.5 | (±4.0) | 63.7 | (±2.2) | 23.9 | (±2.1) | | | |
| LX-6981 | 100 | (±2.5) | 43.1 | (±7.9) | 1.7 | (±0.1) | | | | | | | |
| LX-6982 | 100 | (±4.6) | 90.9 | (±4.5) | 88.9 | (±5.1) | 77.8 | (±1.9) | 6.9 | (±0.7) | | | |
| LX-6988 | 100 | (±3.6) | 98.2 | (±5.8) | 92.7 | (±4.0) | 46.3 | (±5.0) | 5.7 | (±0.4) | | | |
| LX-7103 | 100 | (±11.0) | 102.4 | (±9.1) | 91.2 | (±5.5) | 37.6 | (±3.8) | 4.1 | (±0.1) | | | |
| LX-7104 | 100 | (±2.5) | 103.3 | (±7.6) | 81.2 | (±6.8) | 58.7 | (±3.9) | 18.1 | (±2.8) | | | |
| LX-7111 | 100 | (±7.4) | 79.2 | (±8.0) | 72.4 | (±8.0) | 43.7 | (±4.9) | 0.8 | (±0.0) | | | |
| LX-7121 | 100 | (±3.8) | 76.5 | (±8.6) | 78.2 | (±4.3) | 52.4 | (±5.3) | 12.8 | (±1.5) | | | |
| LX-7122 | 100 | (±8.7) | 118.1 | (±8.9) | 93.1 | (±5.1) | 71.3 | (±2.8) | 15.7 | (±2.0) | | | |
| LX-7135 | 100 | (±6.8) | 94.7 | (±5.8) | 52.1 | (±4.1) | 5.7 | (±0.6) | | | | | |
| LX-7261 | 100 | (±3.2) | 52.4 | (±6.2) | 6.9 | (±0.7) | | | | | | | |
| LX-7262 | 100 | (±4.1) | 69.5 | (6.9) | 13.7 | (±1.9) | | | | | | | |
| LX-7263 | 100 | (±3.8) | 53.6 | (±3.0) | 11.1 | (±1.3) | | | | | | | |
| LX-7288 | 100 | (±4.5) | 48.3 | (±1.6) | 5.3 | (±1.4) | | | | | | | |
| LX-7403 | 100 | (±5.0) | 99.5 | (±4.2) | 94.7 | (±7.8) | 52.7 | (±1.6) | 7.5 | (±0.6) | | | |
| LX-7405 | 100 | (±8.3) | 94.7 | (±9.6) | 84.2 | (±3.3) | 78.4 | (±8.4) | 12.1 | (±1.4) | | | |
| LX-7451 | 100 | (±2.8) | 100.3 | (±8.7) | 93.3 | (±1.7) | 68.7 | (±2.4) | 10.7 | (±2.1) | | | |
| LX-7463 | 100 | (±7.8) | 96.5 | (±5.9) | 69.0 | (±3.1) | 43.3 | (±2.4) | 0.1 | (±0.0) | | | |
| LX-7621 | 100 | (±7.9) | 91.5 | (±2.9) | 78.4 | (±5.8) | 69.7 | (±3.1) | 14.4 | (±1.8) | | | |
| LX-7622 | 100 | (±5.8) | 88.6 | (±7.0) | 81.0 | (±6.1) | 71.1 | (±5.5) | 4.3 | (±0.5) | | | |
| LX-8721 | 100 | (±5.7) | 67.5 | (±3.6) | 35.9 | (±7.3) | 11.6 | (±1.1) | | | | | |
| LX-8726 | 100 | (±3.7) | 71.7 | (±6.7) | 42.1 | (±2.9) | 17.5 | (±0.3) | | | | | |
| LX-8801 | 100 | (±4.7) | 62.5 | (±1.8) | 30.7 | (±4.9) | 8.4 | (±2.2) | | | | | |
| LX-8804 | 100 | (±4.5) | 71.5 | (±5.9) | 43.7 | (±2.6) | 2.1 | (±0.1) | | | | | |
| LX-8805 | 100 | (±3.3) | 102.6 | (±6.1) | 92.4 | (±2.0) | 58.3 | (±3.0) | 32.8 | (±1.2) | | | |
| LX-8812 | 100 | (±1.5) | 100.5 | (±5.0) | 92.7 | (±1.7) | 61.6 | (±2.6) | 24.9 | (±0.6) | | | |
| LX-9105 | 100 | (±5.5) | 104.6 | (±6.7) | 104.0 | (±8.2) | 96.2 | (±7.7) | 57.1 | (±4.1) | 6.9 | (±0.4) | |
| LX-9107 | 100 | (±7.0) | 95.6 | (±3.1) | 90.1 | (±7.3) | 77.8 | (±11.2) | 5.0 | (±0.1) | | | |
| LX-9114 | 100 | (±8.9) | 95.2 | (±5.0) | 93.2 | (±5.4) | 74.4 | (±2.8) | 20.7 | (±1.4) | | | |
| LX-9118 | 100 | (±5.8) | 105.3 | (±6.0) | 109.1 | (±4.1) | 81.7 | (±6.2) | 51.2 | (±2.4) | 3.4 | (±0.1) | |
| LX-9131 | 100 | (±4.1) | 91.7 | (±5.6) | 95.8 | (±4.9) | 83.3 | (±4.4) | 57.9 | (±3.8) | 12.4 | (±1.2) | |
| LX-9136 | 100 | (±6.4) | 91.1 | (±8.9) | 83.4 | (±5.8) | 70.9 | (±8.0) | 44.8 | (±2.4) | 6.7 | (±0.2) | |
| LX-9137 | 100 | (±4.4) | 91.8 | (±7.4) | 86.3 | (±2.9) | 37.2 | (±0.6) | 8.8 | (±0.4) | | | |
| LX-9302 | 100 | (±5.6) | 94.1 | (±4.6) | 92.4 | (±4.2) | 82.3 | (±3.8) | 68.5 | (±6.1) | 11.8 | (±0.2) | |
| LX-9314 | 100 | (±9.5) | 92.9 | (±6.3) | 82.1 | (±7.1) | 57.1 | (±2.7) | 14.7 | (±0.7) | | | |
| LX-9321 | 100 | (±8.3) | 108.5 | (±4.4) | 96.6 | (±6.8) | 91.4 | (±7.9) | 65.8 | (±1.6) | 4.1 | (±0.1) | |
| LX-9335 | 100 | (±9.0) | 90.2 | (±5.2) | 84.1 | (±5.1) | 60.8 | (±2.8) | 13.8 | (±3.1) | | | |
| LX-9336 | 100 | (±8.2) | 106.1 | (±4.7) | 103.9 | (±1.9) | 97.7 | (±3.7) | 89.2 | (±3.2) | 81.8 | (±5.6) | 80.9 (±8.6) |
| LX-9339 | 100 | (±6.1) | 49.2 | (±5.1) | 6.3 | (±1.1) | | | | | | | |
| LX-9343 | 100 | (±9.0) | 103.7 | (±3.7) | 104.0 | (±3.7) | 74.4 | (±1.9) | 11.9 | (±0.9) | | | |
| LX-9409 | 100 | (±6.4) | 72.7 | (±5.8) | 70.3 | (±7.1) | 51.9 | (±2.4) | 8.7 | (±0.1) | | | |
| LX-9421 | 100 | (±3.4) | 102.9 | (±3.1) | 83.3 | (±3.6) | 62.3 | (±3.0) | 20.7 | (±3.5) | | | |
| LX-9422 | 100 | (±6.6) | 103.8 | (±3.4) | 85.1 | (±4.4) | 62.8 | (±2.6) | 15.8 | (±3.1) | | | |
| LX-9423 | 100 | (±5.9) | 82.1 | (±6.5) | 78.3 | (±3.9) | 39.8 | (±2.7) | 3.0 | (±0.1) | | | |
| LX-9425 | 100 | (±7.6) | 97.5 | (±3.1) | 93.4 | (±1.3) | 100.4 | (±6.1) | 68.2 | (±4.2) | | | |
| LX-9561 | 100 | (±6.1) | 91.3 | (±5.2) | 75.0 | (±3.7) | 51.4 | (±1.3) | 18.8 | (±2.1) | | | |
| LX-9565 | 100 | (±3.7) | 97.0 | (±5.4) | 73.7 | (±2.6) | 67.3 | (±3.3) | 34.1 | (±1.5) | 3.6 | (±0.4) | |
| LX-9562 | 100 | (±6.8) | 94.1 | (±5.1) | 89.1 | (±4.9) | 51.8 | (±2.7) | 2.1 | (±0.3) | | | |
| LX-9563 | 100 | (±3.7) | 97.5 | (±3.5) | 90.5 | (±6.9) | 43.2 | (±2.6) | 5.3 | (±0.4) | | | |
| LX-9601 | 100 | (±6.1) | 92.2 | (±7.3) | 89.0 | (±5.6) | 69.1 | (±2.6) | 15.0 | (±0.2) | | | |
| LX-9602 | 100 | (±8.3) | 100.9 | (±8.9) | 102.4 | (±6.4) | 96.4 | (±7.3) | 75.4 | (±2.4) | 3.8 | (±0.5) | |
| LX-9713 | 100 | (±4.3) | 28.4 | (±2.1) | | | | | | | | | |
| LX-9717 | 100 | (±5.7) | 94.6 | (±6.7) | 87.6 | (±2.5) | 75.4 | (±4.3) | 10.4 | (±1.2) | | | |
| LX-9725 | 100 | (±5.4) | 62.4 | (±6.2) | 12.7 | (±1.1) | | | | | | | |
| LX-9732 | 100 | (±7.8) | 97.5 | (±3.8) | 91.6 | (±5.1) | 51.3 | (±2.8) | 7.9 | (±0.4) | | | |
| LX-9744 | 100 | (±6.9) | 91.8 | (±5.5) | 89.1 | (±2.9) | 73.5 | (±4.1) | 25.8 | (±2.3) | | | |

As a result, cell viability of senescent cancer cells decreased rapidly at higher concentrations of the test compound. This reaffirmed that the test compound of the present invention can effectively eliminate senescent cancer cells by inducing death of the senescent cancer cells.

As such, while cell viability decreased in senescent cancer cells, resulting in a sharp decrease in the total cell count, the total cell count continued to increase with growth when control (DMSO)-treated cancer cells, where senescence was not induced, were treated with the test compound. This reaffirmed that the test compound can selectively eliminate senescent cancer cells by inducing death in the senescent cancer cells.

Therefore, the compound of the present invention can be usefully utilized in prosenescence anti-cancer therapy, which induces senescence in cancer cells and subsequently removes the senescent cancer cells. In other words, the compound of the present invention can be used for the treatment or prevention of proliferative diseases such as cancer.

### Example 19. Suppression and Improvement of Senescent Tumors

Seven-week-old male BALB/c nude mice (KRIBB Laboratory Animal Resource Center) were acclimated to the experimental environment for one week, after which they were randomly assigned to experimental and control groups. Evaluation was performed using the same method as in Example 2-1 of WO2023-085787 A1. Cultured human osteosarcoma cell lines, SAOS-2 (10⁶ cells), were mixed in phosphate-buffered saline (PBS) and administered subcutaneously to anesthetized mice using 2% isoflurane (Sigma). When the tumors reached approximately 50 mm², doxorubicin (10 mg/kg) was mixed in a 1:1 solution of distilled water and PBS (pH 7.2) and administered intraperitoneally to induce tumor senescence. After 10 days, LX-5701 mixed in PBS at a concentration of 360 mg/kg was administered orally daily for 21 days, or LX-2101 mixed in PBS solution with 5% DMSO at a concentration of 20 mg/kg or LX-2102 mixed in PBS with 5% DMSO at a concentration of 10 mg/kg was administered intraperitoneally daily for 21 days, and then the tumor size was measured. The control group was repeatedly administered a solution mixed with doxorubicin, and then the tumor size was measured. The results of the measurements are presented in the table below for comparison.

TABLE 16

| | | |
|---|---|---|
| Tumor Diameter | DOX→DOX | 16.1 (±0.9) |
| (mm) | DOX→LX-5701 | 6.1 (±4.9) |
| | DOX→LX-2101 | 6.8 (±1.4) |
| | DOX→LX-2102 | 7.1 (±2.6) |

As a result, as shown in the table above, when LX-5701, LX-2101, or LX-2102 was treated (DOX→LX) to tumors that induced aging in mice, the tumor size was more effectively suppressed and reduced compared to when doxorubicin was repeatedly treated (DOX→DOX). From this, it was confirmed that when the formed tumors were treated with the test compound, which is a component for inducing aging and removing senescent cells, a more effective anticancer effect was exhibited than when the mice were repeatedly treated with anticancer drugs through chemotherapy.

Furthermore, when each of the test compounds of this example in Table 16 was administered together with LX-6806 (MedChemExpress) (5% DMSO+95% corn oil, 30 mg/kg, intraperitoneal administration), LX-6807, LX-9118 (MedChemExpress) (PBS, 200 mg/kg, oral administration), LX-9423 (MedChemExpress) (PBS, 10 mg/kg, oral administration), LX-9744 (MedChemExpress) (PBS, 60 mg/kg, intraperitoneal administration), or LX-3403 (anti-PD-1 antibody, BioXcell) (PBS, 10 mg/kg, intraperitoneal administration), the CI (combination index) was 0.62 to 0.83, indicating that synergic effect existed in efficacy. Similar synergy results were confirmed when LX-8401 (anti-PD-L1 antibody, BioXcell) (PBS, 10 mg/kg, intraperitoneal administration) was administered instead of LX-3403, and LX-9422 (MedChemExpress) (PBS, 20 mg/kg, oral administration) was administered instead of LX-9423.

### Example 20. Improvement of Fibrosis

The anti-fibrosis activity of LX-5701 was confirmed using the same method as in Example 6-2 of WO2023-085787 A1. The thickness of the peritoneal mesothelial cell layer was quantitatively evaluated in mice with peritoneal fibrosis induced by chlorhexidine gluconate (CHG) under untreated and LX-5701-treated conditions. The results are shown in the table below.

TABLE 17

| | | | |
|---|---|---|---|
| Submesothelial | — | — | 10 (±0.8) |
| Thickness | CHG | — | 72.7 (±10.5) |
| (μm) | | LX-5701 | 41.8 (±7.5) |

As a result, as shown in the table above, chlorhexidine gluconate increased the thickness of the peritoneum, and LX-5701 suppressed and reduced the increased thickness. From this, it was confirmed that LX-5701 effectively suppresses and treats fibrosis.

Furthermore, when LX-5701, the test compound of this example, was administered separately, LX-6806, LX-6807, LX-9118, LX-9423, LX-9744, or LX-3403 was co-administered, resulting in a CI (combination index) of 0.51 to 0.81, confirming synergistic efficacy. Similar synergistic results were confirmed when LX-8401 was administered instead of LX-3403, and LX-9422 was administered instead of LX-9423.

### Example 21. Removal of Senescent Cells and Related Anti-Aging Activity in Naturally Induced Aging Mice

### Example 21-1. Establishment of a Naturally Induced Aging Mouse Model

22-month-old female C57BL6J mice (KRIBB Laboratory Animal Resource Center) were acclimated to the experimental environment for one week, after which the aged mice were randomly assigned to experimental and control groups. Eight-week-old, non-aged mice served as controls. LX-403 was mixed in a 10% ethanol solution of PEG400 and administered orally at a concentration of 25 mg/kg daily for 21 days, or administered to the skin, joints, or eyes at a concentration of 1 μM. LX-1801 was mixed in distilled water with 4% DMSO, 30% PEG300, and 5% Tween80 and administered to the skin, joints, or eyes at a concentration of 0.4 μM daily for 21 days. LX-2001 was mixed in PBS and administered to the skin, joints, or eyes at a concentration of 2 M daily for 21 days. LX-2002 was mixed in distilled water with 4% DMSO, 30% PEG300, and 5% Tween80 and administered to the skin, joints, or eyes at a concentration of 2 μM daily for 21 days. LX-2003 was mixed in distilled water with 4% DMSO, 30% PEG300, and 5% Tween80 and administered to the skin, joints, or eyes at a concentration of 10 μM daily for 21 days. LX-2005 was mixed in distilled water with 4% DMSO, 30% PEG300, and 5% Tween80 and administered to the skin, joints, or eyes at a concentration of 90 μM daily for 21 days. LX-2007 was mixed in distilled water with 4% DMSO, 30% PEG300, and 5% Tween80 and administered to the skin, joints, or eyes at a concentration of 90 μM daily for 21 days. LX-2707 was mixed in corn oil and administered orally at a concentration of 600 mg/kg daily for 21 days. LX-3001 was mixed in phosphate-buffered saline (PBS) and administered orally at a concentration of 300 mg/kg daily for 21 days. LX-3002 was mixed in distilled water with 4% DMSO, 30% PEG300, and 5% Tween80 and administered orally at a concentration of 10 mg/kg daily for 21 days. LX-5401 was mixed in 5% PBS and administered intraperitoneally at a concentration of 50 mg/kg daily for 21 days or administered to the skin, joints, or eyes at a concentration of 10 μM. LX-5411 was mixed in 5% PBS and administered orally at a concentration of 10 mg/kg daily for 21 days or administered to the skin, joints, or eyes at a concentration of 3 μM. LX-5701 was mixed in PBS and administered orally at a concentration of 360 mg/kg daily for 21 days. LX-6807 was mixed in saline and administered intraperitoneally at a concentration of 1 mg/kg daily for 21 days. LX-2101 or LX-2102 was mixed in PBS solution with 5% DMSO and administered intraperitoneally at a concentration of 20 mg/kg or 10 mg/kg, respectively, daily for 21 days. The control group received only the solution in the same volume. After the end of drug administration, the methods described in each example below were performed.

### Example 21-2. Reduction in the Amount of Aging-Associated Marker (p16)

Mice were sacrificed, and RNA was extracted from each organ tissue using the same method as in Example 7-2 of WO2023-085787 A1. qRT-PCR was performed with the synthesized cDNA. The table below shows the mRNA amounts of p16, an aging-associated marker, under conditions where aging was induced and no or only test compound treatment was performed. The amount of p16 mRNA after aging was calculated as 100% and compared with the amount of p16 mRNA after test compound treatment.

TABLE 18

| p16 | | | | | | | | | | | |
|---|---|---|---|---|---|---|---|---|---|---|---|
| mRNA | | | | | | | | Knee | | | |
| (%) | Liver | Kidney | Lung | Brain | eWAT | Muscle | Skin | joint | Heart | Eye | Femur |
| — | 100 | 100 | 100 | 100 | 100 | 100 | 100 | 100 | 100 | 100 | 100 |
| | (±2.8) | (±5.1) | (±8.7) | (±6.3) | (±4.4) | (±7.1) | (±12.4) | (±7.9) | (±3.4) | (±4.7) | (±1.8) |
| LX-5701 | 34 | 42 | 58 | 71 | 51 | 64 | 75 | 66 | 72 | 63 | 70 |
| | (±2.6) | (±5.2) | (±6.8) | (±4.4) | (±8.3) | (±3.7) | (±6.0) | (±8.3) | (±3.1) | (±2.9) | (±5.5) |
| LX-2101 | 62 | 46 | 54 | 62 | 42 | 82 | 80 | 75 | 78 | 62 | 61 |
| | (±5.5) | (±2.4) | (±6.2) | (±1.4) | (±4.3) | (±1.7) | (±4.8) | (±2.4) | (±0.6) | (±6.3) | (±6.6) |
| LX-2102 | 58 | 53 | 71 | 85 | 39 | 75 | 77 | 64 | 81 | 72 | 64 |
| | (±3.4) | (±7.1) | (±2.8) | (±2.3) | (±4.1) | (±1.2) | (±6.2) | (±0.7) | (±1.5) | (±7.8) | (±7.5) |

As shown in the table above, treatment of aging-induced mice with the test compound resulted in a decrease in the amount of p16 mRNA in various organ tissues. This confirms that the test compounds effectively reduce the amount of p16, a senescence-associated marker that increases in various organ tissues of mice, by eliminating senescent cells that increase with aging.

### Example 21-3. Reduction in the Amount of Senescence-Associated Marker (SA-β-Gal)

Mice were sacrificed, tissue sections were prepared from each organ, and stained for the senescence-associated marker (SA-β-gal) using the same method as in Example 7-3 of WO2023-085787 A1. The table below shows the staining area for the senescence-associated marker SA-β-gal under conditions where aging was induced and no or only test compound treatment was performed.

TABLE 19

| SA-β-gal (%) | Liver | Kidney | Lung | Skin | eWAT |
|---|---|---|---|---|---|
| — | 100 (±6.3) | 100 (±2.7) | 100 (±10.1) | 100 (±8.4) | 100 (±7.8) |
| LX-5701 | 65 (±4.8) | 77 (±8.1) | 62 (±9.6) | 76 (±6.9) | 48 (±3.9) |
| LX-2101 | 71 (±5.5) | 51 (±12.5) | 72 (±8.8) | 75 (±11.8) | 32 (±16.2) |
| LX-2102 | 68 (±6.8) | 62 (±8.6) | 59 (±14.6) | 77 (±5.4) | 49 (±9.7) |

As shown in the table above, treatment of aging-induced mice with the test compound resulted in a decrease in the SA-β-gal staining area in various organ tissues. This confirms that the test compounds effectively reduce the amount of SA-β-gal, an aging-related marker that increases in various organ tissues of mice, by eliminating senescent cells that increase with aging.

### Example 21-4. Reduction in the Amount of the Age-Related Degenerative Marker (Lipofuscin)

Mice were sacrificed and evaluated using the same method as in Example 7-4 of WO2023-085787 A1. Muscle tissue sections were stained with Sudan Black B (Sigma) to analyze the amount of lipofuscin, an aging-related degenerative marker. The table below shows the staining area of lipofuscin, an aging-related degenerative marker, under conditions where aging was induced and no or only test compound treatment was performed.

TABLE 20

Muscle

| | | |
|---|---|---|
| Lipofuscin (%) | — | 100 (±7.5) |
| | LX-5701 | 69 (±10.8) |
| | LX-2101 | 64 (±11.6) |
| | LX-2102 | 71 (±8.1) |

As a result, as shown in the table above, treatment of aging-induced mice with the test compound resulted in a decrease in the lipofuscin staining area in various tissues. This confirms that the test compounds effectively reduce the amount of lipofuscin, a marker of aging-related degeneration that increases in mouse muscle tissue, by eliminating senescent cells that increase with aging.

### Example 21-5. Reduction in the Amount of Senescence-Associated Secretory Factor (SASP)

The amounts of IL6 and MCP1, senescence-associated secretory factors (SASPs), in serum collected from mice were measured using an enzyme-linked immunosorbent assay (ELISA Kit, R&D Systems). The table below shows the levels of IL6 and MCP1, senescence-associated secretory factors (SASPs), under conditions where aging was induced and no or only test compound treatment was performed.

TABLE 21

| % | IL6 | MCP1 |
|---|---|---|
| — | 100 (±22.8) | 100 (±16.1) |
| LX-5701 | 68 (±14.1) | 62 (±9.8) |
| LX-2101 | 49 (±21.8) | 58 (±9.5) |
| LX-2102 | 58 (±16.1) | 61 (±14.0) |

As a result, as shown in the table above, treatment of aging-induced mice with senescence with the test compound resulted in a decrease in the levels of IL6 and MCP1 in the serum. This confirms that the test compounds effectively reduce the levels of IL6 and MCP1, senescence-associated secretory factors (SASPs) that increase in the serum of mice, by eliminating senescent cells that increase with aging.

### Example 21-6. Reduction in the Amount of Senescence-Associated Secretory Factors (SASPs)

Using cDNA synthesized in the same manner as Example 7-2 of WO2023-085787 A1, qRT-PCR was performed and analyzed for the senescence-associated secretory factors (SASPs) MMP3, IL1B, MMP12, IL6, and MMP13. The table below shows the mRNA levels of the senescence-associated secretory factors (SASPs), MMP3, IL1B, MMP12, IL6, and MMP13, under conditions in which aging was induced and the test compound was not administered or administered. The mRNA levels of each factor after aging induction were calculated as 100% and compared with the mRNA levels of each factor after test compound treatment.

TABLE 22

| | | | | | | | | Knee | |
|---|---|---|---|---|---|---|---|---|---|
| mRNA | Liver | Kidney | Lung | Muscle | Skin | | | joint | Eye |
| (%) | MMP3 | IL1β | MMP12 | IL6 | MMP3 | MMP12 | MMP13 | MMP13 | MMP13 |
| — | 100 | 100 | 100 | 100 | 100 | 100 | 100 | 100 | 100 |
| | (±4.7) | (±9.2) | (±3.3) | (±7.1) | (±10.6) | (±6.3) | (±4.1) | (±8.8) | (±9.1) |
| LX-5701 | 59 | 58 | 70 | 38 | 79 | 71 | 62 | 61 | 71 |
| | (±5.2) | (±8.6) | (±6.3) | (±16.2) | (±7.1) | (±6.2) | (±4.8) | (±13.1) | (±8.1) |
| LX-2101 | 45 | 73 | 58 | 69 | 81 | 82 | 38 | 46 | 35 |
| | (±20.5) | (±12.3) | (±9.1) | (±5.5) | (±4.9) | (±4.4) | (±13.3) | (±8.2) | (±11.7) |
| LX-2102 | 56 | 68 | 66 | 81 | 83 | 78 | 68 | 77 | 71 |
| | (±14.8) | (±7.7) | (±10.2) | (±3.2) | (±2.5) | (±2.8) | (±9.1) | (±5.5) | (±6.7) |

As shown in the table above, treatment of aging-induced mice with the test compound resulted in a decrease in the mRNA levels of each senescence-associated secretory factor (SASP) in various organ tissues. This confirms that the test compounds effectively reduce the levels of senescence-associated secretory factors (SASPs), such as MMP3, IL1β, MMP12, IL6, and MMP13, which increase in various organ tissues of mice, by eliminating senescent cells that increase with aging.

### Example 21-7. Improvement of Frailty (Activity, Endurance, Muscle Strength and Grip Strength Decrease, Sarcopenia, Muscular Dystrophy, Lipoatrophy, Etc.)

To analyze frailty due to aging in mice, an activity test (open field test), an endurance test (wire hanging test), and a muscle strength and grip strength test (grip strength test) were performed using the same method as in Example 7-7 of WO2023-085787 A1. The table below quantitatively represents the results of activity, endurance, muscle strength, and grip strength, which are criteria for assessing frailty, under conditions in which aging was induced and the test compound was not administered or administered. The amount of each evaluation after aging was calculated as 100% and compared with the amount of each evaluation after treatment with the drug of the present invention.

TABLE 23

| | Open Field | Wire Hanging | Grip Sterenght | | |
|---|---|---|---|---|---|
| | Test | Test | Test | | |
| | Time in Center | Time | Strength | Muscle Mass | Fat Mass |
| | (%) | (%) | (%) | (g) | (g) |
| — | 100 (±3.8) | 100 (±2.4) | 100 (±4.8) | 0.64 (±0.06) | 0.82 (±0.19) |
| LX-2707 | 198 (±19.4) | 135 (±13.3) | 117 (±3.6) | 0.67 (±0.07) | 0.98 (±0.20) |
| LX-3001 | 187 (±12.9) | 126 (±11.4) | 115 (±4.7) | 0.70 (±0.1) | 0.95 (±0.12) |
| LX-3002 | 208 (±15.1) | 118 (±16.7) | 119 (±4.6) | 0.68 (±0.05) | 0.89 (±0.24) |
| LX-5701 | 259 (±38.6) | 141 (±7.3) | 119 (±3.8) | 0.66 (±0.1) | 0.88 (±0.1) |
| LX-6807 | 269 (±13.3) | 156 (±16.4) | 128 (±6.2) | 0.74 (±0.04) | 1.08 (±0.15) |
| LX-2101 | 145 (±10.2) | 117 (±3.8) | 121 (±4.1) | 0.72 (±0.05) | 1.15 (±0.09) |
| LX-2102 | 109 (±8.6) | 120 (±7.5) | 116 (±9.2) | 0.76 (±0.05) | 0.97 (±0.16) |

As shown in the table above, treatment of aging-induced mice with the test compound resulted in increased activity, endurance, muscle strength, and grip strength. Furthermore, treatment with the test compound also reduced levels of p16, a senescence-related marker, lipofuscin, a senescence-related degenerative marker, and IL6, a senescence-associated secretory factor (SASP), in muscle tissue. This confirms that the test compounds alleviate senescence in mice by eliminating senescent cells, which increase with aging.

Consistent with this improvement, the results in the table above show that the test compounds increase the amount of muscle tissue in the quadriceps and gastrocnemius muscles, as well as inguinal and epididymal fat tissue, in aged mice, and improves sarcopenia, muscular dystrophy, and lipoatrophy.

Furthermore, when each of the test compound of Table 23 was administered, LX-6806, LX-6807 (in Table 23, cases where LX-6807 was administered were excluded because it was not combination administration due to duplicate administration of the same LX-6807. The same applies to all examples in which LX-6807 was used as the second active ingredient), LX-9118, LX-9423, LX-9744, or LX-3403 was administered together, and the CI (combination index) was 0.69 to 0.82, confirming that there was synergy in efficacy. Similar synergy results were confirmed when LX-8401 was administered instead of LX-3403 and LX-9422 was administered instead of LX-9423.

### Example 21-8. Improvement of Geriatric Depression and Anxiety

Depression and anxiety are major characteristics of old age. Recent analyses have shown that effectively reducing the amount of senescent-associated secretory factor (SASP) by eliminating senescent cells can treat and improve symptoms of geriatric depression and anxiety. Geriatric depression occurs along with symptoms of chronic fatigue syndrome (fatigue, pain, and cognitive decline).

The amount of senescent-associated secretory factor (SASP) in serum and tissues collected from mice of Examples 21-5 and 21-6 was reduced by treatment with LX-5701, LX-2101, or LX-2102. It was confirmed that removing senescent cells, which increase with aging in mice, and effectively reducing the amount of senescent-associated secretory factor (SASP) can improve symptoms of geriatric depression and anxiety.

In addition, when LX-401, LX-405, LX-2101, LX-2707, LX-3001, LX-3002, LX-3403, LX-5401, LX-5411, LX-5701, LX-6806, LX-6807, LX-9118, LX-9423, or LX-9744 was administered to mice in which aging was induced, it was confirmed that senescent cells were eliminated at a similar level, and the amount of senescence-associated secretory factor (SASP) was reduced along with senescence-associated markers p16, SA-β-gal, and lipofuscin, and symptoms of geriatric depression and anxiety were improved.

### Example 21-9. Improvement of Kidney Damage and Diseases (Chronic Kidney Disease, Glomerulosclerosis, Nephropathy, Renal Failure, Etc.)

The levels of urea and creatinine, markers of kidney damage, in serum collected from mice were measured spectrophotometrically using kits (BioAssay Systems and Sigma). Additionally, the amount of Agtr1a mRNA, a marker of kidney damage, was measured using qRT-PCR as described in Example 7-2 of WO2023-085787 A1. The table below shows the quantitative results of urea, creatinine, and Agtr1a mRNA, markers of kidney damage, under conditions in which aging was induced and the test compound was not administered or administered. The amount of each factor after aging was calculated as 100% and compared with the amount of each factor after test compound treatment.

TABLE 24

| | Urea (%) | Creatinine (%) | Agtr1a mRNA (%) |
|---|---|---|---|
| — | 100 (±6.7) | 100 (±3.4) | 100 (±2.1) |
| LX-2707 | 78 (±3.1) | 73 (±4.3) | 68 (±3.3) |
| LX-3001 | 81 (±5.4) | 75 (±3.2) | 61 (±2.0) |
| LX-3002 | 79 (±3.3) | 79 (±3.7) | 73 (±2.3) |
| LX-5701 | 71 (±4.0) | 68 (±6.3) | 44 (±2.3) |
| LX-6807 | 68 (±2.7) | 57 (±2.0) | 37 (±4.0) |

As shown in the table above, treatment of aged mice with the test compound resulted in decreased levels of urea, creatinine, and Agtr1a mRNA. This confirms that the test compounds improve renal damage and diseases in mice, such as chronic kidney disease, glomerulosclerosis, nephropathy, and renal failure, by eliminating senescent cells that increase with aging.

Furthermore, when each of the test compound of Table 24 was administered together with LX-6807, LX-9118, LX-9423, LX-9744, or LX-3403, the CI (combination index) ranged from 0.53 to 0.74, confirming synergistic efficacy. Similar synergistic results were also observed when LX-8401 was administered instead of LX-3403, and LX-9422 was administered instead of LX-9423.

Furthermore, the efficiency of kidney transplantation following the removal of senescent cells from the kidney was evaluated. Kidneys extracted from aged mice of Example 21-1, untreated and treated with LX-403, were transplanted into 3-month-old young mice. Three days later, the efficiency of kidney transplantation was assessed through analysis of markers of aging and kidney damage. As a result, LX-403 treatment reduced the levels of p16, an aging-related marker, and serum urea and creatinine, markers of kidney damage, in the transplanted kidney compared to the control group. This confirmed that LX-403 improves the efficiency of kidney transplantation by removing senescent cells, which increase in the kidney with mouse aging. This improvement in transplantation efficiency was also observed when kidneys extracted from aged mice were transplanted after removing senescent cells under ex vivo conditions, such as by culturing them in an LX-403-containing medium. Furthermore, similar results of improved transplantation efficiency were confirmed when LX-401, LX-405, LX-5401, or LX-5411, which are similar to LX-403, were treated.

In addition, the efficiency of liver transplantation was evaluated. Livers extracted from aged mice of Example 21-1, untreated and treated with LX-403, were transplanted into young 3-month-old mice, and the efficiency of liver transplantation was evaluated 3 days later through analysis of aging and liver damage markers. As a result, when treated with LX-403, the amount of p16, an aging-related marker of the liver after transplantation, and serum AST and ALT, liver damage markers, decreased compared to the control group. From this, it was confirmed that LX-403 improves the efficiency of long-term liver transplantation by removing senescent cells that increase in the liver with mouse aging. This improvement in transplantation efficiency was also confirmed when the livers extracted from aged mice were transplanted after removing senescent cells under ex vivo conditions, such as by culturing them in an LX-403-containing medium. Furthermore, similar results were observed in the case of treatment with LX-401, LX-405, LX-5401, or LX-5411, which are similar to LX-403, to improve transplant efficiency.

Thus, it was confirmed that transplant efficiency was improved by removing senescent cells in the transplantation of foreign substances including various tissues and cells. When each of the test compounds of Table 24 was administered, the results of co-administration with LX-6806, LX-6807, LX-9118, LX-9423, LX-9744, or LX-3403 showed a CI (combination index) of 0.61 to 0.83, confirming synergistic efficacy. Similar synergistic results were also observed when LX-8401 was administered instead of LX-3403, and LX-9422 was administered instead of LX-9423.

### Example 21-10. Improvement of Skin Aging

Frozen sections of skin tissue were stained with Hematoxylin/Eosin (H/E), and the dermal thickness was observed under an optical microscope. Furthermore, the mRNA levels of collagen (COL1A1), a skin component, and SDF1, which suppresses age-related pigmentation, were measured using qRT-PCR as described in Example 7-2 of WO2023-085787 A1. The table below quantitatively shows the degree of skin aging according to dermal thickness and collagen (COL1A1) and SDF1 mRNA levels in mice under conditions in which natural aging was induced and the test compound was not administered or administered. Furthermore, biweekly examinations were performed to assess the onset of hair loss due to aging.

TABLE 25

| | Dermal | Col1a1 | SDF1 | Relative area |
|---|---|---|---|---|
| | Thickness | mRNA | mRNA | of hair loss |
| | (μm) | (Fold) | (fold) | (%) |
| — | 211 (±20.1) | 1 (±0.1) | 1 (±0.0) | 100 (±7.5) |
| LX-403 | 232 (±16.2) | 2.4 (±0.2) | 1.8 (±0.1) | 69 (±2.5) |
| LX-1801 | 222 (±37.4) | 2.0 (±0.2) | 2.1 (±0.2) | 82 (±6.6) |
| LX-2001 | 241 (±46.6) | 2.2 (±0.4) | 1.6 (±0.2) | 73 (±8.3) |
| LX-2002 | 253 (±40.1) | 1.9 (±0.3) | 1.4 (±0.1) | 75 (±8.3) |
| LX-2003 | 235 (±56.3) | 2.3 (±0.4) | 1.7 (±0.1) | 71 (±2.2) |
| LX-2005 | 227 (±28.4) | 1.7 (±0.2) | 1.9 (±0.2) | 80 (±4.9) |
| LX-2007 | 219 (±31.1) | 1.6 (±0.3) | 1.3 (±0.2) | 86 (±5.5) |
| LX-2101 | 224 (±11.1) | 2.2 (±0.2) | 1.9 (±0.3) | 79 (±4.1) |
| LX-2707 | 237 (±19.4) | 2.4 (±0.2) | 1.8 (±0.1) | 75 (±5.4) |
| LX-3001 | 223 (±15.7) | 2.1 (±0.2) | 1.9 (±0.2) | 83 (±6.6) |
| LX-3002 | 219 (±21.3) | 1.8 (±0.2) | 1.6 (±0.1) | 84 (±6.1) |
| LX-5401 | 222 (±26.3) | 1.8 (±0.1) | 1.7 (±0.1) | 73 (±2.1) |
| LX-5411 | 231 (±27.1) | 2.7 (±0.1) | 2.3 (±0.2) | 64 (±4.8) |
| LX-5701 | 239 (±16.4) | 1.9 (±0.1) | 1.7 (±0.3) | 71 (±8.4) |
| LX-6807 | 267 (±22.4) | 3.2 (±0.2) | 2.1 (±0.2) | 72 (±3.8) |
| LX-2101 | 251 (±15.1) | 2.5 (±0.2) | 1.4 (±0.3) | 82 (±8.9) |
| LX-2102 | 234 (±9.6) | 2.0 (±0.2) | 1.3 (±0.1) | 89 (±7.5) |

As a result, as shown in the table above, when the test compound was administered in mice in which aging was induced, the skin thickness, collagen and SDF1 amounts increased, and the hair loss area decreased. In addition, aging-related markers such as p16 and SA-β-gal, aging-related secretory factor (SASP) and collagen-degrading enzymes such as MMP1, MMP12, and MMP13 in skin tissues also decreased by the test compound treatment. From this, it was confirmed that the test compounds improved skin aging and diseases such as epidermal atrophy, loss of elasticity, wrinkles, hyperpigmentation, and hair loss in mice by removing senescent cells that increase with aging. Furthermore, similar improvement results were confirmed when LX-401 or LX-405, which are similar to LX-403, LX-5401, or LX-5411, were treated.

Furthermore, when each of the test compounds of Table 25 was administered together with LX-6806, LX-6807, LX-9118, LX-9423, LX-9744, or LX-3403, the CI (combination index) was 0.52 to 0.77, confirming that there was synergy in efficacy. Similar synergy results were confirmed when LX-8401 was administered instead of LX-3403, and LX-9422 was administered instead of LX-9423.

### Example 21-11. Improved Wound/Injury Healing Ability

Various damages are caused through senescence-associated secretory factors (SASP) from senescent cells that accumulate due to various stresses and stimuli in various tissues. For example, senescent cells accumulate in the skin after laser, filler, botox, thread treatments, tattoos, plastic and cosmetic procedures, and surgeries, as well as exposure to UV rays, burns, or other toxic substances, which delay wound healing and cause damage such as scars.

To evaluate the wound healing ability of aged mice, two wounds of equal size were created on the dorsal skin using a 6 mm punch, and the wound sizes were measured using calipers. The table below shows the degree of wound healing after 7 days in mice naturally aged without and with the test compound.

TABLE 26

| | Wound size (mm) |
|---|---|
| — | 3.2 (±0.4) |
| LX-403 | 1.9 (±0.2) |
| LX-1801 | 2.5 (±0.6) |
| LX-2001 | 2.5 (±0.5) |
| LX-2002 | 2.7 (±0.4) |
| LX-2003 | 2.6 (±0.6) |
| LX-2005 | 2.9 (±0.3) |
| LX-2007 | 3.0 (±0.2) |
| LX-2101 | 1.9 (±0.3) |
| LX-2707 | 2.7 (±0.3) |
| LX-3001 | 2.4 (±0.1) |
| LX-3002 | 2.9 (±0.4) |
| LX-5401 | 2.3 (±0.3) |
| LX-5411 | 1.6 (±0.1) |
| LX-5701 | 1.9 (±0.2) |
| LX-6807 | 1.7 (±0.1) |

As a result, as shown in the table above, when the test compound was administered in mice with induced aging, the healing rate of skin wounds increased. In addition, in skin tissue, aging-related markers such as p16, SA-β-gal, and lipofuscin, as well as aging-related secretory factors (SASPs) and collagen-degrading enzymes such as MMP1, MMP12, and MMP13, were also reduced. From this, it was confirmed that the test compounds improved the wound healing ability of mice by eliminating senescent cells that increase with aging. Furthermore, similar improvement results were confirmed when LX-401 or LX-405, which are similar to LX-403, LX-5401, or LX-5411, were administered.

Furthermore, when each of the test compound of Table 26 was administered together with LX-6806, LX-6807, LX-9118, LX-9423, LX-9744, or LX-3403, the CI (combination index) was 0.69 to 0.78, confirming that there was synergy in efficacy. Similar synergy results were confirmed when LX-8401 was administered instead of LX-3403, and LX-9422 was administered instead of LX-9423.

Furthermore, to evaluate the scar healing ability according to wound, three wounds of the same size were created on the ears of rabbits using an 8 mm punch, and scar formation was induced through natural healing for four weeks. After confirming scar formation, the test compound of this example was administered intra-scar three times a week for four weeks, and the degree of scar healing was assessed visually. As a result, when the test compound of this example was applied to the scar area, the size of the scar was reduced compared to the control group, and the expression of collagen, a fibrosis marker, was also reduced. From this, it was confirmed that the test compounds of this example improved scar-related skin diseases by removing senescent cells that increased in the scar area.

In this way, as in this example, by administering the test compound of Table 26 on areas of treatment and surgery using laser, filler, botox, thread, ultraviolet rays, and burns, and thereby removing senescent cells, the treatment and recovery effects of skin damage (pain, bruises, swelling, embossing, wounds, scars, etc.) were confirmed, and when each of the test compound of Table 26 was administered together with LX-6806, LX-6807, LX-9118, LX-9423, LX-9744, or LX-3403, the CI (combination index) was 0.57 to 0.71, confirming that there was synergy in efficacy. Similar synergy results were confirmed when LX-8401 was administered instead of LX-3403, and LX-9422 was administered instead of LX-9423.

Furthermore, LX-2102, LX-2201, LX-2301, LX-2302, LX-2305, LX-2306, LX-2307, LX-2308, LX-2309, LX-2310, LX-2311, LX-2312, LX-2315, LX-2316, LX-2317, LX-2318, LX-2319, LX-2401, LX-2402, LX-2403, LX-2405, LX-2406, LX-2407, LX-2408, LX-2409, LX-2501, LX-2502, LX-2503, LX-2505, LX-2506, LX-2507, LX-2508, LX-2509, LX-2510, LX-2511, LX-2512, LX-2513, LX-2517, or LX-5433, which are analogs of LX-2002, LX-2003, LX-2005, LX-2007, or LX-2101, also showed similar skin aging improvement (Example 21-10) and damage treatment and recovery (Example 21-11) effects. When LX-6806, LX-6807, LX-9118, LX-9423, LX-9744, or LX-3403 were administered together, the CI (combination index) was 0.61 to 0.89, confirming that there was synergy in efficacy. Similar synergistic results were confirmed when LX-8401 was administered instead of LX-3403 and LX-9422 was administered instead of LX-9423.

### Example 21-12. Improvement of Joint Diseases (Osteoarthritis, Rheumatoid Arthritis, Juvenile Idiopathic Arthritis, Spondyloarthritis, Degenerative Cartilage Disease, Kyphosis, Etc.)

RNA was extracted from joint tissue as described in Example 7-2 of WO2023-085787 A1, and qRT-PCR was performed using the synthesized cDNA. The table below quantitatively shows the degree of joint aging according to the mRNA amount of arthritis markers in aged mice, untreated and treated with the test compound. To analyze joint pain due to joint aging, hotplate analysis was performed. Specifically, thermal pain thresholds were measured using a hot plate. After acclimating mice to a temperature of 30° C. for 10 minutes, the time required for hind paw licking or jumping responses to occur at 55° C. was measured. To prevent tissue damage, the response was measured for less than 30 seconds, and the average value was used from five measurements.

TABLE 27

| | Col2a1 mRNA (fold) | Response time (s) |
|---|---|---|
| — | 1 (±0.0) | 6.9 (±1.8) |
| LX-403 | 2.9 (±0.2) | 5.6 (±0.7) |
| LX-1801 | 2.6 (±0.2) | 4.9 (±1.1) |
| LX-2001 | 1.8 (±0.2) | 5.0 (±0.9) |
| LX-2002 | 1.7 (±0.3) | 5.3 (±0.6) |
| LX-2003 | 1.9 (±0.2) | 5.1 (±0.7) |
| LX-2005 | 2.2 (±0.1) | 4.9 (±1.2) |
| LX-2007 | 1.6 (±0.2) | 5.8 (±0.5) |
| LX-2101 | 1.9 (±0.2) | 5.1 (±0.4) |
| LX-2707 | 3.1 (±0.3) | 5.4 (±0.6) |
| LX-3001 | 2.1 (±0.1) | 6.1 (±0.6) |
| LX-3002 | 1.9 (±0.1) | 6.2 (±0.8) |
| LX-5401 | 2.7 (±0.2) | 5.9 (±0.2) |
| LX-5411 | 3.3 (±0.3) | 4.7 (±0.4) |
| LX-5701 | 2.7 (±0.3) | 4.6 (±1.3) |
| LX-6807 | 3.5 (±0.2) | 4.8 (±0.4) |

As a result, as shown in the table above, when the test compound was administered to mice in which aging was induced, the amount of type 2 collagen, a cartilage-forming gene, increased and the time of pain response to heat decreased. In addition, p16 (a senescence-related marker in joint tissue) and MMP13 (senescence-associated secretory factor (SASP) and a collagen-degrading enzyme) also decreased by treatment with the test compound. From this, it was confirmed that the test compounds improved joint diseases caused by joint aging in mice, such as osteoarthritis, rheumatoid arthritis, juvenile idiopathic arthritis, spondyloarthritis, degenerative cartilage disease, and kyphosis, by eliminating senescent cells that increase with aging in mice. Furthermore, similar improvement results were confirmed when LX-401 or LX-405, which are similar to LX-403, LX-5401, or LX-5411, was administered.

Furthermore, when each of the test compounds of Table 27 was administered together with LX-6806, LX-6807, LX-9118, LX-9423, LX-9744, or LX-3403, the CI (combination index) was 0.70 to 0.85, confirming that there was synergy in efficacy. Similar synergy results were confirmed when LX-8401 was administered instead of LX-3403, and LX-9422 was administered instead of LX-9423.

### Example 21-13. Improvement of Cardiac Disorders and Diseases (Cardiac Load Resistance, Cardiac Hypertrophy, Myocardial Infarction, Heart Failure, Etc.)

At the histological level, cardiac aging is characterized by the loss of ventricular cardiomyocytes due to a decreased ability to replace apoptotic or necrotic cardiomyocytes, leading to hypertrophy of the remaining cardiomyocytes. To assess age-related cardiac hypertrophy, frozen cardiac tissue sections were stained with hematoxylin/eosin (H/E) and the thickness of ventricular cardiomyocytes was observed under an optical microscope.

Furthermore, cardiac aging is characterized by decreased stress tolerance, which is known to be due to decreased expression of ATP-sensitive potassium (KATP) channels. RNA was extracted from heart tissue as described in Example 7-2 of WO2023-085787 A1, and qRT-PCR was performed on Sur2a, a regulatory subunit of the KATP channel, using the synthesized cDNA.

The table below quantitatively shows the degree of cardiac aging according to ventricular cardiomyocyte size and Sur2a mRNA levels in naturally aged mice, untreated and treated with the test compound.

TABLE 28

| | | Cardiomyocyte |
|---|---|---|
| | | cross-sectional |
| | Sur2a mRNA (fold) | area (μm2) |
| — | 1 (±0.1) | 372 (±9) |
| LX-2707 | 2.2 (±0.1) | 331 (±15.7) |
| LX-3001 | 1.8 (±0.2) | 345 (±27.6) |
| LX-3002 | 1.9 (±0.1) | 317 (±26.5) |
| LX-5701 | 2.8 (±0.2) | 297 (±20) |
| LX-6807 | 3.0 (±0.2) | 281 (±47.1) |

As a result, as shown in the table above, when the test compound was administered to mice with induced aging, the mRNA amount of sur2a, an indicator of cardiac stress tolerance, increased and the size of ventricular cardiomyocytes decreased. In addition, p16, an aging-related marker in cardiac tissue, was also reduced by treatment with the test compound. From this, it was confirmed that the test compound improved geriatric cardiac disorders and diseases (cardiac load resistance, cardiac hypertrophy, myocardial infarction, heart failure, etc.) due to cardiac aging in mice by eliminating senescent cells that increase with aging in mice.

Furthermore, when each of the test compounds of Table 28 was administered together with LX-6806, LX-6807, LX-9118, LX-9423, LX-9744, or LX-3403, the CI (combination index) was 0.54 to 0.79, confirming that there was synergy in efficacy. Similar synergy results were confirmed when LX-8401 was administered instead of LX-3403, and LX-9422 was administered instead of LX-9423.

### Example 21-14. Improvement of Bone Diseases (Osteoporosis, Fracture Healing Impairment, Alveolar Bone Loss, Periodontal Disease, Etc.)

Senile osteoporosis is a metabolic bone disease characterized by decreased bone density and a high fracture risk. It occurs in both men and women with aging. The accumulation of senescent cells is known to contribute to osteoporosis by increasing bone resorption by osteoclasts and decreasing bone formation by osteoblasts through the senescence-associated secretory factor (SASP). RNA was extracted from the left femur of mice as described in Example 7-2 WO2023-085787 A1, and qRT-PCR was performed using the synthesized cDNA. The table below quantitatively shows the mRNA levels of osteoclast markers Ctsk, Ocstamp, Oscar, Rank, and Trap in mice naturally aged without and with the test compound. The amount of each factor after aging was induced was calculated as 100% and compared with the amount of each factor after treatment with the test compound.

TABLE 29

| | Ctsk | Ocstamp | Oscar | Rank | Trap |
|---|---|---|---|---|---|
| | mRNA (%) | mRNA (%) | mRNA (%) | mRNA (%) | mRNA (%) |
| — | 100 (±6.9) | 100 (±3.8) | 100 (±9.1) | 100 (±3.2) | 100 (±7.5) |
| LX-2707 | 64 (±8.6) | 73 (±3.2) | 54 (±3.4) | 67 (±3.4) | 51 (±5.2) |
| LX-3001 | 67 (±7.2) | 75 (±3.3) | 61 (±4.7) | 56 (±6.3) | 78 (±6.8) |
| LX-3002 | 73 (±3.8) | 83 (±2.8) | 73 (±5.4) | 72 (±5.7) | 61 (±2.5) |
| LX-5701 | 57 (±13.0) | 70 (±6.7) | 72 (±7.2) | 68 (±10.4) | 63 (±18.4) |
| LX-6807 | 42 (±2.7) | 53 (±5.1) | 37 (±2.0) | 57 (±3.9) | 48 (±2.1) |
| LX-2101 | 61 (±12.1) | 69 (±10.3) | 79 (±5.6) | 75 (±2.7) | 50 (±14.2) |

As a result, as shown in the table above, treatment of aged mice with the test compound decreased the expression of osteoclast markers. Furthermore, p16, a aging-related marker in bone tissue, was also reduced by treatment with the test compound. From this, it was confirmed that the test compounds can improve age-related bone diseases such as osteoporosis, fracture healing disorders, and periodontal diseases such as alveolar bone loss by eliminating senescent cells that increase with age in mice and thereby inhibiting bone resorption due to bone aging in mice.

Furthermore, when each of the test compounds of Table 29 was administered together with LX-6806, LX-6807, LX-9118, LX-9423, LX-9744, or LX-3403, the CI (combination index) was 0.67 to 0.82, confirming a synergistic effect. Similar synergistic results were confirmed when LX-8401 was administered instead of LX-3403, and LX-9422 was administered instead of LX-9423.

### Example 21-15. Improvement of Eye Diseases (Cataracts, Etc.)

Cataracts, which occur with age, are a disease that causes visual impairment due to clouding of the lens and are a major cause of blindness. The mice of Example 21-1 were treated with the test compound mixed in PBS orally twice a week at a concentration of 360 mg/kg. Cataract development was assessed biweekly until death. The table below shows the incidence of cataracts in mice that were naturally aged, untreated and treated with the test compound.

TABLE 30

| | Cataract incidence (%) |
|---|---|
| — | 83 |
| LX-403 | 57 |
| LX-1801 | 68 |
| LX-2001 | 75 |
| LX-2002 | 71 |
| LX-2003 | 69 |
| LX-2005 | 72 |
| LX-2007 | 80 |
| LX-2101 | 77 |
| LX-2707 | 48 |
| LX-3001 | 73 |
| LX-3002 | 77 |
| LX-5401 | 69 |
| LX-5411 | 61 |
| LX-5701 | 64 |
| LX-6807 | 61 |

As a result, as shown in the table above, when the test compound was administered to mice in which aging was induced, the incidence of cataracts decreased. In addition, p16 (a senescence-related marker) and MMP13 (senescence-associated secretory factor (SASP) and a collagen-degrading enzyme) in ocular tissue were also reduced by treatment with the test compound. From this, it was confirmed that the test compounds suppressed the development of cataracts due to ocular aging in mice by eliminating senescent cells that increase with aging. Furthermore, similar improvement results were confirmed when LX-401 or LX-405, which are similar to LX-403, LX-5401, or LX-5411, was administered.

Furthermore, when each of the test compounds of Table 30 was administered together with LX-6806, LX-6807, LX-9118, LX-9423, LX-9744, or LX-3403, the CI (combination index) was 0.59 to 0.78, confirming that there was synergy in efficacy. Similar synergy results were confirmed when LX-8401 was administered instead of LX-3403, and LX-9422 was administered instead of LX-9423.

### Example 22. Removal of Senescent Cells and Related Anti-Aging Activity in Mice Induced by External Stress, Including Anticancer Agents

### Example 22-1. Establishment of an Anticancer Agent-Induced Aging Mouse Model

Eight-week-old male C57BL6J mice (KRIBB Laboratory Animal Resource Center) were acclimated to the experimental environment for one week. Afterwards, the anticancer agent doxorubicin was administered intraperitoneally at a concentration of 10 mg/kg, inducing aging for more than 10 days. The aging-induced mice were randomly assigned to experimental and control groups. LX-403 was mixed with a 10% ethanol solution of PEG400 and administered orally at a concentration of 25 mg/kg or intradermally at a concentration of 1 μM for 21 days. LX-1801 was mixed in distilled water with 4% DMSO, 30% PEG300, and 5% Tween80 and administered intradermally at a concentration of 0.4 μM daily for 21 days. LX-2001 was mixed in PBS and administered intradermally at a concentration of 2 μM daily for 21 days. LX-2002 was mixed in distilled water with 4% DMSO, 30% PEG300, and 5% Tween80 and administered intradermally at a concentration of 2 μM daily for 21 days. LX-2003 was mixed in distilled water with 4% DMSO, 30% PEG300, and 5% Tween80 and administered intradermally at a concentration of 10 μM daily for 21 days. LX-2005 was mixed in distilled water with 4% DMSO, 30% PEG300, and 5% Tween80 and administered intradermally at a concentration of 90 μM daily for 21 days. LX-2007 was mixed in distilled water with 4% DMSO, 30% PEG300, and 5% Tween80 and administered intradermally at a concentration of 90 μM daily for 21 days. LX-2707 was mixed in corn oil and administered orally at a concentration of 600 mg/kg daily for 21 days. LX-3001 was mixed in phosphate-buffered saline (PBS) and administered orally at a concentration of 300 mg/kg daily for 21 days. LX-3002 was mixed in distilled water with 4% DMSO, 30% PEG300, and 5% Tween80 and administered orally at a concentration of 10 mg/kg daily for 21 days. LX-5401 was mixed in 5% PBS and administered intraperitoneally at a concentration of 50 mg/kg daily for 21 days or intradermally at a concentration of 10 μM. LX-5411 was mixed in 5% phosphate-buffered saline (PBS) and administered orally at a concentration of 10 mg/kg daily for 21 days or intradermally at a concentration of 3 μM. LX-5701 was mixed in PBS and administered orally at a concentration of 360 mg/kg daily for 21 days. LX-6807 was mixed in saline and administered intraperitoneally at a concentration of 1 mg/kg daily for 21 days. The control group received only the solution in the same volume. After the end of drug administration, the methods described in each example below were performed.

### Example 22-2. Reduction in the Amount of Senescence-Associated Markers (p16, p21)

Mice were sacrificed using the same method as in Example 7-2 of WO2023-085787 A1. RNA was extracted from organ tissues of each site, and cDNA synthesized was used for qRT-PCR and analysis. The table below shows the mRNA amounts of senescence-associated markers p16 and p21 under conditions in which aging was induced by anticancer treatment and LX-5701 was not administered and was administered. The mRNA amounts of p16 and p21 after aging induction were calculated as 100% and compared with the mRNA amounts of p16 and p21 after LX-5701 treatment.

TABLE 31

| | | Liver | Kidney | eWAT |
|---|---|---|---|---|
| p16 mRNA | — | 100 (±6.8) | 100 (±4.1) | 100 (±7.1) |
| (%) | LX-5701 | 62 (±7.5) | 78 (±7.7) | 55 (±11.5) |
| p21 mRNA | — | 100 (±4.7) | 100 (±7.2) | 100 (±8.9) |
| (%) | LX-5701 | 64 (±11.7) | 75 (±5.8) | 71 (±8.8) |

As a result, as shown in the table above, when LX-5701 was administered to mice induced to undergo aging by anticancer treatment, the mRNA levels of p16 and p21 decreased in various organ tissues. This confirms that LX-5701 effectively reduces the levels of p16 and p21, aging-related markers that increase in various organ tissues of mice, by eliminating senescent cells that increase in mice treated with the anticancer drug.

### Example 22-3. Reduction in the Amount of the Aging-Related Marker (SA-β-Gal)

Mice were sacrificed as described in Example 7-3 of WO2023-085787 A1, and tissue sections were prepared from each organ tissue site and stained for the aging-related marker SA-β-gal. The table below shows the staining area of SA-β-gal, a senescence-related marker, under conditions in which aging was induced by treatment with an anticancer agent and LX-5701 was not administered and was administered.

TABLE 32

| SA-β-gal (%) | Liver | Kidney | Lung | eWAT |
|---|---|---|---|---|
| — | 100 (±3.1) | 100 (±5.7) | 100 (±7.3) | 100 (±7.4) |
| LX-5701 | 43 (±14.2) | 64 (±9.7) | 68 (±14.8) | 66 (±10.5) |

As shown in the table above, when LX-5701 was administered to mice induced to age by anticancer treatment, the area of SA-β-gal staining decreased in various organ tissues. This confirms that LX-5701 effectively reduces the amount of SA-β-gal, an aging marker that increases in various organ tissues of mice, by eliminating senescent cells that increase with age in mice treated with the anticancer agent.

### Example 22-4. Reduction in the Amount of Aging-Related Degenerative Marker (Lipofuscin)

Mice were sacrificed using the same method as in Example 7-4 of WO2023-085787 A1, and muscle tissue sections were prepared and stained for lipofuscin, an aging-related degenerative marker. The table below shows the staining area of lipofuscin, an aging-related degenerative marker, under conditions in which aging was induced by treatment with the anticancer agent and LX-5701 was not administered and was administered.

TABLE 33

Muscle

| | | |
|---|---|---|
| Lipofuscin (%) | — | 100 (±8.3) |
| | LX-5701 | 64 (±13.1) |

As shown in the table above, when LX-5701 was administered to mice induced to age by anticancer treatment, the area of lipofuscin staining in muscle tissue decreased. This confirms that LX-5701 effectively reduces the amount of lipofuscin, a marker of aging-related degeneration that increases in mouse muscle tissue, by eliminating senescent cells that increase with age in mice treated with the anticancer agent.

### Example 22-5. Reduction in the Amount of Senescence-Associated Secretory Factor (SASP)

The amount of IL6, a senescence-associated secretory factor (SASP), was measured in serum collected from mice using the same method as in Example 7-5 of WO2023-085787 A1 using an enzyme-linked immunosorbent assay (ELISA Kit, R&D Systems). The table below shows the amount of IL6, a senescence-associated secretory factor (SASP), under conditions in which aging was induced by anticancer treatment and LX-5701 was not administered and was administered.

TABLE 34

| | | |
|---|---|---|
| IL6 (%) | — | 100 (±18.3) |
| | LX-5701 | 57 (±21.5) |

As a result, as shown in the table above, when LX-5701 was administered to mice induced to age by anticancer treatment, the amount of IL6 in the serum decreased. This confirms that LX-5701 effectively reduces the amount of IL6, a senescence-associated secretory factor (SASP), which increases in serum by eliminating senescent cells that increase with age in mice treated with the anticancer drug.

### Example 22-6. Reduction in the Amount of Senescence-Associated Secretory Factor (SASP)

Using cDNA synthesized in the same manner as in Example 7-6 of WO2023-085787 A1, qRT-PCR was performed and analyzed for senescence-associated secretory factors (SASPs) like IL6, CXCL1, CXCL10, IL1B, etc. The table below shows the mRNA amounts of IL6, CXCL1, CXCL10, and IL1B, which are senescence-associated secretory factors (SASPs), under conditions in which aging was induced by anticancer treatment and LX-5701 was not administered and was administered. The mRNA amount of each factor after aging induction was calculated as 100% and compared with the mRNA amount of each factor after LX-5701 treatment.

TABLE 35

| mRNA (%) | | IL6 | CXCL1 | CXCL10 | IL1β |
|---|---|---|---|---|---|
| Liver | — | 100 (±16.8) | 100 (±15.3) | 100 (±8.5) | 100 (±7.7) |
| | LX-5701 | 64 (±7.5) | 68 (±14.8) | 75 (±4.1) | 73 (±3.5) |
| eWAT | — | 100 (±17.2) | 100 (±13.3) | 100 (±12.1) | 100 (±10.2) |
| | LX-5701 | 55 (±15.1) | 74 (±4.7) | 69 (±7.0) | 56 (±11.2) |

As shown in the table above, when LX-5701 was administered to mice induced to age by anticancer treatment, the mRNA levels of each senescence-associated secretory factor (SASP) decreased in various organ tissues. This confirms that LX-5701 effectively reduces the levels of senescence-associated secretory factors (IL6, CXCL1, CXCL10, and IL13) that increase in various organ tissues of mice by eliminating senescent cells that increase with anticancer treatment.

### Example 22-7. Improvement in Fatigue, Weakness, and Weight Loss

To analyze fatigue and weakness caused by anticancer treatment as side effects in mice, an activity test (open field test), an endurance test (wire hanging test), and a muscle and grip strength test were performed using the same method as in Example 7-7 of WO2023-085787 A1. The table below quantitatively represents the results of activity, endurance, muscle strength, and grip strength, which are criteria for assessing frailty, under conditions in which aging was induced by anticancer treatment and the test compound was not administered and was administered. The amount of each evaluation after aging was calculated as 100% and compared with the amount of each evaluation after test compound treatment.

TABLE 36

| | Open Field | Wire Hanging | Grip Sterenght | Mean Body |
|---|---|---|---|---|
| | Test Time | Test Time | Test Strength | Weight |
| | in Center (%) | (%) | (%) | (g) |
| — | 100 (±15.1) | 100 (±8.4) | 100 (±5.5) | 20.8 (±1.7) |
| LX-2707 | 232 (±33.6) | 192 (±12.7) | 128 (±5.7) | 22.4 (±2.1) |
| LX-3001 | 171 (±22.6) | 127 (±13.3) | 120 (±9.3) | 21.2 (±1.3) |
| LX-3002 | 169 (±21.8) | 143 (±10.4) | 113 (±9.7) | 21.0 (±1.7) |
| LX-5701 | 192 (±24.3) | 139 (±8.2) | 117 (±6.9) | 23.0 (±2.8) |
| LX-6807 | 249 (±34.1) | 209 (±14.5) | 137 (±6.3) | 23.2 (±2.7) |

As a result, as shown in the table above, when the test compound was administered to mice induced to age by anticancer treatment, activity, endurance, muscle strength, and grip strength increased, and body weight loss was improved. In addition, lipofuscin, an aging-related degenerative marker in muscle tissue, was also reduced by treatment with the test compound. From this, it was confirmed that the test compounds improved fatigue and weakness in mice caused by side effects of anticancer treatment by eliminating senescent cells that increase with aging in mice treated with the anticancer drug. Consistent with this improvement effect, it was confirmed that the test compounds increased the body weight of mice aged by treatment with the anticancer drug.

Furthermore, when each of the test compounds of Table 36 was administered together with LX-6806, LX-6807, LX-9118, LX-9423, LX-9744, or LX-3403, the CI (combination index) was 0.64 to 0.78, confirming that there was synergy in efficacy. Similar synergy results were confirmed when LX-8401 was administered instead of LX-3403, and LX-9422 was administered instead of LX-9423.

### Example 22-8. Improvement of Liver Damage

Serum collected from mice in the same manner as in Example 7-5 of WO2023-085787 A1 was used to measure the levels of aspartate aminotransferase (AST) and alanine transaminase (ALT), which are released into the blood when liver damage occurs. The amounts were measured spectrophotometrically using a kit (Sigma) according to the manufacturer's instructions. The table below shows the levels of AST and ALT, markers of liver damage, under conditions in which aging was induced by anticancer treatment and the test compound was not administered or was administered. The amount of each factor after aging was calculated as 100% and compared with the amount of each factor after treatment with the test compound.

TABLE 37

| % | AST | ALT |
|---|---|---|
| — | 100 (±8.3) | 100 (±4.1) |
| LX-2707 | 70 (±3.2) | 76 (±5.6) |
| LX-3001 | 81 (±5.4) | 85 (±3.6) |
| LX-3002 | 79 (±5.9) | 82 (±3.8) |
| LX-5701 | 72 (±13.5) | 64 (±15.7) |
| LX-6807 | 75 (±7.0) | 80 (±6.7) |

As a result, as shown in the table above, when the test compound was administered to mice induced to age by anticancer treatment, the levels of AST and ALT in the serum decreased. This confirms that the test compounds improve liver damage and dysfunction in mice by eliminating senescent cells that increase with anticancer treatment.

Furthermore, when each of the test compounds of Table 37 was administered together with LX-6806, LX-6807, LX-9118, LX-9423, LX-9744, or LX-3403, the CI (combination index) ranged from 0.59 to 0.87, confirming synergistic efficacy. Similar synergistic results were also observed when LX-8401 was administered instead of LX-3403, and LX-9422 was administered instead of LX-9423.

### Example 22-9. Improvement of Kidney Damage

The amounts of urea and creatinine, markers of kidney damage, were measured spectrophotometrically in serum collected from mice using the same method as in Examples 7-8 of WO2023-085787 A1. The table below shows the quantitative results of urea, creatinine, and Agtr1a mRNA, markers of kidney damage, under conditions in which aging was induced by anticancer treatment and the test compound was not administered or was administered. The amount of each factor after aging was calculated as 100% and compared with the amount of each factor after treatment with the test compound.

TABLE 38

| % | Urea | Creatinine | Agtr1a mRNA |
|---|---|---|---|
| — | 100 (±2.8) | 100 (±5.4) | 100 (±7.6) |
| LX-2707 | 80 (±3.3) | 75 (±5.3) | 62 (±3.8) |
| LX-3001 | 76 (±2.8) | 85 (±4.4) | 70 (±5.8) |
| LX-3002 | 77 (±3.3) | 83 (±2.6) | 73 (±2.9) |
| LX-5701 | 75 (±5.9) | 80 (±5.0) | 60 (±10.4) |
| LX-6807 | 72 (±5.8) | 73 (±2.7) | 43 (±3.0) |

As a result, as shown in the table above, when the test compound was administered to mice induced to age by anticancer treatment, the levels of urea, creatinine, and Agtr1a mRNA decreased. This confirms that the test compounds improve renal damage and dysfunction in mice by eliminating senescent cells that increase with anticancer treatment.

Furthermore, when each of the test compounds of Table 38 was administered together with LX-6806, LX-6807, LX-9118, LX-9423, LX-9744, or LX-3403, the CI (combination index) was 0.69 to 0.85, confirming synergistic efficacy. Similar synergistic results were confirmed when LX-8401 was administered instead of LX-3403, and LX-9422 was administered instead of LX-9423.

### Example 22-10. Improvement of Thrombosis

Thrombosis is a common side effect of radiation and genotoxic chemotherapy. It significantly increases the risk of post-treatment mortality and reduces quality of life through symptoms such as acute myocardial infarction, stroke, pulmonary thrombosis, acute peripheral arterial occlusion, deep vein thrombosis, portal vein thrombosis, acute renal vein occlusion, cerebral venous sinus thrombosis, and central retinal vein occlusion.

Using cDNA synthesized using the same method as in Example 7-6 of WO2023-085787 A1, qRT-PCR was performed and analyzed for the liver-produced senescence-associated secretory factor (SASP) and hemostasis-related factors like Serpine1 (PAI1), Plau (Urokinase), and TIMP1. To determine whether the treatment affects in vivo hemostasis, tail bleeding time was analyzed in mice induced to age by chemotherapy. Specifically, for tail bleeding analysis, mice were anesthetized, and the tail was incised 5 mm from the tip with a razor blade. The bleeding tail was then placed in a 15 ml test tube containing saline preheated to 37° C., and the time until blood flow stopped was measured. The bleeding time was determined when bleeding stopped for 10 seconds. The table below shows the results showing the mRNA amounts of hemostasis-related factors and tail bleeding times under conditions in which aging was induced by anticancer treatment and the test compound was not administered and was administered. The qPT-PCR results were compared with the amounts of each factor after aging was induced, calculating the amount of each factor as 100% and then treating it with the test compound.

TABLE 39

| | | | | Tail amputation |
|---|---|---|---|---|
| | Serpine1 | Plau | TIMP1 | bleeding time |
| % | mRNA (%) | mRNA (%) | mRNA (%) | (sec) |
| — | 100 (±21.8) | 100 (±8.7) | 100 (±33.1) | 51 (±3.6) |
| LX-2707 | 52 (±3.5) | 65 (±1.8) | 46 (±1.5) | 80 (±4.3) |
| LX-3001 | 76 (±2.1) | 69 (±2.2) | 73 (±3.1) | 72 (±2.6) |
| LX-3002 | 69 (±3.8) | 75 (±2.9) | 64 (±4.3) | 61 (±4.3) |
| LX-5701 | 45 (±20.1) | 53 (±13.9) | 53 (±11.2) | 89 (±15.2) |
| LX-6807 | 31 (±1.9) | 45 (±3.6) | 59 (±1.9) | 93 (±6.4) |

As a result, as shown in the table above, when the test compound was administered to mice induced to age by anticancer treatment, the levels of Serpine1, Plau, and TIMP1 mRNA decreased, and blood coagulation was actually inhibited, increasing tail amputation bleeding time. From this, it was confirmed that the test compound improved thrombosis, a side effect of anticancer therapy, by eliminating senescent cells that increase with the aging of mice due to anticancer treatment.

Furthermore, when each of the test compounds of Table 39 was administered together with LX-6806, LX-6807, LX-9118, LX-9423, LX-9744, or LX-3403, the CI (combination index) was 0.56 to 0.73, confirming a synergistic effect. Similar synergistic results were confirmed when LX-8401 was administered instead of LX-3403 and LX-9422 was administered instead of LX-9423.

### Example 22-11. Hair Loss Improvement

Alopecia is one of the most troubling side effects of chemotherapy, for which there is currently no treatment. Hair loss in mice induced to age by the anticancer drug was quantified using imaging software such as ImageJ (NIH) from digital photographs taken using a fixed camera position and magnification. The table below shows the percentage of hair loss areas in mice induced to age by the anticancer drug, untreated and treated with the test compound.

TABLE 40

| % | Relative area of hair loss (%) |
|---|---|
| — | 100 (±15.4) |
| LX-403 | 76 (±5.5) |
| LX-1801 | 78 (±5.4) |
| LX-2001 | 80 (±6.9) |
| LX-2002 | 83 (±10.4) |
| LX-2003 | 79 (±6.7) |
| LX-2005 | 82 (±4.6) |
| LX-2007 | 86 (±2.9) |
| LX-2101 | 79 (±3.9) |
| LX-2707 | 67 (±3.8) |
| LX-3001 | 82 (±4.7) |
| LX-3002 | 85 (±4.0) |
| LX-5401 | 81 (±3.7) |
| LX-5411 | 77 (±4.1) |
| LX-5701 | 67 (±17.8) |
| LX-6807 | 59 (±3.2) |

As a result, as shown in the table above, when the test compound was administered to mice induced to age by anticancer treatment, the extent of hair loss was reduced. This confirms that the test compounds improve hair loss in mice by eliminating senescent cells that increase with aging due to anticancer treatment. Furthermore, similar improvement results were observed when LX-401 or LX-405, which are similar to LX-403, LX-5401, or LX-5411, were administered.

Furthermore, when each of the test compounds of Table 40 was administered together with LX-6806, LX-6807, LX-9118, LX-9423, LX-9744, or LX-3403, the CI (combination index) was 0.69 to 0.88, confirming a synergistic effect. Similar synergistic results were observed when LX-8401 was administered in place of LX-3403, and LX-9422 was administered in place of LX-9423.

### Example 22-12. Improvement of Bone Marrow Dysfunction (Myelodysplastic Syndrome, Myelopathy, Bone Marrow Transplant Disorder, Anemia, Etc.)

Damage to the immune system, including the bone marrow, after chemotherapy is known to induce inflammatory responses and contribute to drug resistance and adverse effects. To analyze the function of bone marrow hematopoietic cells in mice induced to age by the anticancer agent, a cobblestone-area-forming-cell (CAFC) assay was performed. Specifically, bone marrow cells (BMCs) were collected from the femurs and tibias of mice, and BM-MNCs (mono-nuclear cells) were cultured in MethoCult™ GF M3434 methylcellulose medium (Stem Cell™ Technologies Inc, Canada). FBMD-1 stromal cells were seeded in each well of a 96-well plate to prepare a support cell stromal layer. After 1 week, BM-MNCs were diluted in CAFC medium and added to the support cell stromal layer for co-culture. After 5 weeks of culture, CAFCs were counted using an optical microscope. The table below shows the values of CAFC per 105 BMCs in mice that were aged with the anticancer agent, untreated and treated with the test compound.

TABLE 41

| % | CAFCs/10⁵ BMCs |
|---|---|
| — | 1.8 (±0.1) |
| LX-2707 | 2.7 (±0.2) |
| LX-3001 | 2.1 (±0.2) |
| LX-3002 | 2.3 (±0.2) |
| LX-5701 | 3.3 (±0.2) |
| LX-6807 | 3.5 (±0.2) |

As a result, as shown in the table above, when the test compound was administered to mice induced to age by anticancer treatment, the occurrence of CAFC, which reflects the degree of hematopoietic stem cell (HSC) proliferation and differentiation, increased. From this, it was confirmed that the test compounds improved the bone marrow function of the mice by eliminating senescent cells that increase with the aging of the mice due to anticancer treatment.

Furthermore, when each of the test compounds of Table 41 was administered together with LX-6806, LX-6807, LX-9118, LX-9423, LX-9744, or LX-3403, the CI (combination index) was 0.65 to 0.84, confirming a synergistic effect. Similar synergistic results were confirmed when LX-8401 was administered instead of LX-3403 and LX-9422 was administered instead of LX-9423.

### Example 23. Removal of Senescent Cells and Related Aging Inhibitory Activity in Mice Induced by Metabolic Disruptions, Including Obesity

### Example 23-1. Establishment of an Obesity-Induced Aging Mouse Model

15-week-old male C57BL6J mice (The Jackson Laboratory) were induced to obesity by a high-fat diet using the same method as in Example 9-1 WO2023-085787 A1. After acclimation to the experimental environment for one week, the mice were randomly assigned to experimental and control groups. LX-2707 was mixed with corn oil and administered orally at a concentration of 600 mg/kg daily for 21 days. LX-3001 was mixed with PBS and administered orally at a concentration of 300 mg/kg daily for 21 days. LX-3002 was mixed in distilled water with 4% DMSO, 30% PEG300, and 5% Tween80 and administered orally at a concentration of 10 mg/kg daily for 21 days. LX-5701 was mixed in PBS and administered orally at a concentration of 360 mg/kg daily for 21 days. LX-6807 was mixed in saline and administered intraperitoneally at a concentration of 1 mg/kg daily for 21 days. The control group received only the solution in the same volume. After the end of drug administration, the methods described in each example below were performed.

### Example 23-2. Reduction in the Amount of Aging-Related Markers (p16, p21)

Mice were sacrificed using the same method as in Example 7-2 of WO2023-085787 A1. RNA was extracted from organ tissues from each site, and the synthesized cDNA was used for qRT-PCR (quantitative real-time PCR) analysis. The table below shows the mRNA levels of aging-related markers p16 and p21 under conditions in which aging was induced by obesity and LX-5701 was not administered or was administered. The mRNA levels of p16 and p21 after obesity induction were calculated as 100% and compared with the mRNA levels of p16 and p21 after LX-5701 treatment.

TABLE 42

| % | | Liver | Muscle | Lung | Skin | eWAT | iWAT | asWAT | iBAT | Heart | Eye |
|---|---|---|---|---|---|---|---|---|---|---|---|
| p16 | — | 100 | 100 | 100 | 100 | 100 | 100 | 100 | 100 | 100 | 100 |
| mRNA | | (±8.6) | (±7.2) | (±7.9) | (±3.7) | (±4.6) | (±4.0) | (±5.1) | (±5.9) | (±2.4) | (±7.5) |
| | LX- | 63 | 75 | 74 | 77 | 65 | 61 | 67 | 72 | 80 | 71 |
| | 5701 | (±8.4) | (±6.6) | (±7.7) | (±5.6) | (±7.2) | (±10.2) | (±3.6) | (±7.1) | (±4.8) | (±6.3) |
| p21 | — | 100 | 100 | 100 | 100 | 100 | 100 | 100 | 100 | 100 | 100 |
| mRNA | | (±1.5) | (±3.5) | (±8.2) | (±7.0) | (±6.2) | (±4.3) | (±5.8) | (±9.1) | (±7.1) | (±5.1) |
| | LX- | 61 | 64 | 77 | 68 | 55 | 51 | 79 | 80 | 81 | 70 |
| | 5701 | (±8.8) | (±11.1) | (±8.5) | (±4.7) | (±11.4) | (±16.2) | (±7.9) | (±6.6) | (±7.8) | (±9.9) |

As a result, as shown in the table above, when LX-5701 was administered to mice with induced aging, the mRNA levels of p16 and p21 decreased in various organ tissues. This confirms that LX-5701 effectively reduces the levels of p16 and p21, aging-related markers that increase in various organ tissues of mice, by eliminating senescent cells that increase in mice due to obesity-induced aging.

### Example 23-3. Reduction in the Level of Senescence-Associated Secretory Factors (SASPs)

The levels of IL6 and activin A, senescence-associated secretory factors (SASP), were measured in serum collected from mice using the same method as in Example 7-5 of WO2023-085787 A1 using an enzyme-linked immunosorbent assay (ELISA Kit, R&D Systems). The table below shows the amounts of IL6 and activin A, senescence-associated secretory factors (SASPs), under conditions in which aging was induced by obesity and LX-5701 was not administered or was administered.

TABLE 43

| % | IL6 | Activin A |
|---|---|---|
| — | 100 (±11.6) | 100 (±7.5) |
| LX-5701 | 53.9 (±15.4) | 61.7 (±14.0) |

As a result, as shown in the table above, when LX-5701 was administered to mice with obesity-induced aging, the levels of IL6 and activin A in the serum decreased. This confirms that LX-5701 effectively reduces the levels of IL6 and activin A, senescence-associated secretory factors (SASPs) that increase in the serum of mice, by eliminating senescent cells that increase with obesity-induced aging.

### Example 23-4. Improvement of Neuropsychiatric Disorders (Anxiety, Depression, Lethargy, Etc.)

It was determined that anxiety and depression caused by obesity are not causally related to increased body weight, but rather are caused by the accumulation of senescent cells. Accordingly, an activity assessment (open field test) was performed using the same method as in Example 7-7 of WO2023-085787 A1 to analyze lethargy caused by anxiety and depression caused by obesity in mice. The table below quantitatively represents activity, a criterion for assessing lethargy due to anxiety and depression, under conditions in which aging was induced by obesity and the test compound was not administered or was administered. The activity assessment after obesity-induced aging was calculated as 100% and compared with the activity assessment after test compound treatment.

TABLE 44

| | | |
|---|---|---|
| Open Field Test | — | 100 (±16.8) |
| Time in Center (%) | LX-2707 | 224 (±12.8) |
| | LX-3001 | 168 (±11.9) |
| | LX-3002 | 173 (±14.4) |
| | LX-5701 | 217 (±20.1) |
| | LX-6807 | 202 (±18.2) |

As a result, as shown in the table above, when the test compound was administered to mice in which aging was induced by obesity, activity increased. From this, it was confirmed that the test compounds improved anxiety, depression, and lethargy in mice caused by obesity by removing senescent cells that increase with aging in mice due to obesity. It was confirmed that when LX-401, LX-405, LX-2101, LX-2707, LX-3403, LX-5401, LX-5411, LX-6806, LX-6807, LX-9118, LX-9423, or LX-9744 was administered, senescent cells were removed, similarly improving geriatric depression.

Furthermore, when each of the test compounds of Table 44 was administered together with LX-6806, LX-6807, LX-9118, LX-9423, LX-9744, or LX-3403, the CI (combination index) was 0.59 to 0.74, confirming that there was synergy in efficacy. Similar synergy results were confirmed when LX-8401 was administered instead of LX-3403, and LX-9422 was administered instead of LX-9423.

### Example 23-5. Improvement of Fatty Liver (Alcoholic/Non-Alcoholic Fatty Liver, Hepatic Steatosis, Etc.)

Mice were sacrificed, and liver sections were prepared using the same method as in Example 7-3 of WO2023-085787 A1. Fat accumulation was stained with Oil Red O. In addition, AST and ALT, indicators of liver damage, were measured using the same method as in Example 8-8 of WO2023-085787 A1. The table below shows the results of the Oil Red O staining area, a criterion for determining fatty liver, and the measured amount of liver damage indicators under conditions in which aging was induced by obesity and the test compound was not administered or was administered. The Oil Red O staining area and the amount of liver damage indicators after obesity-induced aging were calculated as 100% and compared with the values after treatment with the test compound.

TABLE 45

| | Oil Red O (%) | AST (%) | ALT (%) |
|---|---|---|---|
| — | 100 (±21.5) | 100 (±12.7) | 100 (±10.5) |
| LX-2707 | 67 (±1.7) | 75 (±2.1) | 64 (±3.1) |
| LX-3001 | 83 (±4.0) | 81 (±4.7) | 71 (±1.3) |
| LX-3002 | 79 (±4.8) | 85 (±3.0) | 73 (±2.9) |
| LX-5701 | 55 (±17.2) | 69 (±8.8) | 64 (±7.9) |
| LX-6807 | 53 (±3.9) | 66 (±4.4) | 70 (±6.4) |

As a result, as shown in the table above, when the test compound was administered to mice induced to age due to obesity, the staining area of Oil Red O and the levels of serum AST and ALT, indicators of liver damage, decreased. From this, it was confirmed that the test compounds reduced the amount of fat accumulated in the liver tissue of the mice and improved hepatic steatosis by eliminating senescent cells that increase with the aging of the mice due to obesity.

Furthermore, when each of the test compounds of Table 45 was administered together with LX-6806, LX-6807, LX-9118, LX-9423, LX-9744, or LX-3403, the CI (combination index) was 0.64 to 0.89, confirming a synergistic effect. Similar synergistic results were confirmed when LX-8401 was administered instead of LX-3403 and LX-9422 was administered instead of LX-9423.

### Example 23-6. Improvement of Diabetes and Diabetic Complications (Diabetic Ulcers, Diabetic Retinopathy, Diabetic Cardiomyopathy, Diabetic Nephropathy, Etc.)

Serum collected from mice using the same method as in Example 7-5 of WO2023-085787 A1 was used to measure glycated hemoglobin (HbA1c), a marker of hyperglycemia and diabetes. Furthermore, the improvement in diabetic foot ulcers and diabetic retinopathy, major complications of diabetes, was analyzed. Specifically, a diabetic foot ulcer model was created by creating a full-thickness 5 mm×5 mm wound on the left dorsal skin of mice induced with obesity-induced diabetes. The wound size was measured under untreated and treated conditions with the test compound, and the healing effect was analyzed. In addition, the mRNA amount of VEGF, a marker of angiogenesis in retinopathy, was measured from the eye using qRT-PCR as described in Example 7-2 of WO2023-085787 A1. The qRT-PCR results were calculated as 100% for the amount of each factor after aging was induced, and compared with the amount of each factor after treatment with the test compound.

TABLE 46

| | | diabetic foot | VEGF |
|---|---|---|---|
| | HbA1c (%) | ulcer size (mm) | mRNA (%) |
| — | 6.9 (±1.5) | 4.2 (±0.4) | 100 (±6.8) |
| LX-2707 | 6.1 (±0.6) | 3.4 (±0.3) | 76 (±4.0) |
| LX-3001 | 6.4 (±0.4) | 3.5 (±0.2) | 77 (±2.4) |
| LX-3002 | 6.5 (±0.3) | 3.8 (±0.7) | 81 (±6.2) |
| LX-5701 | 5.0 (±1.3) | 3.0 (±0.9) | 61 (±10.6) |
| LX-6807 | 5.4 (±0.5) | 3.3 (±0.5) | 68 (±1.6) |

As a result, as shown in the table above, when the test compound was administered to mice with diabetes induced by obesity, the amount of serum glycated hemoglobin (HbA1c) decreased, the size of the wound on the dorsum of the foot decreased, and the amount of VEFG mRNA in the ocular tissue decreased. In addition, p16 and p21, which are aging-related markers in the skin, heart, and ocular tissues, were also decreased by treatment with the test compound. From this, it was confirmed that the test compounds reduce diabetic symptoms induced in mice and improve diabetic complications such as diabetic ulcers, diabetic retinopathy, diabetic cardiomyopathy, and diabetic nephropathy by eliminating senescent cells that increase with aging in mice due to obesity.

Furthermore, when each of the test compounds of Table 46 was administered together with LX-6806, LX-6807, LX-9118, LX-9423, LX-9744, or LX-3403, the CI (combination index) was 0.72 to 0.85, confirming that there was synergy in efficacy. Similar synergy results were confirmed when LX-8401 was administered instead of LX-3403, and LX-9422 was administered instead of LX-9423.

### Example 24. Improvement of Fibrosis and Sclerosis

### Example 24-1. Improvement of Fibrosis

Eight-week-old male C57BL6J mice (KRIBB Laboratory Animal Resource Center) were acclimated to the experimental environment for one week. They were then administered the anticancer drug bleomyxin via intratracheal inhalation at a concentration of 2.5 mg/kg, inducing aging for more than 10 days. The aging-induced mice were randomly assigned to experimental and control groups. LX-2707 was mixed with corn oil and administered orally at a concentration of 600 mg/kg daily for 21 days. LX-3001 was mixed with PBS and administered orally at a concentration of 300 mg/kg daily for 21 days. LX-3002 was mixed in distilled water with 4% DMSO, 30% PEG300, 5% tween80 and administered orally at a concentration of 10 mg/kg daily for 21 days. LX-5701 was mixed in PBS and administered orally at a concentration of 360 mg/kg daily for 21 days. LX-6807 was mixed in saline and administered intraperitoneally at a concentration of 1 mg/kg daily for 21 days. The control group received only the solution in the same volume. After the end of drug administration, mice were sacrificed in the same manner as in Example 7-2 of WO2023-085787 A1, RNA was extracted from lung tissue, and qRT-PCR (quantitative real-time PCR) was performed and analyzed using the synthesized cDNA. The table below shows the amounts of p16, a senescence-related marker, and IL6 and Col1a1 mRNA, senescence-related secretory factors, under conditions in which aging was induced and the test compound was not administered and was administered. The amount of mRNA after aging was calculated as 100%, and the results after treatment with the test compound were compared. Furthermore, paraffin sections were stained with hematoxylin/eosin (H/E), and the size of alveoli was measured under an optical microscope to assess the degree of lung function impairment due to pulmonary fibrosis.

TABLE 47

| | p16 | IL6 | Col1a1 | Airspace |
|---|---|---|---|---|
| | mRNA (%) | mRNA (%) | mRNA (%) | enlargement (μm) |
| — | 100 (±5.8) | 100 (±11.4) | 100 (±6.7) | 39.8 (±0.4) |
| LX-2707 | 43 (±4.2) | 38 (±4.1) | 70 (±6.5) | 35.4 (±1.8) |
| LX-3001 | 65 (±3.8) | 51 (±3.6) | 64 (±3.9) | 36.2 (±2.2) |
| LX-3002 | 61 (±2.0) | 52 (±3.9) | 62 (±2.8) | 37.5 (±2.9) |
| LX-5701 | 65 (±7.1) | 59 (±13.2) | 52 (±13.9) | 30.1 (±2.7) |
| LX-6807 | 54 (±1.9) | 48 (±2.4) | 56 (±3.6) | 33.8 (±3.6) |

As a result, as shown in the table above, when the test compound was administered to mice with lung aging induced by the anticancer agent, the amounts of aging-related markers, inflammation, and fibrosis markers decreased, and the average size of the alveoli, which had been dilated due to loss of elasticity and resilience in pulmonary fibrosis, decreased. From this, it was confirmed that the test compounds reduced pulmonary fibrosis induced in mice and improved pulmonary dysfunction by eliminating senescent cells that increase with aging.

Furthermore, when each of the test compounds of Table 47 was administered together with LX-6806, LX-6807, LX-9118, LX-9423, LX-9744, or LX-3403, the CI (combination index) was 0.67 to 0.82, confirming a synergistic effect. Similar synergistic results were confirmed when LX-8401 was administered instead of LX-3403 and LX-9422 was administered instead of LX-9423.

### Example 24-2. Improvement of Sclerosis

Eight-week-old male C57BL6J mice (KRIBB Laboratory Animal Resource Center) were acclimated to the experimental environment for one week. Then, 100 μl of the anticancer drug bleomyxin (1 mg/ml) was administered subcutaneously every other day to induce scleroderma for more than three weeks. Mice with scleroderma were randomly assigned to experimental and control groups. LX-403 was mixed with a 10% ethanol solution of PEG400 and administered orally at a concentration of 25 mg/kg daily for 21 days or administered directly to the skin at a concentration of 1 μM. LX-1801 was mixed with a solution of 4% DMSO, 30% PEG300, and 5% Tween80 in distilled water and administered subcutaneously at a concentration of 0.4 μM daily for 21 days. LX-2001 was mixed in PBS and administered intradermally at a concentration of 2 μM daily for 21 days. LX-2002 was mixed in distilled water with 4% DMSO, 30% PEG300, and 5% Tween80 and administered intradermally at a concentration of 2 μM daily for 21 days. LX-2003 was mixed in distilled water with 4% DMSO, 30% PEG300, and 5% Tween80 and administered intradermally at a concentration of 10 μM daily for 21 days. LX-2005 was mixed in distilled water with 4% DMSO, 30% PEG300, and 5% Tween80 and administered intradermally at a concentration of 90 μM daily for 21 days. LX-2007 was mixed in distilled water with 4% DMSO, 30% PEG300, and 5% Tween80 and administered intradermally at a concentration of 90 μM daily for 21 days. LX-2707 was mixed with corn oil and administered orally at 600 mg/kg daily for 21 days. LX-3001 was mixed with phosphate-buffered saline (PBS) and administered orally at 300 mg/kg daily for 21 days. LX-3002 was mixed with distilled water with 4% DMSO, 30% PEG300, 5% tween80 and administered orally at 10 mg/kg daily for 21 days. LX-5401 was mixed with 5% PBS and administered intraperitoneally at 50 mg/kg daily for 21 days or subcutaneously at 10 μM. LX-5411 was mixed with 5% PBS and administered orally at 10 mg/kg daily for 21 days or subcutaneously at 3 μM. LX-5701 was mixed with PBS and administered orally at 360 mg/kg daily for 21 days. LX-6807 was mixed in saline and administered intraperitoneally at a concentration of 1 mg/kg daily for 21 days. The control group was administered only the solution in the same volume. After the end of drug administration, mice were sacrificed in the same manner as in Example 7-2 of WO2023-085787 A1, RNA was extracted from skin tissue, and the synthesized cDNA was used to perform and analyze the mRNA amounts of p16 and collagen (COL1A1) by qRT-PCR (quantitative real-time PCR). In addition, to measure the degree of bleomycin-induced skin hardening, skin frozen sections were stained with H/E (Hematoxylin/Eosin), and the thickness of the dermis was observed under an optical microscope. The table below quantitatively shows the mRNA amounts and dermal thickness in mice with scleroderma induced under conditions in which the test compound was not administered and was administered.

TABLE 48

| | p16 | Col1a1 | Dermal |
|---|---|---|---|
| | mRNA (%) | mRNA (%) | Thickness (μm) |
| — | 100 (±8.8) | 100 (±13.2) | 459 (±38.7) |
| LX-403 | 34.7 (±4.1) | 45.7 (±4.5) | 356 (±36.3) |
| LX-1801 | 35.4 (±5.6) | 47.8 (±4.7) | 418 (±42.3) |
| LX-2001 | 64.2 (±4.4) | 62.8 (±5.6) | 394 (±78.4) |
| LX-2002 | 51.3 (±2.6) | 71.2 (±4.2) | 427 (±60.1) |
| LX-2003 | 72.4 (±5.8) | 73.8 (±3.5) | 411 (±81.3) |
| LX-2005 | 62.8 (±4.6) | 68.2 (±2.4) | 432 (±61.7) |
| LX-2007 | 75.1 (±3.5) | 80.5 (±4.0) | 421 (±83.6) |
| LX-2707 | 67.5 (±4.7) | 64.1 (±2.0) | 389 (±23.6) |
| LX-3001 | 76.1 (±5.5) | 81.4 (±2.5) | 421 (±28.4) |
| LX-3002 | 77.0 (±3.1) | 78.8 (±3.5) | 434 (±37.9) |
| LX-5401 | 61.2 (±6.7) | 74.6 (±2.4) | 401 (±46.3) |
| LX-5411 | 43.8 (±3.0) | 55.3 (±1.6) | 376 (±47.1) |
| LX-5701 | 64 (±9.4) | 59.7 (±17.3) | 358 (±21.4) |
| LX-6807 | 64.7 (±3.5) | 37.6 (±2.5) | 315 (±29.8) |

As a result, as shown in the table above, when the test compound was administered to mice with scleroderma induced by the anticancer agent, the amount of aging-related markers and fibrosis markers decreased, and the thickness of the skin thickened by scleroderma decreased. From this, it was confirmed that the test compounds improved scleroderma induced in mice by eliminating senescent cells that increase with aging. Furthermore, similar improvement results were confirmed when LX-401 or LX-405, which are similar to LX-403, LX-5401, or LX-5411, were administered.

Furthermore, when each of the test compounds of Table 48 was administered together with LX-6806, LX-6807, LX-9118, LX-9423, LX-9744, or LX-3403, the CI (combination index) was 0.61 to 0.76, confirming that there was synergy in efficacy. Similar synergy results were confirmed when LX-8401 was administered instead of LX-3403, and LX-9422 was administered instead of LX-9423.

### Example 25. Improvement of Pulmonary Dysfunction and Diseases (Chronic Obstructive Pulmonary Disease, Emphysema, Etc.)

Eight-week-old male C57BL6J mice (KRIBB Laboratory Animal Resource Center) were acclimated to the experimental environment for one week. After exposure to standard cigarette smoke (costa monitoring cigarette 7) for 5 hours per day, 5 days per week for 6 months, chronic obstructive pulmonary disease (COPD) was induced. Mice with induced COPD were randomly assigned to experimental and control groups. LX-2707 was administered orally at a concentration of 600 mg/kg daily for 21 days in corn oil. LX-3001 was administered orally at a concentration of 300 mg/kg daily for 21 days in PBS. LX-3002 was mixed in distilled water with 4% DMSO, 30% PEG300, 5% tween80 and administered orally at a concentration of 10 mg/kg daily for 21 days. LX-5701 was mixed in PBS and administered orally at a concentration of 360 mg/kg daily for 21 days. LX-6807 was mixed in saline and administered intraperitoneally at a concentration of 1 mg/kg daily for 21 days. The control group received only the solution in the same volume. After the end of drug administration, mice were sacrificed in the same manner as in Example 7-2 of WO2023-085787 A1, RNA was extracted from lung tissue, and qRT-PCR (quantitative real-time PCR) was performed and analyzed using the synthesized cDNA. The table below shows the mRNA amounts of p16, a senescence-related marker, and IL6, a senescence-related secretory factor, under conditions in which aging was induced and the test compound was not administered and was administered. The amount of mRNA after aging was calculated as 100%, and the results after treatment with the test compound were compared. Furthermore, paraffin sections were stained with hematoxylin/eosin (H/E), and the size of alveoli was measured under a light microscope to assess the degree of lung function impairment.

TABLE 49

| | p16 | IL6 | Airspace |
|---|---|---|---|
| | mRNA (%) | mRNA (%) | enlargement (μm) |
| — | 100 (±6.7) | 100 (±19.5) | 38.7 (±0.6) |
| LX-2707 | 78 (±5.5) | 76 (±6.7) | 36.4 (±1.9) |
| LX-3001 | 83 (±5.0) | 79 (±9.9) | 37.1 (±2.7) |
| LX-3002 | 84 (±6.4) | 82 (±1.7) | 35.9 (±3.1) |
| LX-5701 | 70 (±7.8) | 59 (±16.8) | 31.9 (±2.4) |
| LX-6807 | 68 (±2.4) | 48 (±5.6) | 30.4 (±1.5) |

As a result, as shown in the table above, when the test compound was administered to mice with lung disease induced by cigarette smoke, the amount of aging-related and inflammatory markers decreased, and the average size of dilated alveoli decreased. From this, it was confirmed that the test compounds suppressed lung aging and inflammation due to chronic obstructive pulmonary disease induced by cigarette smoke exposure in mice and improved lung function impairment by eliminating senescent cells that increase with aging.

Furthermore, when each of the test compounds of Table 49 was administered together with LX-6806, LX-6807, LX-9118, LX-9423, LX-9744, or LX-3403, the CI (combination index) was 0.61 to 0.82, confirming a synergistic effect. Similar synergistic results were confirmed when LX-8401 was administered instead of LX-3403 and LX-9422 was administered instead of LX-9423.

### Example 26. Improvement of Liver Diseases and Disorders (Liver Fibrosis, Cirrhosis, Hepatitis, Primary Biliary Cirrhosis, Primary Sclerosing Cholangitis, Biliary Atresia, Bile Duct Damage, Etc.)

Eight-week-old male C57BL6J mice (KRIBB Laboratory Animal Resource Center) were acclimated to the experimental environment for one week. Carbon tetrachloride (CCL4) was administered intraperitoneally at a concentration of 0.5 μl/g, and aging was induced for at least six weeks. The aging-induced mice were randomly assigned to experimental and control groups. LX-2707, LX-3001, LX-3002, LX-5701, and LX-6807 were administered in the same manner as in Example 25. The control group received only the solution in the same volume. After the drug administration was terminated, mice were sacrificed, and RNA was extracted from the liver in the same manner as in Example 7-2 of WO2023-085787 A1, and qRT-PCR was performed using the synthesized cDNA. The table below shows the mRNA amounts of p16, a senescence-related marker, and IL6 and Col1a1, senescence-related secretory factors, and serum AST, a liver damage marker, under conditions in which liver damage and disease were induced and the test compound was not administered or was administered. The mRNA and AST amounts after aging was induced were calculated as 100% and compared with the mRNA amount of p16 after treatment with the test compound.

TABLE 50

| | p16 | IL6 | Col1a1 | Serum |
|---|---|---|---|---|
| | mRNA (%) | mRNA (%) | mRNA (%) | AST (%) |
| — | 100 (±7.6) | 100 (±18.7) | 100 (±9.6) | 100 (±3.7) |
| LX-2707 | 68 (±1.6) | 56 (±2.1) | 76 (±2.5) | 79 (±5.6) |
| LX-3001 | 76 (±3.7) | 65 (±3.4) | 71 (±2.4) | 82 (±4.5) |
| LX-3002 | 73 (±2.5) | 68 (±3.5) | 75 (±3.0) | 83 (±6.7) |
| LX-5701 | 70 (±6.2) | 53 (±17.6) | 62 (±6.7) | 77 (±8.0) |
| LX-6807 | 67 (±3.0) | 58 (±2.6) | 51 (±3.1) | 75 (±1.4) |

As shown in the table above, treatment of mice with liver fibrosis induced by the anticancer drug with the test compound resulted in a decrease in the levels of aging-related and fibrosis markers, as well as a decrease in the blood levels of AST, a liver damage marker. This confirms that the test compounds improve liver damage and diseases induced in mice, e.g., with liver fibrosis, cirrhosis, hepatitis, primary biliary cirrhosis, primary sclerosing cholangitis, biliary atresia, or bile duct damage by eliminating senescent cells that increase with aging.

Furthermore, when each of the test compounds of Table 50 was administered together with LX-6806, LX-6807, LX-9118, LX-9423, LX-9744, or LX-3403, the CI (combination index) was 0.68 to 0.87, confirming that there was synergy in efficacy. Similar synergy results were confirmed when LX-8401 was administered instead of LX-3403, and LX-9422 was administered instead of LX-9423.

### Example 27. Improvement of Vascular Disorders and Diseases (Atherosclerosis, Aortic Aneurysm, Coronary Aneurysm, Cerebral Aneurysm, Geriatric Vascular Dysfunction, Etc.)

Fifteen-week-old male C57BL6-ApoE^{-/-}-mice (SLC) with atherosclerosis induced by a high-fat diet in the same manner as in Example 9-1 of WO2023-085787 A1 were acclimated to the experimental environment for one week and then randomly assigned to experimental and control groups. LX-2707, LX-3001, LX-3002, LX-5701, and LX-6807 were administered in the same manner as in Example 25. The control group was administered only the solution in the same volume. After the drug administration was terminated, the mice were sacrificed, and RNA was extracted from the aortic arch of the thoracic aorta using the same method as Example 7-2 of WO2023-085787 A1, and qRT-PCR was performed using the synthesized cDNA. In addition, the remaining part of the thoracic aorta was dissected, fixed flat in 4% paraformaldehyde at room temperature for 12 hours, and the vascular plaque was stained with Oil Red O using the same method as Example 23-5. The table below shows the mRNA amount of p16, a senescence-related marker, and the amount of stained vascular plaque under conditions in which atherosclerosis was induced and the test compound was not administered and was administered. The mRNA amount after aging was induced was calculated as 100% and compared with the mRNA amount of p16 after treatment with the test compound.

TABLE 51

| | p16 mRNA (%) | Plaque number |
|---|---|---|
| — | 100 (±7.6) | 21.4 (±5.7) |
| LX-2707 | 81 (±6.0) | 19.4 (±3.6) |
| LX-3001 | 78 (±7.2) | 17.5 (±4.2) |
| LX-3002 | 71 (±4.9) | 18.2 (±5.0) |
| LX-5701 | 71 (±12.8) | 12.7 (±4.6) |
| LX-6807 | 59 (±4.7) | 15.3 (±2.4) |

As a result, as shown in the table above, when the test compound was administered to mice with atherosclerosis induced by a high-fat diet, the amount of aging-related markers decreased and the amount of vascular plaque in the thoracic aorta decreased. From this, it was confirmed that the test compounds improved vascular aging and diseases induced in mice, such as atherosclerosis, aortic aneurysm, coronary aneurysm, cerebral aneurysm, and age-related vascular dysfunction, by eliminating senescent cells that increase with aging.

Furthermore, when each of the test compounds of Table 51 was administered together with LX-6806, LX-6807, LX-9118, LX-9423, LX-9744, or LX-3403, the CI (combination index) was 0.64 to 0.79, confirming a synergistic effect. Similar synergistic results were confirmed when LX-8401 was administered instead of LX-3403 and LX-9422 was administered instead of LX-9423.

### Example 28. Improvement of Degenerative Brain Diseases (Dementia, Alzheimer's Disease, Memory Loss, (Mild) Cognitive Impairment, Etc.)

Twelve-week-old male SAMP8 mice (SLC), a cognitive impairment model, were acclimated to the experimental environment for one week and then randomly assigned to experimental and control groups. LX-2707, LX-3001, LX-3002, LX-5701, and LX-6807 were administered in the same manner as in Example 25. LX-2101 was mixed with 5% DMSO (Sigma) in PBS and administered intraperitoneally at a concentration of 10 mg/kg daily for 21 days. The control group received only the same volume of solution. After drug administration, the mice were tested in the Y maze test to analyze the degree of cognitive and memory improvement. The Y-maze test is a method to assess short-term memory-type spatial awareness. It is a Y-shaped enclosed maze made of black acrylic to the following specifications (40 cm in length, 8 cm in width, 13 cm in height, with each arm angled 120 degrees). Three arms are designated A, B, and C. A mouse is carefully placed on one arm and allowed to move freely for 8 minutes. The arm into which the mouse enters up to its tail is recorded in order. Each instance of entering each arm sequentially is awarded 1 point (actual alternation). Alternation behavior is defined as entering all arms without overlapping, and spontaneous alternation behavior is calculated using the following formula:

Spontaneous alternation behavior (%)=Actual alternation/Maximum alternation×100
Maximum alternation: Total number of entries-2

After the behavioral evaluation, the mice were sacrificed, and RNA from the hippocampus region of the brain was extracted using the same method as Example 7-2 of WO2023-085787 A1, and qRT-PCR was performed using the synthesized cDNA. The table below quantitatively shows the mRNA of p16, an aging-related marker, and spontaneous altered behavior, a criterion for judging cognitive impairment, in cognitive impairment model mice untreated or treated with the test compound.

TABLE 52

| | | Spontaneous |
|---|---|---|
| | p16 mRNA (%) | alternation (%) |
| — | 100 (±8.4) | 48.4 (±27.3) |
| LX-2707 | 75 (±3.3) | 54.5 (±2.0) |
| LX-3001 | 64 (±2.8) | 50.6 (±3.6) |
| LX-3002 | 79 (±5.4) | 51.1 (±5.4) |
| LX-5701 | 70 (±9.6) | 62.7 (±11.9) |
| LX-6807 | 39 (±6.2) | 60.7 (±4.3) |
| LX-2101 | 82 (±7.1) | 51.1 (±16.8) |

As a result, as shown in the table above, when the test compound was administered to cognitive impairment model mice, the amount of p16, an aging-related marker, decreased and spontaneous alterative behavior increased. From this, it was confirmed that the test compounds improved cognitive and memory impairments induced in mice, such as dementia, Alzheimer's disease, memory loss, and (mild) cognitive impairment, by eliminating senescent cells that increase with aging.

Furthermore, when each of the test compounds of Table 52 was administered together with LX-6806, LX-6807, LX-9118, LX-9423, LX-9744, or LX-3403, the CI (combination index) was 0.74 to 0.87, confirming a synergistic effect. Similar synergistic results were confirmed when LX-8401 was administered instead of LX-3403 and LX-9422 was administered instead of LX-9423.

### Example 29. Improvement of Retinal Degenerative Diseases (Age-Related Macular Degeneration, Macular Edema, Retinopathy, Etc.)

Eight-week-old male C57BL6J mice (KRIBB Laboratory Animal Resource Center) were acclimated to the experimental environment for one week. Then, retinal degenerative diseases were induced for more than 7 days by intravenous administration of 20 mg/kg of NaIO₃, an oxidizing agent that induces retinal pigment epithelial cell death and retinal degeneration. Mice with retinal degenerative diseases were randomly assigned to experimental and control groups. LX-403 was mixed with a 10% ethanol solution of PEG400 and administered orally at a concentration of 25 mg/kg daily for 21 days or administered intraocularly at a concentration of 1 μM. LX-1801 was mixed in distilled water with 4% DMSO, 30% PEG300, and 5% Tween80 and administered intraocularly at a concentration of 0.4 μM daily for 21 days. LX-2001 was mixed in PBS and administered intraocularly at a concentration of 2 μM daily for 21 days. LX-2002 was mixed in distilled water with 4% DMSO, 30% PEG300, and 5% Tween80 and administered intraocularly at a concentration of 2 μM daily for 21 days. LX-2003 was mixed in distilled water with 4% DMSO, 30% PEG300, and 5% Tween80 and administered intraocularly at a concentration of 10 μM daily for 21 days. LX-2005 was mixed in distilled water with 4% DMSO, 30% PEG300, and 5% Tween80 and administered intraocularly at a concentration of 90 μM daily for 21 days. LX-2007 was mixed in distilled water with 4% DMSO, 30% PEG300, and 5% Tween80 and administered intraocularly at a concentration of 90 μM daily for 21 days. LX-2707 was mixed in corn oil and administered orally at a concentration of 600 mg/kg daily for 21 days. LX-3001 was mixed in PBS and administered orally at a concentration of 300 mg/kg daily for 21 days. LX-3002 was mixed in distilled water with 4% DMSO, 30% PEG300, and 5% Tween80 and administered orally at a concentration of 10 mg/kg daily for 21 days. LX-5401 was mixed in 5% phosphate-buffered saline (PBS) and administered intraperitoneally at a concentration of 50 mg/kg daily for 21 days or intraocularly at a concentration of 10 μM. LX-5411 was mixed in 5% phosphate-buffered saline (PBS) and administered orally at a concentration of 10 mg/kg daily for 21 days or intraocularly at a concentration of 3 μM. LX-5701 was mixed in PBS and administered orally at a concentration of 360 mg/kg daily for 21 days. LX-6807 was mixed in saline and administered intraperitoneally at a concentration of 1 mg/kg daily for 21 days. LX-2101 was mixed in 5% DMSO in PBS and administered intraperitoneally at a concentration of 10 mg/kg daily for 21 days. The control group received only the solution in the same volume. After the end of drug administration, the mice were sacrificed, and RNA was extracted from one eye using the same method as in Example 7-2 of WO2023-085787 A1, and qRT-PCR was performed using the synthesized cDNA. In addition, frozen sections of the other eye tissue were stained with H/E (Hematoxylin/Eosin), and the thickness of the outer nuclear layer (ORL) was observed under an optical microscope. The table below shows the mRNA amount of p16, a senescence-related marker, and the thickness of the ORL under conditions with or without the test compound treatment. The mRNA amount after aging induction was calculated as 100% and compared with the mRNA amount of p16 after treatment with the test compound.

TABLE 53

| | p16 | Outer nuclear |
|---|---|---|
| | mRNA (%) | layer thickness (μm) |
| — | 100 (±6.7) | 27.4 (±1.3) |
| LX-403 | 34 (±2.2) | 40.1 (±1.9) |
| LX-1801 | 42 (±4.4) | 32.7 (±2.6) |
| LX-2001 | 61 (±4.1) | 34.1 (±5.1) |
| LX-2002 | 60 (±3.8) | 30.1 (±7.8) |
| LX-2003 | 52 (±6.3) | 31.2 (±8.7) |
| LX-2005 | 71 (±4.9) | 30.9 (±3.8) |
| LX-2007 | 75 (±5.9) | 29.4 (±7.5) |
| LX-2101 | 80 (±2.7) | 29.4 (±7.5) |
| LX-2707 | 76 (±5.4) | 31.8 (±1.7) |
| LX-3001 | 80 (±2.5) | 34.1 (±2.1) |
| LX-3002 | 78 (±3.1) | 30.8 (±3.6) |
| LX-5401 | 54 (±4.4) | 38.2 (±3.4) |
| LX-5411 | 43 (±2.5) | 43.7 (±4.2) |
| LX-5701 | 67 (±7.3) | 52.7 (±3.2) |
| LX-6807 | 64 (±4.4) | 50.8 (±3.2) |
| LX-2101 | 67 (±7.0) | 39 (±4.1) |

As a result, as shown in the table above, when the test compound was administered to the retinal degenerative disease model mouse, the amount of p16, an aging-related marker, was reduced, and the thickness of the outer nuclear layer thinned by NaIO₃ was restored. From this, it was confirmed that the test compounds improved retinal degenerative diseases induced in the mouse, such as age-related macular degeneration, macular edema, and retinopathy, by removing senescent cells that increase with aging. Furthermore, similar improvement results were confirmed when LX-401 or LX-405, which are similar to LX-403, LX-5401, or LX-5411, was administered.

Furthermore, when each of the test compounds of Table 53 was administered together with LX-6806, LX-6807, LX-9118, LX-9423, LX-9744, or LX-3403, the CI (combination index) was 0.61 to 0.79, confirming that there was synergy in efficacy. Similar synergy results were confirmed when LX-8401 was administered instead of LX-3403, and LX-9422 was administered instead of LX-9423.

### Example 30. Improvement of Geriatric Hypertension

Ten-week-old male SHR/Izm rats (SLC), a model of essential hypertension, were acclimated to the experimental environment for one week. After randomization into experimental and control groups, LX-2707, LX-3001, LX-3002, LX-5701, or LX-6807 was administered in the same manner as in Example 25. The control group received only the solution in the same volume. After drug administration, the blood pressure of the experimental animals was measured, and RNA was extracted from the thoracic aorta in the same manner as in Example 7-2 of WO2023-085787 A1. The synthesized cDNA was used for qRT-PCR. Blood pressure measurements were performed three times using a blood pressure monitor (IITC) after the animals were allowed to rest for approximately 10 minutes in a measuring holder with a cuff attached to the caudal vein. The table below shows the mRNA levels of p16, an aging-related marker, and blood pressure changes in untreated and treated animals with the test compound. The mRNA levels after aging were calculated as 100% and compared with the mRNA levels of p16 after treatment with the test compound.

TABLE 54

| | p16 | Blood |
|---|---|---|
| | mRNA (%) | pressure (mmHg) |
| — | 100 (±7.5) | 195 (±13.3) |
| LX-2707 | 64 (±8.5) | 160.1 (±23.1) |
| LX-3001 | 71 (±3.6) | 172.9 (±14.6) |
| LX-3002 | 72 (±2.9) | 177.5 (±17.4) |
| LX-5701 | 73 (±5.2) | 159.8 (±15.5) |
| LX-6807 | 66 (±5.0) | 157.8 (±19.3) |

As a result, as shown in the table above, when the test compound was administered to hypertensive model mice, the amount of p16, a marker associated with aging, decreased, and blood pressure decreased. This confirms that the test compounds improve geriatric hypertension induced in mice by eliminating senescent cells that increase with aging.

Furthermore, when each of the test compounds of Table 54 was administered together with LX-6806, LX-6807, LX-9118, LX-9423, LX-9744, or LX-3403, the CI (combination index) was 0.71 to 0.88, confirming synergistic efficacy. Similar synergistic results were confirmed when LX-8401 was administered instead of LX-3403, and LX-9422 was administered instead of LX-9423.

### Example 31. Removal of Senescent Cells and Related Aging Inhibition Activity in Aged Mice (Improvement of Senescent Inflammation and Geriatric Depression, Frailty, Sarcopenia, Lipoatrophy, Kidney Disease (Kidney Damage, Chronic Kidney Disease, Etc.), Skin Aging and Skin Diseases (Wrinkles, Loss of Elasticity, Hyperpigmentation, Etc.), Osteoarthritis, Cardiac Aging (Reduced Cardiac Load Resistance, Etc.), Bone Disease (Osteoporosis, Etc.), Eye Disease (Cataract, Etc.), Lung Disease (Pulmonary Fibrosis, Etc.), Liver Disease (Liver Damage, Liver Fibrosis, Liver Cirrhosis, Etc.))

In the same manner as Example 7-1 of WO2023-085787 A1, LX-305 (5% DMSO in Corn Oil, 8 mg/kg, intraperitoneal administration), LX-403 (10% ethanol in PEG400, 25 mg/kg, oral administration), LX-801 (5% DMSO in PBS solution, 75 mg/kg, oral administration), LX-905 (5% DMSO in PBS solution, 100 mg/kg, oral administration), LX-1102 (5% DMSO in Corn Oil, 100 mg/kg, oral administration), LX-1103 (PBS, 100 mg/kg, intraperitoneal administration), LX-1302 (50% PEG300 in saline, 320 mg/kg, intraperitoneal administration), LX-1601 (50% PEG300 in saline, 50 mg/kg, oral administration), LX-1603 (5% DMSO in corn oil, 35 mg/kg, oral administration), LX-1801 (5% DMSO in PBS solution, 50 mg/kg, intraperitoneal administration), LX-1902 (5% DMSO in PBS solution, 60 mg/kg, oral administration), LX-1903 (5% DMSO in PBS solution, 10 mg/kg, oral administration), LX-2601 (4% DMSO+30% PEG300+5% Tween80 in saline, 3 mg/kg oral administration), LX-2602 (5% DMSO in corn oil, 75 mg/kg intraperitoneal administration), LX-2702 (5% DMSO in corn oil, 400 mg/kg oral administration), LX-2901 (4% DMSO+30% PEG300+5% Tween80 in water, 2 mg/kg, intraperitoneal administration), LX-3106 (5% DMSO in PBS solution, 50 mg/kg, oral administration), LX-3107 (water, 100 mg/kg, oral administration), LX-3200 (5% DMSO in PBS solution, 10 mg/kg, oral administration), LX-3201 (5% ethanol+10% tween80 in saline, 2.6 mg/kg, subcutaneous administration), LX-3203 (5% DMSO+40% PEG300+5% Tween80 in water, 100 mg/kg oral administration), LX-3303 (10% ethanol in corn oil, LX-5201 (40% PEG400+4% DMSO in PBS solution, 50 mg/kg, intraperitoneal administration), LX-5401 (5% DMSO in PBS solution, 50 mg/kg, intraperitoneal administration), LX-5411 (5% DMSO in PBS solution, 10 mg/kg, intraperitoneal administration), LX-5424 (5% DMSO+45% PEG300+5% Tween80 in saline, 400 mg/kg intraperitoneal administration), LX-5702 (5% DMSO in PBS solution, 360 mg/kg, oral administration), LX-5706 (5% DMSO in PBS solution, 100 mg/kg, intraperitoneal administration), LX-5707 (5% DMSO in PBS solution, 100 mg/kg, intraperitoneal administration), LX-5711 (5% DMSO in PBS solution, 360 mg/kg, oral administration), LX-5721 (5% DMSO in PBS solution, 360 mg/kg, oral administration), LX-5722 (5% DMSO in PBS solution, 360 mg/kg, oral administration), LX-5731 (5% DMSO in PBS solution, 360 mg/kg, oral administration), LX-8401 (PBS, 10 mg/kg, intraperitoneal administration), LX-6501 (4% DMSO+30% PEG300+5% Tween80 in water, 80 mg/kg intraperitoneal administration), LX-6537 (PBS, 40 mg/kg intraperitoneal administration), LX-6800 (5% DMSO in corn oil, 30 mg/kg oral administration), LX-7103 (5% DMSO+45% PEG300+5% Tween80 in saline, 50 mg/kg intraperitoneal administration), LX-8402 (PBS, 10 mg/kg, intraperitoneal administration), LX-8726 (4% DMSO+30% PEG300+5% Tween80 in water, 50 mg/kg oral administration), LX-8801 (PBS, 400 mg/kg oral administration), LX-8805 (4% DMSO+30% PEG300+5% Tween80 in water, 60 mg/kg oral administration), LX-8812 (4% DMSO+30% PEG300+5% Tween80, 200 mg/kg oral administration), LX-9118 (50% PEG300 in saline, 200 mg/kg oral administration), LX-9421 (5% DMSO+45% PEG300+5% Tween80 in saline, 30 mg/kg oral administration), or LX-9744 (5% DMSO+45% PEG300+5% Tween80 in Saline, 60 mg/kg oral administration) was administered to naturally aged mice for 21 days under designated conditions. The control group was administered only the solution in the same volume. After the end of drug administration, the method described in each example below was performed. Liver, kidney, skin, knee joint, heart, and femur tissues were collected from mice in which natural aging was induced in the same manner as in Examples 7-2 and 7-6 of WO2023-085787 A1. Lung tissues were collected from mice in which pulmonary fibrosis was induced using the same method as in Example 24-1. Liver tissues were collected from mice in which hepatic fibrosis was induced using the same method as in Example 26, and total RNA was extracted. qRT-PCR was performed and analyzed using the synthesized cDNA. The table below shows the mRNA amounts of p16, a senescence-related marker, and MMP3 and IL6, senescence-related secreted factors (SASPs), in liver tissues of mice in which natural aging was induced and were not treated or treated with the test compound. The mRNA amounts of p16, MMP3, and IL6 after natural aging was induced were calculated as 100% and compared with the mRNA amounts of p16, MMP3, and IL6 after treatment with the test compound. In addition, an open field test for the analysis of aging-related frailty, agtr1a mRNA analysis for kidney damage and disease evaluation, col1a1 and SDF1 mRNA analysis for skin aging evaluation such as wrinkles/loss of elasticity/hyperpigmentation, col2a1 mRNA analysis for evaluating joint aging such as osteoarthritis, sur2a mRNA analysis for evaluating cardiac aging such as decreased cardiac load resistance, ctsk mRNA analysis for evaluating bone diseases due to aging such as osteoporosis, muscle tissue weight for analyzing sarcopenia, fat tissue weight for analyzing lipoatrophy, and cataract incidence analysis were performed in mice. For cataract incidence analysis, the drug was administered twice a week under the conditions specified above, and biweekly examinations were conducted until death. The table below quantitatively shows the results of each evaluation under conditions in which mice that had undergone natural aging were not treated and were treated with the test compound.

TABLE 55

| | | | | | | | Open Field | | | | | | |
|---|---|---|---|---|---|---|---|---|---|---|---|---|---|
| | Liver | | Liver | | Liver | | Test | | | | | | Cataract |
| | p16 | | MMP3 | | IL6 | | Time in | | Muscle | | Fat Mass | | incidence |
| | mRNA (%) | | mRNA (%) | | mRNA (%) | | Center(%) | | Mass (g) | | (g) | | (%) |
| — | 100 | (±4.8) | 100 | (±6.9) | 100 | (±8.8) | 100 | (±13.1) | 0.64 | (±0.1) | 0.82 | (±0.1) | 83 |
| LX-305 | 69 | (±2.2) | 78 | (±5.1) | 61 | (±3.8) | 157 | (±11.8) | 0.66 | (±0.0) | 0.91 | (±0.2) | 75 |
| LX-403 | 34 | (±2.4) | 45 | (±2.8) | 53 | (±4.8) | 237 | (±16.7) | 0.76 | (±0.1) | 0.98 | (±0.1) | 64 |
| LX-801 | 53 | (±8.1) | 35 | (±15.4) | 64 | (±3.6) | 222 | (±34.3) | 0.77 | (±0.1) | 1.09 | (±0.2) | 57 |
| LX-905 | 67 | (±4.4) | 76 | (±6.5) | 38 | (±28.6) | 241 | (±45.7) | 0.69 | (±0.1) | 1.08 | (±0.2) | 65 |
| LX-1102 | 58 | (±4.1) | 71 | (±2.7) | 74 | (±2.4) | 148 | (±8.1) | 0.65 | (±0.1) | 0.95 | (±0.1) | 77 |
| LX-1103 | 47 | (±2.7) | 68 | (±3.4) | 70 | (±3.0) | 183 | (±10.5) | 0.68 | (±0.1) | 0.89 | (±0.1) | 54 |
| LX-1302 | 55 | (±3.2) | 47 | (±4.7) | 48 | (±2.9) | 201 | (±14.3) | 0.70 | (±0.1) | 0.92 | (±0.2) | 73 |
| LX-1601 | 62 | (±2.1) | 39 | (±2.0) | 43 | (±2.8) | 228 | (±13.2) | 0.74 | (±0.0) | 1.20 | (±0.1) | 62 |
| LX-1603 | 74 | (±2.7) | 73 | (±2.2) | 61 | (±3.3) | 171 | (±10.0) | 0.69 | (±0.0) | 0.92 | (±0.1) | 78 |
| LX-1801 | 42 | (±11.5) | 49 | (±17.8) | 53 | (±13.3) | 287 | (±37.5) | 0.78 | (±0.1) | 1.16 | (±0.1) | 35 |
| LX-1902 | 72 | (±1.9) | 81 | (±7.6) | 79 | (±5.7) | 157 | (±29.9) | 0.68 | (±0.0) | 0.95 | (±0.1) | 76 |
| LX-1903 | 69 | (±7.0) | 75 | (±6.8) | 58 | (±12.7) | 161 | (±20.8) | 0.69 | (±0.1) | 0.97 | (±0.1) | 79 |
| LX-2601 | 73 | (±2.8) | 77 | (±3.0) | 75 | (±2.6) | 166 | (±9.4) | 0.71 | (±0.1) | 0.88 | (±0.1) | 80 |
| LX-2602 | 71 | (±4.1) | 64 | (±3.8) | 80 | (±2.1) | 158 | (±8.8) | 0.69 | (±0.1) | 0.84 | (±0.1) | 81 |
| LX-2702 | 80 | (±4.0) | 73 | (±1.7) | 54 | (±3.7) | 211 | (±11.1) | 0.72 | (±0.1) | 1.13 | (±0.1) | 66 |
| LX-2901 | 64 | (±2.8) | 72 | (±3.2) | 55 | (±4.0) | 167 | (±10.6) | 0.69 | (±0.1) | 0.90 | (±0.1) | 59 |
| LX-3106 | 76 | (±4.3) | 85 | (±6.3) | 71 | (±6.1) | 193 | (±34.8) | 0.72 | (±0.1) | 0.88 | (±0.1) | 71 |
| LX-3107 | 54 | (±5.4) | 68 | (±6.7) | 49 | (±14.8) | 167 | (±24.0) | 0.67 | (±0.1) | 0.99 | (±0.1) | 65 |
| LX-3200 | 77 | (±3.1) | 71 | (±9.6) | 67 | (±9.9) | 188 | (±12.6) | 0.69 | (±0.0) | 1.03 | (±0.1) | 72 |
| LX-3201 | 80 | (±3.7) | 64 | (±3.0) | 71 | (±3.4) | 171 | (±23.7) | 0.71 | (±0.1) | 1.06 | (±0.1) | 76 |
| LX-3203 | 66 | (±2.9) | 62 | (±2.9) | 67 | (±2.5) | 183 | (±12.0) | 0.68 | (±0.1) | 0.85 | (±0.1) | 75 |
| LX-3303 | 63 | (±5.1) | 73 | (±7.7) | 83 | (±4.2) | 186 | (±20.5) | 0.72 | (±0.1) | 1.08 | (±0.2) | 61 |
| LX-5201 | 58 | (±11.7) | 67 | (±8.1) | 43 | (±18.2) | 168 | (±18.3) | 0.77 | (±0.1) | 0.97 | (±0.2) | 49 |
| LX-5401 | 73 | (±5.3) | 81 | (±3.3) | 55 | (±10.8) | 179 | (±23.7) | 0.69 | (±0.1) | 0.89 | (±0.2) | 77 |
| LX-5411 | 65 | (±5.8) | 58 | (±10.5) | 63 | (±8.1) | 214 | (±28.1) | 0.74 | (±0.1) | 1.21 | (±0.1) | 52 |
| LX-5424 | 72 | (±3.3) | 59 | (±2.5) | 45 | (±2.8) | 191 | (±12.6) | 0.67 | (±0.1) | 0.84 | (±0.1) | 76 |
| LX-5702 | 47 | (±5.3) | 55 | (±4.8) | 49 | (±8.2) | 211 | (±23.8) | 0.70 | (±0.1) | 0.85 | (±0.2) | 66 |
| LX-5706 | 64 | (±7.2) | 60 | (±6.7) | 71 | (±5.2) | 171 | (±20.4) | 0.69 | (±0.1) | 0.85 | (±0.1) | 74 |
| LX-5707 | 59 | (±8.8) | 63 | (±7.2) | 61 | (±22.1) | 199 | (±27.8) | 0.70 | (±0.1) | 0.90 | (±0.2) | 70 |
| LX-5711 | 65 | (±6.1) | 61 | (±12.1) | 48 | (±10.7) | 191 | (±29.3) | 0.68 | (±0.0) | 0.93 | (±0.1) | 72 |
| LX-5721 | 41 | (±13.7) | 72 | (±7.3) | 54 | (±21.9) | 218 | (±19.4) | 0.68 | (±0.0) | 0.97 | (±0.1) | 71 |
| LX-5722 | 46 | (±7.6) | 63 | (±6.9) | 73 | (±8.1) | 222 | (±25.1) | 0.70 | (±0.0) | 1.03 | (±0.1) | 69 |
| LX-5731 | 58 | (±10.4) | 77 | (±2.6) | 42 | (±18.7) | 237 | (±33.3) | 0.71 | (±0.1) | 1.01 | (±0.1) | 72 |
| LX-6501 | 38 | (±1.6) | 56 | (±2.9) | 46 | (±2.6) | 214 | (±29.1) | 0.71 | (±0.1) | 0.89 | (±0.1) | 48 |
| LX-6537 | 74 | (±4.3) | 67 | (±5.6) | 48 | (±1.8) | 177 | (±9.6) | 0.68 | (±0.1) | 0.85 | (±0.1) | 72 |
| LX-6800 | 73 | (±4.6) | 77 | (±2.1) | 56 | (±4.1) | 154 | (±15.3) | 0.70 | (±0.1) | 0.91 | (±0.1) | 75 |
| LX-7103 | 65 | (±2.5) | 79 | (±2.5) | 67 | (±1.6) | 218 | (±13.2) | 0.67 | (±0.1) | 0.96 | (±0.1) | 69 |
| LX-8401 | 76 | (±3.8) | 69 | (±7.8) | 77 | (±7.2) | 173 | (±20.4) | 0.75 | (±0.1) | 0.90 | (±0.2) | 60 |
| LX-8402 | 77 | (±5.3) | 71 | (±8.8) | 69 | (±4.1) | 195 | (±18.9) | 0.77 | (±0.1) | 0.91 | (±0.1) | 60 |
| LX-8726 | 61 | (±5.2) | 64 | (±5.2) | 25 | (±3.5) | 276 | (±14.6) | 0.81 | (±0.1) | 1.20 | (±0.2) | 43 |
| LX-8801 | 54 | (±2.6) | 63 | (±3.9) | 33 | (±1.3) | 205 | (±11.9) | 0.69 | (±0.1) | 0.94 | (±0.1) | 59 |
| LX-8805 | 47 | (±4.8) | 67 | (±4.7) | 25 | (±1.2) | 217 | (±11.7) | 0.72 | (±0.1) | 0.87 | (±0.1) | 68 |
| LX-8812 | 59 | (±3.6) | 42 | (±2.8) | 53 | (±2.8) | 188 | (±16.2) | 0.70 | (±0.1) | 0.96 | (±0.1) | 54 |
| LX-9118 | 74 | (±4.5) | 83 | (±3.6) | 62 | (±2.7) | 199 | (±17.3) | 0.73 | (±0.0) | 0.88 | (±0.1) | 62 |
| LX-9421 | 57 | (±3.7) | 32 | (±4.7) | 41 | (±5.3) | 222 | (±14.9) | 0.78 | (±0.1) | 0.99 | (±0.1) | 54 |
| LX-9744 | 75 | (±3.5) | 49 | (±2.9) | 58 | (±2.9) | 171 | (±16.4) | 0.69 | (±0.1) | 0.84 | (±0.0) | 78 |

| | | | Skin | | | | Joint | | Heart | | Femur | |
|---|---|---|---|---|---|---|---|---|---|---|---|---|
| | Kidney | | Col1a1 | | SDF1 | | Col2a1 | | Sur2a | | Ctsk | |
| | Agtr1a | | mRNA | | mRNA | | mRNA | | mRNA | | mRNA | |
| | mRNA (%) | | (Fold) | | (fold) | | (fold) | | (fold) | | (%) | |
| — | 100 | (±7.7) | 1.0 | (±0.1) | 1.0 | (±0.0) | 1.0 | (±0.0) | 1.0 | (±0.1) | 100 | (±8.3) |
| LX-305 | 73 | (±2.2) | 1.7 | (±0.4) | 1.9 | (±0.1) | 2.6 | (±0.2) | 1.8 | (±0.1) | 73 | (±5.8) |
| LX-403 | 64 | (±3.0) | 2.8 | (±0.3) | 2.7 | (±0.3) | 3.4 | (±0.2) | 2.9 | (±0.1) | 22 | (±1.1) |
| LX-801 | 55 | (±13.9) | 2.1 | (±0.1) | 2.1 | (±0.2) | 2.7 | (±0.4) | 2.8 | (±0.3) | 70 | (±6.8) |
| LX-905 | 52 | (±12.5) | 2.0 | (±0.1) | 1.5 | (±0.1) | 2.3 | (±0.0) | 2.4 | (±0.3) | 62 | (±9.5) |
| LX-1102 | 80 | (±3.4) | 2.5 | (±0.3) | 2.3 | (±0.2) | 1.7 | (±0.1) | 1.8 | (±0.1) | 62 | (±1.3) |
| LX-1103 | 76 | (±3.7) | 2.3 | (±0.1) | 1.7 | (±0.3) | 2.8 | (±0.2) | 2.4 | (±0.2) | 78 | (±4.9) |
| LX-1302 | 62 | (±4.2) | 2.4 | (±0.2) | 1.8 | (±0.4) | 2.5 | (±0.3) | 2.3 | (±0.2) | 75 | (±1.4) |
| LX-1601 | 66 | (±2.9) | 1.8 | (±0.1) | 2.1 | (±0.1) | 2.0 | (±0.2) | 2.6 | (±0.1) | 40 | (±3.3) |
| LX-1603 | 77 | (±5.9) | 2.6 | (±0.2) | 1.7 | (±0.2) | 2.3 | (±0.2) | 2.0 | (±0.2) | 64 | (±4.1) |
| LX-1801 | 48 | (±23.9) | 1.9 | (±0.1) | 2.2 | (±0.3) | 2.6 | (±0.3) | 2.7 | (±0.3) | 69 | (±5.8) |
| LX-1902 | 71 | (±3.2) | 1.2 | (±0.1) | 1.6 | (±0.1) | 1.5 | (±0.3) | 1.5 | (±0.3) | 81 | (±2.6) |
| LX-1903 | 78 | (±3.4) | 1.2 | (±0.1) | 1.4 | (±0.1) | 1.7 | (±0.5) | 1.4 | (±0.2) | 77 | (±5.3) |
| LX-2601 | 73 | (±4.6) | 2.3 | (±0.2) | 1.9 | (±0.2) | 2.3 | (±0.2) | 2.7 | (±0.3) | 60 | (±2.7) |
| LX-2602 | 61 | (±2.3) | 2.5 | (±0.2) | 2.0 | (±0.3) | 2.2 | (±0.2) | 1.7 | (±0.3) | 59 | (±3.8) |
| LX-2702 | 57 | (±3.4) | 1.8 | (±0.2) | 2.7 | (±0.2) | 2.8 | (±0.3) | 2.3 | (±0.2) | 50 | (±3.5) |
| LX-2901 | 67 | (±2.0) | 1.7 | (±0.2) | 1.8 | (±0.2) | 4.0 | (±0.4) | 1.7 | (±0.1) | 51 | (±3.9) |
| LX-3106 | 83 | (±1.9) | 1.3 | (±0.0) | 1.3 | (±0.2) | 2.0 | (±0.4) | 1.6 | (±0.1) | 76 | (±6.4) |
| LX-3107 | 75 | (±12.0) | 1.2 | (±0.2) | 1.4 | (±0.1) | 1.1 | (±0.1) | 1.7 | (±0.3) | 81 | (±5.2) |
| LX-3200 | 62 | (±5.3) | 1.4 | (±0.1) | 1.6 | (±0.2) | 1.3 | (±0.2) | 1.4 | (±0.1) | 68 | (±9.8) |
| LX-3201 | 78 | (±8.9) | 1.6 | (±0.2) | 2.0 | (±0.2) | 1.7 | (±0.1) | 2.1 | (±0.0) | 80 | (±6.7) |
| LX-3203 | 68 | (±4.5) | 1.8 | (±0.1) | 2.2 | (±0.2) | 1.6 | (±0.2) | 1.8 | (±0.2) | 55 | (±4.9) |
| LX-3303 | 69 | (±13.8) | 1.2 | (±0.3) | 1.7 | (±0.2) | 2.1 | (±0.2) | 1.9 | (±0.2) | 76 | (±7.2) |
| LX-5201 | 58 | (±15.7) | 2.1 | (±0.1) | 2.3 | (±0.1) | 1.9 | (±0.1) | 1.5 | (±0.1) | 53 | (±6.7) |
| LX-5401 | 73 | (±7.7) | 1.8 | (±0.2) | 2.0 | (±0.1) | 1.6 | (±0.1) | 1.7 | (±0.3) | 42 | (±18.5) |
| LX-5411 | 68 | (±11.4) | 1.6 | (±0.1) | 1.9 | (±0.2) | 2.0 | (±0.2) | 2.5 | (±0.3) | 71 | (±6.4) |
| LX-5424 | 68 | (±5.4) | 1.6 | (±0.2) | 1.7 | (±0.1) | 1.5 | (±0.2) | 2.3 | (±0.2) | 70 | (±4.6) |
| LX-5702 | 49 | (±25.0) | 1.8 | (±0.2) | 2.0 | (±0.2) | 2.4 | (±0.3) | 2.5 | (±0.3) | 61 | (±12.4) |
| LX-5706 | 70 | (±9.1) | 1.6 | (±0.2) | 1.4 | (±0.2) | 1.9 | (±0.4) | 2 | (±0.3) | 68 | (±11.2) |
| LX-5707 | 65 | (±10.8) | 1.7 | (±0.3) | 1.6 | (±0.1) | 2.0 | (±0.3) | 2.3 | (±0.1) | 64 | (±11.8) |
| LX-5711 | 63 | (±11.0) | 1.5 | (±0.1) | 1.8 | (±0.1) | 2.3 | (±0.1) | 2.3 | (±0.1) | 62 | (±10.2) |
| LX-5721 | 59 | (±15.9) | 1.7 | (±0.1) | 1.7 | (±0.1) | 2.0 | (±0.2) | 2.7 | (±0.3) | 63 | (±2.8) |
| LX-5722 | 52 | (±17.5) | 1.8 | (±0.2) | 1.6 | (±0.2) | 2.1 | (±0.1) | 2.4 | (±0.1) | 59 | (±9.1) |
| LX-5731 | 49 | (±21.4) | 2.3 | (±0.3) | 2.0 | (±0.2) | 2.7 | (±0.4) | 2.5 | (±0.1) | 44 | (±15.3) |
| LX-6501 | 49 | (±1.9) | 1.9 | (±0.2) | 2.2 | (±0.3) | 2.7 | (±0.2) | 1.8 | (±0.1) | 64 | (±2.2) |
| LX-6537 | 73 | (±5.9) | 1.9 | (±0.1) | 1.4 | (±0.1) | 2.2 | (±0.2) | 1.6 | (±0.1) | 67 | (±3.8) |
| LX-6800 | 78 | (±4.3) | 2.1 | (±0.1) | 2.0 | (±0.2) | 2.4 | (±0.3) | 2.3 | (±0.2) | 58 | (±2.6) |
| LX-7103 | 67 | (±4.5) | 2.6 | (±0.2) | 2.6 | (±0.2) | 2.3 | (±0.4) | 2.1 | (±0.1) | 65 | (±3.7) |
| LX-8401 | 66 | (±8.5) | 1.9 | (±0.2) | 1.3 | (±0.2) | 1.8 | (±0.2) | 2.1 | (±0.2) | 80 | (±3.3) |
| LX-8402 | 70 | (±10.1) | 2.1 | (±0.2) | 1.4 | (±0.2) | 1.9 | (±0.2) | 2.4 | (±0.3) | 79 | (±2.1) |
| LX-8726 | 48 | (±2.7) | 2.8 | (±0.1) | 2.7 | (±0.3) | 2.4 | (±0.3) | 2.9 | (±0.4) | 25 | (±3.7) |
| LX-8801 | 72 | (±4.6) | 2.1 | (±0.4) | 2.6 | (±0.1) | 1.7 | (±0.1) | 2.9 | (±0.3) | 35 | (±2.2) |
| LX-8805 | 55 | (±2.4) | 1.7 | (±0.1) | 1.9 | (±0.2) | 2.6 | (±0.2) | 2.6 | (±0.2) | 58 | (±4.7) |
| LX-8812 | 51 | (±2.7) | 3.4 | (±0.3) | 2.9 | (±0.1) | 2.6 | (±0.2) | 2.0 | (±0.2) | 62 | (±3.2) |
| LX-9118 | 71 | (±5.6) | 1.9 | (±0.2) | 1.8 | (±0.1) | 1.8 | (±0.1) | 1.7 | (±0.1) | 75 | (±2.6) |
| LX-9421 | 46 | (±4.3) | 2.3 | (±0.2) | 2.6 | (±0.2) | 2.8 | (±0.3) | 2.1 | (±0.2) | 44 | (±1.2) |
| LX-9744 | 65 | (±4.1) | 2.3 | (±0.3) | 1.7 | (±0.2) | 2.4 | (±0.3) | 2.3 | (±0.2) | 57 | (±1.7) |

As a result, as shown in the evaluation results of Examples 7-2, 7-6, and 7-5 of WO2023-085787 A1, when mice aged naturally, the amounts of p16, MMP3, and IL6 in liver tissue increased. However, when treated with the test compound, the mRNA amounts of p16, MMP3, and IL6 decreased. From this, it was confirmed that the test compounds can alleviate senescent inflammation and geriatric depression by eliminating senescent cells that increase in mice due to natural aging, thereby effectively reducing the amounts of p16, a senescence-related marker, and MMP3 and IL6, senescence-associated secretory factors (SASPs).

Furthermore, as shown in the evaluation results of Example 7-7 of WO2023-085787 A1, when mice aged naturally, activity decreased and the amount of muscle and adipose tissue decreased. However, when treated with the test compound, activity and the amount of muscle and adipose tissue increased. Furthermore, mRNA levels of Agtr1a, a marker of renal damage, and ctsk, a marker of osteoclasts in the femur, decreased. mRNA levels of type I collagen in skin, type II collagen, a marker of cartilage formation in joints, SDF1, which inhibits pigmentation, and sur2a, a marker of cardiac stress resistance, increased. Furthermore, the incidence of cataracts decreased.

The table below quantitatively presents the evaluation results for each item in mice with bleomycin-induced pulmonary fibrosis, untreated and treated with the test compound.

TABLE 56

| | Lung p16 | Col1a1 |
|---|---|---|
| | mRNA (%) | mRNA (%) |
| — | 100 (±6.7) | 100 (±7.7) |
| LX-305 | 62 (±3.5) | 73 (±4.1) |
| LX-403 | 32 (±1.4) | 44 (±3.1) |
| LX-801 | 48 (±4.8) | 43 (±10.6) |
| LX-905 | 67 (±5.5) | 59 (±13.3) |
| LX-1102 | 59 (±3.9) | 68 (±4.1) |
| LX-1103 | 73 (±3.2) | 76 (±5.3) |
| LX-1302 | 41 (±1.5) | 59 (±2.6) |
| LX-1601 | 52 (±3.7) | 63 (±4.6) |
| LX-1603 | 78 (±6.1) | 48 (±2.5) |
| LX-1801 | 53 (±13.7) | 38 (±20.8) |
| LX-1902 | 72 (±8.8) | 64 (±8.2) |
| LX-1903 | 73 (±6.6) | 70 (±9.3) |
| LX-2601 | 81 (±5.2) | 67 (±3.0) |
| LX-2602 | 68 (±3.5) | 58 (±2.9) |
| LX-2702 | 53 (±2.8) | 43 (±5.9) |
| LX-2901 | 72 (±5.7) | 61 (±4.3) |
| LX-3106 | 67 (±4.3) | 77 (±3.7) |
| LX-3107 | 81 (±3.9) | 68 (±5.1) |
| LX-3200 | 76 (±5.7) | 63 (±6.9) |
| LX-3201 | 77 (±5.9) | 73 (±3.8) |
| LX-3203 | 69 (±5.0) | 64 (±4.5) |
| LX-3303 | 68 (±6.9) | 78 (±5.1) |
| LX-5201 | 75 (±4.2) | 74 (±6.3) |
| LX-5401 | 67 (±8.1) | 59 (±12.8) |
| LX-5411 | 61 (±13.2) | 44 (±11.1) |
| LX-5424 | 70 (±3.5) | 73 (±2.7) |
| LX-5702 | 61 (±6.2) | 68 (±7.5) |
| LX-5706 | 76 (±6.1) | 79 (±5.2) |
| LX-5707 | 72 (±8.9) | 69 (±7.1) |
| LX-5711 | 70 (±3.8) | 71 (±4.0) |
| LX-5721 | 65 (±5.6) | 78 (±4.7) |
| LX-5722 | 73 (±5.5) | 78 (±7.2) |
| LX-5731 | 69 (±3.7) | 62 (±13.8) |
| LX-6501 | 68 (±4.4) | 75 (±5.3) |
| LX-6537 | 64 (±3.2) | 67 (±3.9) |
| LX-6800 | 78 (±3.8) | 62 (±3.6) |
| LX-7103 | 65 (±2.1) | 66 (±4.5) |
| LX-8401 | 51 (±9.8) | 37 (±18.2) |
| LX-8402 | 49 (±15.7) | 50 (±12.2) |
| LX-8726 | 42 (±2.9) | 58 (±5.3) |
| LX-8801 | 44 (±3.0) | 67 (±4.5) |
| LX-8805 | 40 (±2.1) | 70 (±4.6) |
| LX-8812 | 67 (±5.6) | 77 (±6.2) |
| LX-9118 | 76 (±4.6) | 58 (±3.5) |
| LX-9421 | 35 (±1.6) | 40 (±2.0) |
| LX-9744 | 73 (±3.1) | 64 (±3.3) |

As a result, it was confirmed that the test compounds improved lung diseases such as pulmonary fibrosis, idiopathic pulmonary fibrosis, emphysema, and pulmonary failure by effectively reducing the mRNA levels of p16, a senescent marker, and Cola1, a secretory factor (SASP), by eliminating senescent cells that increased in mice due to the induction of pulmonary fibrosis.

The table below quantitatively shows the evaluation results for each item in mice that had liver fibrosis induced by carbon tetrachloride (CCl₄) under conditions in which the test compound was not administered or was administered.

TABLE 57

| | Liver p16 | Col1a1 |
|---|---|---|
| | mRNA (%) | mRNA (%) |
| — | 100 (±8.7) | 100 (±5.6) |
| LX-305 | 64 (±2.3) | 70 (±3.2) |
| LX-403 | 51 (±3.8) | 48 (±5.8) |
| LX-801 | 57 (±9.3) | 63 (±11.5) |
| LX-905 | 76 (±6.0) | 71 (±7.5) |
| LX-1102 | 76 (±2.8) | 64 (±4.7) |
| LX-1103 | 77 (±3.5) | 60 (±6.9) |
| LX-1302 | 52 (±4.4) | 48 (±3.2) |
| LX-1601 | 48 (±4.0) | 50 (±2.0) |
| LX-1603 | 67 (±3.5) | 54 (±2.9) |
| LX-1801 | 55 (±9.8) | 58 (±7.9) |
| LX-1902 | 77 (±9.6) | 68 (±5.7) |
| LX-1903 | 67 (±9.2) | 62 (±6.9) |
| LX-2601 | 75 (±3.2) | 63 (±2.6) |
| LX-2602 | 77 (±3.9) | 82 (±3.7) |
| LX-2702 | 46 (±3.2) | 53 (±6.1) |
| LX-2901 | 59 (±1.3) | 73 (±3.6) |
| LX-3106 | 81 (±6.5) | 71 (±5.4) |
| LX-3107 | 80 (±3.6) | 69 (±8.0) |
| LX-3200 | 75 (±9.8) | 80 (±9.6) |
| LX-3201 | 71 (±9.4) | 82 (±8.2) |
| LX-3203 | 64 (±4.0) | 66 (±4.5) |
| LX-3303 | 62 (±4.9) | 65 (±6.2) |
| LX-5201 | 64 (±8.4) | 59 (±8.7) |
| LX-5401 | 61 (±13.2) | 72 (±5.9) |
| LX-5411 | 53 (±9.9) | 43 (±16.1) |
| LX-5424 | 79 (±5.6) | 51 (±4.7) |
| LX-5702 | 72 (±3.9) | 65 (±7.2) |
| LX-5706 | 69 (±6.9) | 70 (±4.8) |
| LX-5707 | 64 (±8.3) | 72 (±9.6) |
| LX-5711 | 63 (±4.3) | 74 (±3.0) |
| LX-5721 | 52 (±9.6) | 80 (±9.3) |
| LX-5722 | 69 (±6.2) | 76 (±6.4) |
| LX-5731 | 60 (±6.5) | 64 (±10.4) |
| LX-6501 | 34 (±3.0) | 75 (±2.5) |
| LX-6537 | 77 (±5.7) | 81 (±4.1) |
| LX-6800 | 80 (±4.9) | 76 (±2.7) |
| LX-7103 | 70 (±2.2) | 68 (±2.9) |
| LX-8401 | 51 (±14.6) | 41 (±11.8) |
| LX-8402 | 67 (±6.6) | 52 (±8.2) |
| LX-8726 | 36 (±4.3) | 48 (±4.3) |
| LX-8801 | 49 (±4.3) | 53 (±3.5) |
| LX-8805 | 60 (±3.9) | 36 (±3.4) |
| LX-8812 | 63 (±3.9) | 64 (±3.1) |
| LX-9118 | 69 (±2.4) | 64 (±2.0) |
| LX-9421 | 59 (±5.4) | 34 (±3.7) |
| LX-9744 | 71 (±3.1) | 48 (±3.5) |

As a result, it was confirmed that the test compounds improved liver diseases such as liver fibrosis, cirrhosis, hepatitis, primary biliary cirrhosis, primary sclerosing cholangitis, biliary atresia, and bile duct damage by effectively reducing the mRNA amount of p16, a senescent marker, and Cola1, a secretory factor (SASP), by eliminating senescent cells that increase in mice due to induction of liver fibrosis.

From this, it was confirmed that the test compounds improved senescent inflammation and geriatric depression, frailty, sarcopenia, lipoatrophy, kidney disease (kidney damage, chronic kidney disease, etc.), skin aging (wrinkles, loss of elasticity, hyperpigmentation), osteoarthritis, heart aging (reduced cardiac load resistance), senile bone disease (osteoporosis, etc.), senile eye disease (cataracts, etc.), senile lung disease (pulmonary fibrosis, etc.), and senile liver disease (liver damage, liver fibrosis, cirrhosis, etc.) in mice by eliminating senescent cells in each tissue that increase with aging. A similar improvement effect was confirmed in senescent cells killed by the test compound.

Furthermore, when each of the test compounds of Tables 55, 56, and 57 was administered together with LX-6806, LX-6807, LX-9118, LX-9423, LX-9744, or LX-3403, the CI (combination index) was 0.51 to 0.73, confirming that there was synergy in the anti-aging activity and improvement efficacy. Similar synergy results were confirmed when LX-8401 was administered instead of LX-3403, and LX-9422 was administered instead of LX-9423.

### Example 32. Removal of Aged Human Cells and Cancer Cells by Secretions from Cells Treated with the Compound of the Present Invention

In the same manner as in Example 13, supernatants containing secretions from death-induced cells were obtained by treating the cells with the test compound at high concentrations of 30 M and 90 μM, respectively. The degree of senescent cell death was measured. The cell viability results are shown in the table below.

TABLE 58

| | HMGN1 |
|---|---|
| LX-403 | 35.8 (±3.5) |
| LX-1801 | 37.5 (±3.1) |
| LX-2001 | 61.5 (±4.4) |
| LX-2002 | 58.6 (±4.5) |
| LX-2003 | 67.1 (±2.5) |
| LX-2005 | 63.5 (±1.8) |
| LX-2007 | 65.9 (±2.6) |
| LX-2707 | 54.9 (±3.5) |
| LX-3001 | 45.4 (±3.3) |
| LX-3002 | 51.3 (±4.9) |
| LX-5201 | 58.8 (±3.4) |
| LX-5211 | 56.2 (±3.1) |
| LX-5401 | 54.9 (±3.3) |
| LX-5411 | 42.5 (±4.4) |
| LX-5424 | 65.1 (±2.9) |
| LX-5433 | 46.6 (±6.3) |
| LX-5449 | 36.4 (±7.2) |
| LX-5701 | 42.1 (±3.9) |
| LX-5702 | 40.5 (±6.1) |
| LX-5706 | 53.0 (±8.8) |
| LX-5707 | 48.2 (±7.6) |
| LX-5711 | 51.8 (±6.1) |
| LX-5721 | 44.5 (±4.2) |
| LX-5722 | 50.9 (±3.2) |
| LX-5731 | 37.1 (±6.4) |
| LX-5904 | 61.9 (±3.7) |
| LX-5906 | 58.4 (±7.1) |
| LX-5907 | 57.1 (±6.6) |
| LX-6800 | 54.9 (±5.7) |
| LX-6804 | 56.3 (±4.7) |
| LX-6806 | 62.0 (±5.6) |
| LX-6807 | 48.9 (±7.5) |
| LX-6809 | 57.8 (±6.6) |
| LX-7103 | 60.5 (±3.5) |
| LX-7104 | 35.9 (±4.8) |
| LX-7111 | 43.2 (±4.3) |
| LX-7121 | 51.2 (±5.3) |
| LX-7122 | 41.5 (±5.8) |
| LX-7135 | 42.1 (±7.0) |
| LX-7261 | 67.8 (±8.2) |
| LX-7262 | 67.1 (±4.2) |
| LX-7263 | 68.6 (±4.8) |
| LX-7288 | 55.7 (±6.0) |

As a result, it was confirmed that the medium supernatant containing secretions from cells induced to death by treatment with the test compound could effectively remove senescent cells by inducing their death.

Furthermore, it was confirmed that the medium supernatant containing secretions from cells induced to death by treatment with the test compound could effectively remove senescent cancer cells by inducing their death, as in Example 14.

Furthermore, the medium supernatant obtained through the same process from medium treated with the test compound without culturing cells (no cells) failed to induce death in either proliferating or senescent cells. This confirms that the medium supernatant containing substances secreted from cells induced to death by treatment with the test compound could effectively remove senescent cells by inducing the death of various senescent cells, including cancer cells.

1.-10. (canceled)

11. A method of removing senescent cells, comprising:
contacting with senescent cells or administering or applying to a subject in need of improvement, treatment or prevention of diseases and disorders related to senescent cells a therapeutically effective amount of a compound of Table 1 or its pharmaceutically acceptable salt; secreted fluid from cells killed by treatment with such a compound or its pharmaceutically acceptable salt; or cells killed by treatment with such a compound or its pharmaceutically acceptable salt.

12. The method according to claim 11, wherein the contact with the senescent cells is performed in vitro or ex vivo.

13. The method according to claim 11, wherein the removal of senescent cells improves, treats or prevents diseases and disorders related to senescent cells.

14. The method according to claim 11, wherein the method further comprises contacting with senescent cells or administering or applying to a subject in need of improvement, treatment or prevention of diseases and disorders related to senescent cells at least one active agent selected from the group consisting of Roxadustat, Dexmedetomidine, 5-aminosalicylic acid, Pioglitazone, Rosiglitazone, Setanaxib, and an immune checkpoint inhibitor.

15. The method according to claim 14, wherein the immune checkpoint inhibitor is any one or more of a CTLA4 inhibitor, a PD-1 inhibitor, a PD-L1 inhibitor, and a PD-L2 inhibitor.

16. (canceled)

17. The method according to claim 11, wherein the compound of Table 1 or its pharmaceutically acceptable salt increases the secretion of HMGN1 (High Mobility Group Nucleosome Binding Domain 1) protein (HMG14) in senescent cells.

18. The method according to claim 13, wherein the diseases and disorders related to senescent cells is at least one selected from the group consisting of proliferative diseases and disorders, inflammatory or autoimmune diseases and disorders, neurological diseases and disorders, metabolic diseases and disorders, cardiovascular diseases and disorders, lung diseases and disorders, kidney diseases and disorders, liver diseases and disorders, eye diseases and disorders, skin diseases and disorders, regenerative maintenance-related diseases and disorders, transplantation-related diseases and disorders, fibrosis diseases and disorders, sclerosis diseases and disorders, and β-galactosidase-related diseases and disorders, geriatric diseases and disorders, side effects or aftereffects due to stress and aging-related diseases and disorders resulting therefrom, and rare aging-related diseases and disorders.

19. The method according to claim 18, wherein the proliferative disease or disorder is cancer; the inflammatory or autoimmune disease or disorder is arthritis, osteoarthritis, rheumatoid arthritis, juvenile idiopathic arthritis, spondyloarthritis, degenerative cartilage disease, or kyphosis; the neurological disease or disorder is dementia, Alzheimer's disease, memory loss, or cognitive impairment; the metabolic disease or disorder is obesity, diabetes and diabetic complications (e.g., diabetic ulcer, diabetic retinopathy, diabetic cardiomyopathy, diabetic nephropathy), or age-related bone disease (e.g., osteoporosis, impaired fracture healing, alveolar bone loss, periodontal disease); the cardiovascular disease or disorder is atherosclerosis, aortic aneurysm, coronary aneurysm, cerebral aneurysm, age-related vascular dysfunction, decreased cardiac load resistance, cardiac hypertrophy, myocardial infarction, heart failure, or hypertension; the lung disease or disorder is idiopathic pulmonary fibrosis, chronic pulmonary inflammation, pulmonary dysfunction, pulmonary fibrosis, chronic obstructive pulmonary disease, or emphysema; the kidney disease or disorder is nephropathy, renal failure, glomerulosclerosis, or chronic kidney disease; the liver disease or disorder is fatty liver, hepatopathy, hepatic fibrosis, cirrhosis, hepatitis, primary biliary cirrhosis, primary sclerosing cholangitis, biliary atresia, or bile duct damage; the eye disease or disorder is cataract, or retinal degenerative disease (e.g., age-related macular degeneration, macular edema, retinopathy); the skin disease or disorder is skin aging, epidermal atrophy, loss of elasticity, wrinkles, nevi, macular edema, pigmentation, melasma, blemishes, hair loss, or wound healing disorders; the fibrotic disease or disorder is retroperitoneal fibrosis, idiopathic pulmonary fibrosis, pulmonary fibrosis, or liver fibrosis; the sclerosing disease or disorder is idiopathic pulmonary fibrosis or scleroderma; other geriatric diseases and disorders are frailty, sarcopenia, muscular dystrophy, lipoatrophy, age-related inflammation, or geriatric depression; side effects and sequelae of stress and resulting age-related diseases and disorders are fatigue, weakness, lethargy, weight loss, hepatotoxicity, nephrotoxicity, thrombosis, alopecia, or bone marrow dysfunction (e.g., myelodysplastic syndrome, myelopathy, bone marrow transplant disorder, anemia); or rare diseases are lipodystrophy, idiopathic pulmonary fibrosis, dementia with Lewy bodies, or nonalcoholic steatohepatitis.

20. The method according to claim 13, wherein the diseases and disorders related to senescent cells is cancer, inflammation, fibrosis, sclerosis, frailty, cognitive impairment, sarcopenia, osteoporosis, lipoatrophy, liver damage, kidney damage, skin aging, or side effects of anticancer drugs or obesity.

21. The method according to claim 20, wherein the frailty is decreased activity, decreased endurance, decreased muscle strength, or decreased grip strength.

22. The method according to claim 20, wherein the skin aging is wrinkles, loss of elasticity, hair loss, deterioration of hair skin, freckles, blemishes, skin rash, skin spots, skin discoloration, pigmentation, warts, hives, worsening of pores, or wound/injury healing disorder.

23. The method according to claim 20, wherein the side effects of anticancer drugs or obesity is fatigue, weakness, weight loss, anxiety, depression, lethargy, or fatty liver.
